(19) United States
(12) Patent Application Publication
(10) Pub. No.: US 2026/0021196 A1
(43) Pub. Date: Jan. 22, 2026

(54) HOMOGENEOUS ANTIBODY-CONJUGATES WITH HIGH PAYLOAD LOADING

(71) Applicant: SYNAFFIX B.V., Oss (NL)

(72) Inventors: Elias POST, Oss (NL); Remon VAN GEEL, Oss (NL); Lianne LELIEVELDT, Oss (NL); Sander Sebastiaan VAN BERKEL, Oss (NL); Floris Louis VAN DELFT, Oss (NL)

(21) Appl. No.: 19/286,170

(22) Filed: Jul. 30, 2025

Related U.S. Application Data
(63) Continuation of application No. PCT/EP2024/052430, filed on Jan. 31, 2024.

(30) Foreign Application Priority Data
Jan. 31, 2023 (EP) 23154322.4

(51) Int. Cl. A61K 47/68; A61P 35/00

(52) U.S. Cl. CPC A61K 47/6851; A61K 47/6803; A61P 35/00

(57) ABSTRACT

The invention concerns homogenous antibody-conjugates with high payload loading (high DAR) obtained by site-specific conjugation to a single antibody N-glycan. The conjugates according to the invention are homogeneous, i.e. have a DAR at or close to the theoretical DAR with a narrow distribution, and do not require any genetic modification of the antibody. The invention further concerns a modular, non-genetic preparation method for such conjugates, involving three simple steps and starting from any antibody. These steps are (a) enzymatic remodeling of the glycan to give an antibody functionalized with two or four click probes per antibody, (b) strain-promoted cycloaddition with a multivalent, bifunctional reagent comprising one cyclic alkyne and at least two click probes that are not reactive towards the cyclic alkyne, and (c) inverse electron-demand Diels-Alder cycloaddition of the click probes with branched linker-drug constructs comprising one cyclic alkyne or strained alkene, connected to one or more payloads preferably connected through a cleavable linker. The resulting conjugates, with DAR6 or higher, are rapidly generated with high homogeneity and with surprising stability. In addition, HIC profiles of the resulting ADCs indicate small relative retention time and therefore show high potential in the targeting of tumour cells and/or the treatment of cancer.

### CROSS-REFERENCE TO RELATED APPLICATIONS

This application is a continuation of International Patent Application No. PCT/EP2024/052430, filed Jan. 31, 2024, which claims priority to European Patent Application No. 23154322.4, filed Jan. 31, 2023, the entire disclosures of which are incorporated by reference herein.

### REFERENCE TO SEQUENCE LISTING

The present application contains a Sequence Listing which has been filed electronically in XML format and is hereby incorporated by reference in its entirety. The XML copy created on Jul. 24, 2025, is named 069818-10040_Sequence_Listing and is 9,795 bytes in size.

### FIELD OF THE INVENTION

The present invention is in the field of medicine. More specifically, the present invention relates to antibody-conjugates with a high payload loading, that are homogeneous and do not require genetic antibody modification. Such antibody-conjugates can be applied for the more effective treatment of disease, in particular cancer.

### BACKGROUND

Antibody-drug conjugates (ADC), considered as one of the major classes of targeted therapy, are comprised of an antibody to which is attached a pharmaceutical agent. The antibodies (also known as binding agents or ligands) can be small protein formats (scFv's, Fab fragments, DARPins, Affibodies, etc.) but are generally monoclonal antibodies (mAbs) of IgG type which have been selected based on their high selectivity and affinity for a given antigen, their long circulating half-lives, and little to no immunogenicity. Thus, mAbs as ligands for a carefully selected biological receptor provide an ideal targeting platform for selective delivery of pharmaceutical drugs. For example, a monoclonal antibody known to bind selectively with a specific cancer-associated antigen can be used for delivery of a chemically conjugated cytotoxic agent to the tumour, via binding, internalization, intracellular processing and finally release of active catabolite. The cytotoxic agent may be small molecule toxin, a protein toxin or other formats, like oligonucleotides. As a result, the tumour cells can be selectively eradicated, while sparing normal cells which have not been targeted by the antibody. Similarly, chemical conjugation of an antibacterial drug (antibiotic) to an antibody can be applied for treatment of bacterial infections, while conjugates of anti-inflammatory drugs are under investigation for the treatment of autoimmune diseases and for example attachment of an oligonucleotide to an antibody is a potential promising approach for the treatment of neuromuscular diseases. Hence, the concept of targeted delivery of an active pharmaceutical drug to a specific cellular location of choice is a powerful approach for the treatment of a wide range of diseases, with many beneficial aspects versus systemic delivery of the same drug.

ADCs are prepared by conjugation of a linker-drug to a protein, a process known as bioconjugation. Many technologies are known for bioconjugation, as summarized in G. T. Hermanson, “Bioconjugate Techniques”, Elsevier, 3^{rd} Ed. 2013, incorporated by reference. Conceptually, the method the preparation of an ADC by bioconjugation entails the reaction of x number of reactive moieties F present on the antibody with a complementary reactive moiety Q present on the pharmaceutical drug (the payload), see FIG. 1.

Typically, a chemical linker is present between Q and the payload. This linker needs to possess a number of key attributes, including the requirement to be stable in plasma after drug administration for an extended period of time. A stable linker enables localization of the ADC to the projected site or cells in the body and prevents premature release of the payload in circulation, which would indiscriminately induce undesired biological response of all kinds, thereby lowering the therapeutic index of the ADC. Upon internalization, the ADC should be processed such that the payload is effectively released so it can exert its mode-of-action inside the cell. The linker can also contain a spacer element. There are two families of linkers, non-cleavable and cleavable. Non-cleavable linkers consist of a chain of atoms between the antibody and the payload, which is fully stable under physiological conditions, irrespective of which organ or biological compartment the antibody-drug conjugate resides in. As a consequence, liberation of the payload from an ADC with a non-cleavable linker relies on the complete (lysosomal) degradation of the antibody after internalization of the ADC into a cell. As a result of this degradation, the payload will be released, still carrying the linker, as well as a peptide fragment and/or the amino acid from the antibody the linker was originally attached to. Cleavable linkers utilize an inherent property of a cell or a cellular compartment for selective release of the payload from the ADC, which generally leaves no trace of linker after processing. For cleavable linkers, there are three commonly used mechanisms: (1) susceptibility to specific enzymes, (2) pH-sensitivity, and (3) sensitivity to redox state of a cell (or its microenvironment). The cleavable linker may also contain a self-immolative unit, for example based on a para-aminobenzyl alcohol group or para-hydroxybenzyl alcohol and derivatives and/or analogues thereof or a cyclization linker based on for example 1,2-diaminoethane carbamate derivatives. A linker may also contain an additional element, often referred to as spacer or stretcher unit, to connect the linker with a reactive group for attachment to the antibody via a reactive moiety F present on the antibody.

The reactive moiety F can be naturally present in the antibody, for example the reactive moiety can be the side chain of lysine or cysteine, which can be employed for acylation (lysine side chain) or alkylation (cysteine side chain).

Acylation of the ε-amino group in a lysine side-chain is typically achieved by subjecting the protein to a reagent based on an activated ester or activated carbonate derivative, for example SMCC is applied for the manufacturing of Kadcyla®. Based on the fact that a given antibody may contain 60-90 occurrences of lysine, of which the vast majority will display reactivity to the acylating agent, careful titration of the acylating agent is required which nevertheless results in a highly heterogeneous mixture of conjugation with only an average drug loading based on stochastic distribution of drugs attached to the antibody. For example, Kadcyla® has an average drug-to-antibody ratio (DAR) of approximately four but in fact consists of a mixture of components with DAR 0-12. Addition of a larger quantity of acylating agents will lead to a higher average DAR, for example DAR 6 or DAR8 can be achieved, based on a stochastic distribution containing even higher DAR species (i.e. >DAR 12).

Various reagents are known for alkylation of the thiol group in cysteine side-chain, see FIG. 2. Amongst the cysteine alkylation strategies, the vast majority is based on the use of maleimide reagents, as is for example applied in the manufacturing of Adcetris®, Polivy® and Padcev®. Besides standard maleimide reagents, a range of maleimide variants are also applied for more stable cysteine conjugation, as for example demonstrated by James Christie et al., J. Contr. Rel. 2015, 220, 660-670 and Lyon et al., Nat. Biotechnol. 2014, 32, 1059-1062, both incorporated by reference. Other approaches for cysteine alkylation involve for example nucleophilic substitution of haloacetamides (typically bromoacetamide or iodoacetamide), see for example Alley et al., Bioconj. Chem. 2008, 19, 759-765, incorporated by reference, or various approaches based on nucleophilic addition on unsaturated bonds, such as reaction with acrylate reagents, see for example Bernardim et al., Nat. Commun. 2016, 7, 13128 and Ariyasu et al., Bioconj. Chem. 2017, 28, 897-902, both incorporated by reference, reaction with phosphonamidates, see for example Kasper et al., Angew. Chem. Int. Ed. 2019, 58, 11625-11630, incorporated by reference, reaction with allenamides, see for example Abbas et al., Angew. Chem. Int. Ed. 2014, 53, 7491-7494, incorporated by reference, reaction with cyanoethynyl reagents, see for example Kolodych et al., Bioconj. Chem. 2015, 26, 197-200, incorporated by reference, reaction with vinylsulfones, see for example Gil de Montes et al., Chem. Sci. 2019, 10, 4515-4522, incorporated by reference, or reaction with vinylpyridines, see for example Seki et al, Chem. Sci., 2021, 12, 9060-9068 and https://iksuda.com/science/permalink/ (accessed Jul. 26, 2020).

In terms of DAR, similar to lysine conjugation this is controlled by titration of alkylating reagent for reaction with free cysteine side-chains (liberated by reduction of interchain disulfides with for example TCEP or DTT). The final DAR is typically an average number comprised of a stochastic mixture of different components, again similar to lysine conjugation. However, a few notable differences can be noted: (a) the different DAR species typically consist of a multitude of 2 (i.e. 2, 4, 6, 8) and (b) the maximum DAR that can be achieved is 8 (if all liberated interchain cysteine side-chains have reacted). This also means that by comprehensive alkylation of all interchain cysteine side-chains, a homogeneous DAR8 ADC can be achieved. This is by far the most common method to generate DAR8 ADCs and likely the only method employed for any clinical ADC with DAR8. It must be noted that such approach cannot generate homogeneous DAR6 ADC or ADCs with DAR>8, unless specific cysteines are engineered out or added into the antibody sequence by recombinant DNA technology. It must also be noted that any method involving a reduction step may lead to antibody degradation (reduction of additional disulfide bonds) or fragment scrambling (due to exchange of light chains for example).

An alternative approach to antibody conjugation to interchain disulfide bridges involves the use of a cysteine cross-linking reagent, i.e. a reagent that will react with two cysteine side-chains concurrently. Examples of such cross-linking agents are bis-sulfone reagents, see for example Balan et al., Bioconj. Chem. 2007, 18, 61-76 and Bryant et al., Mol. Pharmaceutics 2015, 12, 1872-1879, both incorporated by reference, mono- or bis-bromomaleimides, see for example Smith et al., J. Am. Chem. Soc. 2010, 132, 1960-1965 and Schumacher et al., Org. Biomol. Chem. 2014, 37, 7261-7269, both incorporated by reference, bis-maleimide reagents, see for example WO2014114207, bis(phenylthio)maleimides, see for example Schumacher et al., Org. Biomol. Chem. 2014, 37, 7261-7269 and Aubrey et al., Bioconj. Chem. 2018, 29, 3516-3521, both incorporated by reference, bis-bromopyridazinediones, see for example Robinson et al., RSC Advances 2017, 7, 9073-9077, incorporated by reference, bis(halomethyl)benzenes, see for example Ramos-Tomillero et al., Bioconj. Chem. 2018, 29, 1199-1208, incorporated by reference or other bis(halomethyl)aromatics, see for example WO2013173391. Typically, ADCs prepared by cross-linking of cysteines have a drug-to-antibody loading of four (DAR4), which is achieved by complete alkylation of all cysteine side-chains liberated by reduction.

Another useful technology for conjugation to a cysteine side chain is by means of formation of a novel disulfide bond, by treatment of a liberated cysteine side-chain with thiolating agent (i.e. a non-symmetrical disulfide bond of which one thiol is part of a good leaving group), leading to a bioactivatable connection that has been utilized for reversibly connecting protein toxins, chemotherapeutic drugs, and probes to carrier molecules (see for example Pillow et al., Chem. Sci. 2017, 8, 366-370, incorporated by reference). Similar to cysteine alkylation, the average DAR of such ADCs can be tailored to around 2-8 in case native interchain disulfide bonds are reduced.

Besides conjugation to the side chains of the naturally present amino acids lysine or cysteine, a range of other conjugation technologies has been explored based on a two-stage strategy involving (a) introduction on a novel reactive group F, followed by (b) reaction with another complementary reactive group Q. For example, a method can be used to introduce a given number of reactive moieties F onto an antibody, which can be two, four or eight, see FIG. 3.

An example of an unnatural reactive functionality F that can be employed for bioconjugation of linker-drugs is the oxime group, suitable for oxime ligation or the azido group, suitable for click chemistry conjugation. The oxime can be installed in the antibody by genetic encoding of a non-natural amino acid, e.g. p-acetophenylalanine, as for example demonstrated by Axup et al. Proc. Nat. Acad. Sci. 2012, 109, 16101-16106, incorporated by reference, or by enzymatic alkylation of a cysteine present in a CAAX sequence with a prenyl group containing a remote keto group, as for example disclosed in WO2012153193. The azide can be installed in the antibody by genetic encoding of p-azidomethyiphenylalanine or p-azidophenylalanine, as for example demonstrated by Axup et al. Proc. Nat. Acad. Sci. 2012, 109, 16101-16106, incorporated by reference. Similarly, Zimmerman et al., Bioconj. Chem. 2014, 25, 351-361, incorporated by reference have employed a cell-free protein synthesis method to introduce p-azidomethylphenylalanine (AzPhe) into monoclonal antibodies for conversion into ADCs by means of metal-free click chemistry. Also, it has also be shown by Nairn et al., Bioconj. Chem. 2012, 23, 2087-2097, incorporated by reference, that a methionine analogue like azidohomoalanine (Aha) can be introduced into protein by means of auxotrophic bacteria and further converted into protein conjugates by means of click chemistry. Finally, genetic encoding of aliphatic azides in recombinant proteins using a pyrrolysyl-tRNA synthetase/tRNA_{CUA} pair was shown by Nguyen et al., J. Am. Chem. Soc. 2009, 131, 8720-8721, incorporated by reference, and labelling was achieved by click chemistry, either by copper-catalyzed alkyne-azide cycloaddition (CuAAC) or strain-promoted alkyne-azide cycloaddition (SPAAC). Besides, CuAAC and SPAAC, bioconjugation of linker-drugs to antibodies (and other biomolecules such as glycans, nucleic acids) can be achieved by a range of other metal-free click chemistries, see e.g. Nguyen and Prescher, Nature Rev. Chem. 2020, 4, 476-489, incorporated by reference. For example, oxidation of a specific tyrosine in a protein can give an ortho-quinone, which readily undergoes cycloaddition with strained alkenes (e.g. TCO) or strained alkynes, see e.g. Bruins et al., Chem. Eur. J. 2017, 24, 4749-4756, incorporated by reference. Besides cyclooctyne, certain cycloheptynes are also suitable for metal-free click chemistry, as reported by Wetering et al. Chem. Sci. 2020, 11, 9011-9016, incorporated by reference. A tetrazine moiety can also be introduced into a protein or a glycan by various means, for example by genetic encoding or chemical acylation, and may also undergo cycloaddition with cyclic alkenes and alkynes. A list of pairs of functional groups F and Q for metal-free click chemistry is provided in FIG. 4.

In a SPAAC bioconjugation, the linker-drug is functionalized with a cyclic alkyne and the cycloaddition with azido-modified antibody is driven by relief of ring-strain. Conversely, the linker-drug can be functionalized with azide and the antibody with cyclic alkyne. Various strained alkynes suitable for metal-free click chemistry are indicated in FIG. 5.

A method of increasing popularity in the field of ADCs is based on enzymatic installation of a non-natural functionality F. For example, Lhospice et al., Mol. Pharmaceut. 2015, 12, 1863-1871, incorporated by reference, employ the bacterial enzyme transglutaminase (BTG or TGase) for installation of an azide moiety onto an antibody. A genetic method based on C-terminal TGase-mediated azide introduction followed by conversion in ADC with metal-free click chemistry was reported by Cheng et al., Mol. Cancer Therap. 2018, 17, 2665-2675, incorporated by reference.

It has been shown in WO2014065661, by van Geel et al., Bioconj. Chem. 2015, 26, 2233-2242, Verkade et al., Antibodies 2018, 7, 12, and Wijdeven at al. MAbs 2022, 14, 2078466, all incorporated by reference, that enzymatic remodelling of the native antibody glycan at N297 enables introduction of an azido-modified sugar, suitable for attachment of cytotoxic payload using metal-free click chemistry, see FIG. 6. Similarly, the enzymatic glycan remodelling protocol can also be employed to install a free thiol group on an antibody for conjugation based on any of the methods described above for cysteine conjugation.

β-Glucuronidase is an enzyme that plays a role in the breakdown of endogenous glucuronic acid-containing glycosaminoglycans and is normally localized to the lysosome of cells, including in the lysosome of first-pass tissues such as the liver and intestine. β-Glucuronidase concentrations in many solid tumors, including lung, breast, and gastrointestinal cancers, as well as in the tumor microenvironment, are reported to be higher than those in normal tissues, and the enzyme is not found in the general circulation. In addition, these enzyme levels are elevated in necrotic regions of tumors due to release of the lysosomal enzyme into the extracellular region from dying cells. Lysosomal concentrations of b-glucuronidase are also high in inflammatory immune cells, including neutrophils and eosinophils, and these cells can release the enzyme at sites of inflammation, such as the necrotic regions of human tumors. Exploitation of β-glucuronidase overexpression in certain tumors is a well-known approach in oncology research, where β-glucuronidase-cleavable triggers have been applied to anthracyclines, auristatins, duocarmycin and camptothecins, as well as in a multi-compound releasing prodrug approach, PBD prodrugs, and ADCs. Similarly, β-galactosidase is an enzyme that is upregulated in tumor microenvironment and can be used for the selective release of payloads capped with a β-galactoside attached to a phenolic position, for example a p-hydroxybenzyloxycarbonyl group.

Other enzymes that are upregulated in the tumor microenvironment have also been considered for release of cytotoxic payloads including cathepsins, matrix metalloproteinases (MMPs), legumain and serine protease elastase, as for example summarized by M. Poreba, FEBS J., 2020, 287, 1936-1969. Investigation into these two TME-related enzymes has led to the development of cleavable linkers based on specific peptide sequences Val-Cit or Val-Ala for cathepsins, Pro-Leu-Gly (PLG) for metalloproteinases (MMPs), Asn-Asn, Ala-Asn or Asn-Ala for legumain and Asn-Pro-Val (NPV) for serine protease elastase, for example.

Currently, cytotoxic payloads include for example microtubule-disrupting agents [e.g. auristatins such as monomethyl auristatin E (MMAE) and monomethyl auristatin F (MMAF), maytansinoids such as DM1 and DM4, tubulysins], DNA-damaging agents [e.g., calicheamicin, pyrrolobenzodiazepine (PBD) dimers, indolinobenzodiapine dimers, duocarmycins, anthracyclines such as PNU-159,682, topoisomerase inhibitors [e.g. DXd, exatecan, SN-38] or RNA polymerase II inhibitors [e.g. amanitin]. ADCs that have reached market approval include for example payloads MMAE, MMAF, DM1, DM4, calicheamicin, SN-38, DXd and a PBD dimer and a pivotal trial is running for an ADC based on duocarmycin. A larger variety of payloads is still under clinical evaluation or has been in clinical trials in the past, e.g. eribulin, indolinobenzodiazepine dimer, PNU-159,682, amanitin, hemi-asterlin and others. Finally, various ADCs in early clinical or late-stage preclinical stage are conjugated to novel payloads for example, KSP inhibitors, MMAD, cryptophycins, and others. Various other payloads have been evaluated in the past as well but have been abandoned due to lack of sufficient efficacy, for example doxorubicin, vinca alkaloids, duocarmycin SA. Even less potent payloads have not even been assessed clinically for the same reason (lack of sufficient efficacy).

One way to widen the scope of payloads employable for ADCs, by employing drugs of lesser potency than the current arsenal, is by increasing the DAR of the ADC, thereby effectively delivering more drug to the tumor per internalization event. For example, Mersana Therapeutics have developed the so-called Fleximer® technology that enables ADCs with DAR12-15, which has been employed in their lead program XMT-1536 (UpRi). To achieve such high DAR, a polymeric, highly polar linker technology was developed first to be able to accommodate the multitude of hydrophobic payloads and to avoid aggregation/degradation of the high DAR ADC, which is essential to avoid severely compromised pharmacokinetic behavior of a high DAR ADC. In fact, it has been convincingly shown by Hamblett et al., Clin. Canc. Res. 2004, 10, 7063-7070, incorporated by reference, that DAR8 ADCs show better in vitro efficacy due to the higher drug loading, while the in vivo performance was worse than the equivalent DAR4 ADC, due to significantly faster clearance of the DAR8 species. Such poor PK properties become apparent by measuring the profile with hydrophilic interaction chromatography (HIC), in particular large relative retention times versus naked antibody can be taken as an indication of poor PK performance, as disclosed by Burke et al., Mol. Canc. Ther. 2017, 16, 116-123, incorporated by reference. Other solutions to mitigate poor PK properties and enable DAR8 ADCs typically involve the insertion of a branched, hydrophilic spacer to shield the hydrophobic payload, for example based on polyethylene glycol (PEG), as disclosed by Burke et al., Mol. Canc. Ther. 2017, 16, 116-123, incorporated by reference, employed in SGN-CD228A and SGN-CD48A, polysarcosine, as disclosed by Viricel et al., Chem. Sci. 2019, 10, 4048-4053, or polysaccharide such as chitosan, as for example disclosed in WO2022058548 and WO2022048883 and employed in M1231.

To date, all high DAR ADCs (˜DAR8 or above) that have reached the clinic are based on a conjugation technology employing cysteine alkylation with a linear linker-drug, which therefore is limited at DAR8 unless additional cysteines are engineered into the antibody by means of recombinant DNA technology. XMT-1536 has a drug loading of 12-15 and is therefore the exception to this rule, however XMT-1536 is also a highly heterogenous ADC, consisting of stochastically conjugated cysteines and a highly heterogeneous polymer attached to each cysteine.

### SUMMARY OF THE INVENTION

The inventors have for the first time been able to prepare homogenous antibody-conjugates with high payload loading (high DAR) by site-specific conjugation to a single antibody N-glycan. The conjugates according to the invention are homogeneous, i.e. have a DAR at or close to the theoretical DAR with a narrow distribution, and do not require any genetic modification of the antibody. The conjugates according to the invention have a payload-to-antibody ratio (DAR) of 6 or higher, preferably 8 or higher. Conjugates with a DAR up to 64 can be readily prepared.

The inventors have developed a modular, non-genetic preparation method for such conjugates, involving three simple steps and starting from any antibody (see FIG. 7). The inventors have developed a modular, non-genetic preparation method for such conjugates, involving three simple steps and starting from any antibody. These steps are (a) enzymatic remodeling of the glycan to give an antibody functionalized with two or four click probes (e.g. an azide) per antibody, (b) a click reaction (e.g. a strain-promoted azide-alkyne cycloaddition) with a multivalent, bifunctional reagent comprising one click probe reactive towards the remodeled antibody (e.g. a cyclic alkyne) and at least two click probes that are not reactive towards the other click probes (e.g. tetrazines) (see FIGS. 8-11), and (c) a separate click reaction (e.g. an inverse electron-demand Diels-Alder cycloaddition) of the click probes with branched linker-drug constructs comprising one click probe reactive towards the previously non-reactive click probes (e.g. cyclic alkyne or strained alkene), connected to one or more payloads preferably connected through a cleavable linker (see FIG. 12-15). The resulting conjugates with DAR6 or higher are rapidly generated with high homogeneity, close to theoretical DAR values and with surprising stability. Moreover, the native glycosylation sites of the antibody are used for site-specific conjugation, such that without the need for genetic engineering of the antibody, conjugates with high DAR can be obtained. In addition, HIC profiles, as well as in vitro and in vivo efficacy and tolerability studies of the resulting ADCs indicate small relative retention time and therefore show high potential in the treatment of cancer.

### DETAILED DESCRIPTION

### Definitions

The verb “to comprise”, and its conjugations, as used in this description and in the claims is used in its non-limiting sense to mean that items following the word are included, but items not specifically mentioned are not excluded.

In addition, reference to an element by the indefinite article “a” or “an” does not exclude the possibility that more than one of the elements is present, unless the context clearly requires that there is one and only one of the elements. The indefinite article “a” or “an” thus usually means “at least one”.

A linker is herein defined as a moiety that connects (covalently links) two or more elements of a compound. A linker may comprise one or more spacer moieties. A spacer-moiety is herein defined as a moiety that spaces (i.e. provides distance between) and covalently links together two (or more) parts of a linker. The linker may be part of e.g. a linker-construct, a linker-conjugate, a linker-payload (e.g. linker-drug) or an antibody-conjugate, as defined below.

A “hydrophilic group” or “polar linker” is herein defined as any molecular structure containing one or more polar functional groups that imparts improved polarity, and therefore improved aqueous solubility, to the molecule it is attached to. Preferred hydrophilic groups are selected from a carboxylic acid group, an alcohol group, an ether group, a polyethylene glycol group, an amino group, an ammonium group, a sulfonate group, a phosphate group, an acyl sulfamide group or a carbamoyl sulfamide group. In addition to higher solubility other effects of the hydrophilic group include improved click conjugation efficiency, and, once incorporated into an antibody-drug conjugate: less aggregation, improved pharmacokinetics resulting in higher efficacy and in vivo tolerability.

The term “salt thereof” means a compound formed when an acidic proton, typically a proton of an acid, is replaced by a cation, such as a metal cation or an organic cation and the like. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt. The term “pharmaceutically accepted” salt means a salt that is acceptable for administration to a patient, such as a mammal (salts with counterions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The term “enediyne” or “enediyne antibiotic” or “enediyne-containing cytotoxin” refers to any cytotoxin characterized by the presence of a 3-ene-1,5-diyne structural feature as part of a cyclic molecule as known in the art and include neocarzinostatin (NCS), C-1027, kedarcidin (KED), maduropeptin (MDP), N1999A2, the sporolides (SPO), the cyanosporasides (CYA and CYN), and the fijiolides, calicheamicins (CAL), the esperamicins (ESP), dynemicin (DYN), namenamicin, shishijimicin, and uncialamycin (UCM).

The term “alkylaminosugar” as used herein means a tetrahydropyranyl moiety connected to an alcohol function via its 2-position, thereby forming an acetal function, and further substituted by (at least) one N-alkylamino group in position 3, 4 or 5. “N-alkylamino group” in this context refers to an amino group having one methyl, ethyl or 2-propyl group.

The term “click probe” refers to a functional moiety that is capable of undergoing a click reaction, i.e. two compatible click probes mutually undergo a click reaction such that they are covalently linked in the product. Compatible probes for click reactions are known in the art, and preferably include (cyclic) alkynes and azides. In the context of the present invention, click probe Q in the compound according to the invention is capable of reacting with click probe F on the (modified) protein, such that upon the occurrence of a click reaction, a conjugate is formed wherein the protein is conjugated to the compound according to the invention. Herein, F and Q are compatible click probes. Click reactions are known in the art and typically refer to cycloaddition reactions such as the [4+2]cycloaddition (e.g. Diels-Alder, inverse electron-demand Diels-Alder) and the [3+2]cycloaddition (e.g. 1,3-dipolar cycloaddition). In the context of the present invention, the term click reaction may also be referred to as cycloaddition.

The term “(hetero)alkyl” refers to alkyl groups and heteroalkyl groups. Heteroalkyl groups are alkyl groups wherein one or more carbon units in the alkyl chain (e.g. CH₂, CH or C) are replaced by heteroatoms, such as O, S, S(O), S(O)₂ or NR⁴. In other words, the alkyl chain is interrupted with one ore more elements selected from O, S, S(O), S(O)₂ and NR⁴. Such interruptions are distinct from substituents, as they occur within the chain of an alkyl group, whereas substituents are pendant groups, monovalently attached to e.g. a carbon atom of an alkyl chain. In a preferred embodiment, the (hetero)alkyl group is an alkyl group, e.g. ethyl (Et), isopropyl (i-Pr), n-propyl (n-Pr), tert-butyl (t-Bu), isobutyl (i-Bu), n-butyl (n-Bu) or n-pentyl.

Likewise, the term “(hetero)aryl” refers to aryl groups and heteroaryl groups. Heteroaryl groups are aryl groups wherein one or more carbon units in the ring (e.g. CH) are replaced by heteroatoms, such as O, S, N or NR⁴.

An “acylsulfamide moiety” is herein defined as a sulfamide moiety (H₂NSO₂NH₂) that is N-acylated or N-carbamoylated on one end of the molecule and N-alkylated (mono or bis) at the other end of the molecule. In the context of the present invention, especially in the examples, this group is also referred to as “HS”.

A “domain” may be any region of a protein, generally defined on the basis of sequence homologies and often related to a specific structural or functional entity. CEACAM family members are known to be composed of Ig-like domains. The term domain is used in this document to designate either individual Ig-like domains, such as “N-domain” or for groups of consecutive domains, such as “A3-B3 domain”.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene” means a DNA sequence that codes for, or corresponds to, a particular sequence of amino acids which comprises all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

The term “glycoprotein” is herein used in its normal scientific meaning and refers to a protein comprising one or more monosaccharide or oligosaccharide chains (“glycans”) covalently bonded to the protein. A glycan may be attached to a hydroxyl group on the protein (O-linked-glycan), e.g. to the hydroxyl group of serine, threonine, tyrosine, hydroxylysine or hydroxyproline, or to an amide function on the protein (N-glycoprotein), e.g. asparagine or arginine, or to a carbon on the protein (C-glycoprotein), e.g. tryptophan. A glycoprotein may comprise more than one glycan, may comprise a combination of one or more monosaccharide and one or more oligosaccharide glycans, and may comprise a combination of N-linked, O-linked and C-linked glycans. It is estimated that more than 50% of all proteins have some form of glycosylation and therefore qualify as glycoprotein. Examples of glycoproteins include PSMA (prostate-specific membrane antigen), CAL (Candida antartica lipase), gp41, gp120, EPO (erythropoietin), antifreeze protein and antibodies.

The term “glycan” is herein used in its normal scientific meaning and refers to a monosaccharide or oligosaccharide chain that is linked to a protein. The term glycan thus refers to the carbohydrate-part of a glycoprotein. The glycan is attached to a protein via the C-1 carbon of one sugar, which may be without further substitution (monosaccharide) or may be further substituted at one or more of its hydroxyl groups (oligosaccharide). A naturally occurring glycan typically comprises 1 to about 10 saccharide moieties. However, when a longer saccharide chain is linked to a protein, said saccharide chain is herein also considered a glycan. A glycan of a glycoprotein may be a monosaccharide. Typically, a monosaccharide glycan of a glycoprotein consists of a single N-acetylglucosamine (GlcNAc), glucose (Glc), mannose (Man) or fucose (Fuc) covalently attached to the protein. A glycan may also be an oligosaccharide. An oligosaccharide chain of a glycoprotein may be linear or branched. In an oligosaccharide, the sugar that is directly attached to the protein is called the core sugar. In an oligosaccharide, a sugar that is not directly attached to the protein and is attached to at least two other sugars is called an internal sugar. In an oligosaccharide, a sugar that is not directly attached to the protein but to a single other sugar, i.e. carrying no further sugar substituents at one or more of its other hydroxyl groups, is called the terminal sugar. For the avoidance of doubt, there may exist multiple terminal sugars in an oligosaccharide of a glycoprotein, but only one core sugar. A glycan may be an O-linked glycan, an N-linked glycan or a C-linked glycan. In an O-linked glycan a monosaccharide or oligosaccharide glycan is bonded to an O-atom in an amino acid of the protein, typically via a hydroxyl group of serine (Ser) or threonine (Thr). In an N-linked glycan a monosaccharide or oligosaccharide glycan is bonded to the protein via an N-atom in an amino acid of the protein, typically via an amide nitrogen in the side chain of asparagine (Asn) or arginine (Arg). In a C-linked glycan a monosaccharide or oligosaccharide glycan is bonded to a C-atom in an amino acid of the protein, typically to a C-atom of tryptophan (Trp).

The term “antibody” (AB) is herein used in its normal scientific meaning. An antibody is a protein generated by the immune system that is capable of recognizing and binding to a specific antigen. An antibody is an example of a glycoprotein. The term antibody herein is used in its broadest sense and specifically includes monoclonal antibodies, polyclonal antibodies, dimers, multimers, multispecific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole antibodies, but also fragments of an antibody, for example an antibody Fab fragment, F(ab′)₂, Fv fragment or Fc fragment from a cleaved antibody, a scFv-Fc fragment, a minibody, a diabody or a scFv. Furthermore, the term includes genetically engineered antibodies and derivatives of an antibody. Antibodies, fragments of antibodies and genetically engineered antibodies may be obtained by methods that are known in the art.

An antibody may be a natural or conventional antibody in which two heavy chains are linked to each other by disulfide bonds and each heavy chain is linked to a light chain by a disulfide bond. There are two types of light chain, lambda (l) and kappa (k). The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties, such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G m3, non-G1 m1 [that, is any allotype other than G1 m1], G1m17, G2m23, G3m21, G3m28, G3m1.1, G3m5, G3m13, G3m14, G3m10, G3m15, G3m16, G3m6, G3m24, G3m26, G3m27, A2m1, A2m2, Km1, Km2 and Km3) of immunoglobulin molecule. Preferred allotypes for administration include a non-G1 ml allotype (nG1m1), such as G1m17,1, G1m3, G1m3.1, G1m3.2 or G1m3.1.2. More preferably, the allotype is selected from the group consisting of the G1m17,1 or G1m3 allotype. The antibody may be engineered in the Fc-domain to enhance or nihilate binding to Fc-gamma receptors, as summarized by Saunders et al. Front. Immunol. 2019, 10, doi: 10.3389/fimmu.2019.01296 and Ward et al., Mol. Immunol. 2015, 67, 131-141. For example, the combination of Leu234Ala and Leu235Ala (commonly called LALA mutations) eliminate FcγRIIa binding. Elimination of binding to Fc-gamma receptors can also be achieved by mutation of the N297 amino acid to any other amino acid except asparagine, by mutation of the T299 amino acid to any other amino acid except threonine or serine, or by enzymatic Deglycosylation or trimming of the fully glycosylated antibody with for example PNGase F or an endoglycosidase. The immunoglobulins can be derived from any species, including human, murine, or rabbit origin. Each chain contains distinct sequence domains.

A percentage of “sequence identity” may be determined by comparing the two sequences, optimally aligned over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. A sequence “at least 85% identical to a reference sequence” is a sequence having, on its entire length, 85%, or more, for instance 90%, 91%, 92%, 93%, 94%, 95%, 96%, 97%, 98% or 99% sequence identity with the entire length of the reference sequence.

The term “CDR” refers to complementarity-determining region: the specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site.

Complementarity Determining Regions or CDRs therefore refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region. “CDR”

The term “monoclonal antibody” or “mAb” as used herein refers to an antibody molecule of a single amino acid sequence, which is directed against a specific antigen, and is not to be construed as requiring production of the antibody by any particular method. A monoclonal antibody may be produced by a single clone of B cells or hybridoma, but may also be recombinant, i.e. produced by protein engineering.

The term “chimeric antibody” refers to an engineered antibody which, in its broadest sense, contains one or more regions from one antibody and one or more regions from one or more other antibodies. In an embodiment, a chimeric antibody comprises a VH domain and a VL domain of an antibody derived from a non-human animal, in association with a CH domain and a CL domain of another antibody, in an embodiment, a human antibody. As the non-human animal, any animal such as mouse, rat, hamster, rabbit or the like can be used. A chimeric antibody may also denote a multispecific antibody having specificity for at least two different antigens.

The term “humanised antibody” refers to an antibody which is wholly or partially of non-human origin and which has been modified to replace certain amino acids, for instance in the framework regions of the VH and VL domains, in order to avoid or minimize an immune response in humans. The constant domains of a humanized antibody are most of the time human CH and CL domains. “Fragments” of (conventional) antibodies comprise a portion of an intact antibody, in particular the antigen binding region or variable region of the intact antibody. Examples of antibody fragments include Fv, Fab, F(ab′)2, Fab′, dsFv, (dsFv)2, scFv, sc(Fv)2, diabodies, bispecific and multispecific antibodies formed from antibody fragments. A fragment of a conventional antibody may also be a single domain antibody, such as a heavy chain antibody or VHH.

The term “multivalent” refers to a linker molecule or linker part of a bigger molecule, with multiple connecting groups. For a linker molecule, these connecting groups are formed by reactive groups capable of covalently attaching to other molecules (e.g. payload or antibody). For a linker part of a bigger molecule, such as a conjugate, the connecting groups are covalent attachment to other parts of the molecule (e.g. payload or antibody). A bivalent linker has two connecting groups, a trivalent linker has three connecting groups, etc. In the context of the current invention, bivalent is also referred to as 2-valent, trivalent is also referred to a 3-valent, etc.

The term “heterofunctional” refers to a linker molecule or linker part of a bigger molecule with connecting groups that are not identical and have different reactivity or are obtained by different reactivity. Typically, such connecting groups in a heterofunctional molecule are mutually non-reactive.

### THE INVENTION

The inventors have for the first time been able to prepare homogenous antibody-conjugates with high payload loading (high DAR) by site-specific conjugation to a single antibody N-glycan. The conjugates according to the invention are homogeneous, i.e. have a DAR at or close to the theoretical DAR with a narrow distribution, and do not require any genetic modification of the antibody. The conjugates according to the invention have a payload-to-antibody ratio (DAR) of 6 or higher, preferably 8 or higher. Conjugates with a DAR up to 64 can be readily prepared.

The inventors have developed a modular, non-genetic preparation method for such conjugates, involving three simple steps and starting from any antibody. These steps are (a) enzymatic remodeling of the glycan to give an antibody functionalized with two or four click probes (e.g. an azide) per antibody, (b) a click reaction (e.g. a strain-promoted azide-alkyne cycloaddition) with a multivalent, bifunctional reagent comprising one click probe reactive towards the remodeled antibody (e.g. a cyclic alkyne) and at least two click probes that are not reactive towards the other click probes (e.g. tetrazines), and (c) a separate click reaction (e.g. an inverse electron-demand Diels-Alder cycloaddition) of the click probes with branched linker-drug constructs comprising one click probe reactive towards the previously non-reactive click probes (e.g. cyclic alkyne or strained alkene), connected to one or more payloads preferably connected through a cleavable linker. The resulting conjugates with DAR6 or higher are rapidly generated with high homogeneity, close to theoretical DAR values and with surprising stability. Moreover, the native glycosylation sites of the antibody are used for site-specific conjugation, such that without the need for genetic engineering of the antibody, conjugates with high DAR can be obtained. In addition, HIC profiles, as well as in vitro and in vivo efficacy and tolerability studies of the resulting ADCs indicate small relative retention time and therefore show high potential in the treatment of cancer.

In a first aspect, the invention concerns a process for preparing an antibody-payload-conjugate having a payload-to-antibody ratio (DAR) of 6 or higher, comprising:
(a) providing a modified antibody having the structure Ab(F¹)_{z}, wherein Ab is an antibody, z is 2 or 4 and F¹ is a click probe;
(b) reacting the modified antibody with z equivalents of Q¹L^{A}(F²)_{y}, wherein Q¹ is a click probe that is reactive towards F¹, L^{A} is a heterobifunctional (y+1)-valent linker, y is 2, 3 or 4, and F¹ is a click probe that is not reactive towards Q¹, to obtain an antibody-linker construct of structure Ab(Z¹L^{A}(F²)_{y})_{z}, wherein Z¹ is a connecting group obtained by reaction of F¹ and Q¹;
(c) reacting the antibody-linker construct with z×y equivalents of Q²(L^{B})Dₓ, wherein Q² is a click probe that is reactive towards F², L^{B} is a (x+1)-valent linker, x is 1, 2, 3 or 4, provided that x+y is at least 4, and D is a payload molecule, to obtain the conjugate of structure Ab(Z¹L^{A}(Z²(L^{B})Dₓ)_{y})_{z}, wherein Z² is a connecting group obtained by reaction of F² and Q².

In a second aspect, the invention concerns conjugates obtainable by the process according to the first aspect of the invention. Alternatively, the conjugates according to the second aspect can also be defined as having structure Ab(Z¹L^{A}(Z²(L^{B})Dₓ)_{y})_{z}, wherein:
Ab is an antibody;
Z¹ and Z² are connecting groups obtainable by reaction between two click probes;
x is 1, 2, 3 or 4;
y is 2, 3 or 4, wherein x+y is at least 4;
z is 2 or 4;
L^{A} is an heterobifunctional (y+1)-valent linker; and
L^{B} is an heterobifunctional (x+1)-valent linker; and
D is a payload.

The conjugates according to the second aspect of the invention are ideally suited for the targeting of tumour cells, in particular for the treatment of cancer. Therefore, the invention also concerns a method for targeting a tumour cell expressing a specific extracellular receptor, comprising contacting the conjugate according to the second aspect of the invention with cells that may possibly express the extracellular receptor, wherein the antibody specifically targets the extracellular receptor. Likewise, the invention also concerns a method for the treatment of cancer, comprising administering to a subject in need thereof the conjugate according to the second aspect of the invention, wherein the cancer cells specifically express an extracellular receptor.

The invention according to the present aspect further concerns a pharmaceutical composition comprising the conjugate according to the second aspect of the invention and a pharmaceutically acceptable carrier.

In a third aspect, the invention concerns heterobifunctional linkers having structure Q¹L^{A}(F²)_{y}, wherein:
Q¹ is a click probe;
L^{A} is a heterobifunctional (y+1)-valent linker;
y is 2, 3 or 4; and
F¹ is a click probe that is not reactive towards Q¹.

The heterobifunctional linkers according to the third aspect of the invention are ideally suited as intermediates in the synthesis of the conjugates according to the second aspect.

Also contemplated within the present invention are salts, preferably pharmaceutically acceptable salts, of the conjugates and heterobifunctional linkers according to the invention.

Here below, the conjugate according to the second aspect is first defined. The structural features of the conjugate according to the invention also apply to the heterobifunctional linkers according to the invention and the process for preparing an antibody-payload-conjugate according to the invention. As the skilled person will appreciate, the structural features of the conjugates according to the invention also apply to the heterobifunctional linkers according to the invention. The skilled person understands that any structure feature that is unchanged in the conjugation reaction is defined equally for each of the molecules according to the invention. In the conjugation reactions, only reactive moieties F and Q are transformed into connecting groups Z. As will be understood by the skilled person, the definition of the chemical moieties, as well as their preferred embodiments, apply to all aspects of the invention.

### Conjugate of General Structure (2)

The invention concerns conjugates of general structure (2):

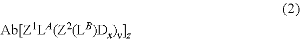

wherein:
Ab is an antibody;
Z¹ and Z² are connecting groups obtainable by reaction between two click probes;
x is 1, 2, 3 or 4;
y is 2, 3 or 4, wherein x+y is at least 4;
z is 2 or 4;
L^{A} is an heterobifunctional (y+1)-valent linker; and
L^{B} is an heterobifunctional (x+1)-valent linker; and
D is a payload.

### The Antibody Ab

Ab is an antibody. Antibodies are known in the art and include IgA, IgD, IgE, IgG, IgM, Fab, VHH, scFv, diabody, minibody, affibody, affylin, affimers, atrimers, fynomer, Cys-knot, DARPin, adnectin/centryin, knottin, anticalin, FN3, Kunitz domain, OBody, bicyclic peptides and tricyclic peptides. Preferably, the antibody is a monoclonal antibody, more preferably selected from the group consisting of IgA, IgD, IgE, IgG and IgM antibodies. Even more preferably Ab is an IgG antibody. The IgG antibody may be of any IgG isotype. The antibody may be any IgG isotype, e.g. IgG1, IgG2, IgI3 or IgG4. Preferably Ab is a full-length antibody, but Ab may also be a Fc fragment.

The antibody Ab is typically specific for an extracellular receptor on a tumour cell, preferably wherein the extracellular receptor on the tumour cell is selected from the group consisting of 5T4, ADAM-9, AMHRII, ASCT2, ASLG659, ASPHD1, av-integrin, Axl, B7-H3, B7-H4, BAFF-R, BCMA, BMPR1B, Brevican, c-KIT, c-Met, C4.4a, CA-IX, cadherin-6, CanAg, CD123, CD13, CD133, CD138/syndecan-1, CD166, CD19, CD20, CD203c, CD205, CD21, CD22, CD228, CD25, CD30, CD324, CD33, CD37, CD38, CD45, CD46, CD48a, CD56, CD70, CD71, CD72, CD74, CD79a, CD79b, CEACAM5, claudin-18.2, claudin-6, CLEC12A, CLL-1, Cripto, CRIPTO, CS1, CXCR5, DLK-1, DLL3, DPEP3, E16, EGFR, ENPP3, EpCAM, EphA2, EphB2R, ETBR, FAP, FcRH1, FcRH2, FcRH5, FGFR2, fibronectin, FLT3, folate receptor alpha, Gal-3BP, GD3, GDNF-Ra1, GEDA, GFRA1, Globo H, gpNMB, GPR172A, GPR19, GPR54, guanyl cyclase C, HER2, HER3, HLA-DOB, IGF-1R, IL13R, IL20Rα, Lewis Y, LGR5, LIV-1, LRRC15, LY64, Ly6E, Ly6G6D, LY6K, MDP, MFI2, MICA/B, MOSPD2, MPF, MSG783, MUC1, MUC16, NaPi2b, NCA, nectin-4, Notch3, P-cadherin, P2X5, PD-L1, PMEL17, PRLR, PSCA, PSCA hlg, PSMA, PTK7, RET, RNF43, RON, ROR1, ROR2, Sema 5b, SLITRK6, SSTR2, STEAP1, STEAP2, TAG72, TENB2, TF, TIM-1, TM4SF, TMEFF, TMEM118, TMEM46, transferrin, TROP-2, TrpM4, TWEAKR, receptor tyrosine kinases (RTK), tenascin.

The antibody may also be specific to an extracellular protein resulting from a viral infection, e.g. human polio virus (HPV), human cytomegalovirus (HCMV) or human papillomavirus (HPV). The antibody may also be specific for a tumour-associated carbohydrate antigen (TACA) that is selected from the group of Tn, STn, T-antigen, LDN, Lewis^{c} (Le^{c}), Sialyl-Lewis^{c} (SLe^{c}), 6-Sialyl-Lewis^{c} (6SLe^{c}), LN, alpha-Gal, 3SLN, 6SLN, H-antigen, A-antigen, B-antigen, Lewis^{a} (Le^{a}), Sialyl-Lewis^{a} (SLe^{a}), 6-Sialyl-Lewis^{a} (6SLe^{a}), Lewis^{b} (Le^{b}), Sialyl-Lewis^{b} (SLe^{b}), 6-Sialyl-Lewis^{b} (6SLe^{b}), Lewis^{x} (Le^{x}), Sialyl-Lewis^{x} (SLe^{x}), 6-Sialyl-Lewis^{x} (6SLe^{x}), Lewis^{y} (Le^{y}), Sialyl-Lewis^{y} (SLe^{y}), 6-Sialyl-Lewis^{y} (6SLe^{y}) and or combinations thereof. The antibody may also be specific to both an extracellular protein and a TACA at the same time.

The number of payloads D attached to a single antibody is known in the art as the DAR (drug-to-antibody ratio). Even though DAR values up to 64 are possible in the context of the present invention, it is preferred that DAR is an even integer in the range 6-32, more preferably 6, 8, 10 or 12, even more preferably 8 or 12, most preferably DAR=8. It will be appreciated that these are theoretical DAR values, and in practice the DAR may slightly deviate from this value, by virtue of incomplete conjugation. Thus, the conjugates may be obtained as mixture of conjugates with varying DAR. In such cases, DAR often refers to the average DAR of the mixture. This is well-known in the art of bioconjugation. The conjugation technique used in the present invention, conjugating site-specifically via the glycan of the antibody and using click reactions, provides conjugates with a narrow distribution with DAR values close to the theoretical value and with narrow standard deviation. For example, when the theoretical DAR is 8, DAR values above 7.4 or even above 7.6 are readily obtained, indicating that most antibodies in the reaction mixture have reacted completely and have a DAR of 8. No antibody conjugates with a DAR above 8 will be present. Thus, the average DAR of the obtained conjugates is close to the theoretical value, preferably the average DAR is at most 15% lower than the theoretical DAR, more preferably the average DAR is at most 10% lower than the theoretical DAR, or even at most 5% lower than the theoretical DAR.

In one embodiment of the conjugates according to the invention, only the conserved glycosylation site is used for conjugation as described below, i.e. z=2, and the antibody is functionalized with 2×x×y occurrences of payload D. Herein, x is 1, 2, 3 or 4 and y is 2, 3 or 4, wherein x+y is at least 4. Thus x+y is an integer in the range 4-8. The exact number of x and y determines the DAR. In one preferred embodiment, y=2 and x=1, 2, 3 or 4, preferably x=2, 3 or 4, more preferably x=2. In another preferred embodiment, x=2 and y=2, 3 or 4, preferably y=2 or 4, more preferably y=2. In another preferred embodiment, x=y, preferably x=y=2 or 4, more preferably x=y=2.

In an alternative embodiment of the conjugates according to the invention, the conserved glycosylation site and a second glycosylation site are both used for conjugation as described below, i.e. z=4, and the antibody is functionalized with 4×x×y occurrences of payload D. Herein, x is 1, 2, 3 or 4 and y is 2, 3 or 4, wherein x+y is at least 4. Thus x+y is an integer in the range 4-8. The exact number of x and y determines the DAR. In one preferred embodiment, y=2 and x=1, 2, 3 or 4, preferably x=1, 2 or 3, more preferably x=2. In another preferred embodiment, x=1 and y=2, 3 or 4, preferably y=2 or 4, more preferably y=2. In another preferred embodiment, x=y, preferably x=y=2 or 4, more preferably x=y=2.

With these values of z, x and y, conjugates with a DAR in the range of DAR 6-64 are readily formed. Conjugates according to the invention have an even DAR. In a preferred embodiment, the DAR=6, with z=2, x=1 and y=3. In another preferred embodiment, the DAR=8, with z=2, x=2 and y=2. In another preferred embodiment, the DAR=8, with z=2, x=1 and y=4. In another preferred embodiment, the DAR=12, with z=2, x=2 and y=3. In another preferred embodiment, the DAR=16, with z=2, x=2 and y=4. In another preferred embodiment, the DAR=32, with z=2, x=4 and y=4. In another preferred embodiment, the DAR=24, with z=2, x=3 and y=4 or with z=2, x=4 and y=3. In another preferred embodiment, the DAR=12, with z=4, x=1 and y=3. In another preferred embodiment, the DAR=16, with z=4, x=2 and y=2. In another preferred embodiment, the DAR=16, with z=4, x=1 and y=4. Preferred conjugates have a DAR in the range of 6-32, more preferably in the range of 6-16, even more preferably in the range of 6-12. In a further preferred embodiment, the DAR is at least 8, preferably the DAR is in the range of 8-32, more preferably in the range of 8-16, even more preferably in the range of 8-12. Most preferably, the DAR=8.

The conjugates according to the invention contain two connecting groups Z¹, which is formed during a click reaction wherein the antibody of structure Ab(F¹)_{z} and a heterobifunctional (y+1)-valent linker construct of structure of Q¹L^{A}(F²)_{y}. In a first click reaction, F¹ and Q¹ react to form a covalent connection between the antibody and z×y moieties F² by forming connecting groups Z¹.

Herein, “(y+1)-valent” refers to the number of connecting points, being either a reactive group F or Q (before reaction) or a connecting group Z (after reaction). Thus, in case y=2, the linker construct contains one click probe Q¹ and two click probes F² and the linker has a valency of 2+1=3. Thus, when y=2, the L^{A} is trivalent. Thus, in case y=3, the linker construct contains one click probe Q¹ and three click probes F² and the linker has a valency of 3+1-4. Thus, when y=3, the L^{A} is tetravalent. Thus, in case y=4, the linker construct contains one click probe Q¹ and four click probes F² and the linker has a valency of 4+1=5. Thus, when y=4, the L^{A} is pentavalent.

Part of the antibody may be a linker L⁶ that connects the click probe F¹ or connecting group Z¹ to the peptide part of the cell-binding agent. Preferably, the connecting group Z¹ is connected to the cell-binding agent CB via a glycan of Ab. Thus, the conjugate according to the invention is preferably represented by:

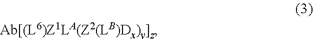

wherein:
L⁶ is -GlcNAc(Fuc)_{w}-(G)ⱼ-S-(L⁷)_{w′}-, wherein G is a monosaccharide, j is an integer in the range of 0-10, S is a sugar or a sugar derivative, GlcNAc is N-acetylglucosamine and Fuc is fucose, w is 0 or 1, w′ is 0 or 1 and L⁷ is —N(H)C(O)CH₂—, —N(H)C(O)CF₂— or —CH₂—.

### Linker L⁶

Linker L⁶ is preferably present, wherein reactive group F¹ may be introduced at a specific position of the antibody. This is for example the case for conjugation via an artificially introduced reactive group F¹, such as for example using transglutaminase, using sortase or by enzymatic glycan modification (e.g. glycosyltransferase or α-1,3-mannosyl-glycoprotein-2-β-N-acetylglucosaminyl-transferase). For example, a modified sugar residue S(F¹)₂ may be introduced at the glycan, extending the glycan with one monosaccharide residue S, which introduces two reactive groups F¹ on the glycan of an antibody. In a most preferred embodiment, conjugation occurs via the glycan of the antibody. The site of conjugation is preferably at the heavy chain of the antibody.

All recombinant antibodies, generated in mammalian host systems, contain the conserved N-glycosylation site at the asparagine residue at or close to position 297 of the heavy chain (Kabat numbering), which is modified by a glycan of the complex type. This naturally occurring glycosylation site of antibodies is preferably used, but other glycosylation sites, including artificially introduced ones, may also be used for the connection of linker L⁶. Thus, in a preferred embodiment, L⁶ is connected to an amino acid of the antibody which is located at a position in the range of 250-350 of the heavy chain, preferably in the range of 280-310 of the heavy chain, more preferably in the range of 295-300 of the heavy chain, most preferably at position 297 of the heavy chain. Using this conserved glycosylation position of the antibody, the obtained conjugates are formed as symmetrical dimers, wherein each half antibody contains one F², and both click probes F² will form an arm with 3 or more payloads. Some antibodies may have a second glycosylation site per half antibody, which is not used as conjugation site in the embodiment where z=2. The skilled person is able to perform the enzymatic conversions in such a way that only the main glycosylation site is utilized for conjugation. Alternatively, the skilled person is able to perform the enzymatic conversion in such a way that also the second glycosylation site is utilized for conjugation, thereby doubling the DAR of the antibody-drug conjugate. In this embodiment, z=4 and each half antibody contains two occurrences of F², and all four click probes F² will form an arm with 3 or more payloads.

L⁶ is a linker that connects Ab to F¹ or Z¹, and is represented by -GlcNAc(Fuc)_{w}-(G)ⱼ-S-(L⁷)_{w′}-, wherein G is a monosaccharide, j is an integer in the range of 0-10, S is a sugar or a sugar derivative, GlcNAc is N-acetylglucosamine and Fuc is fucose, w is 0 or 1, w′ is 0 or 1 and L⁷ is —N(H)C(O)CH₂—, —N(H)C(O)CF₂— or —CH₂—. Typically, L⁶ is at least partly formed by the glycan of an antibody.

The -GlcNAc(Fuc)_{w}-(G)ⱼ- of L⁶ is the glycan, or part thereof. The -GlcNAc(Fuc)_{w}-(G)ⱼ- of the glycan thus typically originates from the original antibody, wherein GlcNAc is an N-acetylglucosamine moiety and Fuc is a fucose moiety. Fuc is typically bound to GlcNAc via an α-1,6-glycosidic bond. Normally, antibodies may (w=1) or may not be fucosylated (w=0). In the context of the present invention, the presence of a fucosyl moiety is irrelevant, and similar effects are obtained with fucosylated (w=1) and non-fucosylated (w=0) antibody conjugates. The GlcNAc residue may also be referred to as the core-GlcNAc residue and is the monosaccharide that is directly attached to the peptide part of the antibody.

S may be directly connected to the core-GlcNAc(Fuc)_{w} moiety, i.e. j=0, meaning that the remainder of the glycan is removed from the core-GlcNAc(Fuc)_{w} moiety before S is attached. Such trimming of glycans is well-known in the art and can be achieved by the action of an endoglycosidase. Alternatively, there are one or more monosaccharide residues present in between the core-GlcNAc(Fuc)_{w} moiety and S, i.e. j is an integer in the range of 1-10, preferably j=1-5. In one preferred embodiment, (G)ⱼ is an oligosaccharide fraction comprising j monosaccharide residues G, wherein j is an integer in the range of 2-5. In another preferred embodiment, (G)ⱼ is a monosaccharide fraction comprising j monosaccharide residues G, wherein j is 1. (G)ⱼ is connected to the GlcNAc moiety of GlcNAc(Fuc)_{w}, typically via a β-1,4 bond. In a preferred embodiment, j is 0, 1, 3, 4 or 5, more preferably, j is 0 or 1, most preferably j is 0.

Although any monosaccharide that may be present in a glycan may be employed as G, each G is preferably individually selected from the group consisting of galactose, glucose, N-acetylgalactosamine, N-acetylglucosamine, mannose and N-acetylneuraminic acid. More preferred options for G are galactose, N-acetylglucosamine, mannose. In case j=1, it is preferred that G=galactose and S=N-acetylneuraminic acid.

When j is 3-10, (G)ⱼ may be linear or branched. Preferred examples of branched oligosaccharides (G)ⱼ are (a), (b), (c), (d), (e), (f), (h) and (h) as shown below.

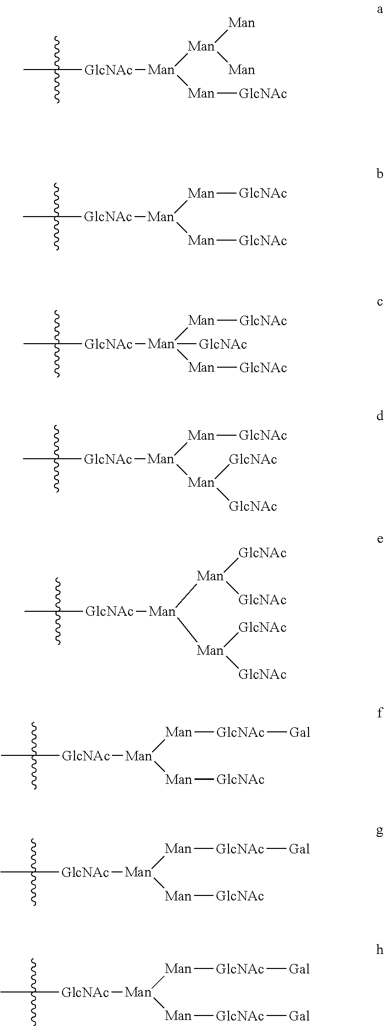

In case (G); is present with j>2, it is preferred that it ends in GlcNAc or Gal, preferably GlcNAc. In other words, the monosaccharide residue directly connected to S is preferably GlcNAc or Gal. The presence of a GlcNAc moiety facilitates the synthesis of the functionalized antibody, as monosaccharide derivative Scan readily be introduced by glycosyltransfer onto a terminal GlcNAc residue. The presence of a Gal moiety facilitates the synthesis of the functionalized antibody, as monosaccharide derivative S sialic acid can readily be introduced by sialyltransferase onto a terminal Gal residue. In the above preferred embodiments for (G)ⱼ, having structure (a)-(h), moiety S may be connected to any of the terminal GlcNAc residues, i.e. not the one with the wavy bond, which is connected to the core GlcNAc residue on the antibody.

Antibodies and antibody conjugates having j=0 or 1 show no or significantly reduced binding to Fc-gamma receptors, while antibodies and antibody conjugates having j in the range of 4-10 do bind to Fc-gamma receptors. Thus, by selecting a certain value for j, the desired extent of binding to Fc-gamma receptors can be obtained. It is thus preferred that j=0, 1, 4, 5, 6, 7, 8, 9 or 10, more preferably j=0, 1, 4 or 5, most preferably the antibody is trimmed and j=0.

S is a sugar or sugar derivative. The term “sugar derivative” is herein used to indicate a derivative of a monosaccharide sugar, i.e. a monosaccharide sugar comprising substituents and/or functional groups. Suitable examples for S include glucose (Glc), galactose (Gal), mannose (Man), fucose (Fuc), amino sugars and sugar acids, e.g. glucosamine (GlcNH₂), galactosamine (GalNH₂) N-acetylglucosamine (GlcNAc), N-acetylgalactosamine (GalNAc), sialic acid (Sia) which is also referred to as N-acetylneuraminic acid (NeuNAc), and N-acetylmuramic acid (MurNAc), glucuronic acid (GlcA) and iduronic acid (IdoA). Preferably, S is selected from Gal, GalNAc and NeuNAc. In an especially preferred embodiment, S is GalNAc.

Connecting group Z¹ or reactive group F¹ may be attached directly to S, or there may be a linker L⁷ present in between S and Z¹ or F¹. Thus, L⁷ may be present (w′=1 or 2) or absent (w′=0). Typically, each moiety Z may be connected to S via a linker L⁷, thus in one embodiment w′=0 of x. Preferably, L⁷ is absent and each connecting moiety Z is directly attached to S. If present, L⁷ may be selected from —N(H)C(O)CH₂—, —N(H)C(O)CF₂— or —CH₂—. In a preferred embodiment, x=1 and w′=0 or 1, most preferably x=1 and w′=0.

Connecting Group Z¹ and Z²

Z¹ and Z² are connecting groups, which covalently connect the antibody with the payloads of the conjugate according to the invention. The term “connecting group” herein refers to the structural element, resulting from a reaction, here between Q and F, connecting one part of the conjugate with another part of the same conjugate. Z¹ is formed by a click reaction between Q¹ and F¹. Likewise, Z² is formed by a click reaction between Q² and F². Herein, Z refers to Z¹ and Z², Q refers to Q¹ and Q², and F refers to F¹ and F²

As will be understood by the person skilled in the art, the exact nature of the connecting group depends on the exact structure of the click probes Q and F. The skilled person is aware of complementary click probes which are reactive towards each other and form suitable reaction partners Q¹/F¹ and Q²/F². For example, when F comprises or is an alkynyl group, complementary groups Q include azido groups. For example, when F comprises or is an azido group, complementary groups Q include alkynyl groups. For example, when F comprises or is a cyclopropenyl group, a trans-cyclooctene group, a cycloheptyne or a cyclooctyne group, complementary groups Q include tetrazinyl groups. In these particular cases, Z is only an intermediate structure and will expel N₂, thereby generating a dihydropyridazine (from the reaction with alkene) or pyridazine (from the reaction with alkyne) as shown in FIG. 4.

Connecting groups Z are obtained by a cycloaddition reaction, preferably wherein the cycloaddition is a [4+2] cycloaddition or a 1,3-dipolar cycloaddition. Conjugation reactions via cycloadditions are known to the skilled person, and the skilled person is capable of selecting appropriate reaction partners F and Q, and will understand the nature of the resulting connecting group Z. Preferred cycloadditions are a [4+2]-cycloaddition (e.g. a Diels-Alder reaction) or a [3+2]-cycloaddition (e.g. a 1,3-dipolar cycloaddition). Preferably, the cycloaddition is the Diels-Alder reaction or the 1,3-dipolar cycloaddition. The preferred Diels-Alder reaction is the inverse electron-demand Diels-Alder cycloaddition. In another preferred embodiment, the 1,3-dipolar cycloaddition is used, more preferably the alkyne-azide cycloaddition. Cycloadditions, such as Diels-Alder reactions and 1,3-dipolar cycloadditions are known in the art, and the skilled person knows how to perform them.

Preferably, Z contains a moiety selected from the group consisting of a triazole, a cyclohexene, a cyclohexadiene, a [2.2.2]-bicyclooctadiene, a [2.2.2]-bicyclooctene, an isoxazoline, an isoxazolidine, a pyrazoline, a piperazine or a pyridazine, more preferably a triazole, an isoxazoline or pyridazine. Triazole moieties are especially preferred to be present in Z. In one embodiment, Z comprises a (hetero)cycloalkene moiety, i.e. formed from Q comprising a (hetero)cycloalkyne moiety. In an alternative embodiment, Z comprises a (hetero)cycloalkane moiety, i.e. formed from Q comprising a (hetero)cycloalkene moiety. Herein, aromatic rings such as a triazole ring are considered a heterocycloalkane ring, since it is formed by reaction of an alkyne moiety and an azide moiety.

In a preferred embodiment, Z has the structure (Z1):

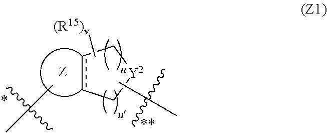

Herein, the bond depicted as [symbol] is a single bond or a double bond. Furthermore:
ring Z is obtained by a cycloaddition, preferably ring Z is selected from (Za)-(Zm) defined below, wherein the carbon atoms labelled with ** correspond to the two carbon atoms of the bond depicted as [symbol] of (Z1) to which ring Z is fused;
R¹⁵ is independently selected from the group consisting of hydrogen, halogen, —OR¹⁶, —NO₂, —CN, —S(O)₂R¹⁸, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S, S⁽⁺⁾R³¹, S(O)R³¹, S(O)—NR³¹ or NR³¹, wherein S⁽⁺⁾ is a cationic sulphur atom counterbalanced by B⁽⁻⁾, wherein B⁽⁻⁾ is an anion, and wherein each R³¹ individually is R¹⁵ or a connection with D, connected via L;
u is 0, 1, 2, 3, 4 or 5;
u′ is 0, 1, 2, 3, 4 or 5, wherein u+u′=0, 1, 2, 3, 4, 5, 6, 7 or 8;
v=an integer in the range 8-16;
Ring Z is formed by the cycloaddition, and is preferably selected from (Za)-(Zm).

In a preferred embodiment, u+u′=0, 4, 5, 6, 7 or 8, more preferably 0, 4 or 5. In case the bond depicted as [symbol] is a double bond, it is preferred that u+u′=4, 5, 6, 7 or 8, more preferably u+u′=4 or 5. In case the bond depicted as [symbol] is a single bond, it is preferred that u+u′=0 or 5. Preferably, the wavy bond labelled with * is connected to CB, optionally via L⁶, and the wavy bond labelled with ** is connected to L.

It is especially preferred that Z comprises a (hetero)cycloalkene moiety, i.e. the bond depicted as [symbol] is a double bond. In a preferred embodiment, Z is selected from the structures (Z2)-(Z20), depicted here below:

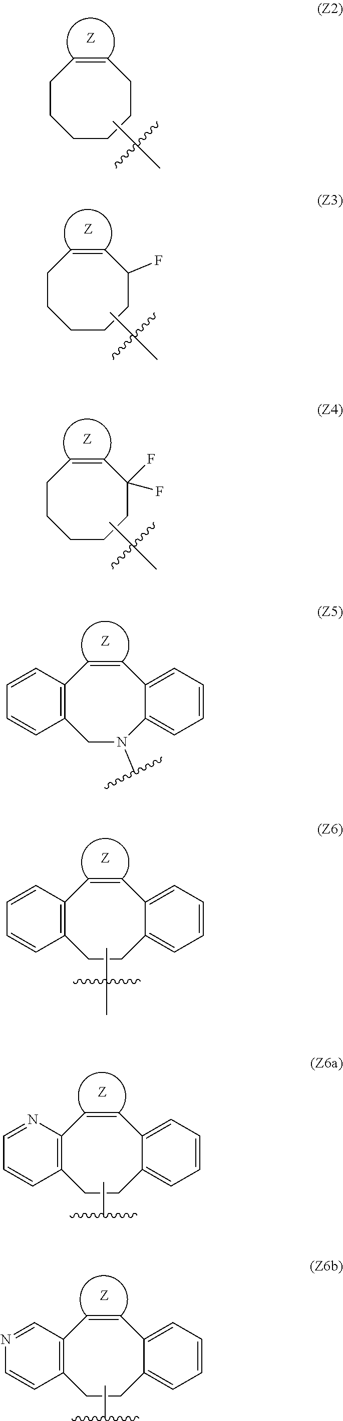

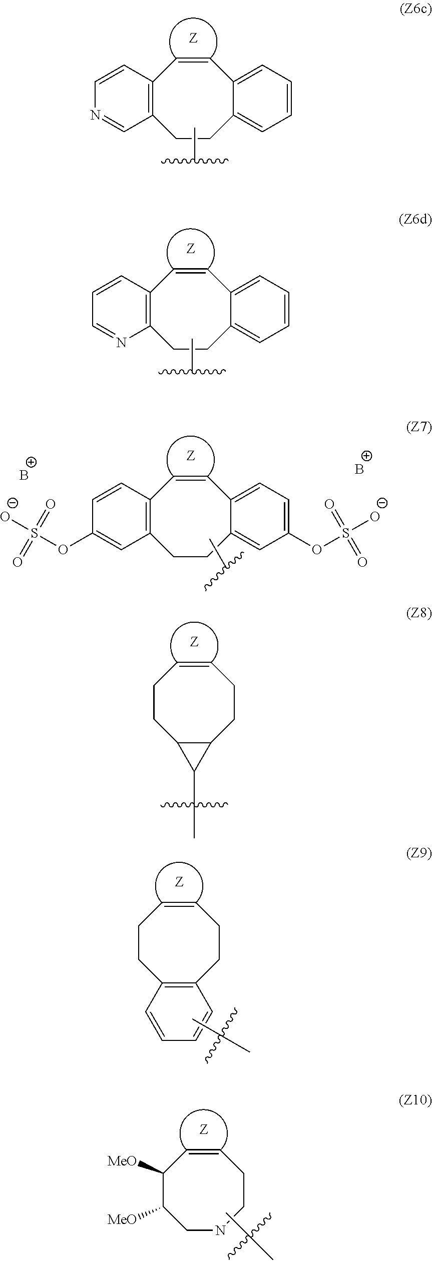

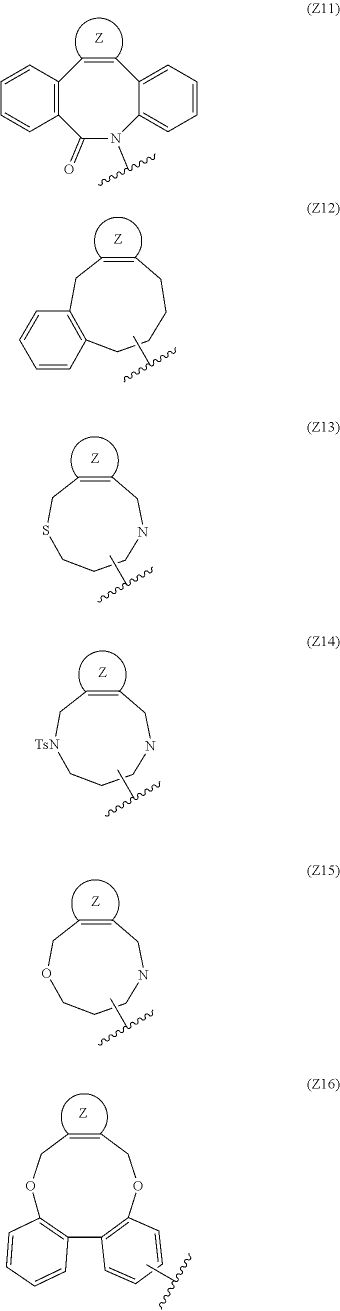

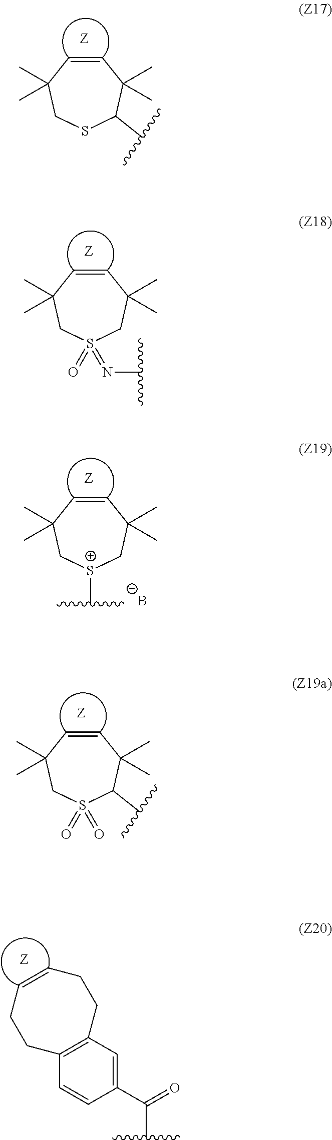

Herein, the connection to L is depicted with the wavy bond. B⁽⁻⁾ is an anion, preferably a pharmaceutically acceptable anion. B⁽⁺⁾ is a cation, preferably a pharmaceutically acceptable cation. Ring Z is formed by the cycloaddition reaction, and preferably is a triazole, a cyclohexene, a cyclohexadiene, a [2.2.2]-bicyclooctadiene, a [2.2.2]-bicyclooctene, an isoxazoline, an isoxazolidine, a pyrazoline or a piperazine. Most preferably, ring Z is a triazole ring. Ring Z may have the structure selected from (Za)-(Zm) depicted below, wherein the carbon atoms labelled with ** correspond to the two carbon atoms of the (hetero)cycloalkane ring of (Z2)-(Z20), to which ring Z is fused. Since the connecting group Z is formed by reaction with a (hetero)cycloalkyne in the context of the present embodiment, the bond depicted above as [symbol] is a double bond.

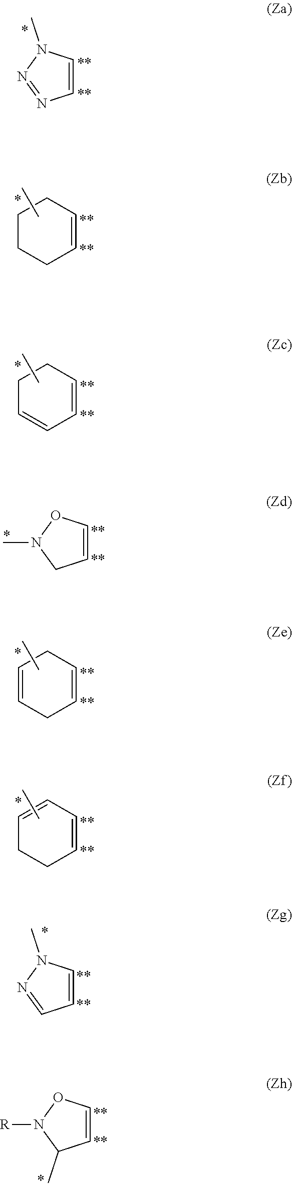

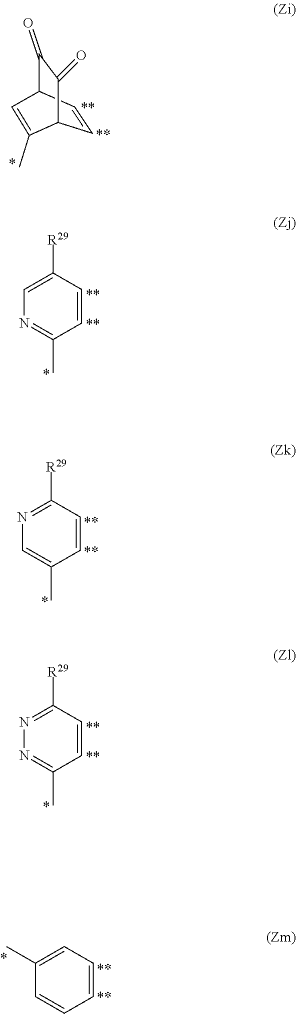

Herein, R²⁹ is selected from hydrogen, C₁₋₆ alkyl, aryl, C(O)—C₁₋₆ alkyl, C(O)-aryl, C(O)—O—C₁₋₆ alkyl, C(O)—O-aryl, C(O)—NR³³—C₁₋₆ alkyl and C(O)—NR³³-aryl, wherein R³³ is H or C₁₋₄ alkyl. Preferably, R²⁹ is selected from hydrogen, methyl, phenyl, pyridyl, pyridinyl and pyrimidinyl. It was found that R²⁹ is hydrogen gave optimal results in reactivity in the cycloaddition reaction, especially in case ring (Zl) is formed. Thus, in a preferred embodiment, ring Z is (Zl) wherein R²⁹ is selected from hydrogen, methyl, phenyl, pyridyl, pyridinyl and pyrimidinyl, more preferably R²⁹ is hydrogen.

In case Z comprises a (hetero)cycloalkene moiety, it is preferred that ring Z is selected from (Za), (Zj), (Zk) or (Zl), more preferably ring Z is according to structure (Za) or (Zl).

In a further preferred embodiment, Z is selected from the structures (Z21)-(Z38a), depicted here below:

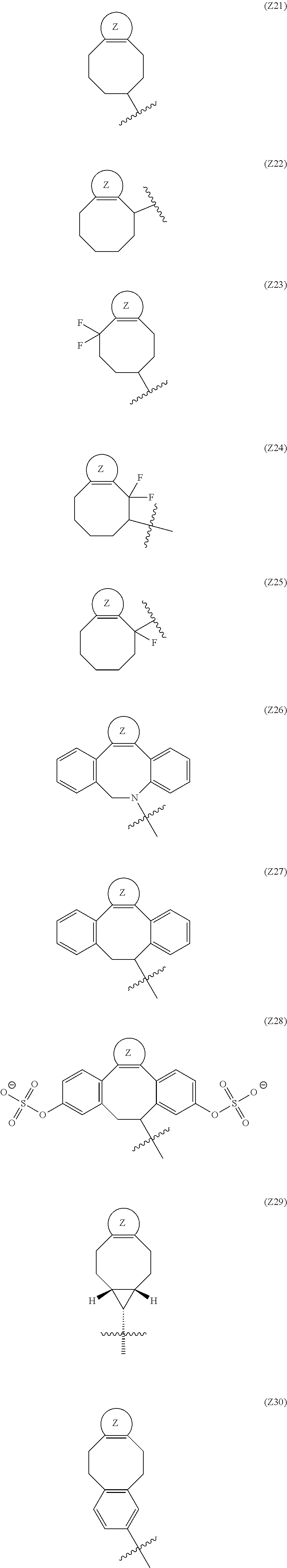

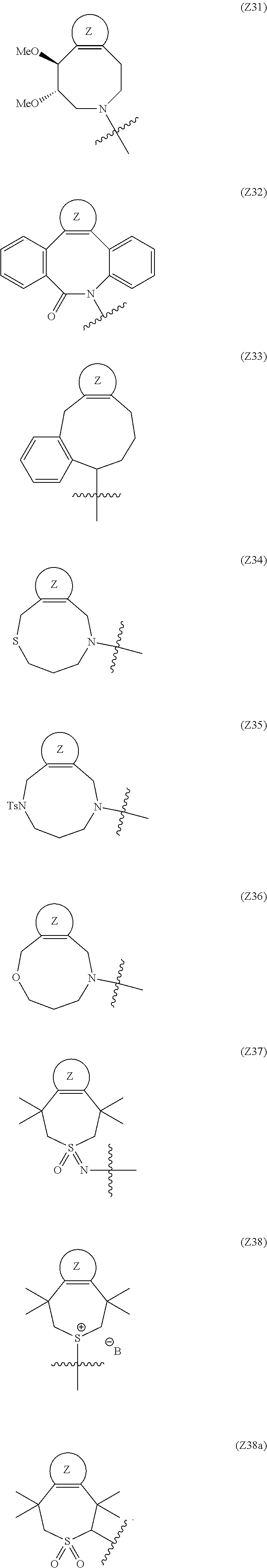

Herein, the connection to L is depicted with the wavy bond. Structure (Z29) can be in endo or exo configuration, preferably it is in endo configuration. In structure (Z38), B⁽⁻⁾ is an anion, preferably a pharmaceutically acceptable anion. Ring Z is selected from structures (Za)-(Zm), as defined above.

In a preferred embodiment, Z comprises a (hetero)cyclooctene moiety or a (hetero)cycloheptene moiety, preferably according to structure (Z8), (Z26), (Z27), (Z28), (Z37) or (Z38a), which are optionally substituted. Each of these preferred options for Z are further defined here below.

Thus, in a preferred embodiment, Z comprises a heterocycloheptene moiety according to structure (Z37), which is optionally substituted. Preferably, the heterocycloheptene moiety according to structure (Z37) is not substituted.

In a preferred embodiment, Z comprises a (hetero)cyclooctene moiety according to structure (Z8), more preferably according to (Z29), which is optionally substituted. Preferably, the cyclooctene moiety according to structure (Z8) or (Z29) is not substituted. In the context of the present embodiment, Z preferably comprises a (hetero)cyclooctene moiety according to structure (Z39) as shown below, wherein V is (CH₂)ₗ and l is an integer in the range of 0 to 10, preferably in the range of 0 to 6. More preferably, l is 0, 1, 2, 3 or 4, more preferably l is 0, 1 or 2 and most preferably l is 0 or 1. In the context of group (Z39), l is most preferably 1. Most preferably, Z is according to structure (Z42), defined further below.

In an alternative preferred embodiment, Z comprises a (hetero)cyclooctene moiety according to structure (Z26), (Z27) or (Z28), which is optionally substituted. In the context of the present embodiment, Z preferably comprises a (hetero)cyclooctene moiety according to structure (Z40) or (Z41) as shown below, wherein Y¹ is O or NR¹¹, wherein R¹¹ is independently selected from the group consisting of hydrogen, a linear or branched C₁-C₁₂ alkyl group or a C₄-C₁₂ (hetero)aryl group. The aromatic rings in (Z40) are optionally 0-sulfated at one or more positions, whereas the rings of (Z41) may be halogenated at one or more positions. Preferably, the (hetero)cyclooctene moiety according to structure (Z40) or (Z41) is not further substituted. Most preferably, Z is according to structure (Z43), defined further below.

In an alternative preferred embodiment, Z comprises a heterocycloheptenyl group and is according to structure (Z37).

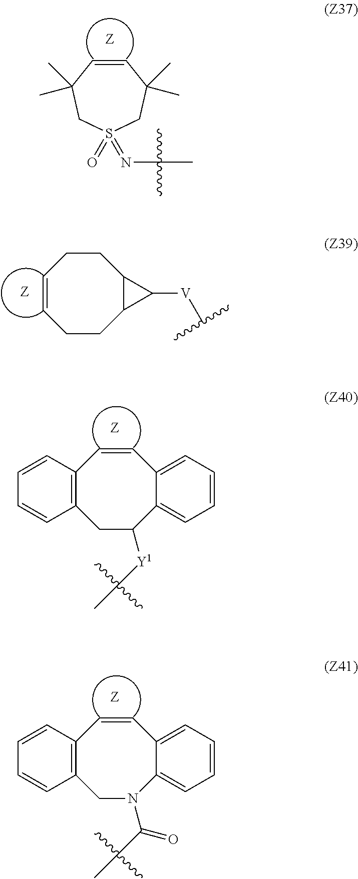

In an especially preferred embodiment, Z comprises a cyclooctenyl group and is according to structure (Z42):

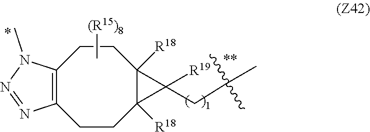

Herein:
the bond labelled with * is connected to CB and the wavy bond labelled with ** is connected to L;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of Z (or Q) or D connected via a spacer moiety; and
l is an integer in the range 0 to 10.

In a preferred embodiment of the group according to structure (Z42), R¹⁵ is independently selected from the group consisting of hydrogen, halogen, —OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the group according to structure (Z42), R¹⁸ is independently selected from the group consisting of hydrogen, C₁-C₆ alkyl groups, most preferably both R¹⁸ are H. In a preferred embodiment of the group according to structure (Z42), R¹⁹ is H. In a preferred embodiment of the group according to structure (Z42), l is 0 or 1, more preferably l is 1.

In an especially preferred embodiment, Z comprises a (hetero)cyclooctenyl group and is according to structure (Z43):

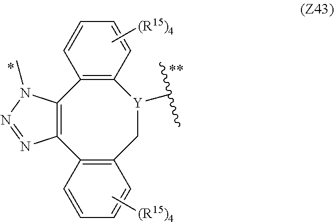

Herein:
the bond labelled with * is connected to CB and the wavy bond labelled with ** is connected to L;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵;
a carbon atom in the fused aromatic rings may be replaced by a nitrogen atom, as in (Z6a)-(Z6d), preferably wherein Y is CR¹⁵.

In a preferred embodiment of the group according to structure (Z43), R¹⁵ is independently selected from the group consisting of hydrogen, halogen, —OR¹⁶, —S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃⁽⁻⁾. In a preferred embodiment of the group according to structure (Z43), Y is N or CH, more preferably Y=N.

In an especially preferred embodiment, Z comprises a heterocycloheptenyl group and is according to structure (Z37) or (Z38a), wherein ring Z is a triazole.

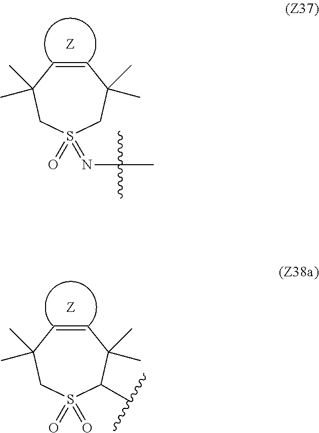

In an alternative preferred embodiment, connecting group Z comprises a (hetero)cycloalkane moiety, i.e. the bond depicted as [symbol] is a single bond. The (hetero)cycloalkane group may also be referred to as a heterocycloalkyl group or cycloalkyl group, preferably a cycloalkyl group, wherein the (hetero)cycloalkyl group is optionally substituted. Preferably, the (hetero)cycloalkyl group is a (hetero)cyclopropyl group, a (hetero)cyclobutyl group, a norbornyl group, a norbornenyl group, a (hetero)cycloheptyl group, a (hetero)cyclooctyl group, which may all optionally be substituted. Especially preferred are (hetero)cyclopropyl groups, (hetero)cycloheptyl group or (hetero)cyclooctyl groups, wherein the (hetero)cyclopropyl group, the (hetero)cycloheptyl group or the (hetero)cyclooctyl group is optionally substituted. Preferably, Z comprises a cyclopropyl moiety according to structure (Z44), a hetereocyclobutane moiety according to structure (Z45), a norbornane or norbornene group according to structure (Z46), a (hetero)cycloheptyl moiety according to structure (Z47) or a (hetero)cyclooctyl moiety according to structure (Z48). Herein, Y³ is selected from C(R²³)₂, NR²³ or O, wherein each R²³ is individually hydrogen, C₁-C₆ alkyl or is connected to L, optionally via a spacer, and the bond labelled [symbol] is a single or double bond. In a further preferred embodiment, the cyclopropyl group is according to structure (Z49). In another preferred embodiment, the (hetero)cycloheptane group is according to structure (Z50) or (Z51). In another preferred embodiment, the (hetero)cyclooctane group is according to structure (Z52), (Z53), (Z54), (Z55) or (Z56).

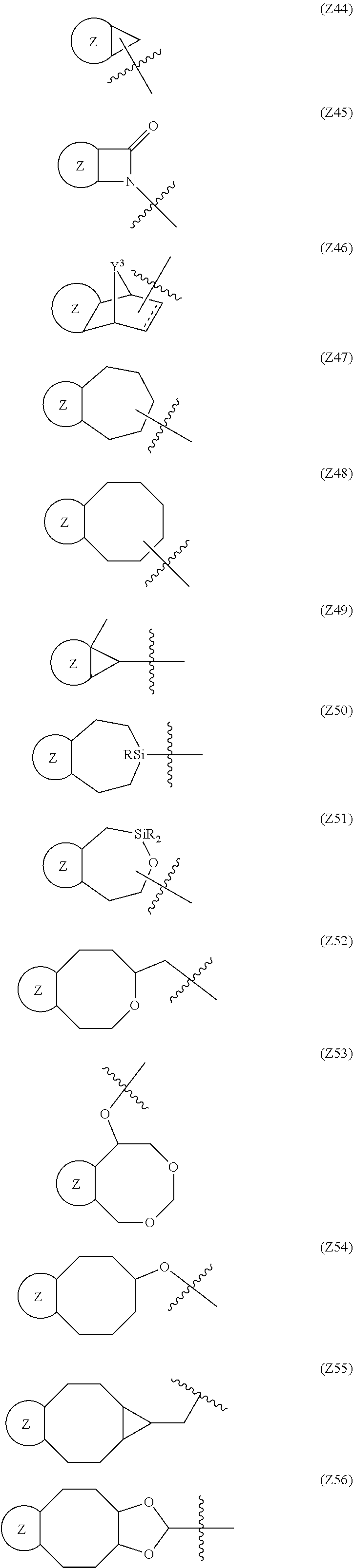

Herein, the R group(s) on Si in (Z50) and (Z51) are typically alkyl or aryl, preferably C₁-C₆ alkyl. Ring Z is formed during the cycloaddition reaction and is typically selected from structures (Zn)-(Zu), wherein the carbon atoms labelled with ** correspond to the two carbon atoms of the (hetero)cycloalkane ring of (Z44)-(Z56) to which ring Z is fused, and the carbon a carbon labelled with * is connected to CB. Since the connecting group Z is formed by reaction with a (hetero)cycloalkene in the context of the present embodiment, the bound depicted above as [symbol] is a single bond.

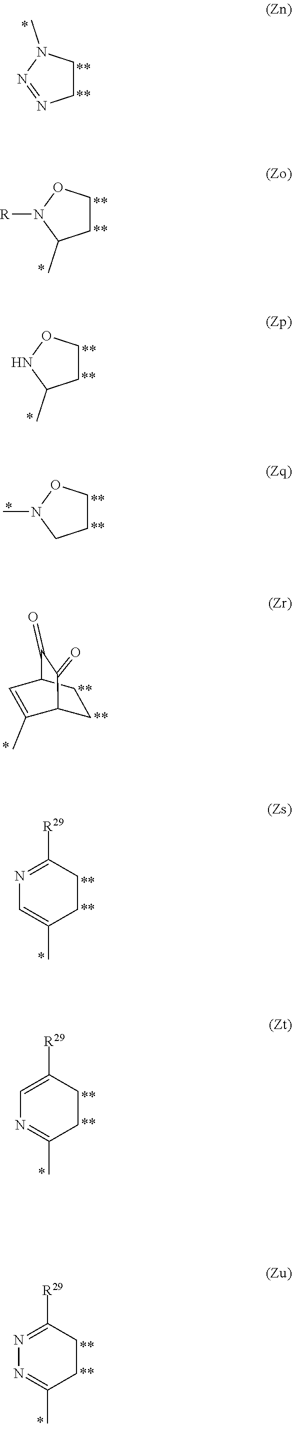

Herein, R²⁹ is selected from hydrogen, C₁₋₆ alkyl, aryl, C(O)—C₁₋₆ alkyl, C(O)-aryl, C(O)—O—C₁₋₆ alkyl, C(O)—O-aryl, C(O)—NR³³—C₁₋₆ alkyl and C(O)—NR³³-aryl, wherein R³³ is H or C₁₋₄ alkyl. Preferably, R²⁹ is selected from hydrogen, methyl, phenyl, pyridyl, pyridinyl and pyrimidinyl. It was found that R²⁹ is hydrogen gave optimal results in reactivity in the cycloaddition reaction, especially in case ring (Zu) is formed. Thus, in a preferred embodiment, ring Z is (Zu) wherein R²⁹ is selected from hydrogen, methyl, phenyl, pyridyl, pyridinyl and pyrimidinyl, more preferably R²⁹ is hydrogen.

In case Z comprises a (hetero)cycloalkane moiety, it is preferred that ring Z is selected from (Zn), (Zs), (Zt) or (Zu), most preferably ring Z is according to structure (Zu).

In a preferred embodiment, connection group Z comprise a moiety selected from (Z1)-(Z56), wherein ring Z is selected from (Za)-(Zu).

In the present invention, the exact structure of Z¹ and Z² typically differ, as Z¹ is formed by reaction of Q¹ and F¹, whereas Z² is formed by reaction of Q² and F². Herein, F² is reactive towards Q² but not towards Q¹, such that Q¹ and Q² should differ.

In a preferred embodiment, F¹ is azide and Q¹ is a benzoannulated or tetramethylated (hetero)cycloalkyne, while F² is tetrazine or nitrone and Q² is bicyclononyne or cycloalkene, such as a trans-cyclooctene or a cyclopropene. More preferably, F¹ is azide and Q¹ is a benzoannulated or tetramethylated (hetero)cycloalkyne, while F² is tetrazine and Q² is bicyclononyne.

Herein, the reaction of F¹ and Q¹ will preferably form a connecting group Z¹ according to structure (Z5), (Z6), (Z7), (Z11), (Z17), (Z18), (Z19) or (Z19a), wherein ring Z is according to structure (Za), preferably according to structure (Z26), (Z27), (Z28), (Z32), (Z37), (Z38) or (Z38a), more preferably according to structure (Z40), (Z41) or (Z43), or according to structure (Z37) or (Z43). Herein, Q² is preferably Herein, the reaction of F² and Q² will preferably form a connecting group Z² according to structure (Z8), (Z44), (Z47), (Z48), (Z49), (Z54), (Z55) or (Z56), more preferably according to structure (Z29), (Z48) or (Z49), most preferably according to structure (Z42). Herein, ring Z is according to structure (Zd), (Zl), (Zq) or (Zu), preferably according to structure (Zl) or (Zu), most preferably according to structure (Zl). Alternatively, connecting group Z² is according to structure (Z8), wherein ring Z is according to structure (Zl), preferably according to structure (Z29), more preferably according to structure (Z42).

### Linker L^{A}

Linker L^{A} connects payloads D, via linker L^{B} and connecting groups Z², with Ab via connecting group Z¹ (in the conjugates according to the invention) or connects reactive group F² with reactive group Q¹ (in the linker according to the invention). Linkers are known in the art and may be cleavable or non-cleavable. Linker L^{A} preferably is not cleavable linker, while linker L^{B} preferably is cleavable.

Linker L^{A} is connected to one occurrence of Z¹ (or Q¹) and y occurrences of Z² (or F²). Thus, the valency of linker L^{A} is y+1 or “(y+1)-valent”. Herein, “(y+1)-valent” refers to the number of connecting points, being either a reactive group F or Q (before reaction) or a connecting group Z (after reaction). Thus, in case y=2, the linker is connected to one Z¹/Q¹ and two Z²/F² and the linker has a valency of 2+1=3. Thus, when y=2, the L^{A} is trivalent. Thus, in case y=3, the linker is connected to one Z¹/Q¹ and three Z²/F² and the linker has a valency of 3+1=4. Thus, when y=3, the L^{A} is tetravalent. Thus, in case y=4, the linker is connected to one Z¹/Q¹ and four Z²/F² and the linker has a valency of 4+1=5. Thus, when y=4, the L^{A} is pentavalent.

Linker L^{A} may be referred to as “heterobifunctional”, which means that is contains two different functionalities, referring to the chemically different connectivities to Z¹/Q¹ and Z²/F².

In a preferred embodiment, L^{A} is a heterobifunctional linker of structure

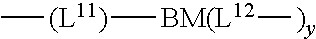

wherein:
L¹¹ is connected to Q¹ or Z¹, and each L¹² is connected to F² or Z², wherein L¹¹ and L¹² each individually consist of one or more building blocks selected from C(R¹³)₂, C(R¹³)—C(R¹³), C≡C, C(O), NR¹³, O, S, S(O), S(O)₂, PR¹³ and P(O)R¹³, preferably the building blocks are selected from CH₂, CH═CH, C(O), NR¹³, O, S, S(O) and S(O)₂,
each R¹³ is as defined below for structure (23), preferably R¹³ is H; and
BM is a branching moiety. The branching moiety is further defined below.

In a more preferred embodiment, L^{A} is a heterobifunctional trivalent linker of structure

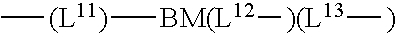

wherein:
L¹¹ is connected to Q¹ or Z¹, and L¹² and L¹³ are connected to F² or Z², wherein L¹¹, L¹² and L¹³ each individually consist of one or more building blocks selected from C(R¹³)₂, C(R¹³)═C(R¹³), C≡C, C(O), NR¹³, O, S, S(O), S(O)₂, PR¹³ and P(O)R¹³, preferably the building blocks are selected from CH₂, CH═CH, C(O), NR¹³, O, S, S(O) and S(O)₂, and
each R¹³ is as defined below for structure (23), preferably R¹³ is H; and
BM is a branching moiety. The branching moiety is further defined below.

L¹¹, L¹² and L¹³ may for example be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)_{y′} and NR²¹, wherein y′ is 0, 1 or 2, preferably y′=2, and R²¹ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups. In one embodiment, the optional substituents may be selected from polar groups, such as oxo groups, (poly)ethylene glycol diamines, (poly)ethylene glycol or (poly)ethylene oxide chains, (poly)propylene glycol or (poly)propylene oxide chains, carboxylic acid groups, carbonate groups, carbamate groups, cyclodextrins, crown ethers, saccharides (e.g. monosaccharides, oligosaccharides), phosphates or esters thereof, phosphonic acid or ester, phosphinic acid or ester, sulfoxides, sulfones, sulfonic acid or ester, sulfinic acid, or sulfenic acid.

In the context of the present invention, a linker construct of structure Q¹L^{A}(F²)_{y} reacts with structure Ab(F¹)_{z}, thereby performing z click reactions between F¹ and Q¹ per antibody Ab, and thus forming z connecting groups Z¹ per antibody Ab. Upon completion of the reaction, an antibody-linker construct is formed containing z×y click probes F² per antibody. The antibody-linker construct thus formed has a structure Ab(Z¹L^{A}(F²)_{y})_{z}.

### The Branching Moiety BM

A “branching moiety” in the context of the present invention refers to a moiety that is embedded in a linker connecting three moieties. In other words, the branching moiety comprises at least three bonds to other moieties.

Any moiety that contains at least three bonds to other moieties is suitable as branching moiety in the context of the present invention. Suitable branching moieties include a carbon atom (BM-1), a nitrogen atom (BM-3), a phosphorus atom (phosphine (BM-5) and phosphine oxide (BM-6)), aromatic rings such as a phenyl ring (e.g. BM-7) or a pyridyl ring (e.g. BM-9), a (hetero)cycle (e.g. BM-11 and BM-12) and polycyclic moieties (e.g. BM-13, BM-14 and BM-15). Preferably, BM is selected from a carbon atom, a nitrogen atom, a phosphorus atom, a (hetero)aromatic ring, a (hetero)cycle or a polycyclic moiety, more preferably BM is a carbon atom or a nitrogen atom. In case BM is a nitrogen atom, the branching nitrogen may be part of an amide group, for example with the connection to Z¹ or Q¹ via the C═O group and the connection to two occurrences of Z² or F² via the two substituents on nitrogen. In case BM is a carbon atom, the carbon atom is preferably part of a trivalent amino acid, such as lysine, aspartic acid or glutamic acid.

Suitable branching moieties BM are selected from structures (BM-1) to (BM-15) depicted here below, wherein the three branches, i.e. bonds to other moieties as defined above, are indicated by (a bond labelled with *).

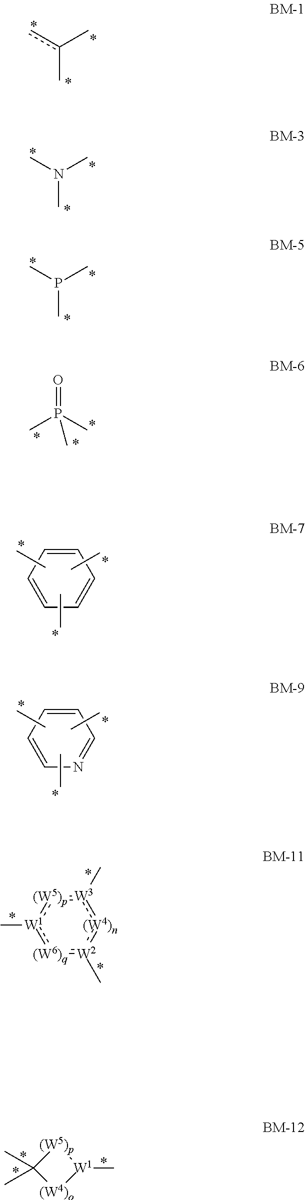

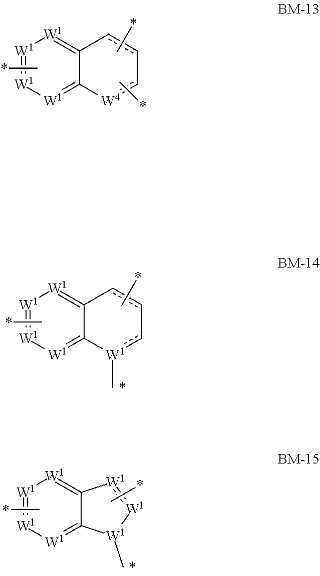

In (BM-1), one of the branches labelled with * may be a single or a double bond, indicated with [symbol] . In (BM-11) to (BM-15), the following applies:
each of n, p, q and q is individually an integer in the range of 0-5, preferably 0 or 1, most preferably 1;
each of W¹, W² and W³ is independently selected from C(R²¹)_{w} and N;
each of W⁴, W⁵ and W⁶ is independently selected from C(R²¹)_{w+1}, N(R²²)_{w}, O and S;
each [symbol] represents a single or double bond;
w is 0 or 1 or 2, preferably 0 or 1;
each R²¹ is independently selected from the group consisting of hydrogen, OH, C₁-C₂₄ alkyl groups, C₁-C₂₄ alkoxy groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, wherein the C₁-C₂₄ alkyl groups, C₁-C₂₄ alkoxy groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups are optionally substituted and optionally interrupted by one or more heteroatoms selected from O, S and NR³ wherein R³ is independently selected from the group consisting of hydrogen and C₁-C₄ alkyl groups; and
each R²² is independently selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, wherein the C₁-C₂₄ alkyl groups, C₁-C₂₄ alkoxy groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups are optionally substituted and optionally interrupted by one or more heteroatoms selected from O, S and NR³ wherein R³ is independently selected from the group consisting of hydrogen and C₁-C₄ alkyl groups.

The skilled person appreciates that the values of wand the bond order of the bonds represented by [symbol] are interdependent. Thus, whenever an occurrence of W is bonded to an endocyclic double bond, w=1 for that occurrence of W, while whenever an occurrence of W is bonded to two endocyclic single bonds, w=0 for that occurrence of W. For BM-12, at least one of o and p is not 0.

Representative examples of branching moieties according to structure (BM-11) and (BM-12) include cyclopropyl, cyclobutyl, cyclopentyl, cyclohexyl, cycloheptyl, cyclooctyl, cyclopropenyl, cyclobutenyl, cyclopentenyl, cyclohexenyl, cycloheptenyl, cyclooctenyl, aziridine, azetidine, diazetidine, oxetane, thietane, pyrrolidine, dihydropyrrolyl, tetrahydrofuranyl, dihydrofuranyl, thiolanyl, imidazolinyl, pyrazolidinyl, oxazolidinyl, isoxazolidinyl, thiazolidinyl, isothiazolidinyl, dioxolanyl, dithiolanyl, piperidinyl, oxanyl, thianyl, piperazinyl, morpholino, thiomorpholino, dioxanyl, trioxanyl, dithyanyl, trithianyl, azepanyl, oxepanyl and thiepanyl. Preferred cyclic moieties for use as branching moiety include cyclopropenyl, cyclohexyl, oxanyl (tetrahydropyran) and dioxanyl. The substitution pattern of the three branches determines whether the branching moiety is of structure (BM-11) or of structure (BM-12).

Representative examples of branching moieties according to structure (BM-13) to (BM-15) include decalin, tetralin, dialin, naphthalene, indene, indane, isoindene, indole, isoindole, indoline, isoindoline, and the like.

In a preferred embodiment, BM is a carbon atom. In case the carbon atom is according to structure (BM-1) and has all four bonds to distinct moieties, the carbon atom is chiral. The stereochemistry of the carbon atom is not crucial for the present invention, and may be S or R. The same holds for the phosphine (BM-6). Most preferably, the carbon atom is according to structure (BM-1). One of the branches indicated with * in the carbon atom according to structure (BM-1) may be a double bond, in which case the carbon atom may be part of an alkene or imine. In case BM is a carbon atom, the carbon atom may be part of a larger functional group, such as an acetal, a ketal, a hemiketal, an orthoester, an orthocarbonate ester, an amino acid and the like. Preferred amino acids in this respect are Asp, Gly, Lys and iGlu. This also holds in case BM is a nitrogen or phosphorus atom, in which case it may be part of an amide, an imide, an imine, a phosphine oxide (as in BM-6) or a phosphotriester.

In a preferred embodiment, BM is a phenyl ring. Most preferably, the phenyl ring is according to structure (BM-7). The substitution pattern of the phenyl ring may be of any regiochemistry, such as 1,2,3-substituted phenyl rings, 1,2,4-substituted phenyl rings, or 1,3,5-substituted phenyl rings. To allow optimal flexibility and conformational freedom, it is preferred that the phenyl ring is according to structure (BM-7), most preferably the phenyl ring is 1,3,5-substituted. The same holds for the pyridine ring of (BM-9).

In the preferred structures for BM defined above, BM typically contains three connection points. The skilled person is capable to determine corresponding BMs with four or five connection points. For example (BM-1) and (BM-7)-(BM-15) would also be suitable as BM with more connection points. Alternatively, the linker may contain more than one BM to create four or five connection points.

In a preferred embodiment, the branching moiety BM is selected from a carbon atom, a nitrogen atom, a phosphorus atom, a (hetero)aromatic ring, a (hetero)cycle or a polycyclic moiety, more preferably a carbon atom or a nitrogen atom.

### Linker L^{B}

Linker L^{B} connects payload D with Ab, via connecting groups Z² and linker L^{A} (in the conjugates according to the invention) or connects payload D with reactive group Q² (in the payload-linker construct). Linkers are known in the art and may be cleavable or non-cleavable. Linker L^{B} preferably is cleavable linker, while linker L^{A} preferably is not cleavable.

Linker L^{B} is connected to one occurrence of Z² (or Q²) and x occurrences of D. Thus, the valency of linker L^{B} is x+1 or “(x+1)-valent”. Herein, “(x+1)-valent” refers to the number of connecting points, being either a payload or reactive group Q² (before reaction) or a connecting group Z² (after reaction). Thus, in case x=1, the linker is connected to one Z²/Q² and one payload D, and the linker has a valency of 1+1=2. Thus, when x=1, the L^{B} is divalent. Thus, in case x=2, the linker is connected to one Z²/Q² and two payloads D, and the linker has a valency of 2+1=3. Thus, when x=2, the L^{B} is trivalent. Thus, in case x=3, the linker is connected to one Z²/Q² and three payloads D, and the linker has a valency of 3+1=4. Thus, when x=3, the L^{B} is tetravalent. Thus, in case x=4, the linker is connected to one Z²/Q² and four payloads D, and the linker has a valency of 4+1=5. Thus, when x=4, the L^{B} is pentavalent.

Linker L^{B} may be referred to as “hetereobifunctional”, which means that is contains two different functionalities, referring to the chemically different connectivities to Z²/Q² and D.

In a preferred embodiment, L^{B} is a heterobifunctional linker of structure

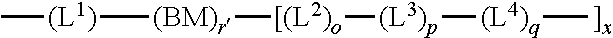

wherein:
L¹ is connected to Q² or Z², and all occurrences of L⁴ are connected to D;
L¹, L², L³ and L⁴ are each individually linkers that together link Q² or Z² to D;
o, p and q are each individually 0 or 1, preferably wherein o=p=1;
r′ is 0 or 1, wherein r′ is 0 when x is 1, or r′1 is 1 when x is 2, 3 or 4;
BM is a branching moiety. The branching moiety is further defined above.

In an alternative preferred embodiment, x=1 and L^{B} is a heterobifunctional divalent linker of structure

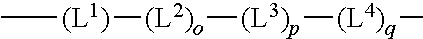

wherein:
L¹ is connected to Q² or Z², and L⁴ is connected to D;
L¹, L², L³ and L⁴ are each individually linkers that together link Q² or Z² to D;
o, p and q are each individually 0 or 1, preferably wherein o=p=1;
BM is a branching moiety. The branching moiety is further defined above.

In a more preferred embodiment, x=2 and L^{B} is a heterobifunctional trivalent linker of structure

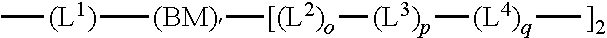

wherein:
L¹ is connected to Q² or Z², and L⁴ is connected to D;
L¹, L², L³ and L⁴ are each individually linkers that together link Q² or Z² to D;
o, p and q are each individually 0 or 1, preferably wherein o=p=1;
BM is a branching moiety. The branching moiety is further defined above.

Alternatively, L^{B} is a heterobifunctional linker of structure

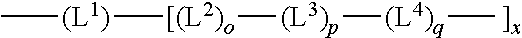

wherein BM is embedded with linker L¹ in case x=2, 3 or 4.

In the context of the present invention, a payload-linker construct of structure Q²(L^{B})Dₓ reacts with an antibody-linker construct of structure Ab(Z¹L^{A}(F²)_{y})_{z}, thereby performing z×y click reactions between F² and Q² per antibody Ab, and thus forming z×y connecting groups Z² per antibody Ab. Upon completion of the reaction, an antibody-conjugate is formed containing z×y×x payloads D per antibody. The antibody-conjugate thus formed has a structure Ab(Z¹L^{A}(Z²(L^{B})Dₓ)_{y})_{z}.

L¹, L², L³ and L⁴ are linkers or linking units and each of o, p and q are individually 0 or 1, preferably wherein o=p=1. In a preferred embodiment, at least linkers L¹ and L² are present (i.e. o=1; p=0 or 1; q=0 or 1), more preferably linkers L¹, L² and L³ are present (i.e. o=1; p=1; q=0 or 1). In one embodiment, L¹, L², L³ and L⁴ each individually consist of one or more building blocks selected from (hetero)aryl, CH₂, CH═CH, C≡C, C(O), NR¹³, O, S, S(O), S(O)₂, PR¹³ and P(O)R¹³, preferably the building blocks are selected from aryl, CH₂, CH═CH, C(O), NR¹³, O, S, S(O) and S(O)₂. Preferred embodiments for each of L¹, L², L³ and L⁴ are provided below.

A linker, especially linker L¹, may contain one or more branch-points for attachment of multiple payloads to a single connecting group. In a preferred embodiment, the linker of the conjugate according to the invention contains a branching moiety. A “branching moiety” in the context of the present invention refers to a moiety that is embedded in a linker connecting three moieties. In other words, the branching moiety comprises at least three bonds to other moieties, typically one bond connecting to Z¹ or Q, one bond to the payload D and one bond to a second payload D. The branching moiety, if present, is preferably embedded in linker L¹, more preferably part of Sp³ or as the nitrogen atom of NR¹³. Any moiety that contains at least three bonds to other moieties is suitable as branching moiety in the context of the present invention. In a preferred embodiment, the branching moiety is selected from a carbon atom, a nitrogen atom, a phosphorus atom, a (hetero)aromatic ring, a (hetero)cycle or a polycyclic moiety. Most preferably, the branching moiety is a nitrogen atom.

### Linker L¹

L¹ may for example be selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀₀ arylalkylene groups, C₈-C₂₀₀ arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)_{y′} and NR²¹, wherein y′ is 0, 1 or 2, preferably y′=2, and R²¹ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups. In one embodiment, the optional substituents may be selected from polar groups, such as oxo groups, (poly)ethylene glycol diamines, (poly)ethylene glycol or (poly)ethylene oxide chains, (poly)propylene glycol or (poly)propylene oxide chains, carboxylic acid groups, carbonate groups, carbamate groups, cyclodextrins, crown ethers, saccharides (e.g. monosaccharides, oligosaccharides), phosphates or esters thereof, phosphonic acid or ester, phosphinic acid or ester, sulfoxides, sulfones, sulfonic acid or ester, sulfinic acid, or sulfenic acid.

In a preferred embodiment, linker L¹ contains a polar group, which may also be present in the chain of L¹. Such a polar group may be selected from (poly)ethylene glycol diamines (e.g. 1,8-diamino-3,6-dioxaoctane or equivalents comprising longer ethylene glycol chains), (poly)ethylene glycol or (poly)ethylene oxide chains, (poly)propylene glycol or (poly)propylene oxide chains and 1,x′-diaminoalkanes (wherein x′ is the number of carbon atoms in the alkane, preferably x′=1-10), —(O)ₐ—C(O)—NH—S(O)₂—NR¹³— (as further defined below, see structure (23)), —C(S(O)₃⁽⁻⁾)—, —C(C(O)₂⁽⁻⁾)—, —S(O)₂—, —P(O)₂⁽⁻⁾, —O(CH₂CH₂O)ₜ—, —NR³⁰(CH₂CH₂NR³⁰)ₜ—, and the following two structures:

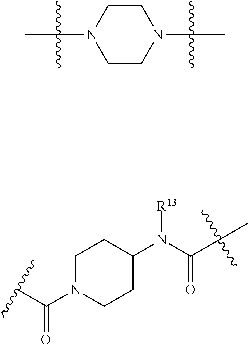

For the polar groups defined here above, it is irrelevant which end is connected to Z¹ and which end to (L²)ₒ.

The polar group may also contain an amino acid, preferably selected from Arg, Glu, Asp, Ser and Thr. Herein, R¹³ is further defined below for structure (23). t is an integer in the range of integer in the range of 0-15, preferably 1-10, more preferably 2-5, most preferably t=2 or 4. Each R³⁰ is individually H, C₁₋₁₂ alkyl, C₁₋₁₂ aryl, C₁₋₁₂ alkaryl or C₁₋₁₂ aralkyl. Linker L¹ may contain more than one such polar group, such as at least two polar groups. The polar group may also be present in a branch of linker L¹, which branches off a branching moiety as defined elsewhere. In the context of L¹, a nitrogen or carbon atom is preferably used as branching moiety. It is especially preferred to have a —O(CH₂CH₂O)ₜ— polar group present in a branch.

In a preferred embodiment, Linker L¹ is or comprises a sulfamide group, preferably a sulfamide group according to structure (23):

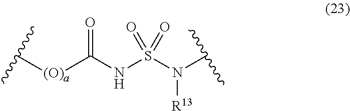

The wavy lines represent the connection to the remainder of the compound, typically to Q² or Z² and to L², L³, L⁴ or D. Preferably, the (O)ₐC(O) moiety is connected to Q² or Z² and the NR¹³ moiety to L², L³, L⁴ or D, preferably to L².

In structure (23), a=0 or 1, preferably a=1, and R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups. Alternatively, R¹³ is D connected to N optionally via a spacer moiety, preferably via Sp² as defined below, in one embodiment D is connected to N via —(B)ₑ-(A)_{f}-(B)_{g}—C(O)—. Alternatively, R¹³ is connected to elsewhere in the linker, optionally via a spacer moiety, to form a cyclic structure. For example, R¹³ may be connected to the linker via a CH₂CH₂ spacer moiety to form a piperazinyl ring, where the connection to D is via the second nitrogen of the piperazinyl ring.

In a preferred embodiment, R¹³ is hydrogen, a C₁-C₂₀ alkyl group, preferably a C₁-C₁₆ alkyl group, more preferably a C₁-C₁₀ alkyl group, or connected to a further occurrence of D or to elsewhere in the linker optionally via a spacer moiety. Herein, the alkyl group is optionally substituted and optionally interrupted by one or more heteroatoms selected from O, S and NR¹⁴, preferably O, wherein R¹⁴ is independently selected from the group consisting of hydrogen and C₁-C₄ alkyl groups. In another preferred embodiment, R¹³ is a C₁-C₂₀ alkyl group, more preferably a C₁-C₁₆ alkyl group, even more preferably a C₁-C₁₀ alkyl group, wherein the alkyl group is optionally interrupted by one or more O-atoms, and wherein the alkyl group is optionally substituted with an —OH group, preferably a terminal —OH group. In this embodiment it is further preferred that R¹³ is a (poly)ethylene glycol chain comprising a terminal —OH group. In another preferred embodiment, R¹³ is selected from the group consisting of hydrogen, methyl, ethyl, n-propyl, i-propyl, n-butyl, s-butyl and t-butyl, or connected to a further occurrence of D or to elsewhere in the linker optionally via a spacer moiety, more preferably from the group consisting of hydrogen, methyl, ethyl, n-propyl and i-propyl, or connected to a further occurrence of D or to elsewhere in the linker optionally via a spacer moiety, and even more preferably from the group consisting of hydrogen, methyl and ethyl, or connected to a further occurrence of D or to elsewhere in the linker optionally via a spacer moiety. Yet even more preferably, R¹³ is hydrogen or connected to a further occurrence of D or to elsewhere in the linker optionally via a spacer moiety, and most preferably R¹³ is hydrogen.

In a preferred embodiment, L¹ is according to structure (24):

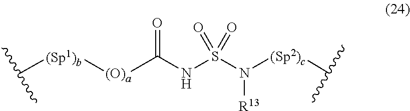

Herein, a and R¹³ are as defined above, Sp¹ and Sp² are independently spacer moieties and b and c are independently 0 or 1. Preferably, b=0 or 1 and c=1, more preferably b=0 and c=1. In one embodiment, spacers Sp¹ and Sp² are independently selected from the group consisting of linear or branched C₁-C₂₀₀ alkylene groups, C₂-C₂₀₀ alkenylene groups, C₂-C₂₀₀ alkynylene groups, C₃-C₂₀₀ cycloalkylene groups, C₅-C₂₀₀ cycloalkenylene groups, C₈-C200 cycloalkynylene groups, C₇-C₂₀₀ alkylarylene groups, C₇-C₂₀₀ arylalkylene groups, C₈-C₂₀₀ arylalkenylene groups and C₉-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

More preferably, spacer moieties Sp¹ and Sp², if present, are independently selected from the group consisting of linear or branched C₁-C₁₀₀ alkylene groups, C₂-C₁₀₀ alkenylene groups, C₂-C₁₀₀ alkynylene groups, C₃-C₁₀₀ cycloalkylene groups, C₅-C₁₀₀ cycloalkenylene groups, C₈-C₁₀₀ cycloalkynylene groups, C₇-C₁₀₀ alkylarylene groups, C₇-C₁₀₀ arylalkylene groups, C₈-C₁₀₀ arylalkenylene groups and C₉-C₁₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Even more preferably, spacer moieties Sp¹ and Sp², if present, are independently selected from the group consisting of linear or branched C₁-C₅₀ alkylene groups, C₂-C₅₀ alkenylene groups, C₂-C₅₀ alkynylene groups, C₃-C₅₀ cycloalkylene groups, C₅-C₅₀ cycloalkenylene groups, C₈-C₅₀ cycloalkynylene groups, C₇-C₅₀ alkylarylene groups, C₇-C₅₀ arylalkylene groups, C₈-C₅₀ arylalkenylene groups and C₉-C₅₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Yet even more preferably, spacer moieties Sp¹ and Sp², if present, are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

In these preferred embodiments it is further preferred that the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are unsubstituted and optionally interrupted by one or more heteroatoms selected from the group of O, S and NR¹⁶, preferably O, wherein R¹⁶ is independently selected from the group consisting of hydrogen and C₁-C₄ alkyl groups, preferably hydrogen or methyl.

Most preferably, spacer moieties Sp¹ and Sp², if present, are independently selected from the group consisting of linear or branched C₁-C₂₀ alkylene groups, the alkylene groups being optionally substituted and optionally interrupted by one or more heteroatoms selected from the group of O, S and NR¹⁶, wherein R¹⁶ is independently selected from the group consisting of hydrogen, C₁-C₂₄ alkyl groups, C₂-C₂₄ alkenyl groups, C₂-C₂₄ alkynyl groups and C₃-C₂₄cycloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR¹⁶, preferably O and/or S—S, wherein R¹⁶ is independently selected from the group consisting of hydrogen and C₁-C₄ alkyl groups, preferably hydrogen or methyl.

Preferred spacer moieties Sp¹ and Sp² thus include —(CH₂)ᵣ—, —(CH₂CH₂)ᵣ—, —(CH₂CH₂O)ᵣ—, —(OCH₂CH₂)ᵣ—, —(CH₂CH₂O)CH₂CH₂—, —CH₂CH₂(OCH₂CH₂)ᵣ—, —(CH₂CH₂CH₂O)ᵣ—, —(OCH₂CH₂CH₂)ᵣ—, —(CH₂CH₂CH₂O)CH₂CH₂CH₂— and —CH₂CH₂CH₂(OCH₂CH₂CH₂)ᵣ—, wherein r is an integer in the range of 1 to 50, preferably in the range of 1 to 40, more preferably in the range of 1 to 30, even more preferably in the range of 1 to 20 and yet even more preferably in the range of 1 to 15. More preferably n is 1, 2, 3, 4, 5, 6, 7, 8, 9 or 10, more preferably 1, 2, 3, 4, 5, 6, 7 or 8, even more preferably 1, 2, 3, 4, 5 or 6, yet even more preferably 1, 2, 3 or 4.

Alternatively, preferred linkers L¹ may be represented by —(W)ₖ-(A)_{d}-(B)ₑ-(A)_{f}-(C(O))_{g}—, wherein:
d=0 or 1, preferably d=1;
e=an integer in the range 0-10, preferably e=0, 1, 2, 3, 4, 5 or 6, preferably an integer in the range 1-10, most preferably e=1, 2, 3 or 4;
f=0 or 1, preferably f=0;
wherein d+e+f is at least 1, preferably in the range 1-5; and preferably wherein d+f is at least 1, preferably d+f=1.
g=0 or 1, preferably g=1;
k=0 or 1, preferably k=1;
A is a sulfamide group according to structure (23);
B is a —CH₂—CH₂—O— or a —O—CH₂—CH₂— moiety, or (B)ₑ is a —(CH₂—CH₂—O)ₑ₁—CH₂—CH₂— or a —(CH₂—CH₂—O)ₑ₁—CH₂— moiety, wherein e1 is defined the same way as e;
W is —OC(O)—, —C(O)O—, —C(O)NH—, —NHC(O)—, —OC(O)NH—, —NHC(O)O—, —C(O)(CH₂)ₘC(O)—, —C(O)(CH₂)ₘC(O)NH— or -(4-Ph)CH₂NHC(O)(CH₂)ₘC(O)NH—, preferably wherein W is —OC(O)NH—, —C(O)(CH₂)ₘC(O)NH— or —C(O)NH—, and wherein m is an integer in the range 0-10, preferably m=0, 1, 2, 3, 4, 5 or 6, most preferably m=2 or 3;
preferably wherein L¹ is connected to Q via (W)ₖ and to L², L³ or D, preferably to L², via (C(O))_{g}, preferably via C(O).

In the context of the present embodiment, the wavy lines in structure (23) represent the connection to the adjacent groups such as (W)ₖ, (B)ₑ and (C(O))_{g}. It is preferred that A is according to structure (23), wherein a=1 and R¹³=H or a C₁-C₂₀ alkyl group, more preferably R¹³=H or methyl, most preferably R¹³=H.

Preferred linkers L¹ have structure —(W)ₖ-(A)_{d}-(B)ₑ-(A)_{f}-(C(O))_{g}—, wherein:
(a) k=0; d=1; g=1; f=0; B=—CH₂—CH₂—O—; e=1, 2, 3 or 4, preferably e=2.
(b) k=1; W=—C(O)(CH₂)ₘC(O)NH—; m≤2; d=0; (B)ₑ=—(CH₂—CH₂—O)ₑ₁—CH₂—CH₂—; f=0; g=1; e1=1, 2, 3 or 4, preferably e=1.
(c) k=1; W=—OC(O)NH—; d=0; B=—CH₂—CH₂—O—; g=1; f=0; e=1, 2, 3 or 4, preferably e=2.
(d) k=1; W=—C(O)(CH₂)ₘC(O)NH—; m=2; d=0; (B)ₑ=—(CH₂—CH₂—O)ₑ₁—CH₂—CH₂—; f=0; g=1; e1=1, 2, 3 or 4, preferably e1=4.
(e) k=1; W=—OC(O)NH—; d=0; (B)ₑ=—(CH₂—CH₂—O)^{e1}—CH₂—CH₂—; g=1; f=0; e1=1, 2, 3 or 4, preferably e1=4.
(f) k=1; W=-(4-Ph)CH₂NHC(O)(CH₂)ₘC(O)NH—, m=3; d=0; (B)ₑ=—(CH₂—CH₂—O)ₑ₁—CH₂—CH₂—; g=1; f=0; e1=1, 2, 3 or 4, preferably e1=4.
(g) k=0; d=0; g=1; f=0; B=—CH₂—CH₂—O—; e=1, 2, 3 or 4, preferably e=2.
(h) k=1; W=—C(O)NH—; d=0; g=1; f=0; B=—CH₂—CH₂—O—; e=1, 2, 3 or 4, preferably e=2.

Herein, it is preferred that when d and/or f=1, than a=1 and R¹³=H. Most preferred is the linker is structure (a).

In a preferred embodiment, linker L¹ comprises a branching nitrogen atom, which is located in the backbone between Q or Z and (L²)ₒ and which contains a further moiety D as substituent, which is preferably linked to the branching nitrogen atom via a linker. An example of a branching nitrogen atom is the nitrogen atom NR¹³ in structure (23), wherein R¹³ is connected to a second occurrence of D via a spacer moiety. Alternatively, a branching nitrogen atoms may be located within L¹ according to structure —(W)ₖ-(A)_{d}-(B)ₑ-(A)_{f}-(C(O))_{g}—. In one embodiment, L¹ is represented by —(W)ₖ-(A)_{d}-(B)ₑ-(A)_{f}-C(O))_{g}-BM[-(A)ₑ-(B)ₑ-(A)_{f}-(C(O))_{g}-]₂, wherein A, B, W, d, e, f, g and k are as defined above and individually selected for each occurrence, and BM is a branching moiety, preferably a branching nitrogen atom, to which two instances of -(A)_{d}-(B)ₑ-(A)_{f}-(C(O))_{g}— are connected. Herein, both (C(O))_{g} moieties are connected to -(L²)ₒ-(L³)ₚ-(L⁴)_{q}-D, wherein L², L³, L⁴, o, p, q and D are as defined above and are each selected individually. In a preferred embodiment, each of L², L³, L⁴, o, p, q and D are the same for both moieties connected to (C(O))_{g}.

Preferred linkers L¹ comprising a branching nitrogen atom have structure —(W)ₖ-(A)_{d}-(B)ₑ-(A)_{f}-(C(O))_{g}—N[-(A′)_{d′}—(B′)_{e′}-(A′)_{f′}—(C(O))_{g′}-]₂ wherein:
(i) k=d=g=e′=1; f=d′=g′=0; W=—C(O)—; B=B′=—CH₂—CH₂—O—; A is according to structure (23) with a=0 and R¹³=H; e=1, 2, 3 or 4, preferably e=2.
(j) k=d=g=e′=g′=1; f=d′=0; W=—C(O)—; B=B′=—CH₂—CH₂—O—; A is according to structure (23) with a=0 and R¹³=H; e=1, 2, 3 or 4, preferably e=2.

### Linker L²

Linker L² is a peptide spacer. Linker L² is either absent (o=0) or present (o=1). Preferably, linker L² is present and o=1. The combination of a peptide spacer L² and a cleavable linker L³ is well-known in the art. Linker L² functions as recognition and cleave site for cleaving-enzymes, more preferably the peptides are recognized by specific cleaving enzymes. This allows for cleavage at specific environments in which these cleaving enzymes are expressed such as specific tumors. Since different peptide sequences are cleaved by different enzymes, the L² group also allows customizing the conjugate for specific treatments. The peptide sequences may be cleaved by intracellular enzymes and/or extracellular enzymes.

The peptide spacer may also be defined by (NH—CR¹⁷—CO)ₙ, wherein R¹⁷ represents an amino acid side chain as known in the art. Also covered within this definition is proline, which has R¹⁷ joined with the nitrogen atom to form a cyclic moiety. Herein, the amino acid may be a natural or a synthetic amino acid. Preferably, the amino acid(s) are all in their L-configuration. n is an integer in the range of 1-5, preferably in the range of 2-4. Thus, the peptide spacer contains 1-5 amino acids. Preferably, the peptide is a dipeptide (n=2), tripeptide (n=3) or tetrapeptide (n=4), most preferably the peptide spacer is a dipeptide. R¹⁷ represents the amino acid side chain, preferably selected from the side chains of alanine, cysteine, aspartic acid, glutamic acid, phenylalanine, glycine, histidine, isoleucine, lysine, acetyllysine, leucine, methionine, asparagine, pyrrolysine, proline, glutamine, arginine, serine, threonine, selenocysteine, valine, tryptophan, tyrosine and citrulline. Preferred amino acid side chains are those of Val, Cit, Ala, Lys, Arg, AcLys, Phe, Leu, lie, Trp, Glu, Asp and Asn, more preferably from the side chains of Val, Cit, Ala, Glu and Lys. Alternatively worded, R¹⁷ is preferably selected from CH₃ (Ala), CH₂CH(CH₃)₂ (Leu), CH₂CH₂CH₂NHC(O)NH₂ (Cit), CH₂CH₂CH₂CH₂NH₂ (Lys), CH₂CH₂CH₂NHC(O)CH₃ (AcLys), CH₂CH₂CH₂NHC(═NH)NH₂ (Arg), CH₂Ph (Phe), CH(CH₃)₂ (Val), CH(CH₃)CH₂CH₃ (Ile), CH₂C(O)NH₂ (Asn), CH₂CH₂C(O)OH (Glu), CH₂C(O)OH (Asp) and CH₂(1H-indol-3-yl) (Trp). Especially preferred embodiments of R¹⁷ are CH₃ (Ala), CH₂CH₂CH₂NHC(O)NH₂ (Cit), CH₂CH₂CH₂CH₂NH₂ (Lys), CH₂CH₂C(O)OH (Glu) and CH(CH₃)₂ (Val). Most preferably, R¹⁷ is CH₃ (Ala), CH₂CH₂CH₂NHC(O)NH₂ (Cit), CH₂CH₂CH₂CH₂NH₂ (Lys), or CH(CH₃)₂ (Val).

Although any peptide spacer may be used, preferably the peptide spacer is selected from Val-Cit, Val-Ala, Val-Lys, Val-Arg, AcLys-Val-Cit, AcLys-Val-Ala, Glu-Val-Ala, Asp-Val-Ala, iGlu-Val-Ala, Glu-Val-Cit, Glu-Gly-Cit, Glu-Gly-Val, Asp-Val-Cit, iGlu-Val-Cit, Phe-Cit, Phe-Ala, Phe-Lys, Phe-Arg, Ala-Lys, Leu-Cit, Ile-Cit, Trp-Cit, Asn-Asn, Ala-Ala-Asn, Ala-Asn, Asn-Ala, Phe-Phe, Gly, Gly-Gly, Gly-Gly-Gly, Gly-Gly-Gly-Gly, Leu-Gly, Tyr-Gly, Ala-Gly, Pro-Gly, Phe-Gly, Phe-Gly, Ser-Gly, Gly-Phe-Gly, Gly-Gly-Phe-Gly, Gly-Phe-Gly-Gly, Phe-Gly-Gly-Gly, Gly-Gly-Gly-Phe, Phe-Phe-Gly-Gly, Gly-Gly-Phe-Phe, Gly-Gly-Gly-Phe-Gly and Lys, more preferably Val-Cit, Val-Ala, Glu-Val-Ala, Val-Lys, Phe-Cit, Phe-Ala, Phe-Lys, Ala-Ala-Asn, more preferably Glu-Val-Ala, Glu-Gly-Cit, Val-Cit, Val-Ala, Asn-Asn, Ala-Ala-Asn, Asn-Ala most preferably Glu-Val-Ala, Glu-Gly-Cit, Val-Cit, Val-Ala or Asn-Ala. Herein, AcLys is e-N-acetyllysine and iGlu is isoglutamate. In one embodiment, L²=Val-Cit. In another embodiment, L²=Val-Ala. In another embodiment, L²=Asn-Ala. In another embodiment, L²=Glu-Gly-Cit. In another embodiment, L²=Glu-Val-Ala.

In one embodiment, the amino acid side chain R¹⁷ is substituted with a polar group, preferably selected from oxo groups, (poly)ethylene glycol diamines, (poly)ethylene glycol or (poly)ethylene oxide chains, (poly)propylene glycol or (poly)propylene oxide chains, carboxylic acid groups, carbonate groups, carbamate groups, cyclodextrins, crown ethers, saccharides (e.g. monosaccharides, oligosaccharides), phosphates or esters thereof, phosphonic acid or ester, phosphinic acid or ester, sulfoxides, sulfones, sulfonic acid or ester, sulfinic acid, or sulfenic acid.

In an especially preferred embodiment, L² comprises the peptide spacer represented by general structure (25), preferably L² is represented by general structure (25):

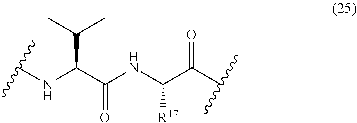

Herein, R¹⁷ is as defined above, preferably R¹⁷ is CH₃ (Ala) or CH₂CH₂CH₂NHC(O)NH₂ (Cit). The wavy lines indicate the connection to (L¹)ₙ and (L³)ₚ, preferably L² according to structure (25) is connected to (L¹)ₙ via NH and to (L³)ₚ via C(O).

### Linker L³

Linker L³ is a self-cleavable spacer, also referred to as self-immolative spacer. Linker L³ is either absent (p=0) or present (p=1). Preferably, linker L³ is present and p=1. Cleavage of L² results in 1,6-β elimination in linker L³, resulting in decarboxylation and the release of the payload, D. This advantageously allows for increased probability of release of the payload in regions wherein enzymes that are able to cleave L² are overexpressed. In addition, release of a payload can induce bystander killing which is advantageous for tumours in which not all cancer cells have overexpression of the targeted receptor. Hence, in this embodiment, it is preferred that both linker L² and L³ are present (o=p=1).

Preferably, L³ is para-aminobenzyloxycarbonyl (PABC) derivative, more preferably a PABC derivative according to structure (26):

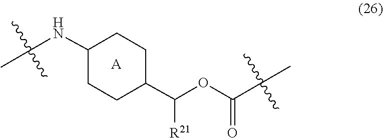

Herein, the wavy lines indicate the connection to L¹ or L², and to L⁴ or D. Typically, the PABC derivative is connected via NH to L¹ or L², preferably to L², and via OC(O) to L⁴ or D.

Ring A is a 5- or 6-membered aromatic or heteroaromatic ring, preferably a 6-membered aromatic or heteroaromatic ring. Suitable 5-membered rings are oxazole, thiazole and furan. Suitable 6-membered rings are phenyl and pyridyl. Ring A may be substituted with a substituent selected from halogen, X²R⁴, N(R⁴)₂, C₁₋₄ alkyl and NO₂. Herein, X² and R⁴ are as defined above, including preferred embodiments thereof. In a preferred embodiment, the optional substituent is selected from F, Cl, Br, OH, OR⁴, SH, NH₂, Et, Me and NO₂. In an especially preferred embodiment, ring A comprises 0-2 substituents, more preferably 0 or 1 substituent, most preferably ring A is not substituted. In a preferred embodiment, ring A is 1,4-phenyl, 1,2-phenyl, 2,5-pyridyl or 3,6-pyridyl. Most preferably, A is 1,4-phenyl.

R²¹ is selected from H, R²⁶, C(O)OH and C(O)R²⁶, wherein R²⁶ is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which are optionally substituted and optionally interrupted by one or more heteroatoms selected from O, S and NR²⁸ wherein R²⁸ is independently selected from the group consisting of hydrogen and C₁-C₄ alkyl groups. Preferably, R²⁶ is C₃-C₁₀ (hetero)cycloalkyl or polyalkylene glycol. The polyalkylene glycol is preferably a polyethylene glycol or a polypropylene glycol, more preferably —(CH₂CH₂O)ₛH or —(CH₂CH₂CH₂O)ₛH. The polyalkylene glycol is most preferably a polyethylene glycol, preferably —(CH₂CH₂O)ₛH, wherein s is an integer in the range 1-10, preferably 1-5, most preferably s=1, 2, 3 or 4. More preferably, R²¹ is H or C(O)R²⁶, wherein R²⁶=4-methyl-piperazine or morpholine. Most preferably, R²¹ is H.

In an alternative embodiment, linker L³ is present (p=1) and linker L² is not (o=0) and L³ is a para-glucuronide-meta-amide-benzyloxycarbonyl derivative, preferably a glucuronide derivative according to structure (27):

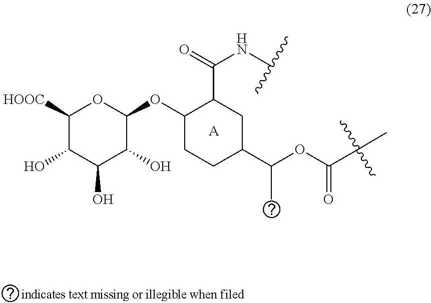

Herein, the wavy lines indicate the connection to L¹, and to L⁴ or D. Typically, the glucuronide derivative is connected via NH to L¹, and via (O)CO to L⁴ or D. Ring A and R²¹ are defined as for the PABC derivative according to structure (26). Preferably, ring A a 6-membered aromatic or heteroaromatic ring, such as oxazole, thiazole, furan, phenyl and pyridyl. In a preferred embodiment, ring A is 1,3,4-phenyl, 2,4,5-pyridyl or 2,5,6-pyridyl. Most preferably, A is 1,3,4-phenyl. Preferably, R²¹ is H or C(O)R²⁶, wherein R²⁶=4-methyl-piperazine or morpholine. Most preferably, R²¹ is H.

Linker L³ according to structure (27) is cleavable by β-glucuronidase, similar to the mechanism in PABC, which results in self-immolation of the para-hydroxybenzyloxy group, decarboxylation and the release of the payload. An ADC comprising the glucuronide derivative according to structure (27) is especially useful for treating cancers having an overexpression of β-glucuronidase. β Glucuronidase concentrations in many solid tumours, including lung, breast, and gastrointestinal cancers, as well as in the tumour microenvironment, are reported to be higher than those in normal tissues, and the enzyme is not found in the general circulation. Thus, it is preferred that the conjugates according to the invention comprising the glucuronide derivative according to structure (27) are used to treat patients suffering from lung, breast, and gastrointestinal cancers.

### Linker L⁴

Linker L⁴ is either absent (q=0) or present (q=1). Preferably, linker L⁴ is present and q=1. Linker L⁴ is selected from:
an aminoalkanoic acid spacer according to the structure —NR²²—(Cₓ-alkylene)-C(O)—, wherein x is an integer in the range 1-20 and R²² is H or C₁-C₄ alkyl;
an ethyleneglycol spacer according to the structure —NR²²—(CH₂—CH₂—O)ₑ₆—(CH₂)ₑ₇—C(O)—, wherein e6 is an integer in the range 1-10, e7 is an integer in the range 1-3 and R² is H or C₁-C₄ alky; and
an diamine spacer according to the structure —NR²²—(Cₓ-alkylene)-NR²²—(C(O))ₕ—, wherein h is 0 or 1, x is an integer in the range 1-20 and R²² is H or C₁-C₄ alkyl.

Linker L⁴ may be an aminoalkanoic acid spacer, i.e. —NR²²—(Cₓ-alkylene)-C(O)—, wherein x is an integer in the range 1 to 20, preferably 1-10, most preferably 1-6. Herein, the aminoalkanoic acid spacer is typically connected to L³ via the nitrogen atom and to D via the carbonyl moiety. Preferred linkers L⁴ are selected from 6-aminohexanoic acid (Ahx, x=5), β-alanine (x=2) and glycine (Gly, x=1), even more preferably 6-aminohexanoic acid or glycine. In one embodiment, L⁴=6-aminohexanoic acid. In one embodiment, L⁴=glycine. Herein, R²² is H or C₁-C₄ alkyl, preferably R²² is H or methyl, most preferably R²² is H.

Alternatively, linker L⁴ may be an ethyleneglycol spacer according to the structure —NR²²—(CH₂—CH₂—O)ₑ₆—(CH₂)ₑ₇—(C(O)—, wherein e6 is an integer in the range 1-10, preferably e6 is in the range 2-6, and e7 is an integer in the range 1-3, preferably e7 is 2. Herein, R²² is H or C₁-C₄ alkyl, preferably R²² is H or methyl, most preferably R²² is H.

Alternatively, linker L⁴ may be a diamine spacer according to the structure —NR²²—(Cₓ-alkylene)-NR²²—(C(O))ₕ—, wherein h is 0 or 1, x is an integer in the range 1-20, preferably an integer in the range 2-6, even more preferably x=2 or 5, most preferably x=2. R²² is H or C₁-C₄ alkyl. Herein, R²² is H or C₁-C₄ alkyl, preferably R²² is H or methyl, most preferably R²² is methyl. Herein, h is preferably 1, in which case linker L⁴ is especially suited for conjugation via a phenolic hydroxyl group present on payload D.

### Payload D

D, also referred to in the art as the “payload”, represents the compound that is or is to be connected to antibody Ab. Payload molecules are well-known in the art, especially in the field of antibody-drug conjugates, as the moiety that is covalently attached to the antibody and that is released therefrom upon uptake of the conjugate and/or cleavage of the linker. In a preferred embodiment, the payload is selected from the group consisting of an active substance, a reporter molecule, a polymer, a solid surface, a hydrogel, a nanoparticle, a microparticle and a biomolecule. Especially preferred payloads are active substances and reporter molecules, in particular active substances.

The term “active substance” herein relates to a pharmacological and/or biological substance, i.e. a substance that is biologically and/or pharmaceutically active, for example a drug, a prodrug, a cytotoxin, a diagnostic agent, a protein, a peptide, a polypeptide, a peptide tag, an amino acid, a glycan, a lipid, a vitamin, a steroid, a nucleotide, a nucleoside, a polynucleotide, RNA or DNA. Examples of peptide tags include cell-penetrating peptides like human lactoferrin or polyarginine. An example of a glycan is oligomannose. An example of an amino acid is lysine.

When the payload is an active substance, the active substance is preferably selected from the group consisting of drugs and prodrugs. More preferably, the active substance is selected from the group consisting of pharmaceutically active compounds, in particular low to medium molecular weight compounds (e.g. about 200 to about 2500 Da, preferably about 300 to about 1750 Da). In a further preferred embodiment, the active substance is selected from the group consisting of cytotoxins, antiviral agents, antibacterial agents, peptides and oligonucleotides.

Preferred cytotoxins are selected from the groups of nitrogen mustards, nitrosoureas, alkylsulphonates, triazenes, platinum containing compounds, plant alkaloids, DNA topoisomerase inhibitors, anti-metabolites, hormonal therapies, kinase inhibitors, antibiotics, and further cytotoxins defined here below.

Suitable nitrogen mustards include chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 (including its adozelesin, carzelesin and bizelesin synthetic analogues); duocarmycin (including the synthetic analogues, KW-2189 and CBI-TMI); benzodiazepine monomers or dimers (e.g., pyrrolobenzodiazepine (PBD), tomaymycin, indolinobenzodiazepine, isoindolinebenzodiazepine, imidazobenzothiadiazepine and oxazolidinobenzodiazepine).

Suitable nitrosoureas include carmustine, lomustine, chlorozotocin, fotemustine, nimustine and ranimustine.

Suitable alkylsulphonates include busulfan, treosulfan, improsulfan and piposulfan.

Suitable triazenes include dacarbazine.

Suitable platinum containing compounds include carboplatin, cisplatin, oxaliplatin; aziridines, such as benzodopa, carboquone, meturedopa, and uredopa; ethylenimines and methylamelamines including altretamine, triethylenemelamine, trietylenephosphoramide, triethylenethiophosphaoramide and trimethylolomelamine.

Suitable plant alkaloids include vinca alkaloids (e.g. vincristine, vinblastine, vindesine, vinorelbine, navelbin), toxoids (e.g. paclitaxel, docetaxel and their analogs), maytansinoids and their analogs (e.g. DM1, DM2, DM3, DM4, maytansine and ansamitocins), cryptophycins (particularly cryptophycin 1 and cryptophycin 8), epothilones, eleutherobin, discodermolide, bryostatins, dolostatins, auristatins, tubulysins, cephalostatins, pancratistatin, sarcodictyin and spongistatin.

Suitable DNA topoisomerase inhibitors include any camptothecin, including 9-aminocamptothecin, exatecan, DXd (DX-8951 derivative), crisnatol, daunomycin, etoposide, etoposide phosphate, irinotecan, mitoxantrone, novantrone, retinoic acids (retinols), teniposide, topotecan, 9-nitrocamptothecin (RFS 2000); mitomycins and mitomycin C.

Suitable anti-metabolites include anti-folate, DHFR inhibitors (e.g. methotrexate, trimetrexate, denopterin, pteropterin, aminopterin (4-aminopteroic acid) or other folic acid analogues), IMP dehydrogenase inhibitors (e.g. mycophenolic acid, tiazofurin, ribavirin, EICAR), ribonucleotide reductase inhibitors (e.g. hydroxyurea, deferoxamine), pyrimidine analogs (e.g. uracil analogs such as ancitabine, azacitidine, 6-azauridine, capecitabine (Xeloda), carmofur, cytarabine, dideoxyuridine, doxifluridine, enocitabine, 5-fluorouracil, floxuridine, ratitrexed (Tomudex); cytosine analogs such as cytarabine, cytosine arabinoside, fludarabine; purine analogs such as azathioprine, fludarabine, mercaptopurine, thiamiprine, thioguanine), folic acid replenisher (e.g. frolinic acid).

Suitable hormonal therapies include receptor antagonists (e.g. anti-estrogen such as megestrol, raloxifene, tamoxifen, LHRH agonists such as goscrclin, leuprolide acetate; antiandrogens such as bicalutamide, flutamide, calusterone, dromostanolone propionate, epitiostanol, goserelin, leuprolide, mepitiostane, nilutamide, testolactone, trilostane and other androgens inhibitors), retinoids/deltoids (e.g. Vitamin D3 analogs such as CB 1093, EB 1089 KH 1060, cholecalciferol, ergocalciferol), photodynamic therapies (e.g. verteporfin, phthalocyanine, photosensitizer Pc4, demethoxy-hypocrellin A).

Suitable kinase inhibitors include BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib and ispinesib.

Suitable antibiotics include the enediyne antibiotics (e.g. calicheamicins, especially calicheamicin .γ1, δ1, α1 and β1), dynemicin (e.g. dynemicin A and deoxydynemicin), esperamicin, kedarcidin, C-1027, maduropeptin, as well as neocarzinostatin chromophore and related chromoprotein enediyne antibiotic chromomophores, aclacinomysins, actinomycin, authramycin, azaserine, bleomycins, cactinomycin, carabicin, carminomycin, carzinophilin; chromomycins, dactinomycin, daunorubicin, nemorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin and zorubicin.

Suitable polyketides include acetogenins (in particular bullatacin and bullatacinone), gemcitabine, epoxomicins (e. g. carfilzomib), bortezomib, thalidomide, lenalidomide, pomalidomide, tosedostat, zybrestat, PLX4032, STA-9090, Stimuvax, allovectin-7, Xegeva and Provenge.

The cytotoxin may further be selected from isoprenylation inhibitors (such as lovastatin), dopaminergic neurotoxins (such as 1-methyl-4-phenylpyridinium ion), cell cycle inhibitors (such as staurosporine), actinomycins (such as actinomycin D and dactinomycin), bleomycins (such as bleomycin A2, bleomycin B2, peplomycin), anthracyclines (e.g. daunorubicin, doxorubicin (adriamycin), idarubicin, epirubicin, pirarubicin, zorubicin, mitoxantrone), MDR inhibitors (e.g. verapamil), Ca2+ ATPase inhibitors (e.g. thapsigargin), histone deacetylase inhibitors (e.g. Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A), thapsigargin, Celecoxib, glitazones, epigallocatechin gallate, disulfiram, salinosporamide A; anti-adrenals (e.g. aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2, 2′,2″-trichlorotriethylamine; trichothecenes (especially T-2 toxin, verrucarin A, roridin A and anguidine).

The term “reporter molecule” herein refers to a molecule whose presence is readily detected, for example a diagnostic agent, a dye, a fluorophore, a radioactive isotope label, a contrast agent, a magnetic resonance imaging agent or a mass label.

A wide variety of fluorophores, also referred to as fluorescent probes, is known to a person skilled in the art. Several fluorophores are described in more detail in e.g. G. T. Hermanson, “Bioconjugate Techniques”, Elsevier, 3^{rd} Ed. 2013, Chapter 10: “Fluorescent probes”, p. 395-463, incorporated by reference. Examples of a fluorophore include all kinds of Alexa Fluor (e.g. Alexa Fluor 555), cyanine dyes (e.g. Cy3 or Cy5) and cyanine dye derivatives, coumarin derivatives, fluorescein and fluorescein derivatives, rhodamine and rhodamine derivatives, boron dipyrromethene derivatives, pyrene derivatives, naphthalimide derivatives, phycobiliprotein derivatives (e.g. allophycocyanin), chromomycin, lanthanide chelates and quantum dot nanocrystals.

Examples of a radioactive isotope label include ^{99m}Tc, ¹¹¹In, ^{114m}In, ¹¹⁵In, ¹⁸F, ¹⁴C, ⁶⁴Cu, ¹³¹I, ¹²⁵I, ¹²³I, ²¹²Bi, ⁸⁸Y, ⁹⁰Y, ⁸⁷Cu, ¹⁸⁶Rh, ¹⁸⁸Rh, ⁶⁶Ga, ⁶⁷Ga and ¹⁰B, which is optionally connected via a chelating moiety such as e.g. DTPA (diethylenetriaminepentaacetic anhydride), DOTA (1,4,7,10-tetraazacyclododecane-N,N′,N″,N′″-tetraacetic acid), NOTA (1,4,7-triazacyclononane N,N′,N″-triacetic acid), TETA (1,4,8,11-tetraazacyclotetradecane-N,N′,N″,N′″-tetraacetic acid), DTTA (N¹-(p-isothiocyanatobenzyl)-diethylenetriamine-N¹,N²,N³,N³-tetraacetic acid), deferoxamine or DFA (N′-[5-[[4-[[5-(acetylhydroxyamino)pentyl]amino]-1,4-dioxobutyl]hydroxyamino]pentyl]-N-(5-aminopentyl)-N-hydroxybutanediamide) or HYNIC (hydrazinonicotinamide). Isotopic labelling techniques are known to a person skilled in the art, and are described in more detail in e.g. G. T. Hermanson, “Bioconjugate Techniques”, Elsevier, 3^{rd} Ed. 2013, Chapter 12: “Isotopic labelling techniques”, p. 507-534, incorporated by reference.

Polymers suitable for use as a payload D in the compound according to the invention are known to a person skilled in the art, and several examples are described in more detail in e.g. G. T. Hermanson, “Bioconjugate Techniques”, Elsevier, 3^{rd} Ed. 2013, Chapter 18: “PEGylation and synthetic polymer modification”, p. 787-838, incorporated by reference. When payload D is a polymer, payload D is preferably independently selected from the group consisting of a poly(ethyleneglycol) (PEG), a polyethylene oxide (PEO), a polypropylene glycol (PPG), a polypropylene oxide (PPO), a 1,q-diaminoalkane polymer (wherein q is the number of carbon atoms in the alkane, and preferably q is an integer in the range of 2 to 200, preferably 2 to 10), a (poly)ethylene glycol diamine (e.g. 1,8-diamino-3,6-dioxaoctane and equivalents comprising longer ethylene glycol chains), a polysaccharide (e.g. dextran), a poly(amino acid) (e.g. a poly(L-lysine)) and a poly(vinyl alcohol).

Solid surfaces suitable for use as a payload D are known to a person skilled in the art. A solid surface is for example a functional surface (e.g. a surface of a nanomaterial, a carbon nanotube, a fullerene or a virus capsid), a metal surface (e.g. a titanium, gold, silver, copper, nickel, tin, rhodium or zinc surface), a metal alloy surface (wherein the alloy is from e.g. aluminum, bismuth, chromium, cobalt, copper, gallium, gold, indium, iron, lead, magnesium, mercury, nickel, potassium, plutonium, rhodium, scandium, silver, sodium, titanium, tin, uranium, zinc and/or zirconium), a polymer surface (wherein the polymer is e.g. polystyrene, polyvinylchloride, polyethylene, polypropylene, poly(dimethylsiloxane) or polymethylmethacrylate, polyacrylamide), a glass surface, a silicone surface, a chromatography support surface (wherein the chromatography support is e.g. a silica support, an agarose support, a cellulose support or an alumina support), etc. When payload D is a solid surface, it is preferred that D is independently selected from the group consisting of a functional surface or a polymer surface.

Hydrogels are known to the person skilled in the art. Hydrogels are water-swollen networks, formed by cross-links between the polymeric constituents. See for example A. S. Hoffman, Adv. Drug Delivery Rev. 2012, 64, 18, incorporated by reference. When the payload is a hydrogel, it is preferred that the hydrogel is composed of poly(ethylene)glycol (PEG) as the polymeric basis.

Micro- and nanoparticles suitable for use as a payload D are known to a person skilled in the art. A variety of suitable micro- and nanoparticles is described in e.g. G. T. Hermanson, “Bioconjugate Techniques”, Elsevier, 3^{rd} Ed. 2013, Chapter 14: “Microparticles and nanoparticles”, p. 549-587, incorporated by reference. The micro- or nanoparticles may be of any shape, e.g. spheres, rods, tubes, cubes, triangles and cones. Preferably, the micro- or nanoparticles are of a spherical shape. The chemical composition of the micro- and nanoparticles may vary. When payload D is a micro- or a nanoparticle, the micro- or nanoparticle is for example a polymeric micro- or nanoparticle, a silica micro- or nanoparticle or a gold micro- or nanoparticle. When the particle is a polymeric micro- or nanoparticle, the polymer is preferably polystyrene or a copolymer of styrene (e.g. a copolymer of styrene and divinylbenzene, butadiene, acrylate and/or vinyltoluene), polymethylmethacrylate (PMMA), polyvinyltoluene, poly(hydroxyethyl methacrylate (pHEMA) or poly(ethylene glycol dimethacrylate/2-hydroxyethylmethacrylate) [poly(EDGMA/HEMA)]. Optionally, the surface of the micro- or nanoparticles is modified, e.g. with detergents, by graft polymerization of secondary polymers or by covalent attachment of another polymer or of spacer moieties, etc.

Payload D may also be a biomolecule. Biomolecules, and preferred embodiments thereof, are described in more detail below. When payload D is a biomolecule, it is preferred that the biomolecule is selected from the group consisting of proteins (including glycoproteins such as antibodies), polypeptides, peptides, glycans, lipids, nucleic acids, oligonucleotides, polysaccharides, oligosaccharides, enzymes, hormones, amino acids and monosaccharides.

In the context of the present invention, cytotoxic payloads are especially preferred. Thus, D is preferably, a cytotoxin, more preferably selected from the group consisting of colchicine, vinca alkaloids, anthracyclines, camptothecins, doxorubicin, daunorubicin, taxanes, calicheamicins, tubulysins, irinotecans, an inhibitory peptide, amanitins, amatoxins, duocarmycins, epothilones, mytomycins, combretastatins, maytansines, auristatins, enediynes, pyrrolobenzodiazepines (PBDs) or indolinobenzodiazepine dimers (IGN). The present invention is especially suitable for cytotoxic payloads with a moderate cytotoxicity. Normally, conjugates of such payloads need to be administered in a higher dose, but in view of the high DAR (high payload loading) of the conjugates of the present invention, even conjugates of moderately toxic payloads can be administered in a desirably low dose. In other words, D is preferably a payload that enables a moderate to high administered dose in patients (3-20 mg/kg) even at high DAR (DAR>>6). In view of the high payload loading in the conjugates of the present invention, it is especially preferred that the payload (in unconjugated form) has an average cytotoxicity (IC₅₀) of >50 nM. As such, even payloads with a lower cytotoxicity can be employed in antibody-conjugates with high efficacy.

### Preferred Linkers L^{A}

Especially preferred linkers L^{A} have the structure -(L¹¹)-BM(L¹²-)(L¹³-), wherein L¹¹ is according to any one of (i)-(x):
(i) L¹¹ is *—C(O)—(CH₂)_{a′}—C(O)—**, wherein the bond labelled * is connected to Q¹ or Z¹ and the bond labelled ** is connected to BM, wherein a′ is an integer in the range 1-5, preferably a′=3. Further, it is preferred that BM is a nitrogen atom (for y=2) or carbon atom (for y=3). Most preferably, BM=N.
(ii) L¹¹ is —NH—C(O)—(CH₂)_{a′}—**, wherein the bond labelled * is connected to Q¹ or Z¹ and the bond labelled ** is connected to BM, wherein a′ is an integer in the range 0-5, preferably a′=0. Further, it is preferred that BM is a nitrogen atom (for y=2) or carbon atom (for y=3). Most preferably, BM=N.
(iii) L¹¹ is *═N—C(O)—(CH₂)_{a′}—**, wherein the double bond labelled * is connected to Q¹ or Z¹ and the bond labelled ** is connected to BM, wherein a′ is an integer in the range 0-5, preferably a′=0. Further, it is preferred that BM is a nitrogen atom (for y=2) or carbon atom (for y=3). Most preferably, BM=N.
(iv) L¹¹ is —NH—C(O)—(CH₂)_{a′}—C(O)—**, wherein the bond labelled * is connected to Q¹ or Z¹ and the bond labelled ** is connected to BM, wherein a′ is an integer in the range 1-5, preferably a′=3. Further, it is preferred that BM is a nitrogen atom (for y=2) or carbon atom (for y=3). Most preferably, BM=N.
(v) L¹¹ is *═N—C(O)—(CH₂)_{a′}—C(O)—**, wherein the double bond labelled * is connected to Q¹ or Z¹ and the bond labelled ** is connected to BM, wherein a′ is an integer in the range 1-5, preferably a′=3. Further, it is preferred that BM is a nitrogen atom (for y=2) or carbon atom (for y=3). Most preferably, BM=N.
(vi) L¹¹ is *—C(O)—**, wherein the bond labelled * is connected to Q¹ or Z¹ and the bond labelled ** is connected to BM, wherein it is preferred that BM is a nitrogen atom (for y=2) or carbon atom (for y=3). Most preferably, BM=N.
(vii) L¹¹ is *-(23)-(CH₂CH₂O)_{a′}—(C(O))_{a″}—**, wherein the bond labelled * is connected to Q¹ or Z¹ and the bond labelled ** is connected to BM, and wherein (23) refers to a group according to structure (23). Further, a′ is an integer in the range 0-5, preferably a′=1-4, most preferably a′=2, and a″ is 0 or 1, preferably a″ is 1. Further, it is preferred that BM is a nitrogen atom (for y=2) or carbon atom (for y=3). Most preferably, BM=N.
(viii) L¹¹ is *-(23)-(CH₂CH₂O)_{a′}-(23)-**, wherein the bond labelled * is connected to Q¹ or Z¹ and the bond labelled ** is connected to BM, and wherein (23) refers to a group according to structure (23). Further, a′ is an integer in the range 0-5, preferably a′=1-4, most preferably a′=2. Further, it is preferred that BM is a nitrogen atom (for y=2) or carbon atom (for y=3). Most preferably, BM=N.
(ix) L¹¹ is *—C(O)—NH—(CH₂CH₂O)_{a′}—C(O)—**, wherein the bond labelled * is connected to Q¹ or Z¹ and the bond labelled ** is connected to BM, wherein a′ is an integer in the range 1-5, preferably a′=3. Further, it is preferred that BM is a nitrogen atom (for y=2) or carbon atom (for y=3). Most preferably, BM=N.
(x) L¹¹ is *—C(O)—NH—(CH₂CH₂O)_{a′}—O—C(O)—NH—, wherein the bond labelled * is connected to Q¹ or Z¹ and the bond labelled ** is connected to BM, wherein a′ is an integer in the range 1-5, preferably a′=3. Further, it is preferred that BM is a nitrogen atom (for y=2) or carbon atom (for y=3). Most preferably, BM=N.

Especially preferred linkers L^{A} have the structure -(L¹¹)-BM(L¹²)(L¹³-), wherein L¹² and L¹³ are according to any one of (xi)-(xv):
(xi) L¹² and L¹³ both are *—(CH₂)_{a′}—O—C(O)—NH)—(CH₂CH₂O)_{a″}—CH₂—CH₂—N—C(O)—**, wherein the bond labelled * is connected to BM and the bond labelled ** is connected to F², wherein a′ is an integer in the range 0-4, preferably a′=2, and a″ is an integer in the range 0-4, preferably a″ is 2. Further, it is preferred that BM is a nitrogen atom.
(xii) L¹² and L¹³ both are *—(CH₂CH₂O)_{a′}—CH₂—CH₂—C(O)—NH—(CH₂)_{a″}-Ph-*, wherein the bond labelled * is connected to BM and the bond labelled ** is connected to F², wherein a′ is an integer in the range 0-5, preferably a′=2, and a″ is an integer in the range 0-4, preferably a″ is 1. Preferably, Ph is 1,4-Ph. Further, it is preferred that BM is a nitrogen atom.
(xiii) L¹² and L¹³ both are *—(CH₂CH₂O)_{a′}—CH₂—CH₂—C(O)—NH—(CH₂)_{a″}—NH—C(O)-Ph-**, wherein the bond labelled * is connected to BM and the bond labelled ** is connected to F², wherein a′ is an integer in the range 0-5, preferably a′=2, and a″ is an integer in the range 0-4, preferably a″ is 2 or 3. Preferably, Ph is 1,4-Ph. Further, it is preferred that BM is a nitrogen atom.
(xiv) L¹² and L¹³ both are *—(CH₂CH₂O)_{a′}—CH₂—CH₂—C(O)—NH—(CH₂)_{a″}—O-Ph-**, wherein the bond labelled * is connected to BM and the bond labelled ** is connected to F², wherein a′ is an integer in the range 0-5, preferably a′=2, and a″ is an integer in the range 0-4, preferably a″ is 2 or 3. Preferably, Ph is 1,4-Ph. Further, it is preferred that BM is a nitrogen atom.
(xv) L¹² and L¹³ both have the structure -(L¹⁴)-BM′(L¹⁵-)(L¹⁶-), wherein:
   L¹⁴ is as defined for L¹¹, preferably according to any one of (i) to (x) as defined above or, L¹⁴ is *—(CH₂CH₂O)_{a′}—CH₂—CH₂—(C(O))_{a″}—**, wherein the bond labelled * is connected to BM and the bond labelled ** is connected to BM′, and wherein a′ is an integer in the range 0-5, preferably a′=1-4, most preferably a′=2, and a″ is 0 or 1, preferably a″ is 1.
   BM and BM′ are branching moieties, preferably both are nitrogen atoms. With one occurrence of BM and two occurrences of BM′ (in each of L¹⁵ and L¹⁶), y=4.
   L¹⁵ and L¹⁶ are as defined for L¹² and L¹³. Preferably, according to (xiv) as defined above.

Preferably, linkers L¹² and L¹³ both have the same structure. In an especially preferred embodiment of L^{A}, linker L¹¹ is according to any one of (i)-(x) and linkers L¹² and L¹³ are according to any one of (xi)-(xv).

Such preferred linkers L^{A} can be employed in the linker-construct according to the invention, in the conjugate according to the invention and in the intermediate antibody-linker construct according to the invention. In the context of this preferred embodiment, Q¹ is preferably according to (Q26) or (Q37) and F² is preferably according to (F8a), wherein R²⁹ is hydrogen or methyl. Alternatively, it is preferred that Z¹ is preferably according to (Z26) or (Z37), wherein ring Z is according to (Za), and Z² is preferably according to (Z29), wherein ring Z is according to (Zj), wherein R²⁹ is hydrogen or methyl.

### Process for Synthesising the Conjugate According to General Structure (2)

In a further aspect, the present invention relates to a process for the preparation of the conjugate according to the invention, the process comprising:
(a) providing a modified antibody having the structure Ab(F¹)_{z}, wherein Ab is an antibody, z is 2 or 4, and F¹ is a click probe;
(b) reacting the modified antibody with z equivalents of Q¹L^{A}(F²)_{y}, wherein Q1 is a click probe that is reactive towards F¹, L^{A} is a heterobifunctional (y+1)-valent linker, y is 2, 3 or 4, and F² is a click probe that is not reactive towards Q¹, to obtain an antibody-linker construct of structure Ab(Z¹L^{A}(F²)_{y})_{z}, wherein Z¹ is a connecting group obtained by reaction of F¹ and Q¹;
(c) reacting the antibody-linker construct with z×y equivalents of Q²(L^{B})Dₓ, wherein Q² is a click probe that is reactive towards F², L^{B} is a (x+1)-valent linker, x is 1, 2, 3 or 4, provided that x+y is at least 4, and D is a payload molecule, to obtain the conjugate of structure Ab(Z¹L^{A}(Z²(L^{B})Dₓ)_{y})_{z}, wherein Z² is a connecting group obtained by reaction of F² and Q².

The reactions performed in steps (b) and (c) are click reactions, wherein click probes F react with click probes Q to form connecting groups Z. This conjugation technique is known to the skilled person. The present reactions occur under conditions such that Q is reacted with F to form covalent connections. In the process according to the invention, Q reacts with F, forming a covalent connection between the antibody and the payloads. Complementary reactive groups Q and reactive groups F are known to the skilled person and are described in more detail below.

Thus, the reactions in the process according to the invention, conjugation is accomplished via a cycloaddition. Preferred cycloadditions are a (4+2)-cycloaddition (e.g. a Diels-Alder reaction) or a [3+2]-cycloaddition (e.g. a 1,3-dipolar cycloaddition). The reaction of step (b) is preferably a [3+2]-cycloaddition, more preferably a 1,3-dipolar cycloaddition. The 1,3-dipolar cycloaddition is preferably the alkyne-azide cycloaddition, and most preferably wherein Q is or comprises an alkyne group and F is an azido group. The reaction of step (c) is a [4+2]-cycloaddition, preferably a Diels-Alder reaction. The preferred Diels-Alder reaction is the inverse-electron demand Diels-Alder cycloaddition. Cycloadditions, such as Diels-Alder reactions and 1,3-dipolar cycloadditions are known in the art, and the skilled person knowns how to perform them.

### Step (a)

In step (a), an antibody having the structure Ab(F¹)_{z} is provided. Typically, two or four click probes F¹ are chemically or enzymatically introduced onto the antibody. Preferably, z=2 and two click probes are introduced onto the antibody.

In a preferred embodiment, an antibody comprising two or four, preferably two, core N-acetylglucosamine moieties is contacted with a compound of the formula S(F¹)—P in the presence of a catalyst, wherein S(F¹) is a sugar derivative comprising two reactive groups F¹ capable of reacting with a reactive group Q¹, and P is a nucleoside mono- or diphosphate, and wherein the catalyst is capable of transferring the S(F¹) moiety to the core-GlcNAc moiety. Herein, the antibody is typically an antibody that has been trimmed to a core-GlcNAc residue as described further below.

The starting material, i.e. the antibody comprising two or four, preferably two, core-GlcNAc substituents, is known in the art and can be prepared by methods known by the skilled person. In one embodiment, the process according to the invention further comprises the deglycosylation of an antibody glycan having a core N-acetylglucosamine, in the presence of an endoglycosidase, in order to obtain an antibody comprising a core N-acetylglucosamine substituent, wherein said core N-acetylglucosamine and said core N-acetylglucosamine substituent are optionally fucosylated. Depending on the nature of the glycan, a suitable endoglycosidase may be selected. The endoglycosidase is preferably selected from the group consisting of EndoS, EndoA, EndoE, EfEndo18A, EndoF, EndoM, EndoD, EndoH, EndoT and EndoSH and/or a combination thereof, the selection of which depends on the nature of the glycan. EndoSH is described in PCT/EP2017/052792, see Examples 1-3, and SEQ. ID No: 1, which is incorporated by reference herein.

Structural feature S is defined above for the conjugate according to the invention, which equally applies to the present aspect. Compounds of the formula S(F¹)—P, wherein a nucleoside monophosphate or a nucleoside diphosphate P is linked to a sugar derivative S(F¹), are known in the art. For example Wang et al., Chem. Eur. J. 2010, 16, 13343-13345, Piller et al., ACS Chem. Biol. 2012, 7, 753, Piller et al., Bioorg. Med. Chem. Lett. 2005, 15, 5459-5462 and WO 2009/102820, all incorporated by reference herein, disclose a number of compounds S(F¹)—P and their syntheses. In a preferred embodiment nucleoside mono- or diphosphate P in S(F¹)—P is selected from the group consisting of uridine diphosphate (UDP), guanosine diphosphate (GDP), thymidine diphosphate (TDP), cytidine diphosphate (CDP) and cytidine monophosphate (CMP), more preferably P is selected from the group consisting of uridine diphosphate (UDP), guanosine diphosphate (GDP) and cytidine diphosphate (CDP), most preferably P=UDP. Preferably, S(F¹)—P is selected from the group consisting of GalNAz-UDP, F₂-GalNAz-UDP (N-(azidodifluoro)acetyl-galactosamine), 6-AzGal-UDP, 6-AzGalNAc-UDP (6-azido-6-deoxy-N-acetylgalactosamine-UDP), 4-AzGalNAz-UDP, 6-AzGalNAz-UDP, GlcNAz-UDP, 6-AzGlc-UDP, 6-AzGlcNAz-UDP and 2-(but-3-yonic acid amido)-2-deoxy-galactose-UDP. Most preferably, S(F¹)—P is GalNAz-UDP or 6-AzGalNAc-UDP.

Suitable catalyst that are capable of transferring the S(F¹) moiety to the core-GlcNAc moiety are known in the art. A suitable catalyst is a catalyst wherefore the specific sugar derivative nucleotide S(F¹)—P in that specific process is a substrate. More specifically, the catalyst catalyses the formation of a β(1,4)-glycosidic bond. Preferably, the catalyst is selected from the group of galactosyltransferases and N-acetylgalactosaminyltransferases, more preferably from the group of β(1,4)—N-acetylgalactosaminyltransferases (GalNAcT) and β(1,4)-galactosyltransferases (GalT), most preferably from the group of β(1,4)—N-acetylgalactosaminyltransferases having a mutant catalytic domain. Suitable catalysts and mutants thereof are disclosed in WO 2014/065661, WO 2016/022027 and WO 2016/170186, all incorporated herein by reference. In one embodiment, the catalyst is a wild-type galactosyltransferase or N-acetylgalactosaminyltransferase, preferably an N-acetylglucosaminyl-transferase. In an alternative embodiment, the catalyst is a mutant galactosyltransferase or N-acetylgalactosaminyltransferases, preferably a mutant N-acetylgalactosaminyltransferase. Mutant enzymes described in WO 2016/022027 and WO 2016/170186 are especially preferred. These galactosyltransferase (mutant) enzyme catalysts are able to recognize internal sugars and sugar derivatives as an acceptor. Thus, sugar derivative S(F¹) is linked to the core-GlcNAc substituent in step (a), irrespective of whether said GlcNAc is fucosylated or not.

Step (a) is preferably performed in a suitable buffer solution, such as for example phosphate, buffered saline (e.g. phosphate-buffered saline, tris-buffered saline), citrate, HEPES, tris and glycine.

Suitable buffers are known in the art. Preferably, the buffer solution is phosphate-buffered saline (PBS) or tris buffer. Step (i) is preferably performed at a temperature in the range of about 4 to about 50° C., more preferably in the range of about 10 to about 45° C., even more preferably in the range of about 20 to about 40° C., and most preferably in the range of about 30 to about 37° C. Step (i) is preferably performed a pH in the range of about 5 to about 9, preferably in the range of about 5.5 to about 8.5, more preferably in the range of about 6 to about 8. Most preferably, step (i) is performed at a pH in the range of about 7 to about 8.

### Step (b)

In step (b), the modified antibody Ab(F¹)_{z} is reacted with a linker construct according to the invention, comprising a reactive group Q¹ capable of reacting with reactive group F¹, to obtain an antibody-linker construct, containing connecting group Z¹ resulting from the reaction between Q¹ and F¹. Such reaction occurs under condition such that reactive group Q1 is reacted with the reactive group F¹ of the antibody to covalently link the antibody to the linker-construct. In step (b), the reaction occurs with z equivalents of Q¹L^{A}(F²)_{y}, although more equivalents of Q¹L^{A}(F²)_{y} may be present in the reaction mixture in order to ensure complete reaction. The skilled person is able to determine the optimal reaction conditions and stoichiometry of the reactants in order to obtain an optimal yield.

In a preferred embodiment, in step (b) an azide on an azide-modified antibody reacts with a benzoannulated or tetramethylated (hetero)cycloalkyne group, preferably wherein Q1 is according to structure (Q5), (Q6), (Q6a), (Q6b), (Q6c), (Q6d), (Q7), (Q11), (Q17), (018), (Q19) or (Q19a), preferably according to structure (Q26), (Q27), (Q28), (Q32), (Q37), (Q38) or (Q38a), more preferably according to structure (040), (041) or (043) or according to structure (Q37) or (Q43), via a cycloaddition reaction. Using such benzoannulated or tetramethylated (hetero)cycloalkyne groups, the presence of a catalyst is not required, and the cycloaddition reaction may even occur spontaneously by a reaction called strain-promoted azide-alkyne cycloaddition (SPAAC). This is one of the reactions known in the art as “metal-free click chemistry”.

### Step (c)

In step (c), the modified antibody-linker construct Ab(Z¹L^{A}(F²)_{y})_{z} is reacted with a payload-linker, comprising a reactive group Q² capable of reacting with reactive group F², to obtain an antibody-conjugate, containing connecting group Z² resulting from the reaction between Q² and F². Such reaction occurs under condition such that reactive group Q² is reacted with the reactive group F² to covalently link the antibody to the payloads. Step (c) may also be referred to as the conjugation reaction. In step (c), the reaction occurs with z×y equivalents of Q²(L^{B})Dₓ, although more equivalents of Q²(L^{B})Dₓ may be present in the reaction mixture in order to ensure complete reaction. The skilled person is able to determine the optimal reaction conditions and stoichiometry of the reactants in order to obtain an optimal yield.

In step (c), the conjugate is formed by covalently connecting multiple payloads to an antibody. This, in this step the DAR of the conjugate is established. The conjugation step of the present invention is especially effective, as it affords conjugates with an average DAR close to the theoretical value. As such, the conjugates according to the present invention exhibit high homogeneity. The inventors further found that a negative charge at the antibody-side of the conjugation reaction, i.e. in Ab(Z¹L^{A}(F²)_{y})_{z}, typically within L^{A}, negatively affects the conjugation reaction, such that lower DAR values may be obtained. However, the process according to the present invention is able to deal with such negatively charged modified antibody-linker constructs and to afford conjugates with unprecedented DAR values. A negatively charge in L^{A} is present at the process conditions of step (c) and may for example come from deprotonation of a carboxylic acid group or sulfamide group (e.g. according to structure (23)). Thus, in one embodiment, linker L^{A} is not negatively charged, and preferably does not comprises a carboxylic acid group and a sulfamide group. In an alternative embodiment, linker L^{A} is negatively charged, and preferably comprises a carboxylic acid group and/or a sulfamide group.

In a preferred embodiment, in step (c) a 1,2,4,5-tetrazine on the antibody-linker construct reacts with a bicyclononyne group, preferably wherein Q1 is according to structure (Q8), more preferably according to structure (Q29), most preferably according to structure (Q42), via a cycloaddition reaction. Using such bicyclononyne groups, the presence of a catalyst is not required, and the cycloaddition reaction may even occur spontaneously by a reaction called strain-promoted azide-alkyne cycloaddition (SPAAC). This is one of the reactions known in the art as “metal-free click chemistry”.

### Reactive Moiety Q

Reactive moieties Q¹ and Q² are click probes. In the context of the present invention, Q refers to Q¹ and Q². In the context of the present invention, the term “reactive moiety” may refer to a chemical moiety that comprises a reactive group, but also to a reactive group itself. For example, a cyclooctynyl group is a reactive group comprising a reactive group, namely a C—C triple bond. However, a reactive group, for example an azido reactive group, may herein also be referred to as a reactive moiety.

Q is reactive towards and complementary to F. Herein, a reactive group is denoted as “complementary” to a reactive group when said reactive group reacts with said reactive group selectively, optionally in the presence of other functional groups. Complementary reactive click probes are known to a person skilled in the art, and are described in more detail below. The exact nature of Q, and F, depends on the type of click reaction that is employed. The click probe is reactive in a cycloaddition (click reaction) and is preferably selected from an azide, a tetrazine, a triazine, a nitrone, a nitrile oxide, a nitrile imine, a diazo compound, an ortho-quinone, a dioxothiophene, a sydnone, an alkene moiety and an alkyne moiety. Preferably, click probe Q comprises or is an alkene moiety or an alkyne moiety, more preferably wherein the alkene is a (hetero)cycloalkene and/or the alkyne is a terminal alkyne or a (hetero)cycloalkyne.

In an especially preferred embodiment, Q comprises a cyclic (hetero)alkyne moiety. The alkynyl group may also be referred to as a (hetero)cycloalkynyl group, i.e. a heterocycloalkynyl group or a cycloalkynyl group, wherein the (hetero)cycloalkynyl group is optionally substituted. Preferably, the (hetero)cycloalkynyl group is a (hetero)cycloheptynyl group, a (hetero)cyclooctynyl group, a (hetero)cyclononynyl group or a (hetero)cyclodecynyl group. Herein, the (hetero)cycloalkynes may optionally be substituted. Preferably, the (hetero)cycloalkynyl group is an optionally substituted (hetero)cycloheptynyl group or an optionally substituted (hetero)cyclooctynyl group. Most preferably, the (hetero)cycloalkynyl group is a (hetero)cyclooctynyl group, wherein the (hetero)cyclooctynyl group is optionally substituted.

In an especially preferred embodiment, Q comprises a (hetero)cycloalkynyl or (hetero)cycloalkenyl group and is according to structure (Q1):

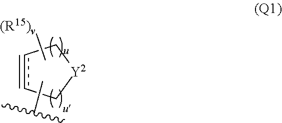

Herein:
the bond depicted as [symbol] is a double bond or a triple bond;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S, S⁽⁺⁾R³¹, S(O)R³¹, S(O)—NR³¹ or NR³¹, wherein S⁽⁺⁾ is a cationic sulphur atom counterbalanced by B⁽⁻⁾, wherein B⁽⁻⁾ is an anion, and wherein each R³¹ individually is R¹⁵ or a connection with D, connected via L;
u is 0, 1, 2, 3, 4 or 5;
u′ is 0, 1, 2, 3, 4 or 5, wherein u+u′=0, 1, 2, 3, 4, 5, 6, 7 or 8;
v=an integer in the range 0-16;

Typically, v=(u+u′)×2 (when the connection to L, depicted by the wavy bond, is via Y²) or [(u+u′)×2]−1 (when the connection to L, depicted by the wavy bond, is via one of the carbon atoms of u and u′).

In a preferred embodiment of structure (Q1), reactive group Q comprises a (hetero)cycloalkynyl group and is according to structure (Q1a):

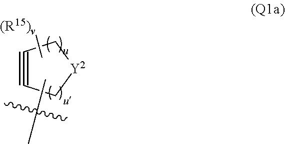

### Herein,

R¹⁵ and Y² are as defined above
u is 0, 1, 2, 3, 4 or 5;
u′ is 0, 1, 2, 3, 4 or 5, wherein u+u′=4, 5, 6, 7 or 8;
v=an integer in the range 8-16.

In a preferred embodiment, u+u′=4, 5 or 6, more preferably u+u′=5.

In a preferred embodiment, v=8, 9 or 10, more preferably v=9 or 10, most preferably v=10.

In a preferred embodiment, Q is a (hetero)cycloalkynyl group selected from the group consisting of (Q2)-(Q20) depicted here below.

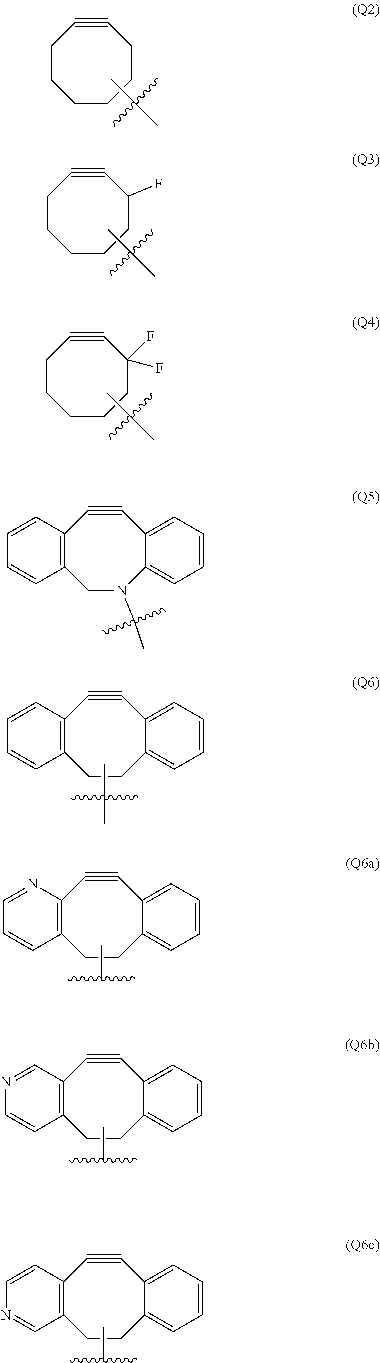

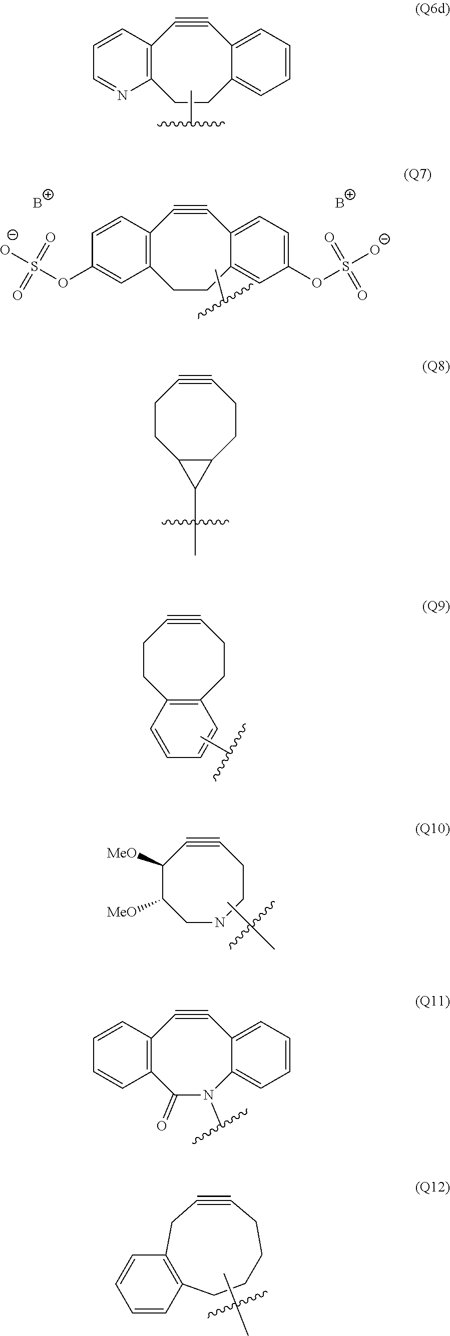

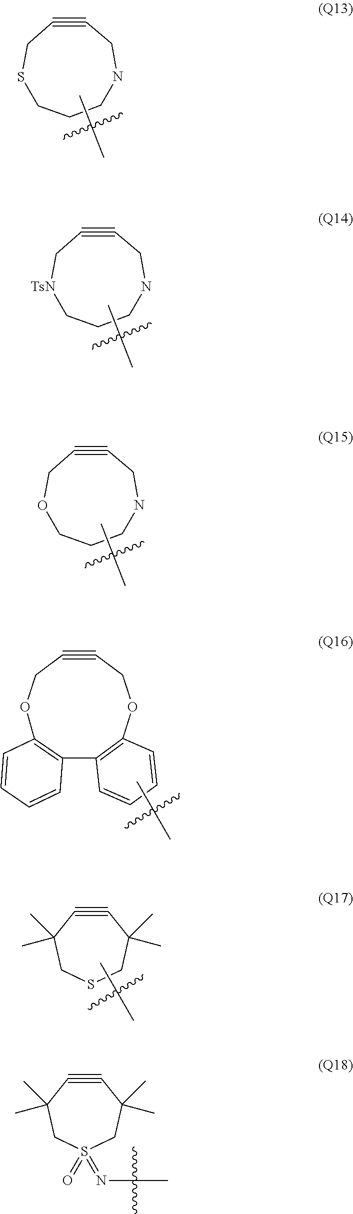

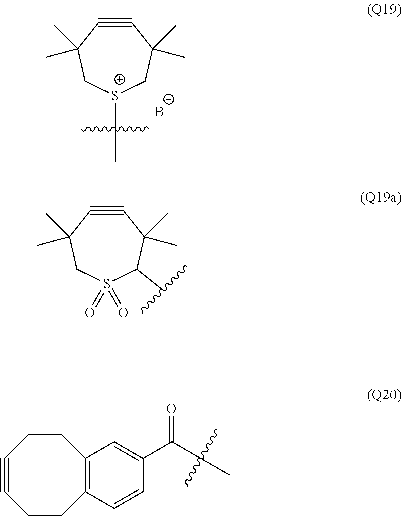

Herein, the connection to L, depicted with the wavy bond, may be to any available carbon or nitrogen atom of Q. The nitrogen atom of (Q10), (Q13), (Q14) and (Q15) may bear the connection to L, or may contain a hydrogen atom or be optionally functionalized. B⁽⁻⁾ is an anion, which is preferably selected from ⁽⁻⁾OTf, Cl⁽⁻⁾, Br⁽⁻⁾ or I⁽⁻⁾, most preferably B⁽⁻⁾ is ⁽⁻⁾OTf. B⁽⁺⁾ is a cation, preferably a pharmaceutically acceptable cation. In the conjugation reaction, B⁽⁻⁾ does not need to be a pharmaceutically acceptable anion, since B⁽⁻⁾ will exchange with the anions present in the reaction mixture anyway. In case (Q19) is used for Q, the negatively charged counter-ion is preferably pharmaceutically acceptable upon isolation of the conjugate according to the invention, such that the conjugate is readily useable as medicament.

In a further preferred embodiment, Q is a (hetero)cycloalkynyl group selected from the group consisting of (Q21)-(Q38a) depicted here below.

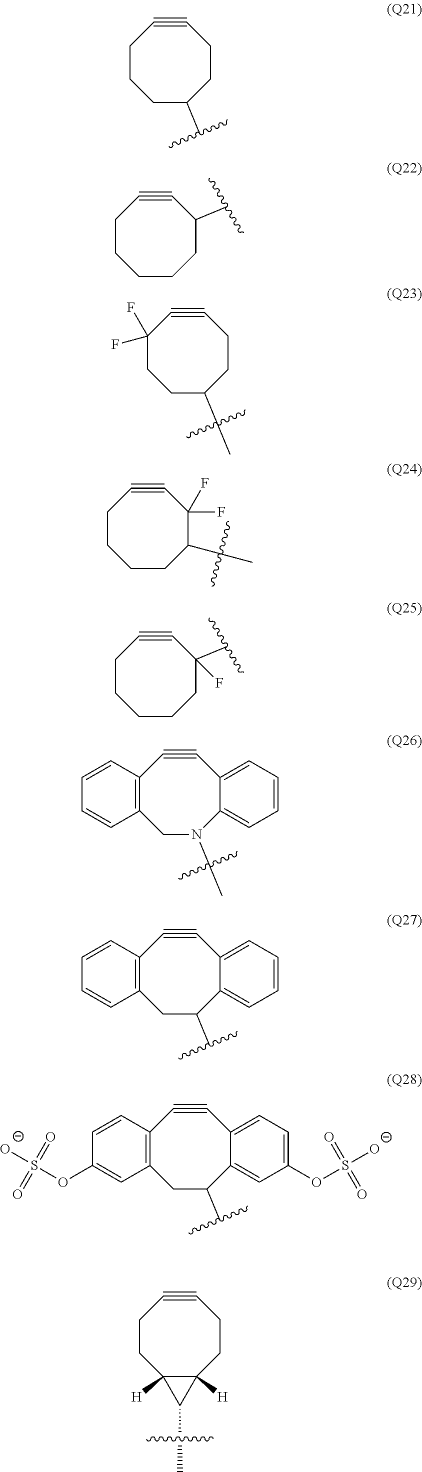

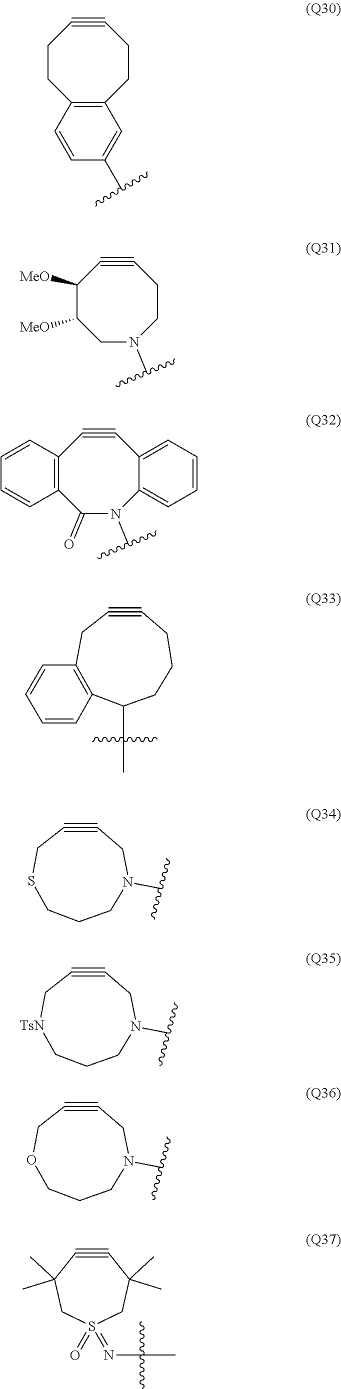

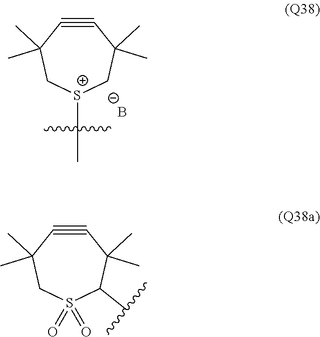

In structure (Q38), B⁽⁻⁾ is an anion, which is preferably selected from ⁽⁻⁾OTf, Cl⁽⁻⁾, Br⁽⁻⁾ or I⁽⁻⁾, most preferably B⁽⁻⁾ is ⁽⁻⁾OTf.

In a preferred embodiment, Q comprises a (hetero)cyclooctyne moiety or a (hetero)cycloheptyne moiety, preferably according to structure (Q8), (Q26), (Q27), (Q28), (Q37) or (Q38a), which are optionally substituted. Each of these preferred options for Q are further defined here below.

Thus, in a preferred embodiment, Q comprises a heterocycloheptyne moiety according to structure (Q37), also referred to as a TMTHSI, which is optionally substituted. Preferably, the heterocycloheptyne moiety according to structure (Q37) is not substituted.

In an alternative preferred embodiment, Q comprises a cyclooctyne moiety according to structure (Q8), more preferably according to (Q29), also referred to as a bicyclo[6.1.0]non-4-yn-9-yl] group (BCN group), which is optionally substituted. Preferably, the cyclooctyne moiety according to structure (Q8) or (Q29) is not substituted. In the context of the present embodiment, Q preferably is a (hetero)cyclooctyne moiety according to structure (Q39) as shown below, wherein V is (CH₂)ₗ and l is an integer in the range of 0 to 10, preferably in the range of 0 to 6. More preferably, l is 0, 1, 2, 3 or 4, more preferably l is 0, 1 or 2 and most preferably l is 0 or 1. In the context of group (Q39), l is most preferably 1. Most preferably, Q is according to structure (Q42), defined further below.

In an alternative preferred embodiment, Q comprises a (hetero)cyclooctyne moiety according to structure (Q26), (Q27) or (Q28), also referred to as a DIBO, DIBAC, DBCO or ADIBO group, which are optionally substituted. In the context of the present embodiment, Q preferably is a (hetero)cyclooctyne moiety according to structure (040) or (Q41) as shown below, wherein Y¹ is O or NR¹¹, wherein R¹¹ is independently selected from the group consisting of hydrogen, a linear or branched C₁-C₁₂ alkyl group or a C₄-C₁₂ (hetero)aryl group. The aromatic rings in (Q40) are optionally O-sulfonylated at one or more positions, whereas the rings of (Q41) may be halogenated at one or more positions. Preferably, the (hetero)cyclooctyne moiety according to structure (Q40) or (Q41) is not further substituted. Most preferably, Q is according to structure (Q43), defined further below.

In an alternative preferred embodiment, Q comprises a heterocycloheptynyl group and is according to structure (Q37).

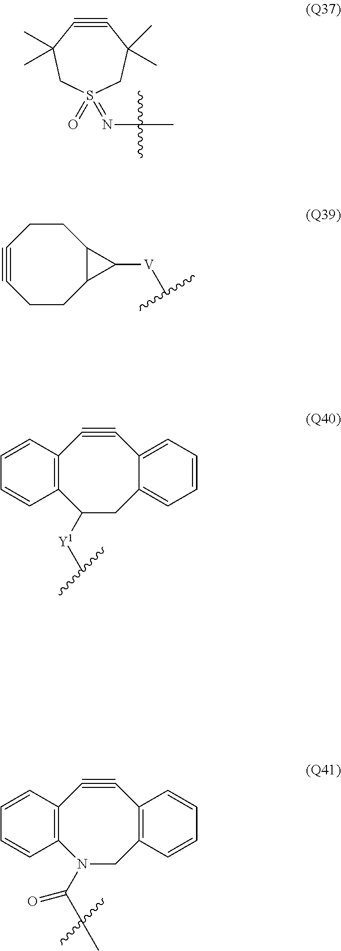

In an especially preferred embodiment, Q comprises a cyclooctynyl group and is according to structure (Q42):

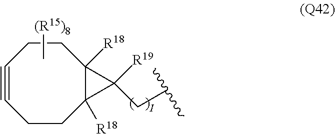

Herein:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted, or R¹⁹ is a second occurrence of Q or D connected via a spacer moiety; and
l is an integer in the range 0 to 10.

In a preferred embodiment of the reactive group according to structure (Q42), R¹⁵ is independently selected from the group consisting of hydrogen, halogen, —OR¹⁶, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C₆ alkyl, most preferably all R¹⁵ are H. In a preferred embodiment of the reactive group according to structure (Q42), R¹⁸ is independently selected from the group consisting of hydrogen, C₁-C₆ alkyl groups, most preferably both R¹⁸ are H. In a preferred embodiment of the reactive group according to structure (Q42), R¹⁹ is H. In a preferred embodiment of the reactive group according to structure (Q42), l is 0 or 1, more preferably l is 1.

In an especially preferred embodiment, Q comprises a (hetero)cyclooctynyl group and is according to structure (Q43):

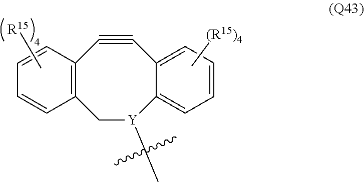

### Herein:

R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵;
a carbon atom in the fused aromatic rings may be replaced by a nitrogen atom, as in (Q6a)-(Q6d), preferably wherein Y is CR¹⁵.

In a preferred embodiment of the reactive group according to structure (Q43), R¹⁵ is independently selected from the group consisting of hydrogen, halogen, —OR¹⁶, —S(O)₃⁽⁻⁾, C₁-C₆ alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃⁽⁻⁾. In a preferred embodiment of the reactive group according to structure (043), Y is N or CH, more preferably Y=N.

In an alternative preferred embodiment, Q comprises a cyclic alkene moiety. The alkenyl group Q may also be referred to as a (hetero)cycloalkenyl group, i.e. a heterocycloalkenyl group or a cycloalkenyl group, preferably a cycloalkenyl group, wherein the (hetero)cycloalkenyl group is optionally substituted. Preferably, the (hetero)cycloalkenyl group is a (hetero)cyclopropenyl group, a (hetero)cyclobutenyl group, a norbornene group, a norbornadiene group, a trans-(hetero)cycloheptenyl group, a trans-(hetero)cyclooctenyl group, a trans-(hetero)cyclononenyl group or a trans-(hetero)cyclodecenyl group, which may all optionally be substituted. Especially preferred are (hetero)cyclopropenyl groups, trans-(hetero)cycloheptenyl group or trans-(hetero)cyclooctenyl groups, wherein the (hetero)cyclopropenyl group, the trans-(hetero)cycloheptenyl group or the trans-(hetero)cyclooctenyl group is optionally substituted. Preferably, Q comprises a cyclopropenyl moiety according to structure (Q44), a hetereocyclobutene moiety according to structure (Q45), a norbornene or norbornadiene group according to structure (Q46), a trans-(hetero)cycloheptenyl moiety according to structure (Q47) or a trans-(hetero)cyclooctenyl moiety according to structure (Q48). Herein, Y³ is selected from C(R²³)₂, NR²³ or O, wherein each R²³ is individually hydrogen, C₁-C₆ alkyl or is connected to L, optionally via a spacer, and the bond labelled [symbol] is a single or double bond. In a further preferred embodiment, the cyclopropenyl group is according to structure (Q49). In another preferred embodiment, the trans-(hetero)cycloheptene group is according to structure (Q50) or (Q51). In another preferred embodiment, the trans-(hetero)cyclooctene group is according to structure (Q52), (Q53), (Q54), (Q55) or (Q56).

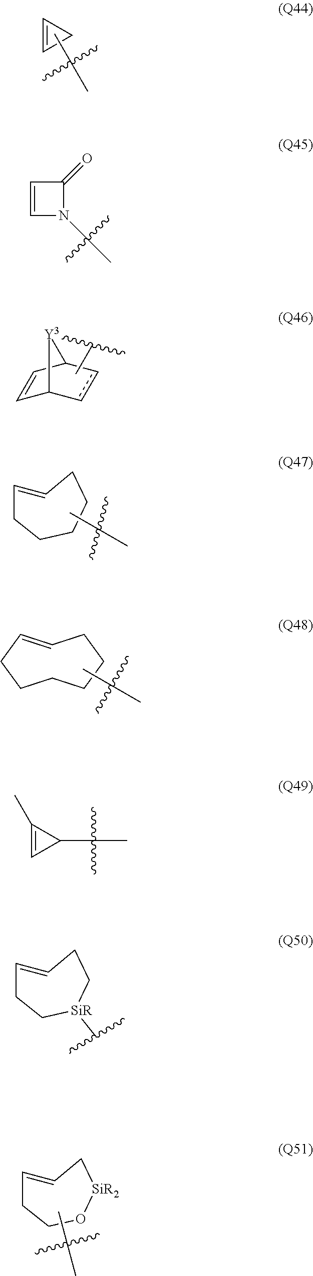

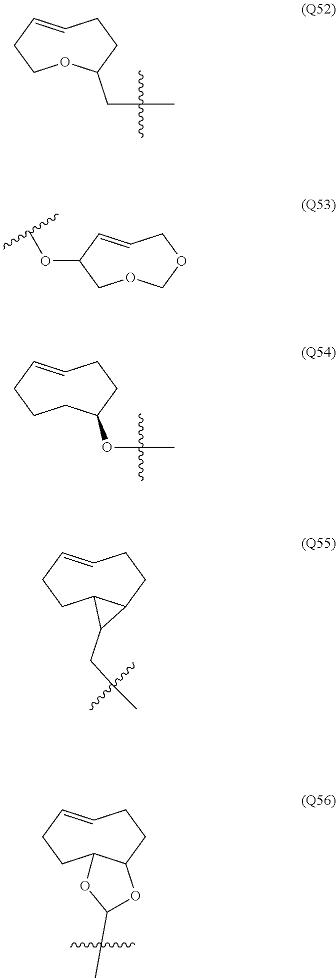

Herein, the R group(s) on Si in (Q50) and (Q51) are typically alkyl or aryl, preferably C₁-C₆ alkyl.

In a preferred embodiment, click probes Q comprise a moiety selected from (Q1)-(Q56), more preferably is a moiety selected from (Q1)-(Q56).

In the present invention, the exact structure of Q¹ and Q² should differ, as Q1 is not reactive towards F², whereas Q² is reactive towards F².

In a preferred embodiment, F¹ is azide and Q¹ is a benzoannulated or tetramethylated (hetero)cycloalkyne, while F² is tetrazine or nitrone and Q² is bicyclononyne or cycloalkene, such as a trans-cyclooctene or a cyclopropene. More preferably, F¹ is azide and Q¹ is an benzoannulated or tetramethylated (hetero)cycloalkyne, while F² is tetrazine and Q² is bicyclononyne. Herein, Q¹ is preferably according to structure (Q5), (Q6), (Q6a), (Q6b), (Q6c), (Q6d), (Q7), (Q11), (Q17), (018), (Q19) or (Q19a), more preferably according to structure (Q26), (Q27), (Q28), (Q32), (Q37), (Q38) or (Q38a), most preferably according to structure (Q40), (Q41) or (043) or according to structure (Q37) or (043). Herein, Q² is preferably according to structure (Q8), (Q44), (Q47), (Q48), (049), (Q54), (Q55) or (Q56), more preferably according to structure (Q29), (048) or (049), most preferably according to structure (042). Alternatively, Q² is preferably according to structure (Q8), more preferably according to structure (Q29), most preferably according to structure (Q42).

### Reactive Moiety F

Reactive moieties F¹ and F² are click probes. In the context of the present invention, F refers to F¹ and F². F is reactive towards and complementary to Q. Herein, a reactive group is denoted as “complementary” to a reactive group when said reactive group reacts with said reactive group selectively, optionally in the presence of other functional groups. Complementary reactive click probes are known to a person skilled in the art, and are described in more detail below. The exact nature of Q, and F, depends on the type of click reaction that is employed. The click probe is reactive in a cycloaddition (click reaction) and is preferably selected from an azide, a tetrazine, a triazine, a nitrone, a nitrile oxide, a nitrile imine, a diazo compound, an ortho-quinone, a dioxothiophene, a sydnone, an alkene moiety and an alkyne moiety. Preferably, click probe F comprises or is an azide moiety, a nitrone moiety or a tetrazine moiety.

F is reactive towards Q in the conjugation reaction defined below, preferably wherein the conjugation reaction is a cycloaddition or a nucleophilic reaction. As the skilled person will understand, the options for F are the same as those for Q, provided that F and Q are reactive towards each other. The click probe is reactive in a cycloaddition (click reaction) and is preferably selected from an azide, a tetrazine, a triazine, a nitrone, a nitrile oxide, a nitrile imine, a diazo compound, an ortho-quinone, a dioxothiophene, a sydnone, an alkene moiety and an alkyne moiety. Preferably, the click probe comprises or is an azide, a tetrazine, a triazine, a nitrone, a nitrile oxide, a nitrile imine, a diazo compound, an ortho-quinone, a dioxothiophene or a sydnone, most preferably an azide.

The reactive group F on the antibody are typically introduced by a specific technique, for example a (bio)chemical or a genetic technique. The reactive group that is placed in the antibody is prepared by chemical synthesis, for example an azide or a terminal alkyne. Methods of preparing modified antibodies are known in the art, e.g. from WO 2014/065661, WO 2016/170186 and WO 2016/053107, which are incorporated herein by reference. From the same documents, the conjugation reaction between the modified antibody and a linker-toxin-construct is known to the skilled person.

Preferably, F is a click probe reactive towards a (hetero)cycloalkene and/or a (hetero)cycloalkyne, and is typically selected from the group consisting of azide, tetrazine, triazine, nitrone, nitrile oxide, nitrile imine, diazo compound, ortho-quinone, dioxothiophene and sydnone. Preferred structures for the reactive group are structures (F1)-(F10) depicted here below.

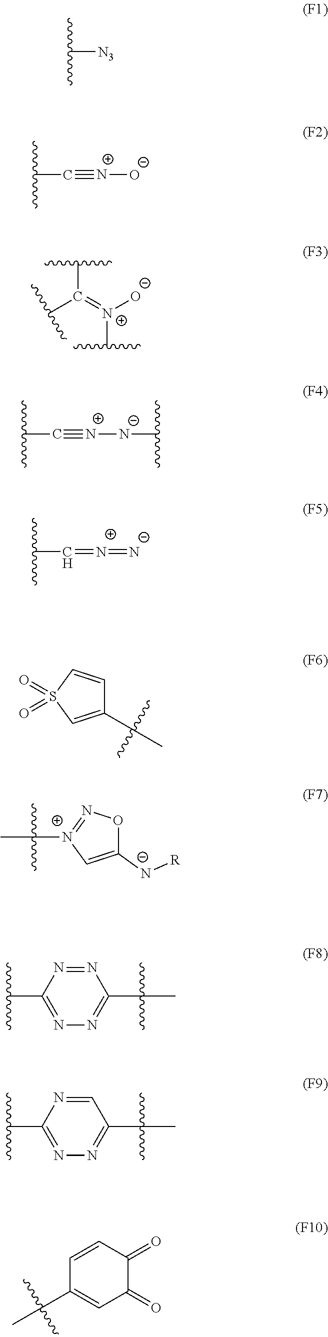

Herein, the wavy bond represents the connection to Ab or L^{A}. For (F3), (F4), (F8) and (F9), the payload can be connected to any one of the wavy bonds. The other wavy bond may then be connected to an R group selected from hydrogen, C₁-C₂₄ alkyl groups, C₂-C₂₄ acyl groups, C₃-C₂₄ cycloalkyl groups, C₂-C₂₄ (hetero)aryl groups, C₃-C₂₄ alkyl(hetero)aryl groups, C₃-C₂₄ (hetero)arylalkyl groups and C₁-C₂₄ sulfonyl groups, each of which (except hydrogen) may optionally be substituted and optionally interrupted by one or more heteroatoms selected from O, S and NR³² wherein R³² is independently selected from the group consisting of hydrogen and C₁-C₄ alkyl groups. The skilled person understands which R groups may be applied for each of the groups F. For example, the R group connected to the nitrogen atom of (F3) may be selected from alkyl and aryl, and the R group connected to the carbon atom of (F3) may be selected from hydrogen, alkyl, aryl, acyl and sulfonyl. Preferably, the reactive moiety F is selected from azides or tetrazines.

In an especially preferred embodiment, F is a tetrazine according to structure (F8a):

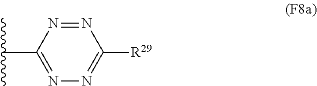

Herein, R²⁹ is selected from hydrogen, C₁₋₆ alkyl, aryl, C(O)—C₁₋₆ alkyl, C(O)-aryl, C(O)—O—C₁₋₆ alkyl, C(O)—O-aryl, C(O)—NR³³—C₁₋₆ alkyl and C(O)—NR³³-aryl, wherein R³³ is H or C₁₋₄ alkyl. Preferably, R²⁹ is selected from hydrogen, methyl, phenyl, pyridyl, pyridinyl and pyrimidinyl. It was found that R²⁹ is hydrogen gave optimal results in reactivity in the cycloaddition reaction. Thus, in a preferred embodiment, ring F, in particular F², is (F8a) wherein R²⁹ is selected from hydrogen, methyl, phenyl, pyridyl, pyridinyl and pyrimidinyl, more preferably R²⁹ is hydrogen or methyl, most preferably R²⁹ is methyl.

In a preferred embodiment, click probes F¹ and F² are selected from the group consisting of azide, tetrazine, triazine, nitrone, nitrile oxide, nitrile imine, diazo compound, dioxothiophene, sydnone, iminosydnone and catechol. Note that catechol in situ oxidized to an ortho-quinone group, which is reactive as click probe. Likewise, the term “tetrazine” also encompasses “hydrotetrazine”, a known precursor that forms tetrazine upon in situ oxidation. Such precursors of click probes, which in situ form reactive groups, are also covered in the present invention. FIG. 11 gives some known examples of in situ formed click probes by oxidation. Preferred click probes F¹ and F² are selected from the group consisting of azide, tetrazine, ortho-quinone and nitrone, more preferably from azide, tetrazine and nitrone. In a preferred embodiment, F¹ is an azide or nitrone, and F² is iminosydnone, catechol, which forms a ortho-quinone group in situ, or tetrazine. More preferably, F² is iminosydnone according to structure (F7), catechol, which forms structure (F10) in situ, or tetrazine according to structure (F8). Even more preferably, F¹ is an azide according to structure (F1) or nitrone according to structure (F3) and F² is a tetrazine according to structure (F8a). Most preferably, F¹ is an azide according to structure (F1) and F² is a tetrazine according to structure (F8a).

### Linker-Construct According to General Structure (4)

In a further aspect, the invention concerns a linker-construct. The linker-construct according to the invention is a heterobifunctional (y+1)-valent linker of structure (4):

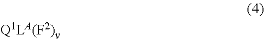

Herein, Q¹ is a click probe, L^{A} is a linker, y is 2, 3 or 4, and F² is a click probe that is not reactive towards Q¹. Q¹, L^{A}, y and F² are defined elsewhere, and those definitions and preferred embodiments thereof are equally applicable to the linker-construct according to the invention.

In a preferred embodiment of the linker-construct according to the present aspect, Q¹ is a benzoannulated or tetramethylated (hetero)cycloalkyne. Such a click probe Q¹ is preferably combined with a tetrazine as click probe F². As such, both Q¹ and F² are click probes reactive in a cycloaddition reaction, but are not reactive towards each other.

Q¹ is preferably selected from (Q5), (Q6), (Q6a), (Q6b), (Q6c), (Q6d), (Q7), (Q11), (Q17) (Q18), (Q19) and (Q19a) as defined above, more preferably selected from (Q5), (Q6), (Q6a), (Q6b), (Q6c), (Q6d), (Q7), (Q18) and (Q19a), even more preferably according to structure (Q26), (Q27), (Q28), (Q32), (Q37), (Q38) or (Q38a), most preferably according to structure (Q37), (Q40), (Q41) or (Q43).

F² is preferably a tetrazine according to structure (F8), most preferably according to structure (F8a):

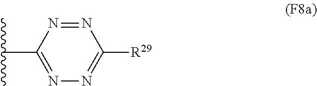

Herein, R²⁹ is selected from hydrogen, C₁₋₆ alkyl, aryl, C(O)—C₁₋₆ alkyl, C(O)-aryl, C(O)—O—C₁₋₆ alkyl, C(O)—O-aryl, C(O)—NR³³—C¹⁻⁶ alkyl and C(O)—NR³³-aryl, wherein R³³ is H or C₁₋₄ alkyl, preferably wherein R²⁹ is selected from hydrogen, methyl, phenyl, pyridyl, pyridinyl and pyrimidinyl. In a preferred embodiment, R³³ is hydrogen or methyl, most preferably R³³ is methyl.

Integer y is 2, 3 or 4, preferably y is 2 or 3, most preferably y is 2.

### Application

The conjugates of the present invention, having DAR6 or higher, have a high homogeneity and a DAR value close to theoretical DAR value. The conjugates of the present invention are further characterized by a high stability, a low tendency to aggregate and excellent therapeutic efficacy and tolerability. The conjugates of the present invention are therefore especially suitable in the treatment of cancer. In that light, the invention further concerns a method for the treatment of cancer, comprising administering to a subject in need thereof the conjugate according to the invention. The subject in need thereof is typically a cancer patient. The use of conjugates, such as antibody-drug conjugates, is well-known in the field of cancer treatment, and the conjugates according to the invention are especially suited in this respect. The method as described is typically suited for the treatment of cancer. In the method according to this aspect, the antibody-conjugate is typically administered in a therapeutically effective dose. The present aspect of the invention can also be worded as a conjugate according to the invention for use in the treatment of cancer. In other words, this aspect concerns the use of a conjugate according to the invention for the preparation of a medicament or pharmaceutical composition for use in the treatment of cancer. In the present context, treatment of cancer is envisioned to encompass treating, imaging, diagnosing, preventing the proliferation of, containing and reducing tumours.

This aspect of the present invention may also be worded as a method for targeting a tumour cell expressing a specific extracellular receptor, comprising contacting the conjugate according to the invention with cells that may possibly express the extracellular receptor, and wherein the antibody specifically targets the extracellular receptor. The method according to this aspect is thus suitable to determine whether the cells are expressing the desired extracellular receptor. These tumour cells may be present in a subject, in which case the method comprises administering to a subject in need thereof the conjugate according to the invention. Alternatively, the method occurs ex vivo or in vitro. In a preferred embodiment, the cells that may possibly express the extracellular receptor are cells that express the extracellular receptor. The targeting of tumour cells preferably includes one or more of treating, imaging, diagnosing, preventing the proliferation of, containing and reducing the tumour cells.

In the context of diagnosis, it is typically unknown whether the cells that are being contacted in fact express the specific extracellular receptor that is being investigated. For example, in the diagnosis of HER2-positive breast cancer, a conjugate containing an antibody that targets HER2, such as trastuzumab, may be contacted with the cells. In case the tumour cells are in fact HER2-expressing, the conjugate will target the cells, while in case the tumour cells are not HER2-expressing, the conjugate will not target the cells. Likewise, in the treatment of a cancer cells specifically expressing an extracellular receptor, the skilled person will understand that a cell-binding agent, such as an antibody, is to be used that targets that specific extracellular receptor.

In the methods of the present invention, it is preferred that the extracellular receptor is selected from the group consisting of 5T4, ADAM-9, AMHRII, ASCT2, ASLG659, ASPHD1, av-integrin, Axl, B7-H3, B7-H4, BAFF-R, BCMA, BMPR1B, Brevican, c-KIT, c-Met, C4.4a, CA-IX, cadherin-6, CanAg, CD123, CD13, CD133, CD138/syndecan-1, CD166, CD19, CD20, CD203c, CD205, CD21, CD22, CD228, CD25, CD30, CD324, CD33, CD37, CD38, CD45, CD46, CD48a, CD56, CD70, CD71, CD72, CD74, CD79a, CD79b, CEACAM5, claudin-18.2, claudin-6, CLEC12A, CLL-1, Cripto, CRIPTO, CS1, CXCR5, DLK-1, DLL3, DPEP3, E16, EGFR, ENPP3, EpCAM, EphA2, EphB2R, ETBR, FAP, FcRH1, FcRH2, FcRH5, FGFR2, fibronectin, FLT3, folate receptor alpha, Gal-3BP, GD3, GDNF-Ra1, GEDA, GFRA1, Globo H, gpNMB, GPR172A, GPR19, GPR54, guanyl cyclase C, HER2, HER3, HLA-DOB, IGF-1R, IL13R, IL20Rα, Lewis Y, LGR5, LIV-1, LRRC15, LY64, Ly6E, Ly6G6D, LY6K, MDP, MFI2, MICA/B, MOSPD2, MPF, MSG783, MUC1, MUC16, NaPi2b, NCA, nectin-4, Notch3, P-cadherin, P2X5, PD-L1, PMEL17, PRLR, PSCA, PSCA hlg, PSMA, PTK7, RET, RNF43, RON, ROR1, ROR2, Sema 5b, SLITRK6, SSTR2, STEAP1, STEAP2, TAG72, TENB2, TF, TIM-1, TM4SF, TMEFF, TMEM118, TMEM46, transferrin, TROP-2, TrpM4, TWEAKR, receptor tyrosine kinases (RTK), tenascin. Likewise, it is preferred that the tumour cells express an extracellular receptor selected from the same group. The skilled person is capable of matching the desired extracellular receptor with a suitable cell-binding agent capable of targeting that extracellular receptor.

The conjugates of the present invention are also especially suitable as antibiotic, antiviral, anti-inflammatory and anti-autoimmune agent. Thus, in an alternative embodiment, the invention concerns a method for the treatment of infection, inflammation of an autoimmune disorder, comprising administering to a subject in need thereof the conjugate according to the invention. In the method according to this aspect, the antibody-conjugate is typically administered in a therapeutically effective dose. The present aspect of the invention can also be worded as a conjugate according to the invention for use in the treatment of infection, inflammation of an autoimmune disorder. In other words, this aspect concerns the use of a conjugate according to the invention for the preparation of a medicament or pharmaceutical composition for use in the treatment of infection, inflammation of an autoimmune disorder. Herein, the infection may be bacterial or viral.

In the present embodiment, the antibody is preferably specific to an extracellular protein resulting from a viral infection and/or tumour-associated carbohydrate antigen. Herein, the extracellular protein resulting from a viral infection may be human polio virus (HPV), human cytomegalovirus (HCMV) or human papillomavirus (HPV). The tumour-associated carbohydrate antigen (TACA) may be selected from the group of Tn, STn, T-antigen, LDN, Lewis^{c} (Le^{c}), Sialyl-Lewis^{c} (SLe^{c}), 6-Sialyl-Lewis^{c} (6SLe^{c}), LN, alpha-Gal, 3SLN, 6SLN, H-antigen, A-antigen, B-antigen, Lewis^{a} (Le^{a}), Sialyl-Lewis^{a} (SLe^{a}), 6-Sialyl-Lewis^{a} (6SLe^{a}), Lewis^{b} (Le^{b}), Sialyl-Lewis^{b} (SLe^{b}), 6-Sialyl-Lewis^{b} (6SLe^{b}), Lewis^{x} (Le^{x}), Sialyl-Lewis^{x} (SLe^{x}), 6-Sialyl-Lewis^{x} (6SLe^{x}), Lewis^{y} (Le^{y}), Sialyl-Lewis^{y} (SLe^{y}), 6-Sialyl-Lewis^{y} (6SLe^{y}) and or combinations thereof. The antibody may also be specific to both an extracellular protein and a TACA at the same time.

In this light, the invention also concerns a pharmaceutical composition comprising the conjugate according to the invention and a pharmaceutically acceptable carrier. The pharmaceutical composition typically contains the conjugate according to the invention in a pharmaceutically effective dose.

### DESCRIPTION OF THE FIGURES

FIG. 1 shows the general scheme for preparation of antibody-drug conjugates by reaction of a monoclonal antibody (in most cases a symmetrical dimer) containing an x number of functionalities F. By incubation of antibody-(F)ₓ with excess of a linker-drug construct (0-spacer-linker-payload) a conjugate is obtained by reaction of F with Q, forming connecting group Z.

FIGS. 2A-2B depict a range of reagents suitable for reaction with cysteine side-chains. Reagents may be monoalkylation type (A) or may be a cross-linker (B) for reaction with two cysteine side-chains.

FIG. 3 shows the general process for non-genetic conversion of a monoclonal antibody (mAb) into an antibody containing probes for click conjugation (F). The click probe may be on various positions in the antibody, depending on the technology employed. For example, the antibody may be converted into an antibody containing two click probes (structure on the left) or four click probes (bottom structure) or eight probes (structure on the right) for click conjugation.

FIG. 4 shows a representative (but not comprehensive) set of functional groups (F) that can be introduced into an antibody by engineering, by chemical modification, or by enzymatic means, which upon metal-free click reaction with a complementary reactive group Q lead to connecting group Z. Functional group F may be artificially introduced (engineered) into an antibody at any position of choice. Some functional groups F (e.g. nitrile oxide, quinone), may besides strained alkynes also react with strained alkenes, which as an example is depicted for triazine or tetrazine (bottom line). The pyridine or pyridazine connecting group is the product of the rearrangement of the tetrazabicyclo[2.2.2]octane connecting group, formed upon reaction of triazine or tetrazine with alkyne (but not alkene), respectively, with loss of N2. Connecting groups Z depicted in FIG. 4 are preferred connecting groups to be used in the present invention.

FIG. 5 shows cyclic alkynes suitable for metal-free click chemistry, and preferred embodiments for reactive moiety Q. The list is not comprehensive, for example alkynes can be further activated by fluorination, by substitution of the aromatic rings or by introduction of heteroatoms in the aromatic ring.

FIG. 6 depicts a specific example of site-specific conjugation of a payload based on glycan remodeling of a full-length IgG followed by azide-cyclooctyne click chemistry. The IgG is first enzymatically remodeled by endoglycosidase-mediated trimming of all different glycoforms, followed by glycosyltransferase-mediated transfer of azido-sugar onto the core GlcNAc liberated by endoglycosidase. In the next step, the azido-remodeled IgG is subjected to an immune cell-engaging polypeptide, which has been modified with a single cyclooctyne for metal-free click chemistry (SPAAC), leading to a bispecific antibody of 2:2 molecular format. It is also depicted that the cyclooctyne-polypeptide construct will have a specific spacer between cyclooctyne and polypeptide, which enables tailoring of IgG-polypeptide distance or impart other properties onto the resulting bispecific antibody.

FIG. 7 describes the two-stage process as applied herein, whereby a glycan-remodeled antibody with functionality F₁ is reacted with a bifunctional linker Q₁-L-F₂ (wherein the click probes Q₁ and F₂ are mutually non-reactive), thereby undergoing a metal-free click chemistry reaction to form bond Z₁. The bifunctional linker may comprise 2 to 4 occurrences of F₂ (i.e. trivalent, tetravalent or pentavalent linker). In the second step, the antibody containing the reactive group F₂ is reacted with click probe Q₂, which is part of the linker-drug containing 1-3 occurrences of drug.

FIGS. 8A-8B depict three examples of a bifunctional linker Q₁-L-F₂ that fulfill the condition that Q₁ and F₂ are not mutually reactive, where Q₁ is either DBCO (as in A) or TMTHSI (as in B and C) and F₂ is a tetrazine analogue (as in A-C).

FIG. 9 shows two examples of a bifunctional linker Q₁-L-F₂ that fulfill the condition that Q₁ and F₂ are not mutually reactive, where Q₁ is either TMTHSI (as in D) or DBCO (as in E), the tetrazine is a alkyl, methyltetrazine as in D) or a phenyltetrazine (as in E) and where the linker is either tetravalent (as in D) or pentavalent (as in E).

FIG. 10 depicts an example of a bifunctional linker Q₁-L-F₂ (F), where Q₁ is DBCO and F₂ is an iminosydnone variant known to react with DBCO extremely slowly (k<0.001 M⁻¹s⁻¹).

FIG. 11 shows three examples of a bifunctional linker Q₁-L-F₂, whereby F₂ is a latent click-reactive group (i.e. not reactive as such but requiring chemical or enzymatic conversion to a reactive click probe), such as phenol or catechol in compound G (can be converted into ortho-quinone upon treatment with tyrosinase or NaIO₄, respectively), such as serine in compound H (can be converted into a nitrone upon treatment with NaIO₄, then N-methylhydroxylamine), such as tetrazole in compound I (can be converted into nitrile imine upon treatment with 200 nm light) or such as dihydrotetrazine in compound M (can be converted into tetrazine upon treatment with horseradish peroxidase or 660 nm light).

FIG. 12 shows several examples of preferred linker-constructs Q₁-L-F₂.

FIGS. 13, 14 and 15 show several examples of preferred linker-payload constructs Q²(L^{B})Dₓ, with x=1 (FIG. 13), x=2 (FIG. 14) and x=3 (FIG. 15).

FIG. 16 depicts the relative retention times (rrt) of various ADCs versus trastuzumab measured with HIC-HPLC, showing rrt=1.14 for DAR4 and rrt=1.20 for DAR8, while Enhertu® as comparator shows rrt=1.25.

FIG. 17 depicts monomer levels of various ADCs at physiological conditions, PBS at 37° C.

FIG. 18A shows the RP-UPLC spectrum of intact trastuzumab-(tetrazine)₄ [trastuzumab-(45)₂] and FIG. 18B shows the shift in retention time for the DAR 2, 4 and 6 species when conjugated to 98.

FIGS. 19A-19B show the stability of the DAR 8 ADCs over time in PBS at 37° C. (percentage is compared to the DAR at t=0). FIG. 19A shows that the decrease in DAR is equal for all ADCs, independent of the linker used. The decrease of <20% is very low for a period of 28 days, so the constructs are stable. FIG. 19B shows that no aggregation is observed in these 28 days.

FIGS. 20A-20B show the stability of the DAR 8 ADCs over time in human serum at 37° C. Again, all ADCs based on different linkers showed equal DAR decrease (A) (percentage compared to DAR at t=0) and aggregate formation (B).

FIGS. 21A-21B show the stability of the DAR8 ADCs overtime in mice serum at 37° C. Again, all ADCs based on different linkers showed equal DAR decrease (A) (percentage compared to DAR at t=0) and aggregate formation (B) (even though this showed a large variety over time, the trends are the same).

FIGS. 22A-22C show the in vitro potency data of NCI-N87 (A), BT-474 (B) and MDA-MB231 (C) cells treated with increasing concentrations of the ADCs trastuzumab-(3)₂-(59)₄, trastuzumab-(3)₂-(63)₄ and the corresponding free payloads mitomycin and gefitinib. Both DAR8 ADCs show improved N87 cell killing with IC₅₀ values of 0.2 nM and 0.5 nM respectively compared to 94 nM and 997 nM for the corresponding free payloads. In BT-474 cells, a similar trend was seen with IC₅₀ values of 0.2 nM and 0.5 nM respectively compared to 905 nM ad 410 nM for the corresponding free payloads. Negative control cell line, MDA-MB-231 showed no efficacy and no discrimination between the ADCs and the free payloads.

FIGS. 23A-23B show the in vitro potency data of NCI-N87 (A) and MDA-MB231 (B) cells treated with increasing concentrations of the DAR8 ADCs with varying payloads. All ADCs show similar cell killing on NCI-N87 cells with IC₅₀ values between 0.08 and 0.34 nM. Negative control cell line, MDA-MB-231 showed no discrimination between the different ADCs and no cell killing at all.

FIGS. 24A-24B show the in vitro DAR comparison between mitomycin ADCs. FIG. 24A shows a shift in efficacy for the higher DAR species compared to the lower DAR species. FIG. 24B shows the negative control cell line, MDA-MB-231, wherein no sensible difference was seen between the ADCs.

FIG. 25A shows the in vivo efficacy data on the EMT6-hHER2 expressing model over time. The anti-tumor response of DAR4 compared to DAR8 is similar with a correction in dose level for the payload. FIG. 25B shows the body weight of mice over time.

FIG. 26A shows the in vivo efficacy data of the low dose treatment (2 mg/kg) on the EMT6-hHER2 expressing tumor model over time. All DAR8 ADCs according to the invention show a similar anti-tumor response that is improved compared to the Benchmark Enhertu at the same dose level. FIG. 26B shows the body weight of mice over time for the low dose treatment. FIG. 26C shows the plot with the high dose treatment (6 mg/kg) of three ADCs. All three tested DAR8 ADCs according to the invention show complete response until day 30. FIG. 26D shows the body weight of mice over time for the high dose treatment. FIG. 26E shows the survival in a Kaplan-Meier plot of the test items over time, for both the low and high dose treatments. A clear effect is observed comparing the Benchmark Enhertu to the new DAR8 ADCs according to the invention.

FIG. 27 shows the in vivo tolerability data in female CD-1 mice over time. Only at the highest concentration one mice was sacrificed due to severe body weight loss (>20%6). Therefore the maximum tolerated dose is at least 80 mg/kg.

FIGS. 28A-28C show the RP-UPLC spectra of intact trastuzumab-(tetrazine)₄ [trastuzumab-(3)₂](A), and the same construct conjugated to 7 (B) or 9 (C). The spectrum of FIG. 28B was generated before purification, and contained some remaining compound 3. The spectra of FIGS. 28B and 28C show that the DAR8 constructs are almost exclusively formed, with only very minor further peaks.

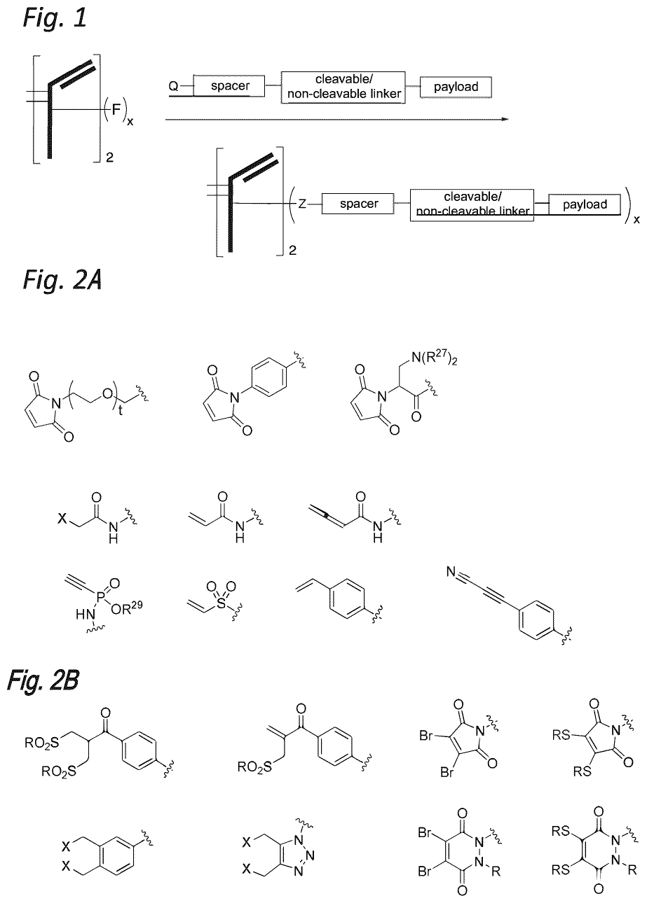

### EXAMPLES

The invention is illustrated by the following examples.

### General Reagents and Analytics

General procedure for analytical RP-UPLC (DTT treated samples): Prior to RP-UPLC analysis, IgG (10 μL, 1 mg/mL in PBS pH 7.4) was added to 12.5 mM DTT, 100 mM TrisHCl pH 8.0 (40 μL) and incubated for 15 minutes at 37° C. The reaction was quenched by adding 49% acetonitrile, 49% water, 2% formic acid (50 μL). RP-UPLC analysis was performed on a Waters Acquity UPLC-SQD. The sample (5 μL) was injected with 0.4 mL/min onto Bioresolve RP mAb 2.1 *150 mm 2.7 μm (Waters) with a column temperature of 70° C. A linear gradient was applied in 9 minutes from 30 to 54% acetonitrile in 0.1% TFA and water. Absorbance of eluted peaks was measured at 215 nm followed by automated integration (MassLynx, Waters) to determine reaction conversion.

General procedure for mass spectral analysis of (modified) monoclonal antibodies: Prior to mass spectral analysis, IgG was treated with IdeS, which allows analysis of the Fc/2 fragment. For analysis of both light and heavy chain, a solution of 20 μg (modified) IgG was incubated for 5 minutes at 37° C. with 100 mM DTT in a total volume of 4 μL. If present, azide-functionalities are reduced to amines under these conditions. For analysis of the Fc/2 fragment, a solution of 20 μg (modified) IgG was incubated for 1 hour at 37° C. with IdeS/Fabricator™ (1.25 U/μL) in phosphate-buffered saline (PBS) pH 6.6 in a total volume of 10 μL. Samples were diluted to 80 μL followed by analysis electrospray ionization time-of-flight (ESI-TOF) on a JEOL AccuTOF. Deconvoluted spectra were obtained using Magtran software.

General procedure for analytical SEC analysis: HPLC-SEC analysis was performed on an Agilent 1100 series (Hewlett Packard) using an Xbridge BEH200A (3.5 μM, 7.8×300 mm, PN 186007640 Waters) column. The sample was diluted to 1 mg/mL in PBS and measured with 0.86 mL/min isocratic method (100 mM sodium phosphate (NaHPO₄/Na₂PO₄), 200 mM NaCl, pH 6.8, containing 10% isopropanol) for 17 minutes.

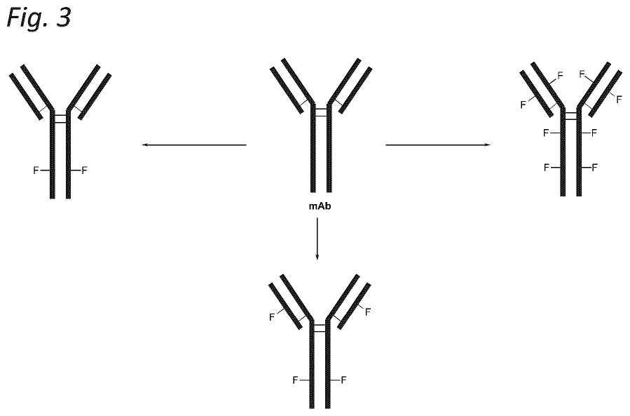

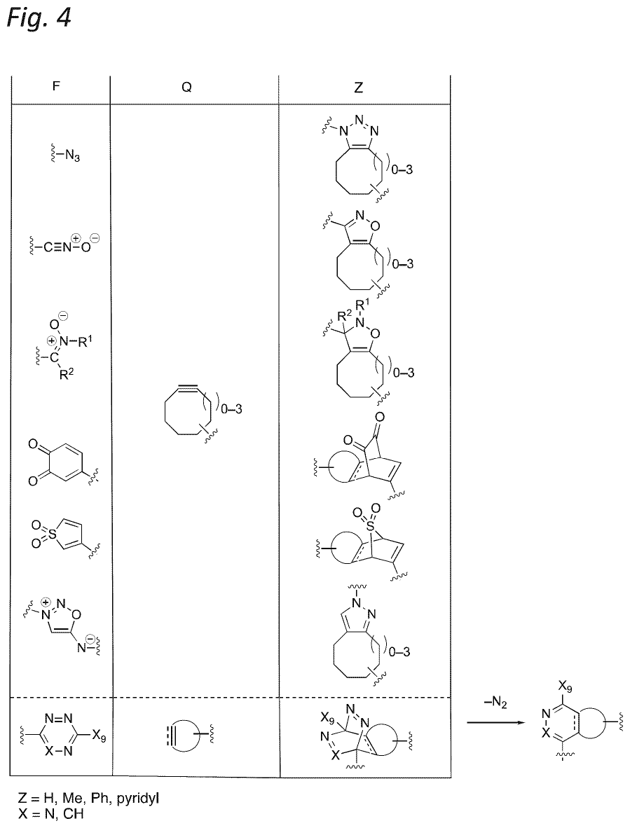

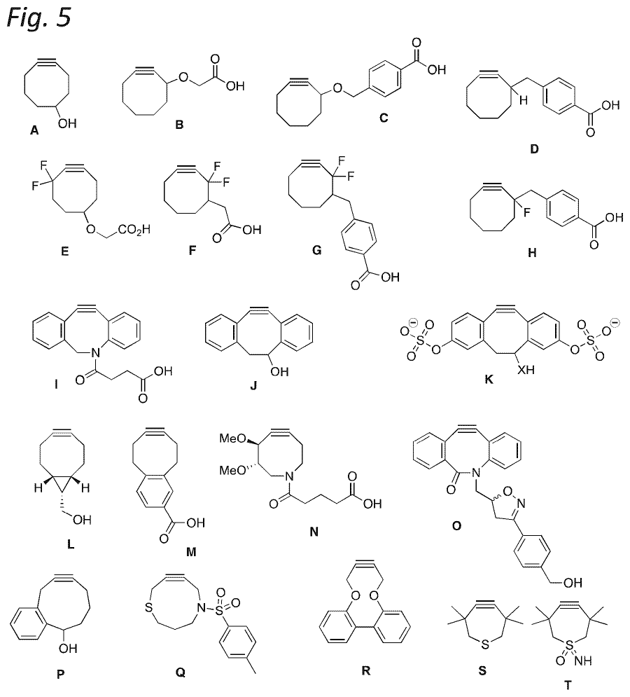

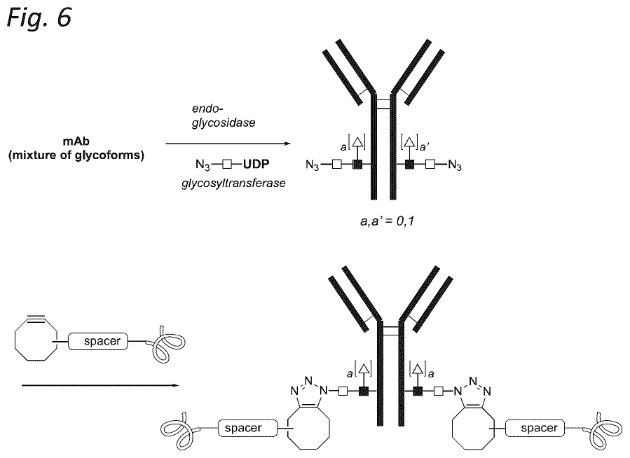

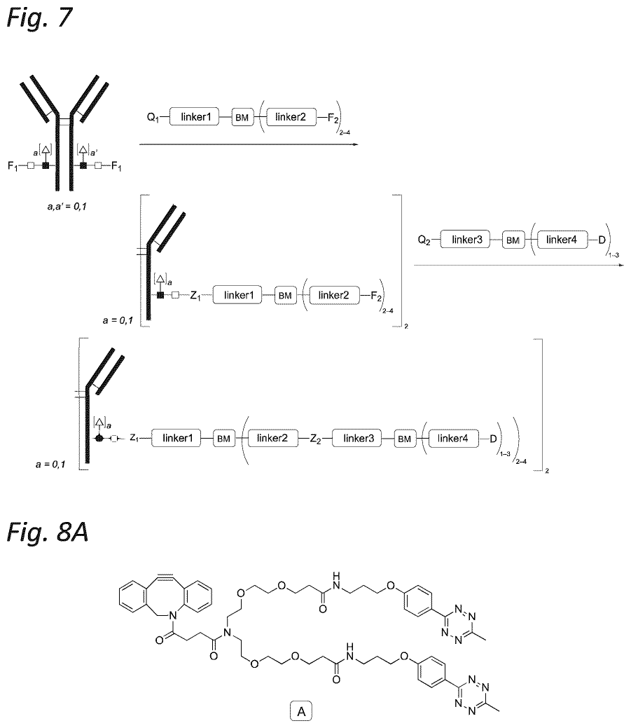

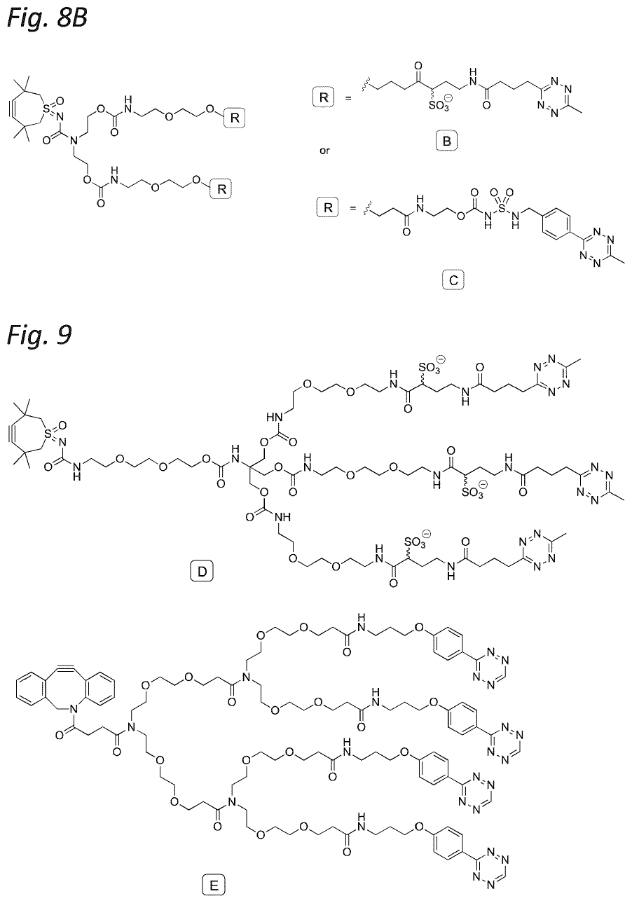

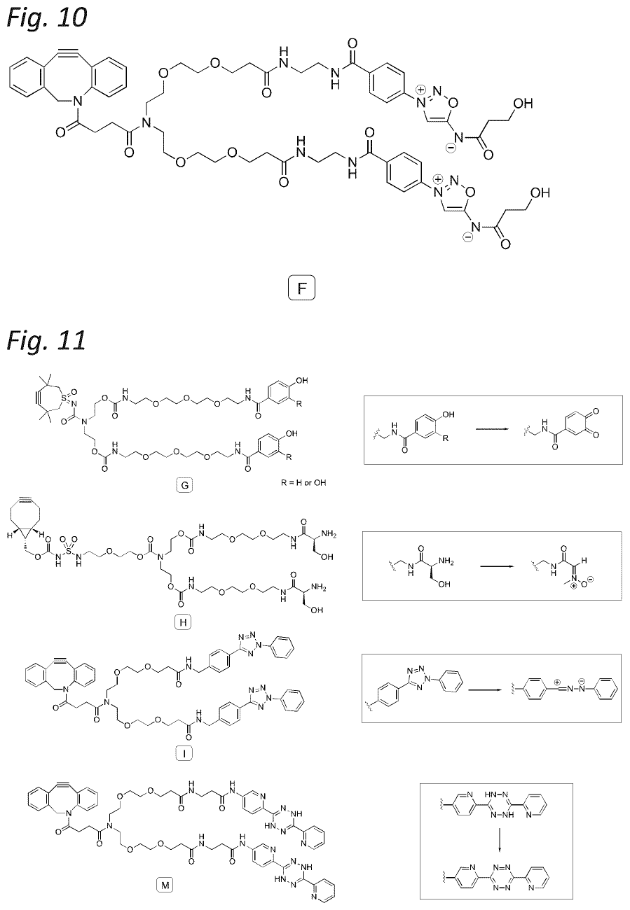

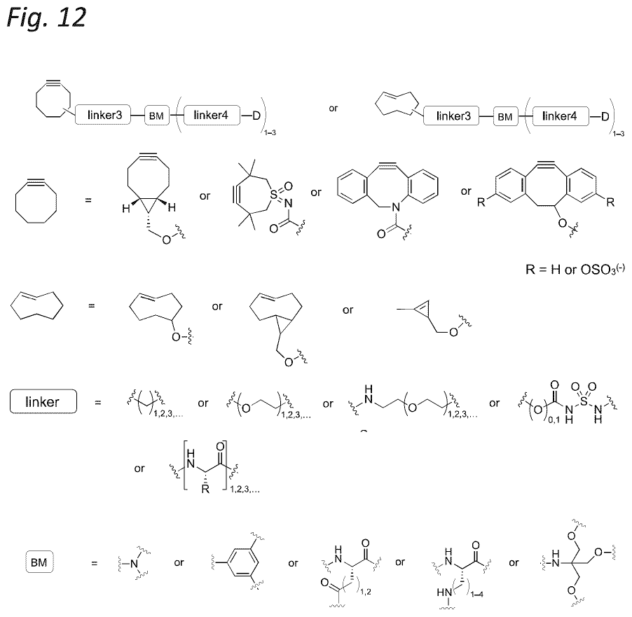

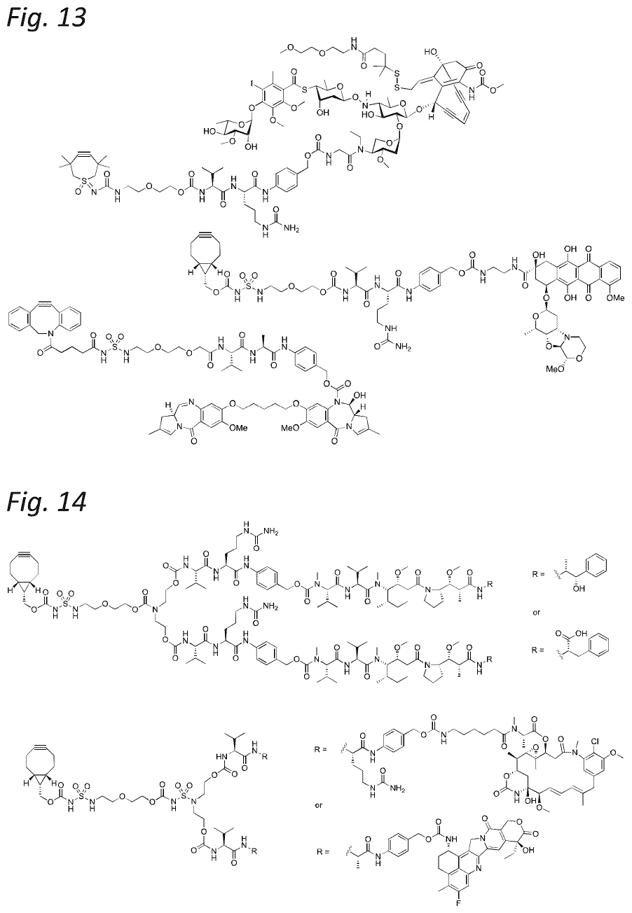

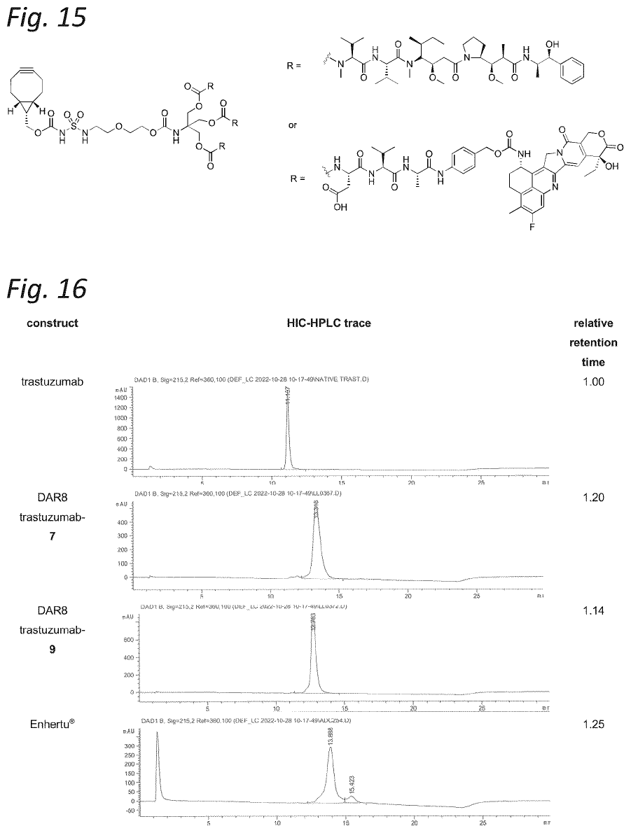

General procedure for enzymatic remodeling of IgG to mAb-(6-N₃-GalNAc)₂: IgG (15 mg/mL) was incubated with 1% w/w EndoSH (as described in PCT/EP2017/052792, see Examples 1-3, and SEQ. ID No: 1, which is incorporated by reference herein), 3% w/w His-TnGalNAcT (as described in PCT/EP2016/059194, see Examples 3 and 4, and SEQ. ID No: 49, which is incorporated by reference herein), 0.01% AP (Roche) and UDP 6-N₃-GalNAc (compound 2d in FIG. 3, 10 eq compared to IgG) in 6 mM MnCl₂ and TBS for 16 hours at 30° C. Next, the functionalized IgG was purified using a HiTrap MabSelect sure 5 mL column. After loading of the reaction mixture the column was washed with TBS+0.2% triton and TBS. The IgG was eluted with 0.1 M AcOH pH 2.7 and neutralized with 2.5 M Tris-HCl pH 8.8. After three times dialysis to 20 mM histidine, 150 mM NaCl pH 7.5, the IgG was concentrated to 15-20 mg/mL using a Vivaspin Turbo 15 ultrafiltration unit (Sartorius).

### Examples 1-49: Synthesis of Click Probes and Linker Conjugates

### Example 1: Preparation of Compound 3

To a solution of compound 1 (10.0 mg, 1 Eq, 12.2 μmol) in dry DMF (850 μL) was added compound 2 (10.3 mg, 3 Eq, 36.6 μmol), followed by triethylamine (6.18 mg, 8.51 μL, 5 Eq, 61.1 μmol). The reaction mixture was stirred at room temperature for 2 hours. The reaction mixture was purified by prep-HPLC; (30%→100%, MeCN/Water+10 mM NH₄HCO₃, runtime 21 minutes, Column Xbridge prep C18 5 μm OBD, 30×100 mm) to afford compound 3 (5 mg, 4.7 μmol, 38%) as a pink solid. LCMS (ESI+) calculated for C₅₇H₆₇N₁₂O₁₀⁺ (M+H)⁺ 1079.5. found 1079.9.

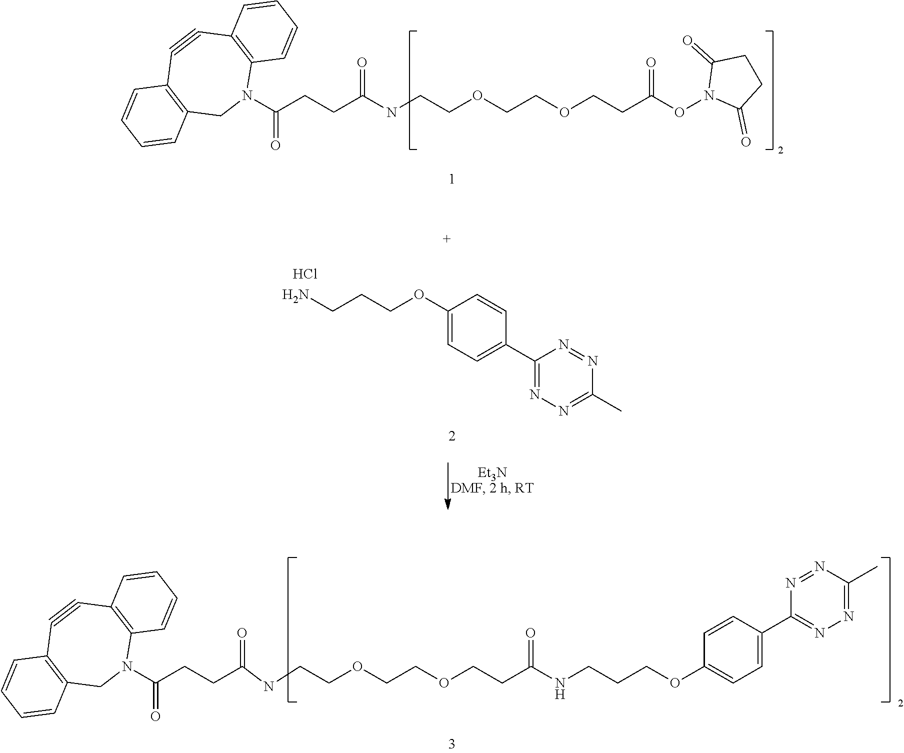

### Example 2: Preparation of Compound 5

Compound 4 (163 mg, 240 mol) was added to a mixture of exatecan mesylate (125 mg, 235 μmol) and DIPEA (61 mg, 82 μL, 0.47 mmol) in dry DMF (0.9 mL). After 20 hours, the reaction mixture was diluted to 9 mL DCM and purified by flash column chromatography over silicagel (0→40% MeOH/DCM) to afford 5 (155 mg, 159 μmol, 68%). LCMS (ESI+) calculated for C₅₅H₅₄FN₆O₁₀⁺ (M+H)⁺ 977.39. found 977.72. In addition to 5, free base of exatecan (82.4 mg, 189 mol, 20%) was recovered. LCMS (ESI+) calculated for C₂₄H₂₃FN₃O₄⁺ (M+H)⁺ 436.5. found 436.5.

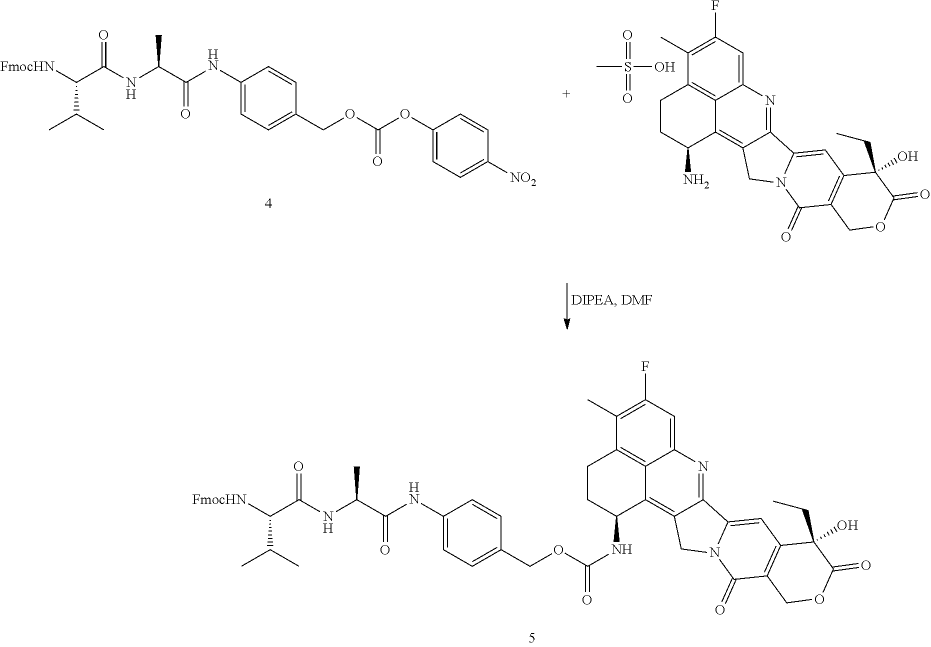

### Example 3. Preparation of Compound 7

The synthesis of BCN—HS-PEG₂-HS-(va-PABC-Ex)₂ (7 is also described in PCT/EP2021/075401 (example 4), incorporated herein. To a solution of compound 5 (155 mg, 159 μmol) in DMF (1.6 mL) were added Et₃N (73 mg, 101 μL, 0.72 mmol) and a solution of compound 6 (65 mg, 72 μmol) in DMF (1.4 mL). The reaction mixture was stirred for 18 h, diluted with DCM (20 mL) and purified by flash column chromatography over silicagel (0→40% MeOH/DCM) to afford 7 as a pale-yellow solid (94 mg, 44 μmol, 28%). LCMS (ESI+) calculated for C₁₀₂H₁₁₈F₂N₁₆O₂₉S₂²⁺ (M/2+H)⁺ 1066.9. found 1067.1.

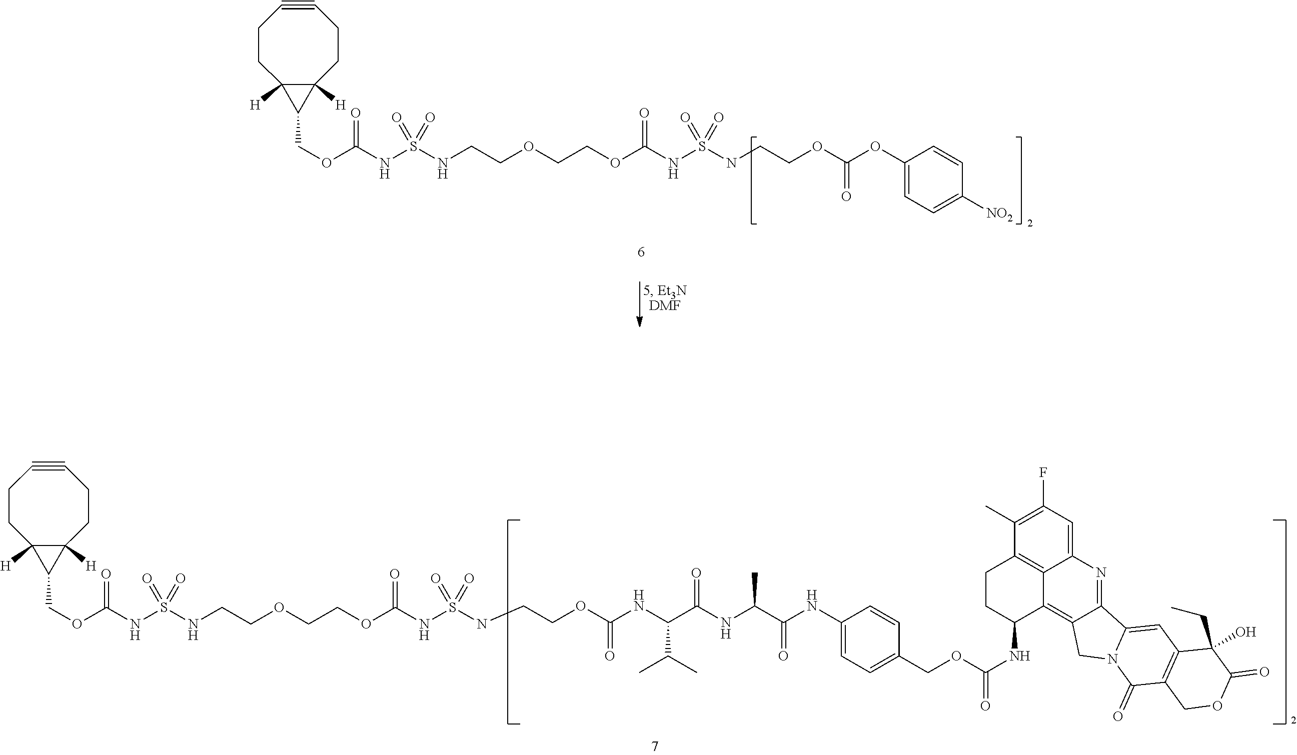

### Example 4. Preparation of Compound 9

The synthesis of BCN—HS-PEG₂-(eva-PABC-Ex)₂ (9) is also described in PCT/EP2021/075401 (example 11), incorporated herein by reference. A solution of BCN—HS-PEG₂-b-(Glu(OFm)—OH)₂ (8, 12.1 mg, 10 μmol, 1.0 eq) dissolved in anhydrous DMF (180 μL) was added to a solution of NH₂-Val-Ala-PABC-exatecan (5b, Fmoc-deprotected 5, 19 mg, 25 μmol, 2.5 eq) in anhydrous DCM (180 μL), DIPEA (11 μL, 63 μmol, 6.2 eq) and HATU (8.9 mg, 23 μmol, 2.3 eq). After stirring for 2 hours at room temperature, the reaction mixture was further diluted with DCM (800 μL) and purified by flash column chromatography over silicagel (0%→20% MeOH in DCM) to give the product as a clear oil (difficult to determine yield due to presence of DMF). LCMS (ESI+) calculated for C₁₄₀H₁₅₀F₂N₁₇O₃₃S⁺² (M/2+H)⁺ 1334.0. found 1334.8.

This compound was dissolved in DMF (300 μL) and triethylamine (21 μL, 150 μmol, 15 eq) was added. After 17 hours at room temperature, the reaction mixture was diluted with DCM (700 μL) and purified by flash column chromatography over silicagel (0%→45% MeOH in DCM) to afford compound 9 (10.2 mg, 4.4 μmol, 44%) as a yellow solid. LCMS (ESI+) calculated for C₁₁₂H₁₃₀F₂N₁₇O₃₃S⁺² (M/2+H)⁺ 1156.2. found 1156.7.

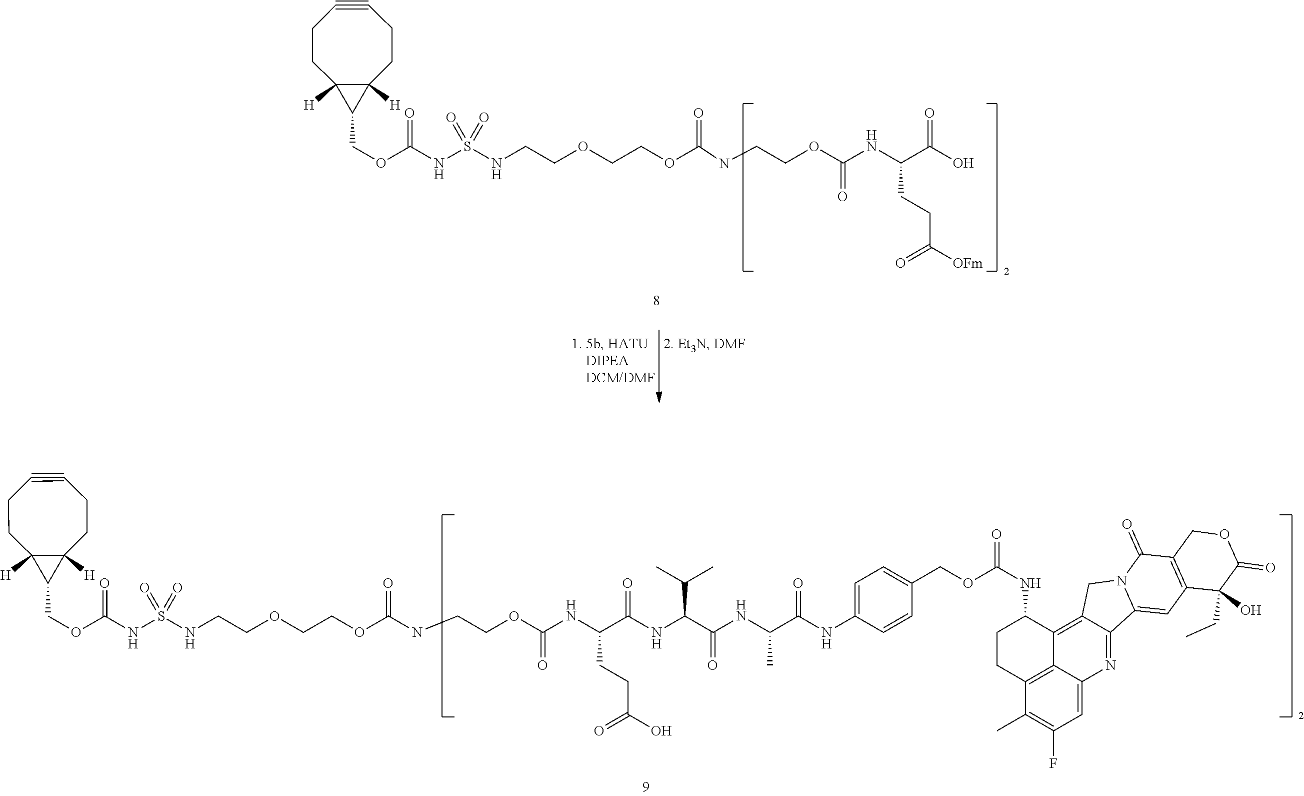

### Example 5. Preparation of Compound 11

Diethanolamine (894.1 mg, 816.5 μL, 1.1 Eq, 8.504 mmol) and sodium carbonate (1.188 g, 1.45 Eq, 11.21 mmol) were added to 1,4-dioxane (20 mL) and water (20 mL). The mixture was stirred at room temperature to form a suspension. (9H-fluoren-9-yl)methyl carbonochloridate (2000 mg, 1 Eq, 7.731 mmol) was dissolved in toluene (5 mL) and added dropwise to the above reaction solution (the internal temperature was controlled to not exceed 25° C. during the dropwise addition) and stirring was continued for 2 hours. The pH was adjusted to 3 with 4 M HCl(aq) and the aqueous phase was extracted twice with ethyl acetate (50 mL). The organic phase was washed saturated brine (2×50 mL), dried over anhydrous sodium sulfate and concentrated in vacuo to afford compound 10 (3.13 g, 7.6 mmol, 99%, 80% Purity) as an opaque oil. LCMS (ESI+) calculated for C₁₉H₂₂NO₄⁺ (M+H)⁺ 328.2. found 928.3.

Compound 10 (1260 mg, 1 Eq, 3.849 mmol) was dissolved in dry DCM (50 mL). Then, bis(perfluorophenyl) carbonate (3.185 g, 2.1 Eq, 8.082 mmol) was added followed by DIPEA (1.492 g, 2.01 mL, 3 Eq, 11.55 mmol) and stirring was continued for 3 hours. The residue was then purified by by flash column chromatography over silicagel (0→10% Acetone in DCM) to afford compound 11 (2.5 g, 3.3 mmol, 87%, 100% Purity) as a clear oil. LCMS (ESI+) calculated for C₃₃H₂₀F₁₀NO₈⁺ (M+H)⁺ 748.1. found 748.4.

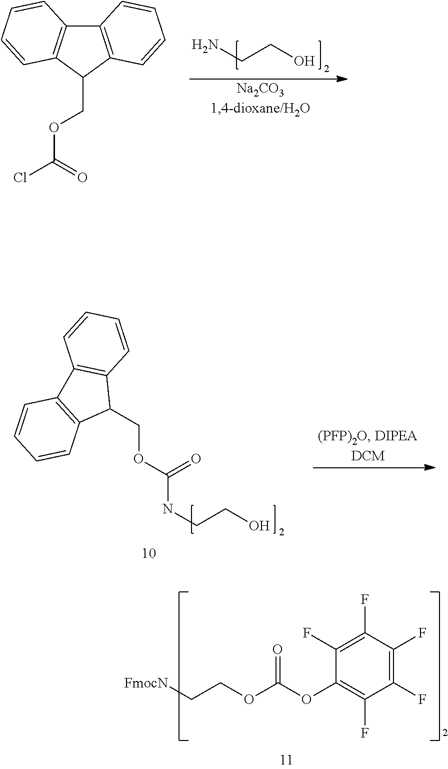

### Example 6. Preparation of Compound 13

A solution of 2,2′-(ethane-1,2-diylbis(oxy))bis(ethan-1-amine) (3.40 g, 3.35 mL, 10 Eq, 22.9 mmol) in DCM (40 mL) was cooled to 0° C. To this di-tert-butyl dicarbonate (500 mg, 1 Eq, 2.29 mmol) was added and the mixture was stirred at room temperature for 18 hours. The organic phase was washed with water (3×40 mL), until all the unreacted 2,2′-(ethane-1,2-diylbis(oxy))bis(ethan-1-amine) was extracted. The organic phase was dried (Na₂SO₄) and concentrated in vacuo to afford 12 (361 mg, 1.3 mmol, 57%, 90% Purity) as a yellow oil. LCMS (ESI+) calculated for C₁₁H₂₅N₂O₄⁺ (M+H)⁺ 249.2. found 249.4.

To a solution of amine 12 (150.0 mg, 1 Eq, 200.7 μmol) in dry DCM (5.00 mL) was added compound 11 (109.6 mg, 2.2 Eq, 441.5 μmol), followed by DIPEA (129.7 mg, 175 μL, 5 Eq, 1.003 mmol). The reaction mixture was stirred at room temperature for 1 hour. The reaction mixture was concentrated in vacuo. The resulting crude was dissolved in DCM (1 mL) and was loaded onto the column for purification flash column chromatography over silicagel (0→20% MeOH in DCM) afford the Boc-protected intermediate (218 mg, 0.20 mmol, 99%, 80% Purity) as a colorless oil. LCMS (ESI+) calculated for C₄₃H₆₆N₅O₁₄⁺ (M+H)⁺ 876.5. found 876.7.

To a solution of the intermediate (176 mg, 1 Eq, 201 μmol) in dry DCM (5.00 mL) was added HCl in dioxane (147 mg, 1.00 mL, 4 molar, 20 Eq, 4.02 mmol). The reaction mixture was stirred at room temperature for 18 hours and subsequently concentrated in vacuo to afford the HCl salt 13 (150 mg, 200 μmol, 99%) as an off-white foam. LCMS (ESI+) calculated for C₃₃H₅₀N₅O₁₀⁺ (M+H)⁺ 676.4. found 676.6.

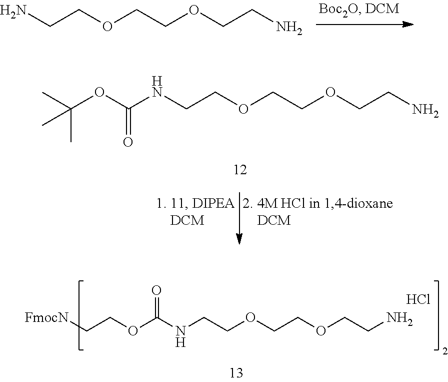

### Example 7. Preparation of Compound 15

To a solution of sulfo-D-alanine hydrate (554 mg, 1 Eq, 2.96 mmol) and triethylamine (599 mg, 825 μL, 2 Eq, 5.92 mmol) in DMF (15 mL) was added di-tert-butyl dicarbonate (775 mg, 1.2 Eq, 3.55 mmol). The reaction mixture was stirred at room temperature for 3 hours. The solvent was evaporated in vacuo. The residue was redissolved in CH₂Cl₂ (10 mL) and treated with Et₂O (30 mL) under vigorous stirring. The organic solvents were decanted and discarded. The oily residue was washed with Et₂O (20 mL) and dried in vacuo to give compound 14 (1134 mg, 2.9 mmol, 100%, 70% Purity) as a colorless oil. LCMS (ESI−) calculated for C₈H₁₄NO₇S⁻ (M−H)⁻ 268.1. found 268.3.

To a solution of N-Boc-cysteic acid 14 (54 mg, 3 Eq, 0.20 mmol) in DMF (1 mL) was added compound 13 (50 mg, 1 Eq, 67 μmol), HATU (76 mg, 3 Eq, 0.20 mmol) and DIPEA (52 mg, 70 μL, 6 Eq, 0.40 mmol) and stirring was continued for 2 hours. The mixture was concentrated in vacuo and the crude dissolved in DCM (10 ml). The mixture was purified by flash column chromatography over silicagel (0%→70% MeOH in DCM) to afford Boc-protected intermediate (50 mg, 36 μmol, 53%, 84% Purity) as a colorless oil. LCMS (ESI+) calculated for C₄₉H₇₅N₇O₂₂S₂⁺ (M+H)⁺ 1178.4. found 1178.7.

To a solution of the Boc-protected intermediate (50 mg, 1 Eq, 42 μmol) in dry DCM (2.00 mL) and MeOH (1.00 mL) was added HCl in 1,4-dioxane (46 mg, 0.32 mL, 4 molar, 30 Eq, 1.3 mmol). The reaction mixture was stirred at room temperature for 26 hours after which additional HCl in 1,4-dioxane (46 mg, 0.32 mL, 4 molar, 30 Eq, 1.3 mmol) was added to the reaction mixture. Stirring was continued for an additional hour upon which the reaction mixture was concentrated in vacuo to afford compound 15 (45 mg, 43 μmol, 100%) as an off-white solid. LCMS (ESI+) calculated for C₃₉H₆₀N₇O₁₈S₂⁺ (M+H)⁺ 978.3. found 978.6.

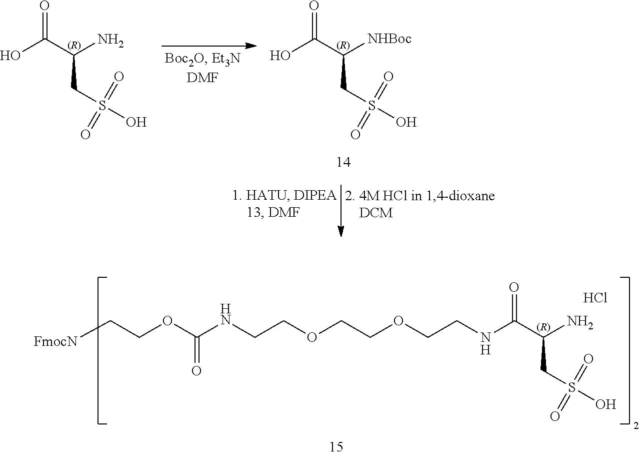

### Example 8. Preparation of Compound 17

To a solution of 4-(6-methyl-1,2,4,5-tetrazin-3-yl)butanoic acid (4.3 mg, 2.5 Eq, 24 μmol) in DMF (1 mL) was added HATU (11 mg, 3 Eq, 29 μmol) and DIPEA (7.4 mg, 9.9 μL, 6 Eq, 57 μmol). After 10 minutes HCl salt 15 (10 mg, 1 Eq, 9.5 μmol) was added and stirring was continued for 1 hour. The reaction mixture was concentrated in vacuo and the crude dissolved in MeCN (1 ml). The solution was purified by reverse phase chromatography (10%→100%, MeCN in water) to afford 16 (7 mg, 5 μmol, 50%, 95% Purity) as a pink solid. LCMS (ESI+) calculated for C₅₃H₇₆N₁₅O₂₀S₂⁺ (M+H)⁺ 1306.5. found 1306.7.

Triethylamine (11 mg, 15 μL, 20 Eq, 0.11 mmol) was added to a solution of compound 16 (7.0 mg, 1 Eq, 5.4 μmol) in DMF (0.200 mL) and H₂O (10 μL). The reaction mixture was stirred for 18 hours until complete Fmoc deprotection was observed. TMTHSI-OSu (2.7 mg, 1.5 Eq, 8.0 μmol) was added to the reaction mixture and stirring was continued for 4 days. The reaction mixture was purified by prep-HPLC; (30%→100%, MeCN/Water+1% AcOH, runtime 21 minutes, Column Xbridge prep C18 5 μm OBD, 30×100 mm) to afford 17 (2.5 mg, 1.9 μmol, 36%) as a pink solid. LCMS (ESI−) calculated for C₄₈H₇₈N₁₆O₂₀S₃⁻² (M/2−H)⁻ 653.2. found 653.6.

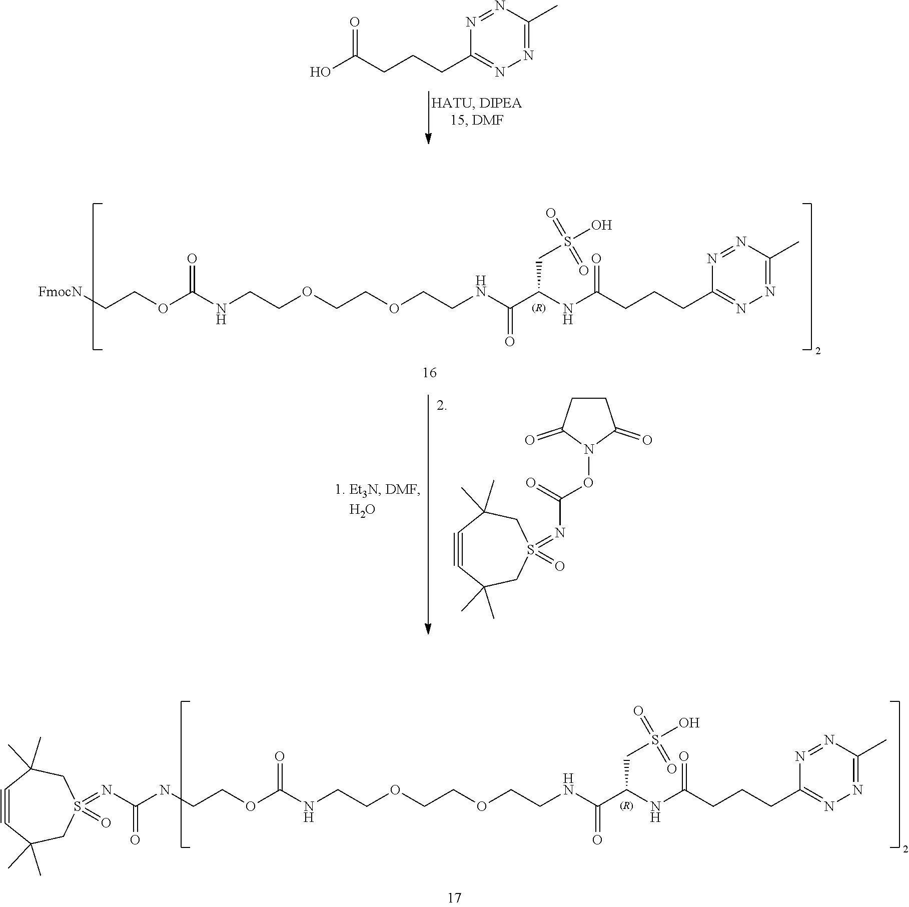

### Example 9. Preparation of Compound 19

To a solution of compound 11 (50.0 mg, 1 Eq. 66.9 μmol) in dry DMF (1.00 mL) was added 3-(2-(2-aminoethoxy)ethoxy)propanoic acid (26.1 mg, 2.2 Eq, 147 μmol), followed by DIPEA (43.2 mg, 334 μL, 5 Eq, 334 μmol). After stirring for 18 hours, the reaction mixture was concentrated in vacuo. The resulting crude was dissolved in DCM (3 mL) and was loaded onto the column for purification by flash column chromatography over silicagel (0-10% MeOH in DCM with 1% AcOH) to afford 18 (55.3 mg, 75.4 μmol, 113%) as a colorless oil. LCMS (ESI+) calculated for C₃₅H₄₈N₃O₁₄⁺ (M+H)⁺ 734.3. found 734.6.

DCC (32.3 mg, 2.3 Eq, 157 μmol) and 1-hydroxypyrrolidine-2,5-dione (18.0 mg, 2.3 Eq, 157 μmol) were added to a solution of compound 18 (50.0 mg, 1 Eq, 68.1 μmol) dissolved in DCM (2 ml-) and DMF (2 mL). The reaction mixture was stirred for 18 hours at room temperature. sulfo-L-alanine (34.6 mg, 3 Eq. 204 μmol) and DIPEA (105.6 mg, 142.4 μL, 12 Eq. 818 μmol) were added to the reaction mixture and stirring was continued for 24 hours. The mixture was filtered, and the filtrate was concentrated in vacuo. The resulting crude was dissolved in water and MeCN and was purified by reverse phase chromatography (0→100% MeCN in H₂O+1% AcOH) to afford compound 19 (73.1 mg, 70.6 μmol, 104%) as a colorless oil. LCMS (ESI−) calculated for C₄₁H₅₆N₅O₂₂S₂⁻ (M−H)⁻ 1034.3. found 1034.7.

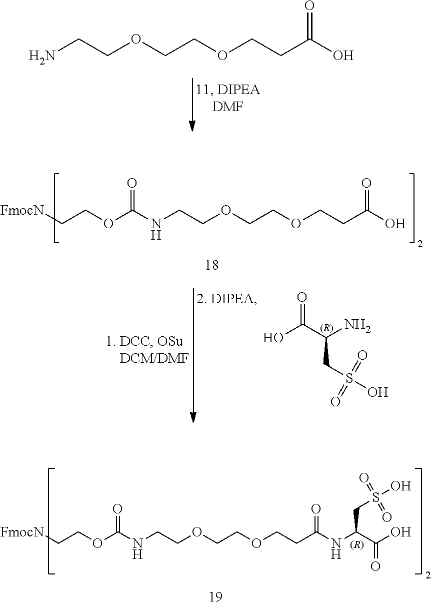

### Example 10. Preparation of Compound 21

DIPEA (7.49 mg, 10.1 μL, 6 Eq, 57.9 μmol) and HATU (9.18 mg, 2.5 Eq, 24.1 μmol) were added to compound 19 (10.0 mg, 1 Eq, 9.65 μmol) in DMF (0.5 mL). Subsequently, (4-(6-methyl-1,2,4,5-tetrazin-3-yl)phenyl)methanamine hydrochloride (5.05 mg, 2.2 Eq, 21.2 μmol) was added. The mixture was stirred for 1 hour, concentrated in vacuo and the resulting crude dissolved in DCM (10 ml). The solution was purified by flash column chromatography over silicagel (0%→30% MeOH in DCM) to afford compound 20 (2.4 mg, 1.7 μmol, 18%) as a pink solid. LCMS (ESI−) calculated for C₆₁H₇₄N₁₅O₂₀S₂⁻ (M−H)⁻ 1400.5. found 1400.7.

Triethylamine (1.7 mg, 2.4 μL, 10 Eq, 17 μmol) was added to compound 20 (2.4 mg, 1.0 Eq, 1.7 μmol) in DMF (0.1 mL). The reaction mixture was stirred for 42 hours until Fmoc deprotection was observed. TMTH-SI-OSu (0.70 mg, 1.2 Eq, 2.1 μmol) was added to the reaction mixture and stirring was continued for 92 hours. LCMS analysis indicated formation of 21. LCMS (ESI−) calculated for C₅₇H₇₉N₁₆O₂₀S₃⁻ (M−H)⁻ 1403.5. found 1403.6.

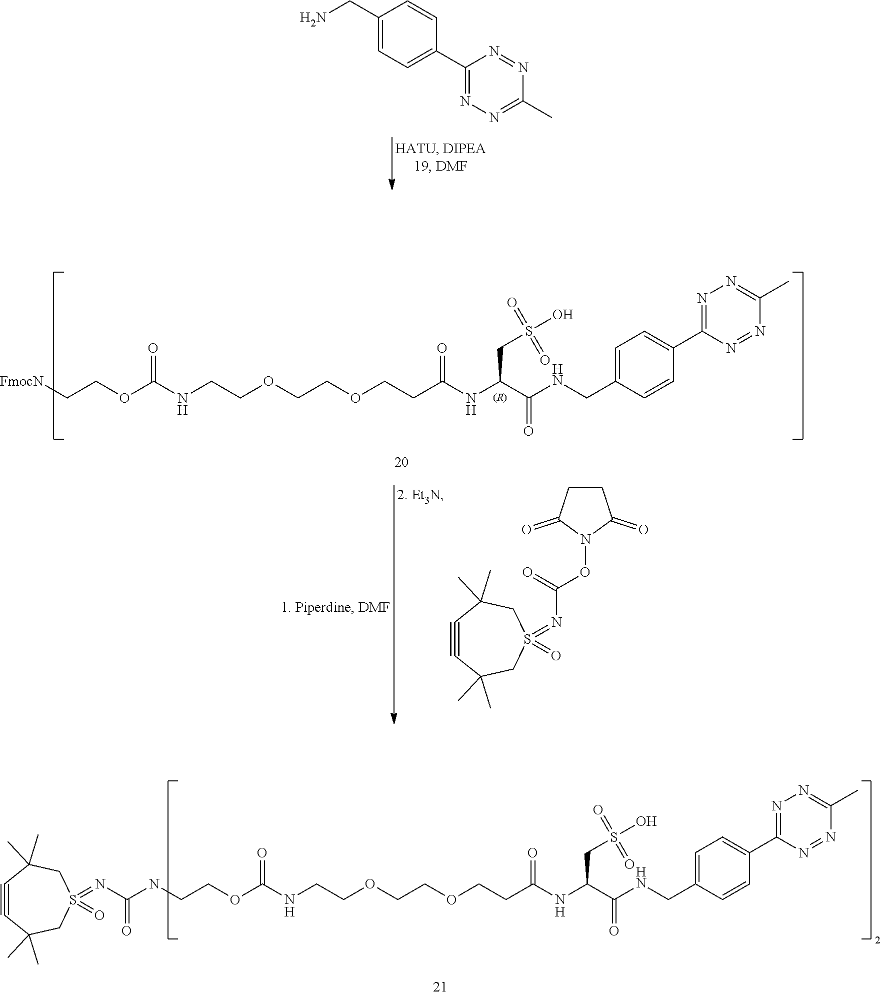

### Example 11. Preparation of Compound 23

To a solution of 4-(6-methyl-1,2,4,5-tetrazin-3-yl)butanoic acid (24 mg, 2.5 Eq, 0.13 mmol) in DMF (1 mL) was added HATU (61 mg, 3 Eq, 0.16 mmol) and DIPEA (55 mg, 74 μL, 8 Eq, 0.43 mmol). After 10 minutes compound 13 (40 mg, 1 Eq, 53 μmol) was added and stirring was continued for 1 hour. The reaction mixture was purified by flash column chromatography over silicagel (0%→10% MeOH in DCM) to yield Fmoc-protected intermediate (82 mg, 53 μmol, 97%, 65% purity) as a pink solid. LCMS (ESI+) calculated for C₄₇H₆₆N₁₃O₁₂⁺ (M+H)⁺ 1004.5. found 1004.8.

To a solution of the Fmoc-protected intermediate (82 mg, 65% Wt, 1 Eq, 53 μmol) in DCM (1.0 mL) was added piperidine (0.18 g, 0.21 mL, 40 Eq, 2.1 mmol) and stirring was continued for 22 hours. The reaction mixture was extracted with sat. NaHCO₃, dried and concentrated in vacuo to afford 42 mg of pink crude. The crude was dissolved in DCM (1 ml) and purified by flash column chromatography over silicagel (0%→15% MeOH in DCM) to yield compound 22 (11.7 mg, 15.0 μmol, 28%) as a pink solid. LCMS (ESI+) calculated for C₃₂H₅₆N₁₃O₁₀⁺ (M+H)⁺ 782.4. found 782.7.

To a solution of compound 22 (5.8 mg, 1 Eq, 7.4 μmol) in DMF (0.50 mL) was added DBCO-C6-OSu (4.8 mg, 1.5 Eq, 11 μmol), HOBt (1.7 mg, 1.5 Eq, 11 μmol) and triethylamine (3.8 mg, 5.2 μL, 5 Eq, 37 μmol). The resulting reaction mixture was left to stand for 3 days after which it was diluted to 550 μL of DMF and was purified by prep-HPLC (injection volume 600 μL DMF; 5%→95%, MeCN/Water+10 mM NH₄HCO₃, runtime 21 minutes, Column Xbridge prep C18 5 μm OBD, 30×100 mm) to afford compound 23 (1.6 mg, 1.5 μmol, 20%) as a pink oil. LCMS (ESI+) calculated for C₅₃H₇₃N₁₄O₁₂⁺ (M+H)⁺ 1097.5. found 1097.8.

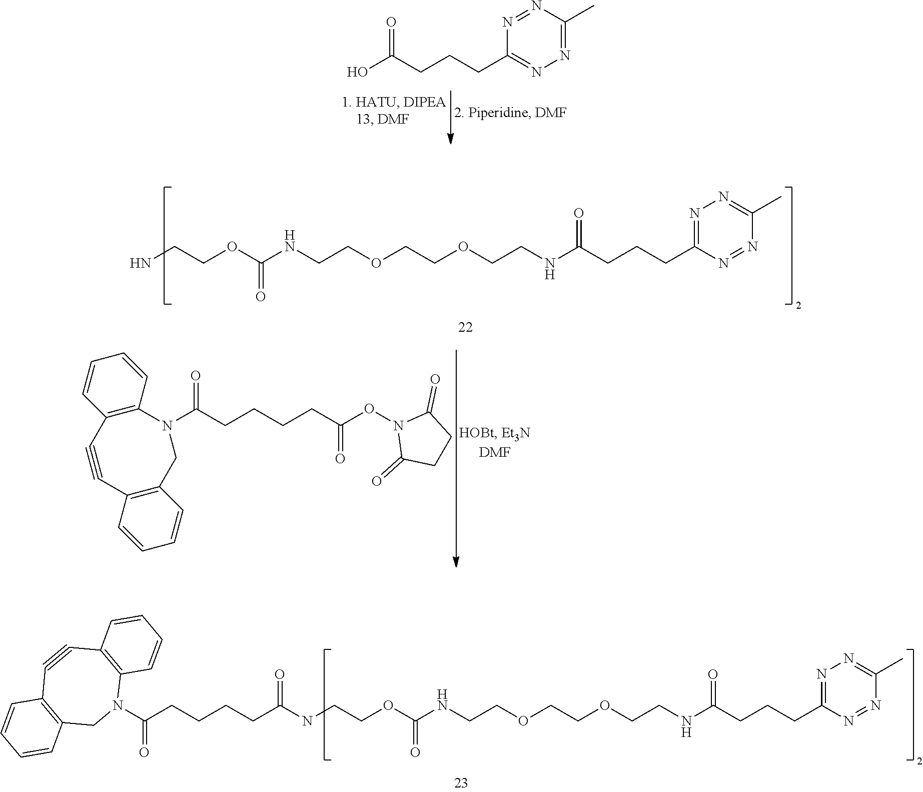

### Example 12. Preparation of Compound 25

2-(2-aminoethoxy)ethan-1-ol (185.9 mg, 177.4 μL, 3 Eq, 1.768 mmol) was added to a solution of TMTHSI-OSu (200.6 mg, 1 Eq, 589.3 μmol) in DCM (3 mL). The reaction mixture was stirred for 21 hours and subsequently purified by flash column chromatography over silicagel (0%→60% Aceton in DCM) to yield unactivated alcohol (168.2 mg, 509.0 μmol, 86.37%) as a colorless oil. LCMS (ESI+) calculated for C₁₅H₂₇N₂O₄S⁺ (M+H)⁺ 331.2. found 331.1.

The unactivated alcohol (168.2 mg, 1.0 Eq, 509.0 μmol) was dissolved in DCM (3.00 mL) followed by addition of bis(4-nitrophenyl) carbonate (309.7 mg, 2 Eq, 1.018 mmol) and triethylamine (154.5 mg, 213 μL, 3.0 Eq, 1.527 mmol). The solution was stirred for 2 hours and subsequently purified by flash column chromatography over silicagel (0%→30% Aceton in DCM) to yield compound 24 (262.5 mg, 0.41 mmol, 80%, 77% Purity). LCMS (ESI+) calculated for C₂₂H₃₀N₃O₈S⁺ (M+H)⁺ 496.2. found 496.2.

To a solution of compound 22 (5.8 mg, 1 Eq, 7.4 μmol) in DMF (0.50 mL) was added compound 24 (5.5 mg, 1.5 Eq, 11 μmol), HOBt (1.1 mg, 1 Eq, 7.4 μmol) and triethylamine (3.8 mg, 5.2 μL, 5 Eq, 37 μmol). The resulting reaction mixture was stirred for 18 hours and subsequently purified by prep-HPLC (injection volume 600 μL DMF; 30%→100%, MeCN/Water+10 mM NH₄HCO₃, runtime 21 minutes, Column Xbridge prep C18 5 μm OBD, 30×100 mm) to afford compound 25 (3.9 mg, 3.4 μmol, 46%) as a pink oil. LCMS (ESI+) calculated for C₄₈H₈₀N₁₅O₁₅S⁺ (M+H)⁺ 1138.6. found 1138.8.

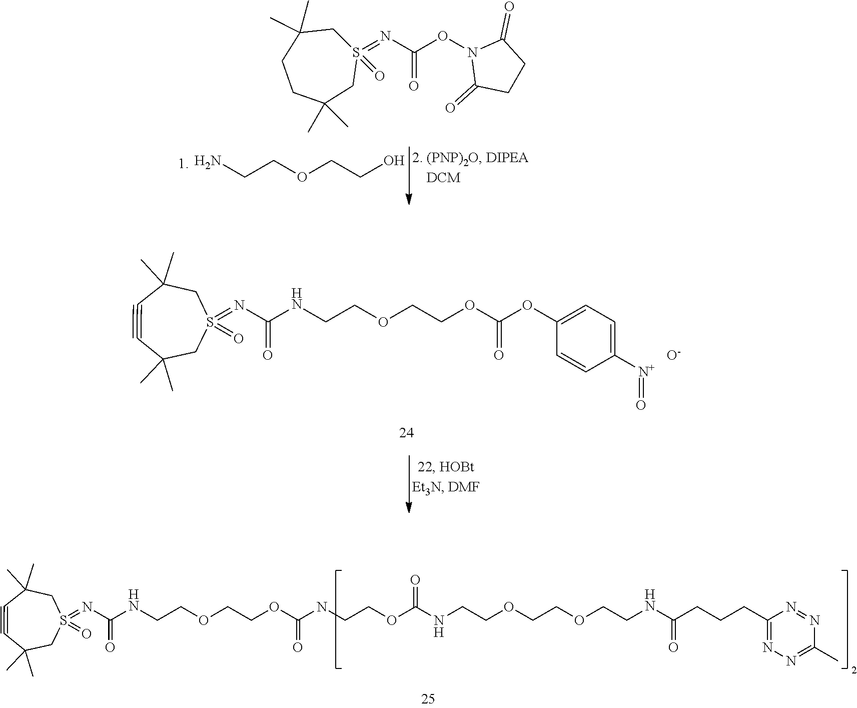

### Example 13. Preparation of Compound 26

To a solution of compound 1 (9.35 mg, 1 Eq. 11.4 μmol) in anhydrous DMF (500 uL) was added the HCl salt of methyltetrazine-PEG4-Amine (9.8 mg, 2.1 Eq, 25 μmol), followed by triethylamine (5.2 mg, 7.2 μL, 4.5 Eq, 52 μmol). After stirring for 2 hours at room temperature, the reaction mixture was further diluted with 200 uL DMF and purified by prep-HPLC; (30%→70%, MeCN/Water+10 mM NH₄HCO₃, runtime 23 minutes, Column Xbridge prep C18 5 μm OBD, 30×100 mm) to afford compound 26 (9.1 mg, 6.9 μmol, 61%) as a pink oil. LCMS (ESI+) calculated for C₆₇H₃₇N₁₂O₁₆⁺ (M+H)⁺ 1315.6. found 1315.8.

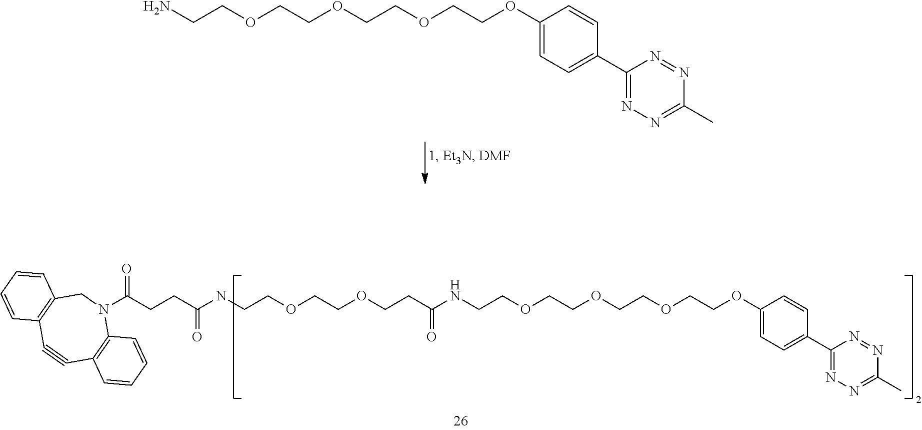

### Example 14. Preparation of Compound 27

To a solution of compound 1 (8.5 mg, 1.0 Eq, 10 μmol) in dry DMF (500 μL) was added HCl salt of methyltetrazine-benzylamine (7.4 mg, 3.0 Eq, 31 μmol), followed by triethylamine (5.3 mg, 7.2 μL, 5.0 Eq, 52 μmol). The reaction mixture was stirred at room temperature for 75 minutes and was subsequently purified by prep-HPLC; (30%→95%, MeCN/Water+10 mM NH₄HCO₃, runtime 12 minutes, Column Xbridge prep C18 5 μm OBD, 30×100 mm) to afford compound 27 (6.1 mg, 5.9 μmol, 57%, 97% purity) as a pink solid. LCMS (ESI+) calculated for C₅₃H₅₉N₁₂O₈⁺ (M+H)⁺ 991.5. found 991.7.

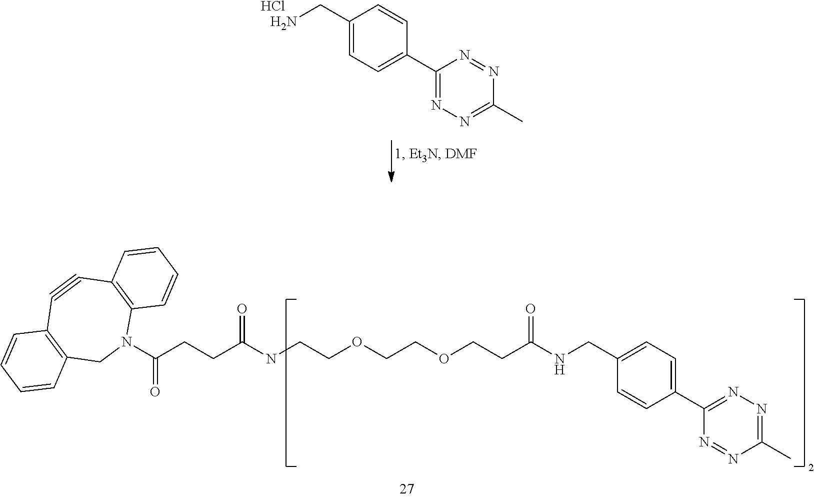

### Example 15. Preparation of Compound 29

To a solution of compound 24 (49.4 mg, 80% Wt, 1 Eq. 79.7 μmol) in anhydrous DCM (500 μL) was added diethanolamine (8.91 mg, 1.06 Eq, 84.7 μmol) and triethylamine (49 mg, 67 μL, 6.0 Eq, 0.48 mmol). After stirring at room temperature for 21 hours, bis(4-nitrophenyl) carbonate (65.4 mg, 2.70 Eq, 215 μmol) was added to the reaction mixture. After stirring for an addition 2 hours, the reaction mixture was purified by flash column chromatography over silicagel (0%→20% Aceton in DCM) to afford compound 28 (40.9 mg, 35 μmol, 43%, 67% purity) as a yellow oil. LCMS (ESI+) calculated for C₃₄H₄₂N₅O₁₅S⁺ (M+H)⁺ 792.2. found 792.5.

Compound 28 (7.9 mg, 67% WI, 1 Eq, 6.7 μmol) was dissolved in anhydrous DMF followed by the addition of compound 2 (4.5 mg, 2.4 Eq, 16 μmol) and triethylamine (3.4 mg, 4.7 μL, 5 Eq. 33 μmol). After stirring for 24 hours at room temperature, the reaction mixture was purified by prep-HPLC; (5%→95%, MeCN/Water+10 mM NH₄HCO₃, runtime 12 minutes, Column Xbridge prep C18 5 μm OBD, 30×100 mm) to afford compound 29 (5.3 mg, 5.3 μmol, 79%) as a pink oil. LCMS (ESI+) calculated for C₄₆H₆₂N₁₃O₁₁⁺ (M+H)⁺ 1004.4. found 1004.6.

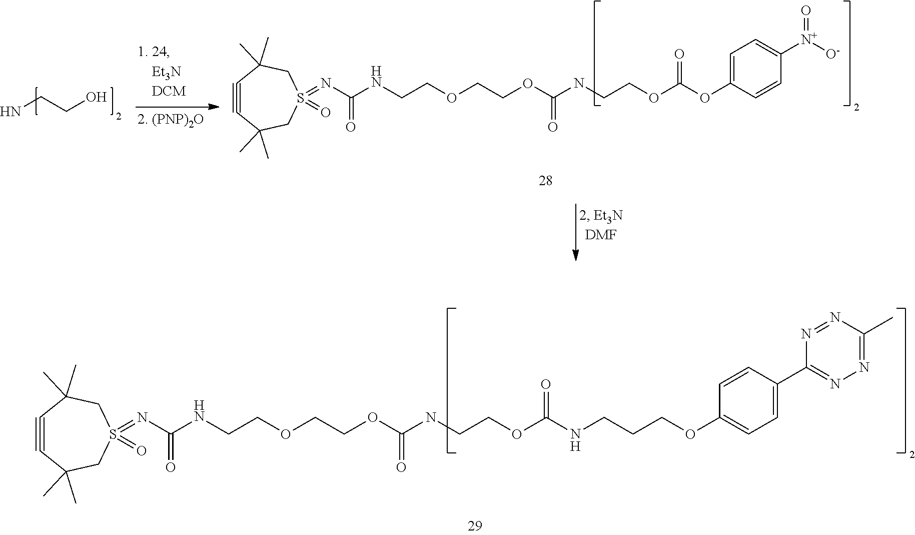

### Example 16. Preparation of Compound 31

A solution of Boc-Glu(OFm)—OH (639 mg, 1 Eq, 1.50 mmol) in DCM (5 mL) was cooled to 0° C. followed by the addition of trifluoroacetic acid (3.08 g, 2.08 mL, 18 Eq, 27.0 mmol). The reaction mixture was stirred for 19 hours and subsequently concentrated in vacuo and co-evaporated with DCM (6×5 mL) to afford the TFA salt of NH-Glu(Fm)—OH (569.9 mg, 1.30 mmol, 86.6%) as an off-white solid. LCMS (ESI+) calculated for C₁₉H₂₀NO₄⁺ (M+H)⁺ 326.2. found 326.3.

To a solution of compound 28 (10.0 mg, 1 Eq. 13 μmol) in anhydrous DMF (250 μL) and anhydrous DCM (250 μL) was added the TFA salt of NH-Glu(Fm)—OH (14 mg, 2.5 Eq, 32 μmol) and DIPEA (9.8 mg, 13 μL, 6 Eq, 76 μmol). After stirring for 15 hours at room temperature, the reaction mixture was purified by flash column chromatography over silicagel (0%→30% MeOH in DCM) to afford compound 30 (14.2 mg, 12 μmol, 95%, 98% Purity). LCMS (ESI+) calculated for C₆₀H₇₀N₅O₁₇S⁺ (M+H)⁺ 1164.5. found 1164.7.

HATU (5.1 mg, 2.1 Eq, 13 μmol) and DIPEA (4.9 mg, 6.6 μL, 6 Eq, 38 μmol) were added to a solution of compound 30 (7.3 mg, 1 Eq, 6.3 μmol) in DCM (500 μL). After 5 minutes of stirring at room temperature, the HCl salt of methyltetrazine-benzylamine (3.4 mg, 2.3 Eq, 14 μmol) and anhydrous DMF (200 μL) were added to the reaction mixture. After stirring for 3 hours at room temperature, the reaction mixture was purified by flash column chromatography over silicagel (0→70% EtOAc in heptane, followed by 0%→10% MeOH in DCM) to afford the Fmoc-protected tetrazine intermediate (3.6 mg, 1.5 μmol, 23%, 62% Purity). LCMS (ESI+) calculated for C₈₀H₈₈N₁₅O₁₅S⁺ (M+H)⁺ 1530.6. found 1530.8.

The Fmoc-protected tetrazine intermediate (3.6 mg, 1.5 μmol, 23%, 62% Purity) was dissolved in anhydrous DMF (300 μL) followed by the addition of piperidine (8.0 mg, 40 Eq, 94 μmol). After stirring for 2 hours at room temperature, the reaction mixture was further diluted with DMF (200 μL) and purified by prep-HPLC; (5%→95%, MeCN/Water+10 mM NH₄HCO₃, runtime 23 minutes, Column Xbridge prep C18 5 μm OBD, 30×100 mm) to afford compound 31 (0.6 mg, 0.5 μmol, 20%, 95% Purity) as a pink oil. LCMS (ESI+) calculated for C₅₂H₆₈N₁₅O₁₅S⁺ (M+H)⁺ 1174.5. found 1174.6.

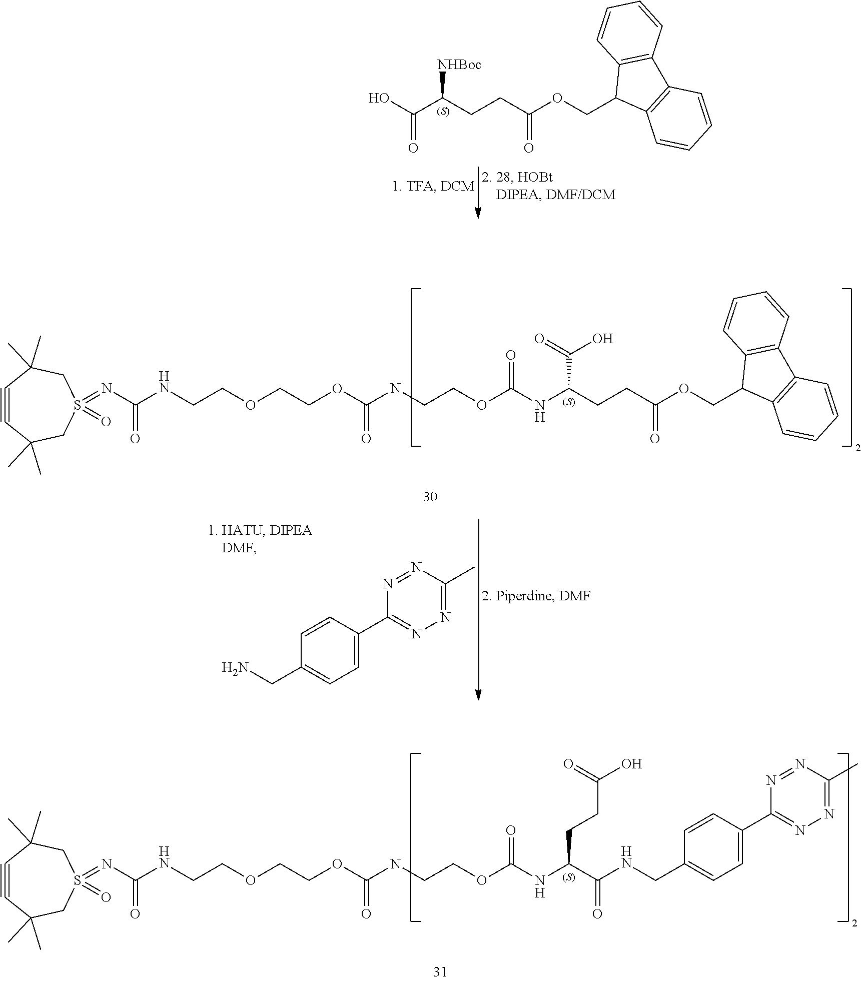

### Example 17. Preparation of Compound 33

To a solution of compound 14 (11.4 mg, 1.7 Eq, 29 mol) in dry DMF (200 μL) was added HATU (22 mg, 3.4 Eq, 58 μmol), followed by DIPEA (17 mg, 24 μL, 8.0 Eq, 134 mol). After 30 minutes, the HCl salt of methyltetrazine-benzylamine (4.0 mg, 1.0 Eq, 17 μmol) was added. The reaction mixture was stirred at room temperature for 5 hours, concentrated in vacuo and purified by flash column chromatography over silicagel (0%→20% MeOH in DCM) to give the Boc-protected compound (4.5 mg, 9.9 μmol, 59%, 97% purity) as a pink solid. LCMS (ESI−) calculated for C₁₈H₂₃N₆O₆S⁻ (M−H)⁻ 451.1. found 451.1.

The Boc-protected compound (2.2 mg, 97% Wt, 1.0 Eq, 4.9 μmol) was dissolved in DCM (200 μL) and TFA (22 mg, 15 μL, 40 Eq, 190 μmol) was added. The reaction mixture was stirred at room temperature for 1 hour, concentrated in vacuo to afford compound 32 (2.3 mg, 4.9 mol, 100%. 100% purity) as a pink oil. Yield was assumed quantitative and compound 32 was used without further purification in the next step. LCMS (ESI−) calculated for C₁₃H₁₅N₆O₄S⁻ (M−H)⁻ 351.1. found 351.0.

To a solution of compound 28 (3.5 mg, 1.0 Eq, 4.4 mol) in dry DMF (300 L) was added compound 32 (3.8 mg, 1.8 Eq, 8.1 μmol), followed by Et₃N (8.8 mg, 12 μL, 20 Eq, 20 μmol) and HOBt (0.7 mg, 1.0 Eq, 4.4 μmol). The reaction mixture was stirred at room temperature for 20 hours and was subsequently purified by prep-HPLC; (30%→95%, MeCN/Water+10 mM NH₄HCO₃, runtime 12 minutes, Column Xbridge prep C18 5 μm OBD, 30×100 mm) to afford compound 33 (0.5 mg, 0.4 μmol, 9%, 100% purity) as a pink solid. LCMS (ESI−) calculated for C₄₈H₆₂N₁₅O₁₇S₃⁻ (M−H)⁻ 1216.4. found 1216.7.

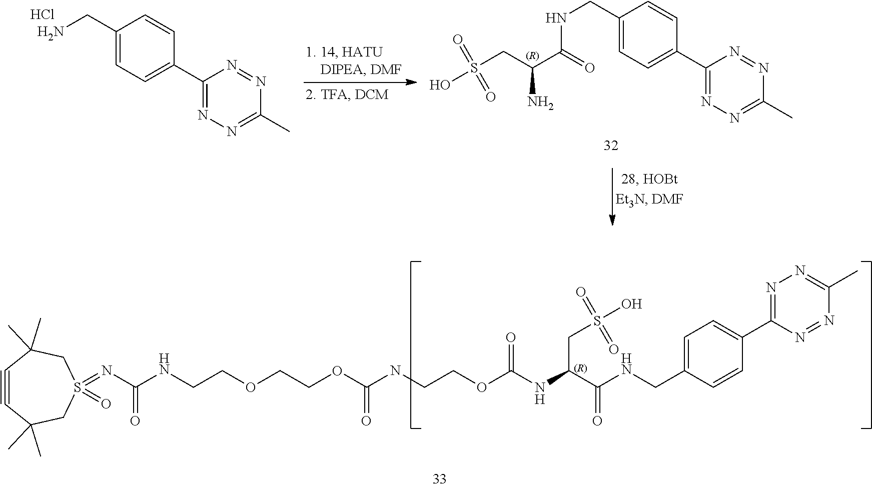

### Example 18. Preparation of Compound 35

N-Boc-ethylenediamine (10.3 mg, 10.2 μL, 6.85 Eq, 64.4 μmol) was added to a solution of compound 28 (10.2 mg, 73% Wt, 1 Eq, 9.40 μmol) in anhydrous DCM (500 μL). After stirring for 1 hour at room temperature, the reaction mixture was purified by flash column chromatography over silicagel (0%→30% EtOAc in DCM followed by 0%→10% MeOH in DCM) to afford the Boc-protected intermediate (7.7 mg, 8.7 μmol, 92%, 93.7% Purity). LCMS (ESI+) calculated for C₃₆H₆₄N₇O₁₃S⁺ (M+H)⁺ 834.4. found 834.3.

The Boc-protected intermediate (7.2 mg, 1 Eq, 8.6 μmol) was dissolved in anhydrous DCM (300 μL), followed by the addition of TFA (20 mg, 13 μL, 20 Eq, 0.17 mmol). The mixture was stirred at room temperature for 1 hour and subsequently concentrated in vacuo and co-evaporated with toluene to afford compound 34 (7.2 mg, 8.7 μmol, 100%). Yield was assumed quantitative and the product was used in the next step without any further purification. LCMS (ESI+) calculated for C₂₆H₄₈N₇O₉S⁺ (M+H)⁺ 634.3. found 634.3.

HATU (4.4 mg, 2.7 Eq, 12 μmol) and DIPEA (3.4 mg, 4.5 μL, 6 Eq, 26 μmol) were added to a solution of 4-(6-methyl-1,2,4,5-tetrazin-3-yl)butanoic acid (2.0 mg, 2.5 Eq, 11 μmol) in anhydrous DMF (150 μL). After stirring at room temperature for 30 minutes, a solution of compound 34 (3.6 mg, 1 Eq, 4.3 μmol) in anhydrous DMF (150 μL) was added. After an additional 45 minutes, the reaction mixture was purified by prep-HPLC; (5%→95%, MeCN/Water+10 mM NH₄HCO₃, runtime 12 minutes, Column Xbridge prep C18 5 μm OBD, 30×100 mm) to afford compound 35 (2.2 mg, 1.7 μmol, 39%, 74% Purity) as a pink oil. LCMS (ESI+) calculated for C₄₀H₆₄N₁₅O₁₁S⁺ (M+H)⁺ 962.5. found 962.7.

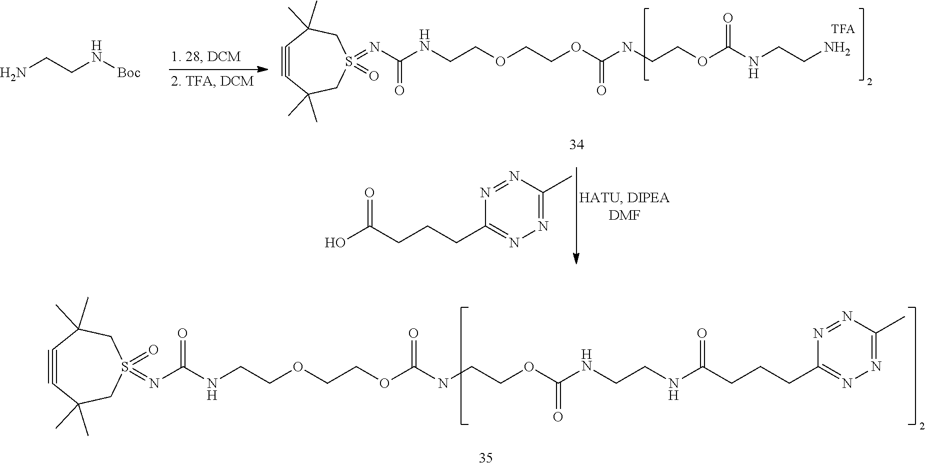

### Example 19. Preparation of Compound 37

The HCl salt of EDC (77.1 mg, 2.1 Eq, 338 μmol) and HOBt (49.3 mg, 2 Eq, 322 μmol) were added to a solution of Fmoc-Ser-OH (52.7 mg, 1 Eq, 161 μmol) in anhydrous DMF (1 mL). After 10 minutes of stirring at room temperature, N-Boc-ethylenediamine (4.0 mg, 3.9 μL, 1.2 Eq, 25 μmol) was added. The reaction mixture was stirred for 1 hour at room temperature and was subsequently purified by flash column chromatography over silicagel (0%→5% MeOH in DCM). The residue was triturated with DCM to afford the Boc-protected intermediate (53.7 mg, 85.2 μmol, 52.9%, 74.5% Purity) as a white solid. LCMS (ESI+) calculated for C₂₅H₃₂N₃O₆⁺ (M+H)⁺ 470.2. found 470.5.

The Boc-protected intermediate (53.7 mg, 74.5% Wt, 1 Eq, 85.2 μmol) was dissolved in anhydrous DCM (2 mL), followed by the addition of trifluoroacetic acid (194 mg, 131 μL, 20 Eq, 1.70 mmol). The mixture was stirred at room temperature for 2.5 hours and subsequently concentrated in vacuo and co-evaporated with toluene to afford compound 36 ((39.8 mg, 85.1 μmol, 100%). Yield was assumed quantitative, and the product was used in the next step without any further purification. LCMS (ESI+) calculated for C₂₀H₂₄N₃O₄⁺ (M+H)⁺ 370.2. found 370.2.

To a solution of compound 36 (9.5 mg, 2.5 Eq, 20 μmol) in anhydrous DCM (580 μL) were added compound 1 (12.8 mg, 1.9 Eq, 15.7 μmol) and DIPEA (5.2 mg, 7.1 μL, 5 Eq, 41 μmol). Complete conversion to the Fmoc-protected DBCO intermediate was observed after 75 minutes of stirring at room temperature, upon which piperidine (14 mg, 16 μL, 20 Eq, 0.16 mmol) was added to the reaction mixture. After stirring for 4 hours, the reaction mixture was concentrated in vacuo and the residue was partitioned between water and DCM. The water layer was evaporated in vacuo and the crude transferred to an Eppendorf tube. The residue was dissolved in water (500 μL) and DMF (400 μL), the resulting solution was centrifuged, and the supernatant was purified by prep-HPLC; (5%→50%, MeCN/Water+10 mM NH₄HCO₃, runtime 12 minutes, Column Xbridge prep C18 5 μm OBD, 30×100 mm) to afford compound 37 (1.4 mg, 1.4 μmol, 17%, 86% Purity). LCMS (ESI−) calculated for C₄₃H₆₁N₈O₁₂⁻ (M−H)⁻ 881.4. found 881.3.

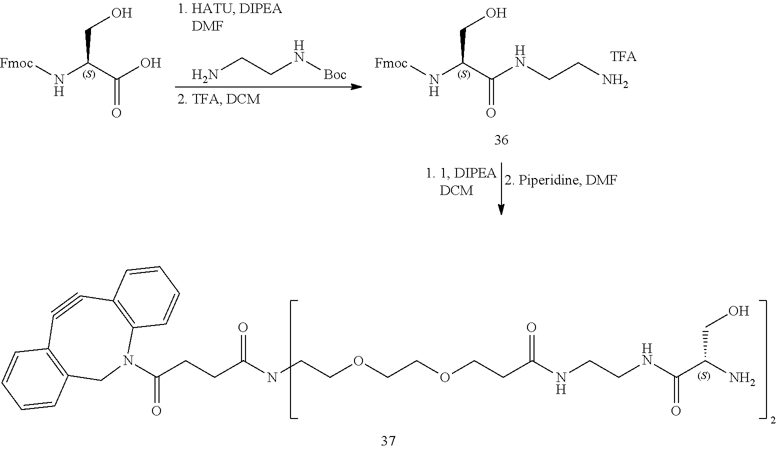

### Example 20. Preparation of Compound 36

To a solution of compound 28 (17 mg, 61% Wt, 1 Eq, 13 μmol) in anhydrous DCM (500 μL) was added a solution of compound 36 (22.5 mg, 3.75 Eq, 47.6 μmol) in anhydrous DCM (750 μL), followed by the addition of DIPEA (14.7 mg, 20 μL, 8.75 Eq, 0.11 mmol). After stirring for 26 hours at room temperature, the solution was concentrated in vacuo and the residue was dissolved in DMF (600 μL) and purified by prep-HPLC; (5%→95%, MeCN/Water+10 mM NH₄HCO₃, runtime 12 minutes, Column Xbridge prep C18 5 μm OBD, 30×100 mm) to afford the Fmoc-protected TMTHSI intermediate (8.5 mg, 6.8 μmol, 53%). LCMS (ESI+) calculated for C₆₂H₇₈N₉O₁₇S⁺ (M+H)⁺ 1252.5. found 1252.8.

The Fmoc-protected TMTHSI intermediate (8.5 mg, 1 eq. 6.8 μmol) was dissolved in anhydrous DMF (450 μL), followed by the addition of piperidine (23 mg, 27 μL, 40 Eq, 0.27 mmol). After stirring for 2 hours, the reaction mixture was purified by reverse phase chromatography (10%→100%, MeCN/Water+10 mM NH₄HCO₃) to afford compound 38 (1.8 mg, 2.2 μmol, 33%). LCMS (ESI+) calculated for C₃₂H₅₈N₉O₁₃S⁺ (M+H)⁺ 808.4. found 808.7.

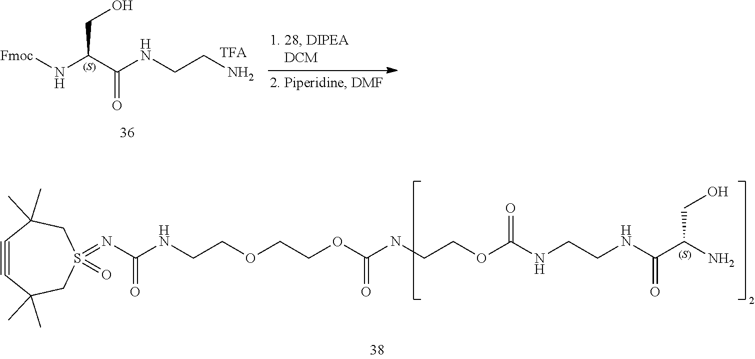

### Example 21. Preparation of Compound 41

To a solution of tert-butyl (R)-(1-((tert-butyldimethylsilyl)oxy)-3-hydroxypropan-2-yl) carbamate (509 mg, 1.0 Eq, 1.67 mmol) in dry DCM (10 mL) was added CSI (260 mg, 159 μL, 1.1 Eq, 1.83 mmol) and the reaction mixture was stirred at room temperature for 10 minutes. Et₃N (506 mg, 697 μL, 3.0 Eq, 5.00 mmol) was added, followed by m-PEG₄-NH₂ (380 mg, 1.1 Eq, 1.83 mmol). The reaction mixture was stirred at room temperature for 3 hours and was subsequently purified by flash column chromatography over silicagel (0%→25% MeOH in DCM) to give the Boc and TBS protected compound (1.01 g, 1.5 mmol, 90%, 92% purity) as a clear oil. LCMS (ESI+) calculated for C₁₉H₄₄N₃O₁₁SSi⁺ (M+H-Boc)⁺ 518.3. found 518.5.

MeOH (8.7 g, 11 mL, 5·10² Eq, 273 mmol) was cooled on ice, followed by dropwise addition of acetyl chloride (1.28 g, 1.16 mL, 30 Eq, 16.3 mmol). The reaction mixture was stirred at 0° C. for 5 minutes. The ice bath was removed, and a solution of the Boc and TBS protected compound (337 mg, 92% Wt, 1.0 Eq, 545 μmol) in dry DCM (1.5 mL) was added. The reaction mixture was stirred at room temperature for 18 hours and concentrated in vacuo to afford compound 39 (196 mg, 446 μmol, 82%. 100% purity). LCMS (ESI+) calculated for C₁₃H₃₀N₃O₉S⁺ (M+H)⁺ 404.1. found 404.4.

To a solution of compound 11 (60 mg, 1.0 Eq, 80 μmol) in dry DCM (3000 μL) was added compound 39 (177 mg, 5.0 Eq, 402 μmol), followed by DIPEA (62 mg, 84 μL, 6.0 Eq, 482 μmol). The reaction mixture was stirred at room temperature for 22 hours and was subsequently purified by flash column chromatography over silicagel (0%→10% MeOH in DCM) to give the diol compound (72 mg, 53 μmol, 66%, 87% purity). LCMS (ESI+) calculated for C₄₇H₇₆N₇O₂₄S₂⁺ (M+H)⁺ 1186.4. found 1186.7.

To a solution of the diol compound (5.0 mg, 87% Wt, 1.0 Eq, 3.7 μmol) in dry DCM (200 μL) was added bis-PNP carbonate (3.3 mg, 3.0 Eq, 11 μmol), followed by DIPEA (2.8 mg, 3.8 μL, 6.0 Eq, 22 μmol). The reaction mixture was stirred at room temperature for 19 hours. The HCl salt of methyltetrazine-benzylamine (2.4 mg, 2.8 Eq, 10 μmol) was added. The reaction mixture was stirred at room temperature for 3 hours and subsequently purified by flash column chromatography over silicagel (0%→15% MeOH in DCM) to afford compound 40 (2.6 mg, 1.6 μmol, 43%, 100% purity). LCMS (ESI+) calculated for C₆₉H₉₇N₁₈O₂₆S₂⁺ (M+NH₄)⁺ 1657.6. found 1658.0.

To a solution of compound 40 (2.6 mg, 1.0 Eq, 1.6 μmol) in DCM (200 μL) was added piperidine (5.4 mg, 6.3 μL, 40 Eq, 63 μmol). The reaction mixture was stirred at room temperature for 4 hours. It was purified by flash column chromatography over silicagel (0%→20% MeOH in DCM) to give the Fmoc-deprotected compound (2.5 mg, 1.6 μmol, 100%, 92% purity). LCMS (ESI+) calculated for C₅₄H₉₇N₁₇O₂₄S₂⁺ (M+H)⁺ 1418.5. found 1419.0.

To a solution of the Fmoc-deprotected compound (2.5 mg, 1.0 Eq, 1.8 μmol) in DMF (200 μL) was added compound 24 (1.3 mg, 1.5 Eq, 2.6 μmol), followed by Et₃N (1.8 mg, 2.4 μL, 10 Eq, 18 μmol) and HOBt (0.4 mg, 1.5 Eq, 2.6 μmol). The reaction mixture was stirred at room temperature for 23 hours and was subsequently purified by prep-HPLC; (5%→95%, MeCN/Water+10 mM NH4HCO3, runtime 12 minutes, Column Xbridge prep C18 5 μm OBD, 30×100 mm) to afford compound 41 (0.6 mg, 0.3 μmol, 20%, 100% purity) as a pink solid. LCMS (ESI+) calculated for C₇₀H₁₀₉N₁₉O₂₉S₃⁺² (M/2+H)⁺ 887.8. found 887.9.

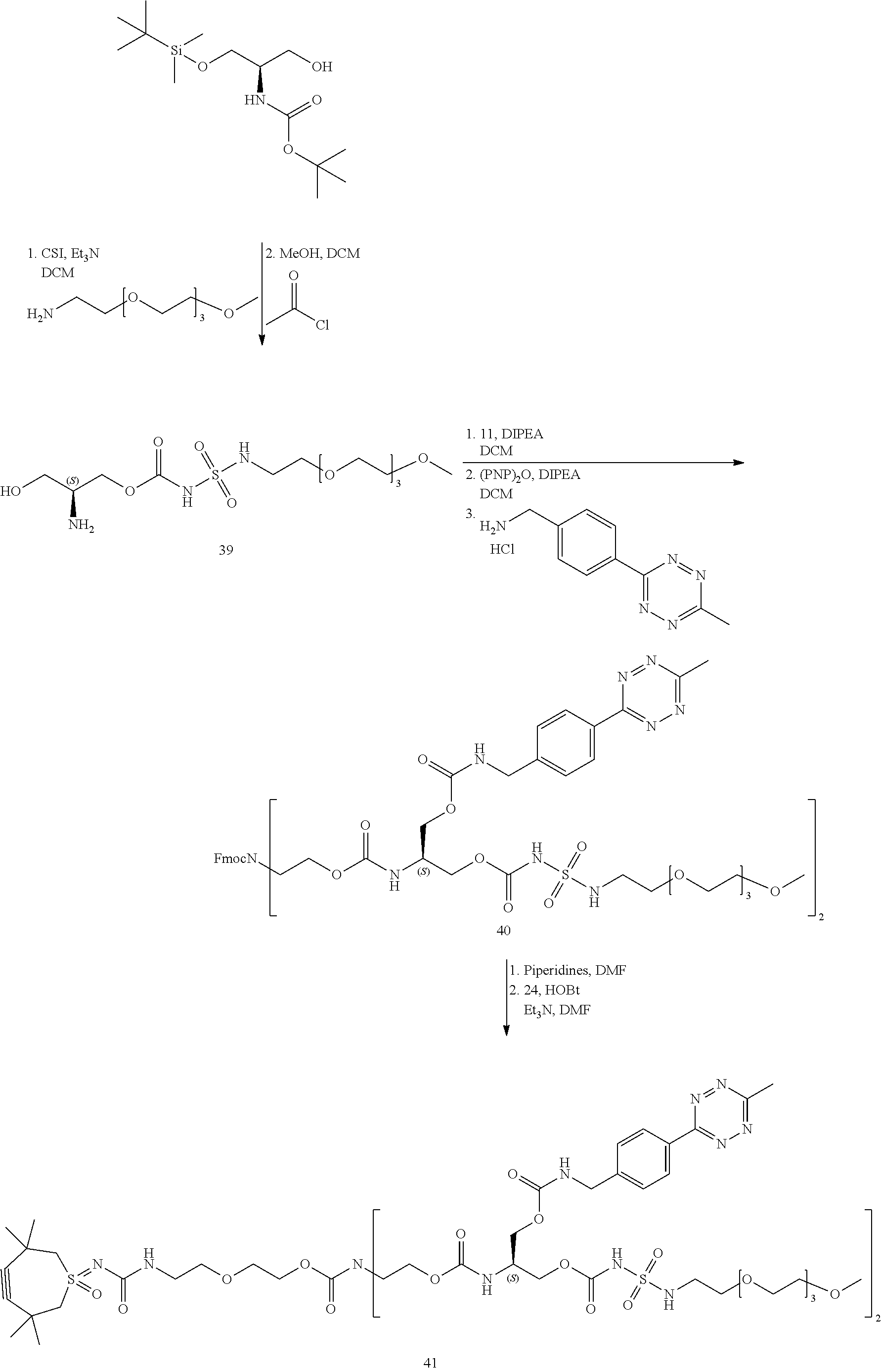

### Example 22. Preparation of Compound 45

The HCl salt of EDC (228.7 mg, 1.2 Eq, 1.002 mmol) and HOAt (34.10 mg, 250.5 μL, 1 molar, 0.3 Eq, 250.5 μmol) were added to a solution of Fmoc-NH-PEG₂-propionic acid (400.3 mg, 1.2 Eq, 1.002 mmol) in anhydrous DCM (5 mL). After stirring at room temperature for 30 minutes, a solution of tris[[2-(tert-butoxycarbonyl)ethoxy]methyl]methylamine (400.3 mg, 1.2 Eq, 1.002 mmol) in anhydrous DMF (1 mL) was added. The reaction mixture was stirred for 75 minutes at room temperature, upon which the DCM was evaporated, and the residue was purified by reverse phase chromatography (10%→100%, MeCN/Water+10 mM NH₄HCO₃) to afford compound 42 (585.4 mg, 628 μmol, 75.2%, 95.2% Purity) as a colourless oil. LCMS (ESI+) calculated for C₄₇H₇₁N₂O₁₄⁺ (M+H)⁺ 887.5. found 887.8.

Compound 42 (585.4 mg, 95% Wt, 1 Eq, 626.9 μmol) was dissolved in anhydrous DCM (1.5 mL), followed by the addition of trifluoroacetic acid (2.2 g, 1.5 mL, 31 Eq, 19 mmol). After 2 hours of stirring at room temperature, DCM (10 mL) was added and the reaction mixture was extracted with 1M HCl (3×10 mL). The combined aqueous layers were extracted with DCM (2×20 mL). The combined organic layers were dried over anhydrous sodium sulfate, filtered and concentrated in vacuo to afford compound 43 (327.7 mg, 0.41 mmol, 65%, 90% Purity) as a light-yellow gel. LCMS (ESI+) calculated for C₃₅H₄₇N₂O₁₄⁺ (M+H)⁺ 719.3. found 719.7.

The HCl salt of EDC (24.8 mg, 4.09 Eq, 109 μmol) and HOBt (16.7 mg, 4.11 Eq, 109 μmol) were added to a solution of compound 43 (21.2 mg, 90% Wt, 1.0 Eq, 26.5 μmol) in anhydrous DCM (400 μL). After stirring at room temperature for 90 minutes, the HCl salt of methyltetrazine-benzylamine (25.2 mg, 3.99 Eq, 106 μmol) was added. The reaction mixture was stirred for 15 minutes and was subsequently purified by flash column chromatography over silicagel (0%→15% MeOH in DCM) to afford the Fmoc-protected tetrazine intermediate (15.5 mg, 11 μmol, 43%, 94% Purity) as a pink oil. LCMS (ESI+) calculated for C₆₅H₇₄N₁₇O₁₁⁺ (M+H)⁺ 1268.6. found 1269.1.

The Fmoc-protected tetrazine intermediate (15.5 mg, 95.0% Wt, 1 Eq, 11.6 μmol) was dissolved in anhydrous DCM (200 μL), followed by the addition of piperidine (43 mg, 50 μL, 44 Eq, 0.51 mmol). After 90 minutes of stirring at room temperature, the reaction mixture was purified by flash column chromatography over silicagel (0%→35% MeOH in DCM) to afford compound 44 (11.8 mg, 9.9 μmol, 86%, 88% Purity) as a pink solid. LCMS (ESI+) calculated for C₅₀H₆₄N₁₇O₉⁺ (M+H)⁺ 1046.5. found 1046.9.

To a solution of compound 44 (15 mg, 92% Wt, 1 Eq, 13 μmol) in anhydrous DMF (450 μL) was added DBCO-NHS ester (8.0 mg, 1.5 Eq, 20 μmol) followed by the addition of DIPEA (8.5 mg, 11 μL, 5 Eq, 66 μmol). After 1 hour of stirring at room temperature, the reaction mixture was further diluted with 250 μL DMF and purified by prep-HPLC; (30%→95%, MeCN/Water+10 mM NH₄HCO₃, runtime 23 minutes, Column Xbridge prep C18 5 μm OBD, 30×100 mm). The obtained residue was dissolved in DCM (1 mL) and was precipitated by the addition of diethyl ether (15 mL). The precipitate was washed with diethyl ether (3×5 mL) and concentrated in vacuo to afford compound 45 (9.5 mg, 7.1 μmol, 54%) as a pink solid. LCMS (ESI+) calculated for C₆₉H₇₇N₁₈O₁₁⁺ (M+H)⁺ 1333.6. found 1333.7.

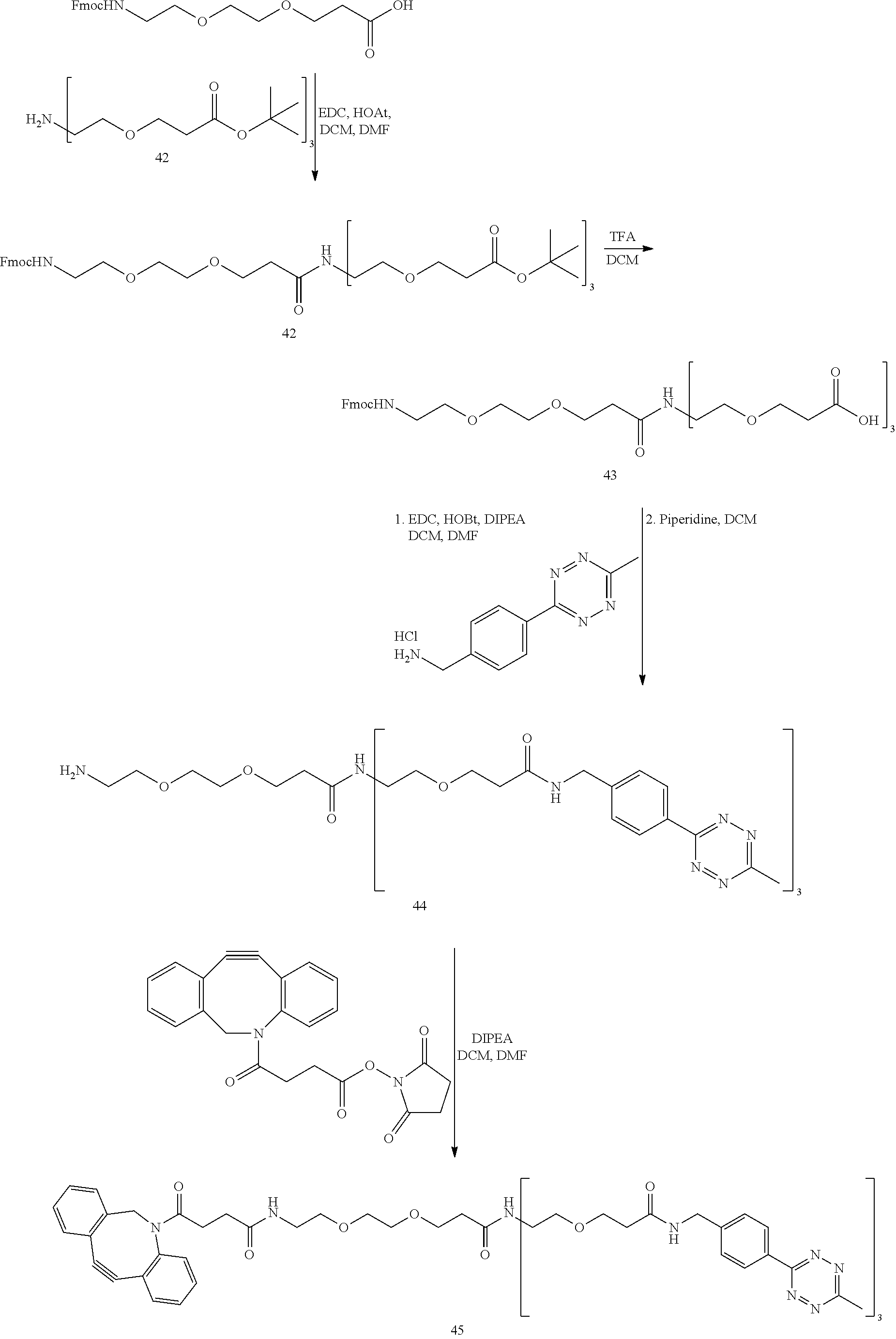

### Example 23. Preparation of Compound 46

To a solution of compound 44 (3.0 mg, 1.0 Eq, 2.7 μmol) in dry DMF (200 μL) was added TMTHSI-OSu (3.3 mg, 3.6 Eq, 9.6 μmol), followed by triethylamine (2.6 mg, 3.8 μL, 10 Eq, 26 μmol) and HOBt (0.5 mg, 1.2 Eq, 3.2 μmol). The reaction mixture was stirred at room temperature for 26 hours and was subsequently purified by prep-HPLC; (30%→95%, MeCN/Water+10 mM NH₄HCO₃, runtime 12 minutes, Column Xbridge prep C18 5 μm OBD, 30×100 mm) to afford compound 46 (0.6 mg, 0.4 μmol, 20%, 93% purity) as a pink solid. LCMS (ESI+) calculated for C₆₁H₇₉N₁₈O₁₁S⁺ (M+H)⁺ 1271.6. found 1271.8.

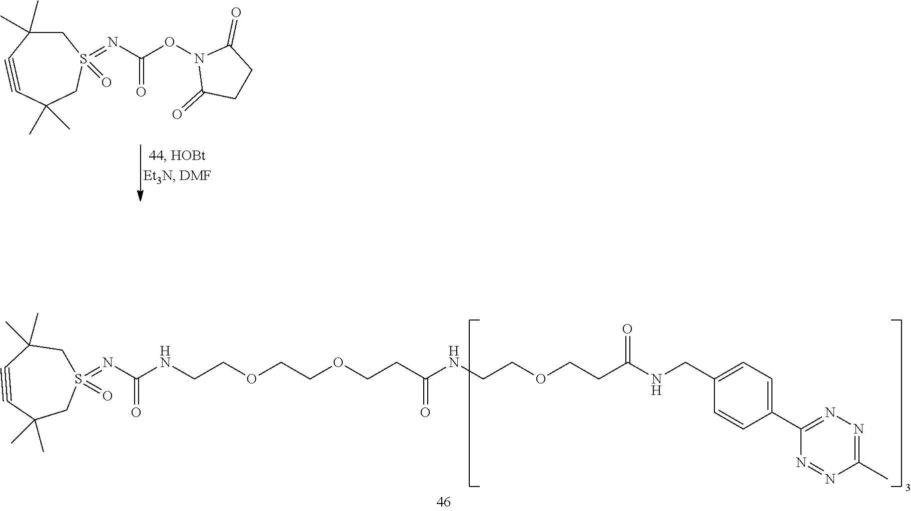

### Example 24. Preparation of Compound 49

To a solution of compound 47 (25.4 mg, 1 Eq, 24.0 μmol, commercially obtained from GenScript Biotech) in anhydrous DMF (500 μL) was added methyltetrazine-NHS ester (20.3 mg, 2.59 Eq, 62.0 μmol) followed by the addition of DIPEA (21.5 mg, 29.0 μL, 6.95 Eq, 166 μmol). After stirring for 30 minutes, the desired product was precipitated by the addition of diethyl ether (1 mL). The precipitate was washed with diethyl ether (3×1.5 mL) and concentrated in vacuo to afford Alloc-protected tetrazine intermediate (57.0 mg, 21.9 μmol, 91.2%, 49.4% Purity) as a pink solid. LCMS (ESI+) calculated for C₆₃H₇₀N₁₇O₁₄⁺ (M+H)⁺ 1288.5. found 1288.9.

The Alloc-protected tetrazine intermediate (22.9 mg, 49.4% Wt, 1 Eq, 8.78 μmol) was dissolved in anhydrous DMF (500 μL), followed by the addition of tetrakis(triphenylphosphine)palladium(0) (1.26 mg, 179 μL, 6.1 mmolar, 0.124 Eq, 1.09 μmol) and pyrrolidine (2.0 mg, 2.3 μL, 3.2 Eq, 28 μmol). After 10 minutes, the reaction mixture was purified by reverse phase chromatography (10%→100%, MeCN/Water+10 mM NH₄HCO₃) to afford compound 48 (13.2 mg, 5.9 μmol, 67%, 54% Purity) as a pink solid. LCMS (ESI+) calculated for C₅₇H₆₁N₁₇O₁₄⁺ (M+H)⁺ 1208.5. found 1209.0.

Compound 48 (13.2 mg, 54% Wt, 1 Eq, 5.90 μmol) was dissolved in anhydrous DMF (350 μL) followed by the addition of piperidine (10.0 mg, 11.7 μL, 20 Eq, 118 μmol). After 35 minutes, the reaction mixture was purified by reverse phase chromatography (10%→100%, MeCN/Water+10 mM NH₄HCO₃) to afford the Fmoc-deprotected intermediate (3.4 mg, 3.3 μmol, 56%) as a pink solid. LCMS (ESI+) calculated for C₄₂H₅₂N₁₇O₁₂⁺ (M+H)⁺ 986.4. found 986.7.

To a solution of the Fmoc-deprotected intermediate (3.4 mg, 1 Eq, 3.3 μmol) in anhydrous DMF (200 μL) were added DBCO-NHS ester (3.1 mg, 2.3 Eq, 7.7 μmol) and DIPEA (2.1 mg, 2.9 μL, 5 Eq, 17 μmol). After 1 hour, the reaction mixture was purified by prep-HPLC; (5%→95%, MeCN/Water+10 mM NH₄HCO₃, runtime 12 minutes, Column Xbridge prep C18 5 μm OBD, 30×100 mm) to afford compound 49 (0.7 mg, 0.5 μmol, 20%, 94% Purity) as a pink oil. LCMS (ESI+) calculated for C₆₁H₆₅N₁₈O₁₄⁺ (M+H)⁺ 1273.5. found 1273.8.

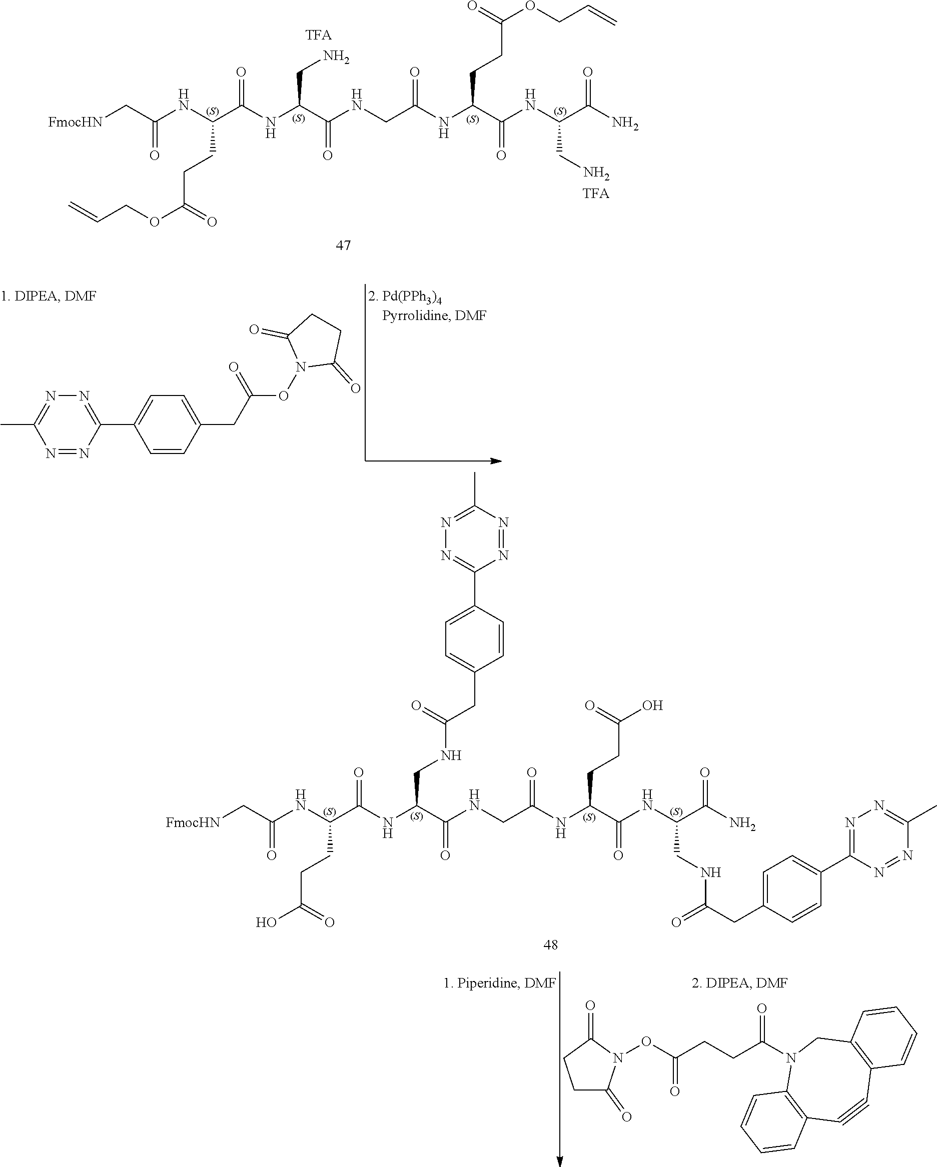

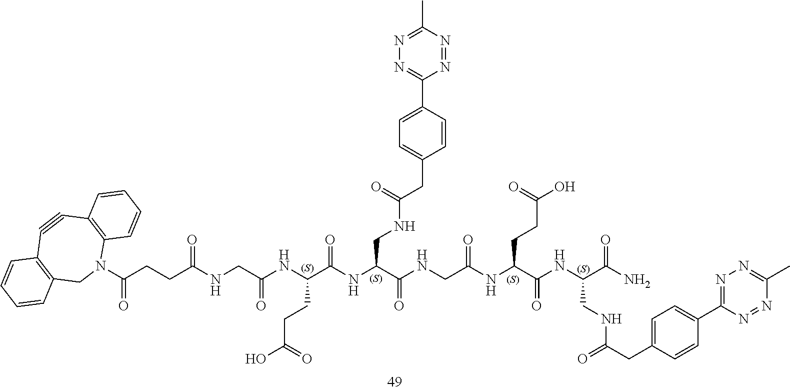

### Example 25. Preparation of Compound 51

To a solution of compound 48 (7.1 mg, 1 Eq, 5.9 μmol) in anhydrous DMF (300 μL) was added Amino-PEG8-OH (6.5 mg, 3 Eq, 18 μmol), followed by the addition of DIPEA (4.6 mg, 6.1 μL, 6 Eq, 35 μmol) and HATU (4.9 mg, 2.2 Eq, 13 μmol). After 45 minutes, the desired product was precipitated by the addition of diethyl ether (1 mL). The precipitate was washed with diethyl ether (3×1.5 mL) and concentrated in vacuo to afford compound 50 (31.4 mg, 10 μmol, 170%, 62% Purity) as a pink solid. Yield was assumed quantitative and the product was used in the next step without any further purification. LCMS (ESI−) calculated for C₈₉H₁₂₆N₁₉O₂₈⁻ (M−H)⁻ 1908.9. found 1908.6.

Compound 50 (11 mg, 1 Eq, 5.8 μmol) was dissolved in anhydrous DMF (300 μL), followed by the addition of piperidine (9.8 mg, 11 μL, 20 Eq, 0.12 mmol). After 2.5 hours, the desired product was precipitated by the addition of diethyl ether (1 mL). The precipitate was washed with diethyl ether (3×1.5 mL) and concentrated in vacuo to afford the Fmoc-deprotected intermediate (8.0 mg, 3.2 μmol, 55%, 67% Purity) as a pink solid. LCMS (ESI−) calculated for C₇₄H₁₁₆N₁₉O₂₆⁻ (M−H)⁻ 1686.8. found 1687.2.

To a solution of the Fmoc-deprotected intermediate (8.0 mg, 1 Eq, 4.7 μmol) in anhydrous DMF (350 μL) were added DBCO-NHS ester (2.9 mg, 1.5 Eq, 7.1 μmol) and DIPEA (3.1 mg, 4.1 μL, 5 Eq, 24 μmol). After 1 hour, the reaction mixture was purified by prep-HPLC; (30%→95%, MeCN/Water+10 mM NH₄HCO₃, runtime 12 minutes, Column Xbridge prep Cia 5 μm OBD, 30×100 mm. The residue was dissolved in DMF (500 μL) and was precipitated by the addition of diethyl ether (1 mL). The precipitate was washed with diethyl ether (3×1.5 mL) and concentrated in vacuo to afford compound 51 (2.8 mg, 1.3 μmol, 27%, 90% Purity) as a purple solid. LCMS (ESI+) calculated for C₉₃H₁₃₂N₂₀O₂₈⁺² (M/2+H)⁺ 988.5. found 988.7.

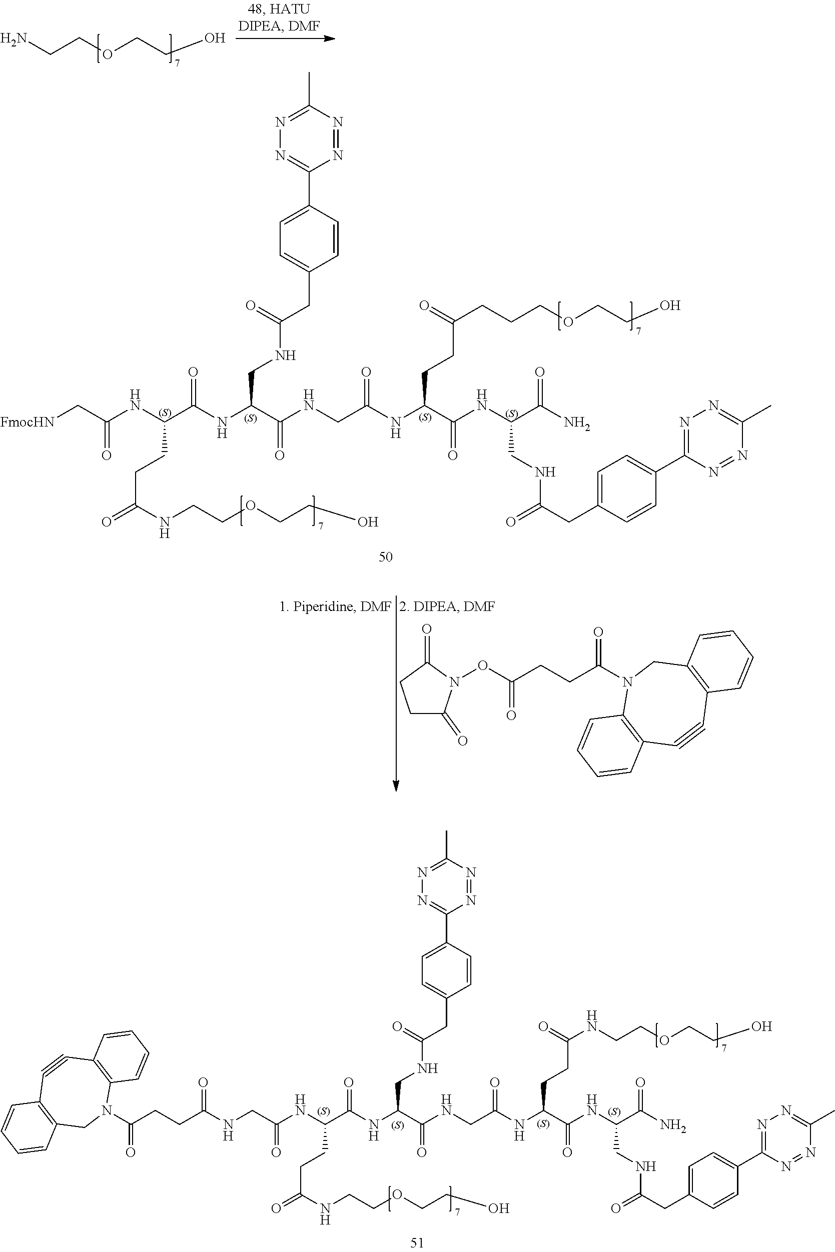

### Example 26. Preparation of Compound 54

To a solution of compound 52 (25.0 mg, 1 Eq, 17.0 μmol, commercially obtained from GenScript Biotech) in anhydrous DMF (500 μL) was added methyltetrazine-NHS ester (19.6 mg, 3.52 Eq, 59.9 μmol) followed by the addition of DIPEA (15.4 mg, 20.7 μL, 7 Eq, 119 μmol). After stirring for 2 hours, the reaction mixture was diluted with DMF (4 mL) and subsequently the desired product was precipitated by the addition of diethyl ether (10 mL). The precipitate was washed with diethyl ether (3×5 mL) and concentrated in vacuo to afford the Alloc-protected tetrazine intermediate (73.9 mg, 29 μmol, 170%, 70% Purity) as a pink solid. LCMS (ESI+) calculated for C₈₇H₉₈N₂₅O₂₀⁺ (M+H)⁺ 1812.7. found 1813.0.

The Alloc-protected tetrazine intermediate (30.8 mg, 1 Eq, 17.0 μmol) was dissolved in anhydrous DMF (3 mL), followed by the addition of tetrakis(triphenylphosphine)palladium(0) (1.96 mg, 218 μL, 7.8 mmolar, 0.1 Eq, 1.70 μmol) and pyrrolidine (3.62 mg, 4.19 μL, 3 Eq, 51.0 μmol). After 10 minutes the reaction mixture was purified by reverse phase chromatography (10%→100%, MeCN/Water+10 mM NH₄HCO₃) to afford compound 53 (21.0 mg, 11 μmol, 63%, 86% Purity) as a pink solid. LCMS (ESI+) calculated for C₇₈H₈₆N₂₅O₂₀⁺ (M+H)⁺ 1692.6. found 1692.9.

Compound 53 (21 mg, 86% A, 1 Eq, 11 μmol) was dissolved in anhydrous DMF (2 mL) followed by the addition of piperidine (18 mg, 21 μL, 20 Eq, 0.21 mmol). After 10 minutes, the desired product was precipitated by the addition of diethyl ether (10 mL). The precipitate was washed with diethyl ether (3×5 mL) and concentrated in vacuo afford the Fmoc-deprotected intermediate (24.9 mg, 15 μmol, 140%, 89% Purity) as a pink solid. Yield was assumed quantitative, and the product was used in the next step without any further purification. LCMS (ESI+) calculated for C₆₃H₇₆N₂₅O₁₈⁺ (M+H)⁺ 1470.6. found 1470.9.

To a solution of the Fmoc-deprotected intermediate (8.2 mg, 89% Wt, 1 Eq, 5.0 μmol) in anhydrous DMF (500 μL) were added DBCO-NHS ester (6.0 mg, 3 Eq, 15 μmol) and DIPEA (1.9 mg, 2.6 μL, 3 Eq, 15 μmol). After 45 minutes, the reaction mixture was purified by prep-HPLC; (5%→95%, MeCN/Water+10 mM NH₄HCO₃, runtime 12 minutes, Column Xbridge prep C18 5 μm OBD, 30×100 mm) and the product containing fractions were freeze dried to afford compound 54 (4.1 mg, 1.8 μmol, 36%, 76% Purity) as a pink solid. LCMS (ESI−) calculated for C₈₂H₈₇N₂₆O₂₀⁻ (M−H)⁻ 1755.7. found 1755.2.

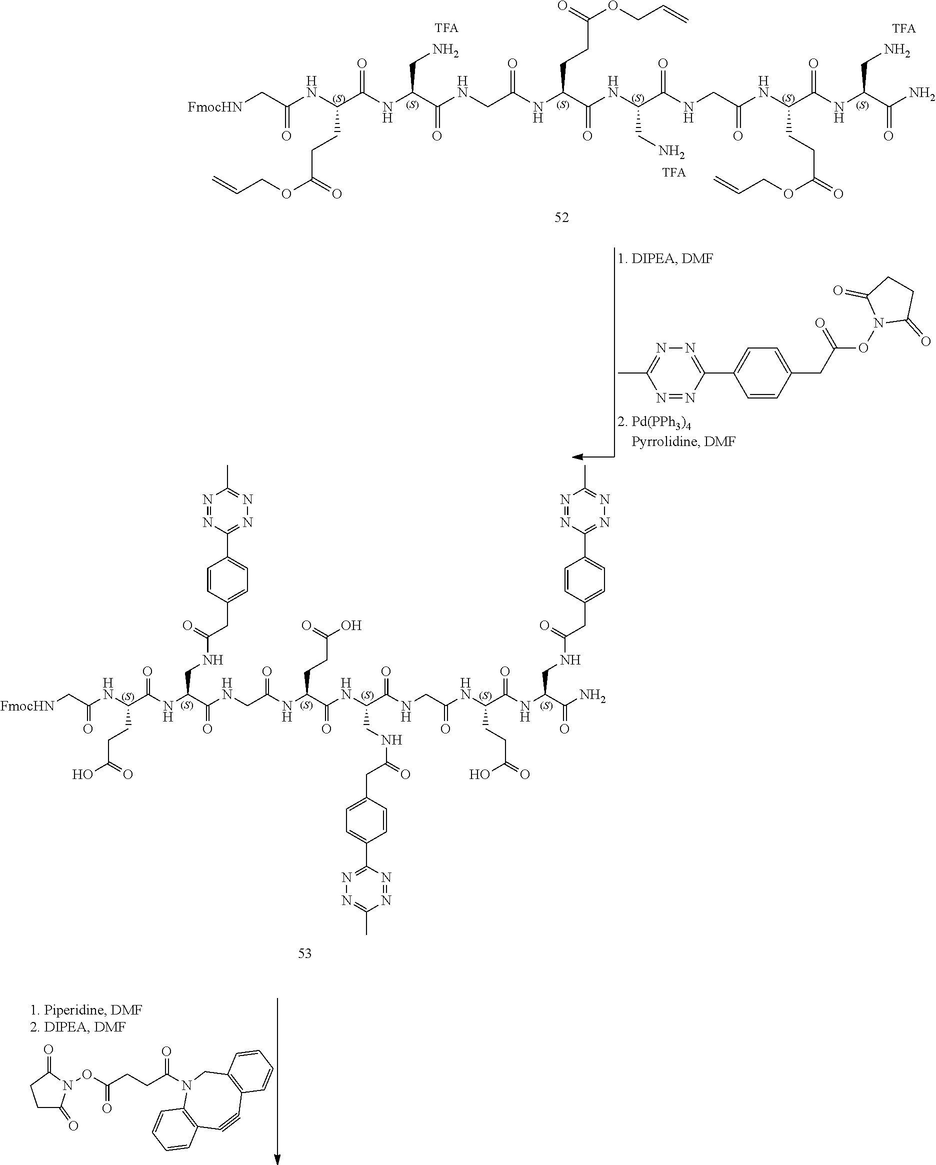

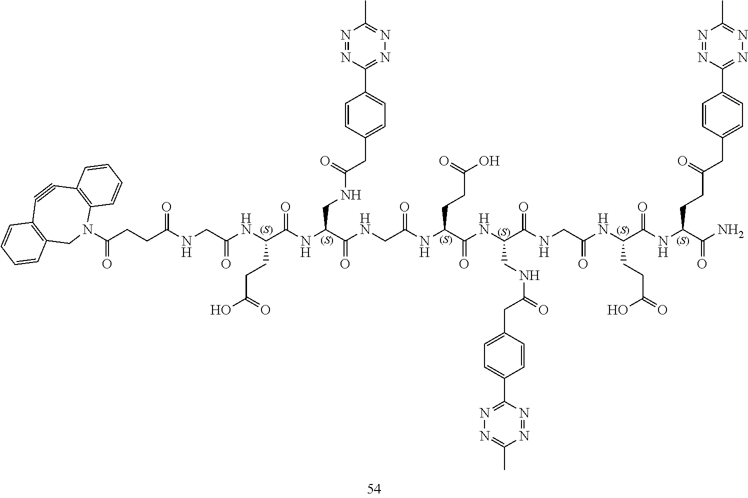

### Example 27. Preparation of Compound 55

To a solution of compound 54 (8.00 mg, 1 Eq, 4.55 μmol) in anhydrous DMF (500 μL) was added 1-aminooctaethylene glycol (8.1 mg, 4.8 Eq, 22 μmol) followed by the addition of HATU (6.5 mg, 3.8 Eq, 17 μmol) and DIPEA (3.53 mg, 4.76 μL, 6 Eq, 27.3 μmol). After 10 minutes, the desired product was precipitated by the addition of diethyl ether (10 mL). The precipitate was washed with diethyl ether (3×5 ml) and was diluted with DMF (600 μL) and DMSO (300 μL). The reaction mixture was purified by prep-HPLC; (5%→95%, MeCN/Water+10 mM NH₄HCO₃, runtime 12 minutes, Column Xbridge prep C18 5 μm OBD, 30×100 mm) and the product containing fractions were freeze dried to afford compound 55 (0.6 mg 0.2 μmol, 5%). LCMS (ESI+) calculated for C₁₃₀H₁₈₉N₂₉O₄₁²⁺ (M/2+H)⁺ 1406.7. found 1406.8.

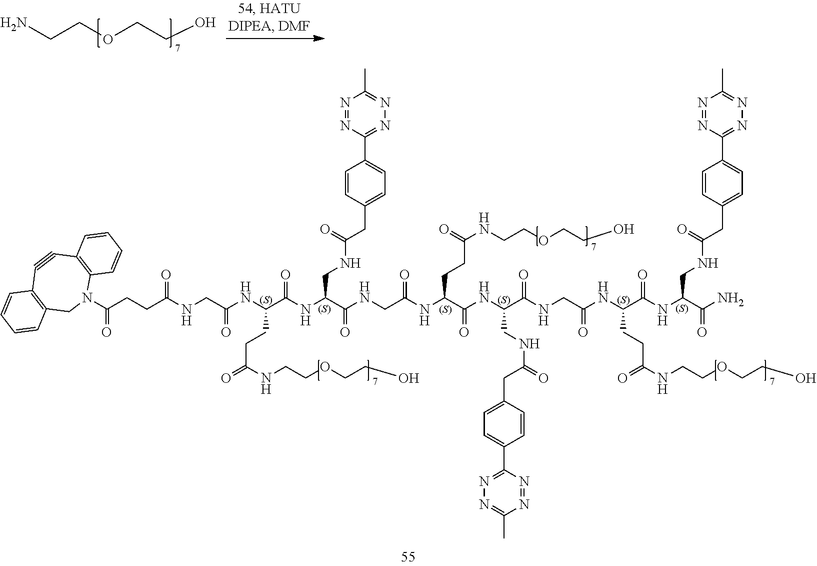

### Example 28. Preparation of Compound 57

To a solution of compound 6 (500 mg, 5.55 mL, 100 mmolar, 1 Eq, 555 μmol) in DMF (5 mL) was added triethylamine (281 mg, 387 μL, 5 Eq, 2.78 mmol followed by Val-Ala-PABC (375 mg, 2.3 Eq, 1.28 mmol). The resulting solution was stirred for 4 hours and was subsequently purified by flash column chromatography over silicagel (0%→20% MeOH in DCM) to afford diol intermediate (431 mg, 356 μmol, 64.2%) as a white solid. LCMS (ESI+) calculated for C₅₂H₇₇N₁₀O₁₉S₂⁺ (M+H)⁺ 1209.5. found 1210.1.

To a solution of diol intermediate (157 mg, 1 Eq, 130 μmol) in DMF (2 mL) was added bis 4-nitrophenyl carbonate (118 mg, 3 Eq, 389 μmol), followed by DIPEA (83.9 mg, 113 μL, 5 Eq, 649 μmol). After stirring for 2 hours, additional bis 4-nitrophenyl carbonate (118 mg, 3 Eq, 389 μmol) was added to the reaction mixture. After stirring for a total of 4 hours, the reaction mixture was purified by flash column chromatography over silicagel (0%→10% MeOH in DCM) to afford compound 56 (178 mg, 116 μmol, 89.1%) as a white solid. LCMS (ESI+) calculated for C₆₆H₈₃N₁₂O₂₇S₂⁺ (M+H)⁺ 1539.5. found 1540.0.

To a stock solution of compound 56 (4.10 mg, 26.6 μL, 100 mmolar, 1 Eq, 2.66 μmol) in DMF was added triethylamine (1.35 mg, 1.85 μL, 5 Eq, 13.3 μmol) and DMAP (650 μg, 5.32 μL, 1000 mmolar, 2 Eq, 5.32 μmol), followed by a stock solution of Paclitaxel (5.00 mg, 58.6 μL, 100 mmolar, 2.2 Eq, 5.86 μmol) in DMF. The resulting solution was left to stand at room temperature for 25 hours and subsequently purified by prep-HPLC; (5%→95%, MeCN/Water+10 mM NH₄HCO₃, runtime 12 minutes, Column Xbridge prep C18 5 μm OBD, 30×100 mm) to afford compound 57 (1.4 mg, 0.41 μmol, 16%, 88% Purity) as a white solid. LCMS (ESI+) calculated for C₁₄₈H₁₇₆N₁₂O₄₉S₂⁺² (M/2+H)⁺ 1485.0. found 1485.5.

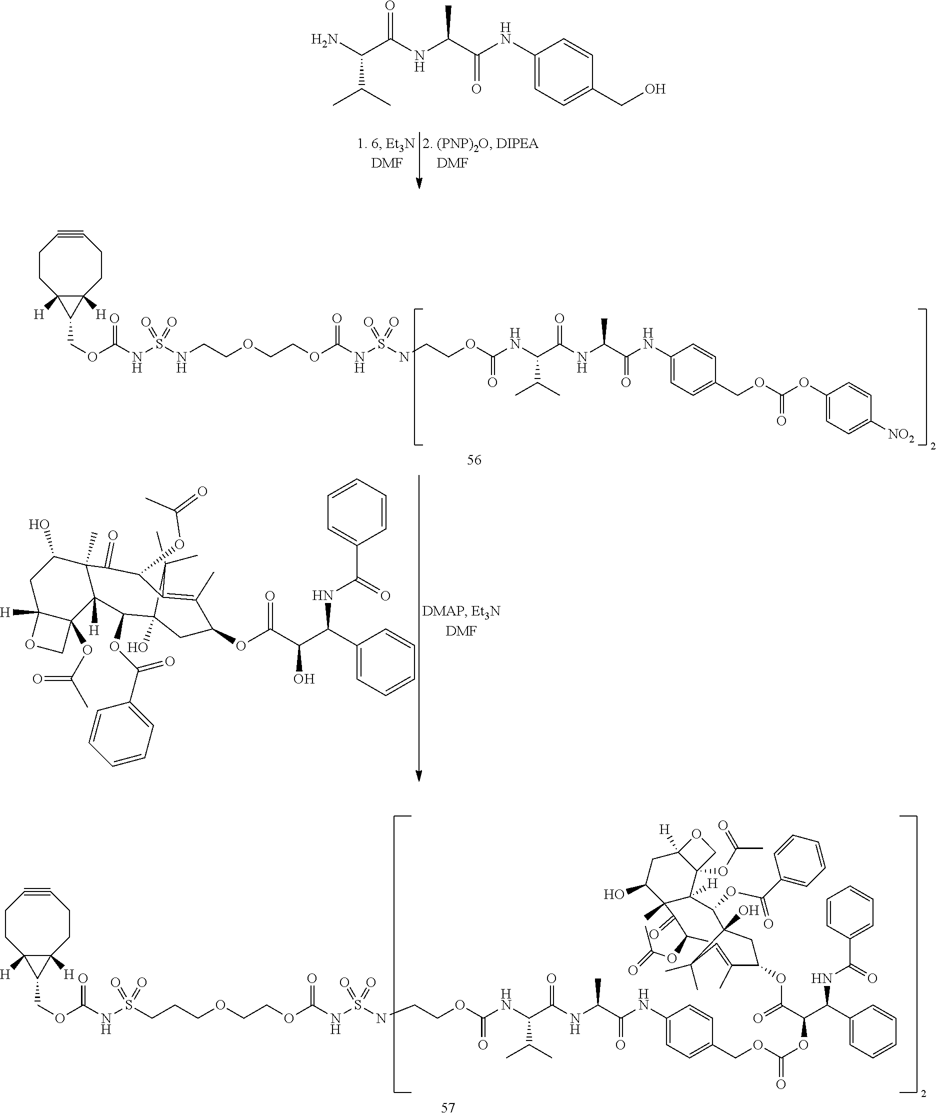

### Example 9. Preparation of Compound 59

To a solution of Fmoc-Val-Ala-PABC-PNP 4 (152.7 mg, 1.5 Eq. 224.3 μmol) in dry DCM (500 NL) was added a stock solution of Mitomycin C (50.00 mg, 1.496 mL, 100 m molar, 1 Eq, 149.6 μmol) in DMF, DIPEA (57.99 mg, 78.2 μL, 3 Eq, 448.7 μmol) and HOBt (34.35 mg, 224.3 μL, 1000 mmolar, 1.5 Eq, 224.3 μmol). After standing for 5 hours, the reaction mixture was purified by flash column chromatography over silicagel (0%→10% MeOH in DCM) to afford Fmoc-protected intermediate (115 mg, 91.9 μmol, 62%, 70% purity) as a brown solid. LCMS (ESI+) calculated for C₄₆H₅₃N₈O₁₁⁺ (M+NH₄)⁺ 893.4. found 894.0.

Fmoc-protected intermediate (115 mg, 70% Wt, 1 Eq, 91.9 μmol) was dissolved in DMF (500 μL) and piperidine (78.3 mg, 90.8 μL, 10 Eq, 919 μmol) was added. The resulting solution was stirred for 1 hour and was subsequently purified by flash column chromatography over silicagel (0%→30% MeOH in DCM) to afford compound 58 (72.9 mg, 112 μmol, 121%) as a brown solid. LCMS (ESI+) calculated for C₃₁H₄₀N₇O₉⁺ (M+H)⁺ 654.3. found 654.8.

Compound 58 (5.0 mg, 70% Wt, 3 Eq, 5.4 μmol) was dissolved in DCM (100 μL), subsequently compound 6 (1.6 mg, 18 μL, 100 mmolar, 1 Eq, 1.8 μmol) and DIPEA (3.0 mg, 4.0 μL, 13 Eq, 23 μmol) were added. The resulting solution left to stand for 4 days and was purified by flash column chromatography over silicagel (0%→10% MeOH in DCM). The obtained crude was subsequently purified by prep-HPLC; (5%→95%, MeCN/Water, runtime 12 minutes, Column Xbridge prep C18 5 μm OBD, 30×100 mm) to afford compound 59 (3.4 mg, 1.7 μmol, 93%, 94% Purity) as a brown solid. LCMS (ESI−) calculated for C₈₄H₁₀₈N₁₈O₃₁S₂⁻ M^{·-} 1928.7. found 1928.5.

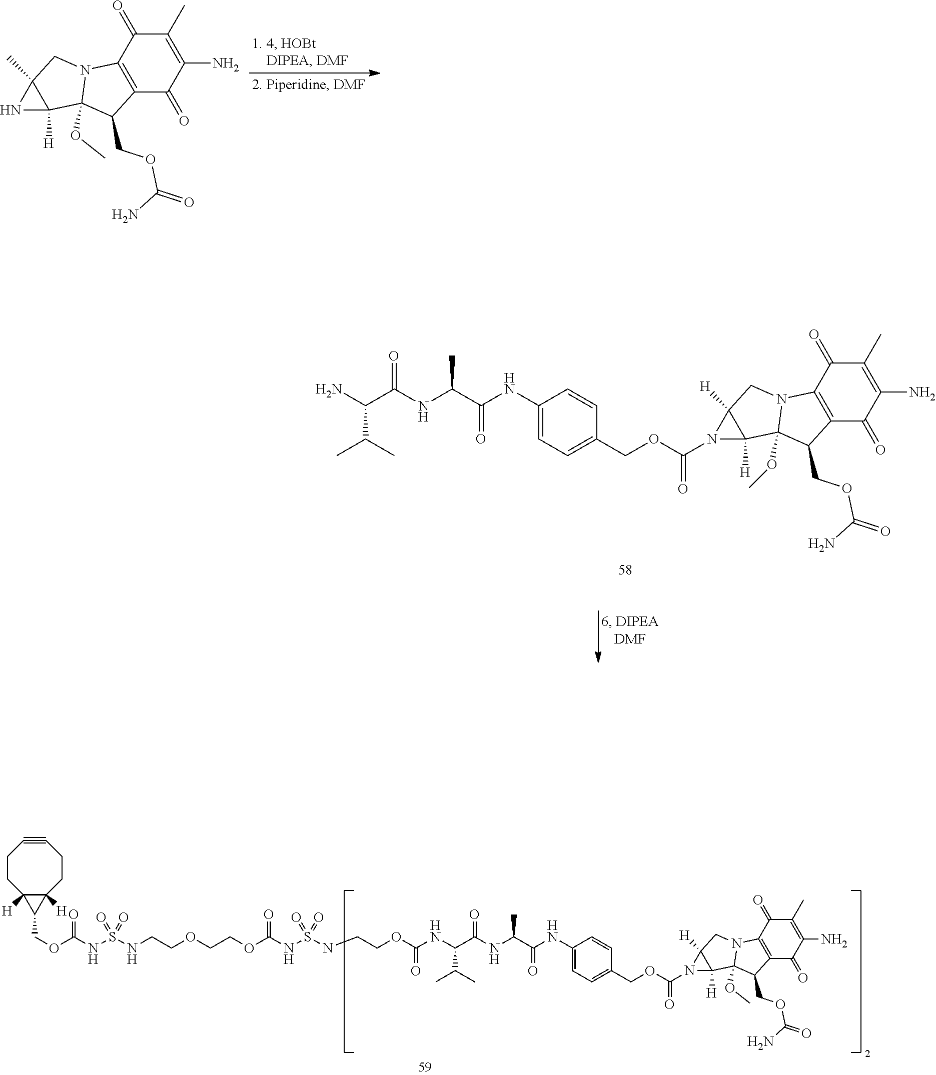

### Example 30. Preparation of Compound 61

To a solution of Fmoc-Val-Ala-PABC-PNP 4 (155.2 mg, 1.2 Eq, 228.0 μmol) in dry DCM (500 μL) was added a stock solution of Gemcitabine (50.00 mg, 1.900 mL, 100 mmolar, 1 Eq, 190.0 μmol) in DMF, DIPEA (73.66 mg, 99.3 μL, 3 Eq, 569.9 μmol) and DMAP (11.60 mg, 94.98 μL, 1000 mmolar, 0.5 Eq, 94.98 μmol). After standing for 3 hours, the reaction mixture was purified by flash column chromatography over silicagel (0%→20% MeOH in DCM) and the obtained crude was subsequently purified by prep-HPLC; (5%→95%, MeCN/Water+1% AcOH, runtime 12 minutes, Column Xbridge prep C18 5 μm OBD, 30×100 mm) to afford Fmoc-protected intermediate (15 mg, 19 μmol, 9.8%) as a white solid. LCMS (ESI+) calculated for C₄₀H₄₃F₂N₆O₁₀⁺ (M+H)⁺ 805.7. found 805.3.

Fmoc-protected intermediate (15 mg, 1 Eq, 19 μmol) was dissolved in DMF (200 μL) and triethylamine (57 mg, 78 μL, 30 Eq, 0.56 mmol) was added. The resulting solution was left to stand for 22 hours and was subsequently purified by flash column chromatography over silicagel (0%→100% MeOH in DCM) to afford compound 60 (8.9 mg, 15 μmol, 82%) as a white solid. LCMS (ESI+) calculated for C₂₅H₃₃F₂N₆O₈⁺ (M+H)⁺ 583.2. found 583.6.

Compound 60 (3.8 mg, 2.1 μmol, 42%) was dissolved in DMF (250 μL), subsequently compound 6 (4.6 mg, 51 μL, 100 mmolar, 1 Eq, 5.1 μmol) and triethylamine (5.2 mg, 7.1 μL, 10 Eq, 51 μmol) were added. The resulting solution was left to stand for 22 hours and subsequently purified by prep-HPLC; (5%→95%, MeCN/Water+1% AcOH, runtime 12 minutes, Column Xbridge prep C18 5 μm OBD, 30×100 mm) to afford compound 61 (3.8 mg, 2.1 μmol, 42%) as a white solid. LCMS (ESI+) calculated for C₇₂H₉₆F₄N₁₆O₂₉S₂⁺² (M/2+H)⁺ 894.3. found 895.0.

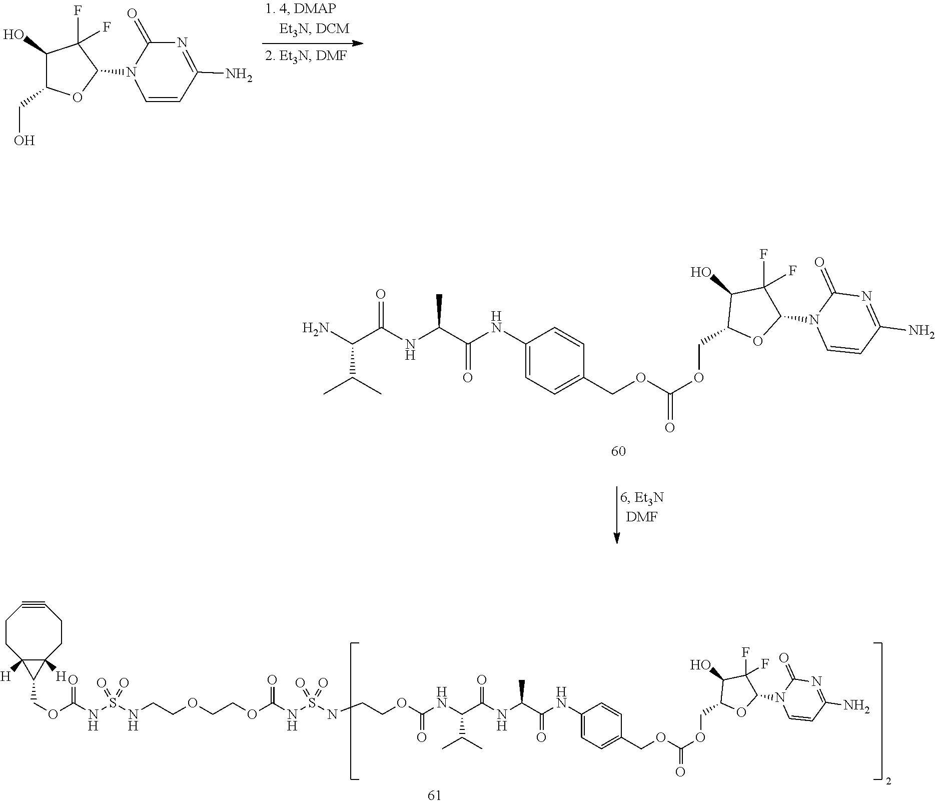

### Example 31. Preparation of Compound 63

To a solution of Gefitinib (50.0 mg, 1.0 Eq, 112 μmol) in dry DMF (1.1 mL) and dry DCM (2.5 mL) cooled at 0° C. was added sodium hydride (9.0 mg, 60% Wt, 2.0 Eq, 224 μmol). The reaction mixture was stirred at 0° C. for 1 hour and was then allowed to warm up to room temperature after which compound 4 (114 mg, 1.5 Eq, 168 μmol) was added. The reaction mixture was stirred at room temperature for 21 hours and was subsequently purified by flash column chromatography over silicagel (0→10% MeOH in DCM) to afford the Fmoc-protected intermediate (16.0 mg, 15 μmol, 14%, 95% purity). LCMS (ESI−) calculated for C₅₃H₅₆ClFN₇O₉⁻ (M−H+HCOOH)⁻ 988.4. found 988.8.

To a solution of the Fmoc-protected intermediate (16.0 mg, 95% Wt, 1.0 Eq, 15.4 μmol) in dry DMF (200 μL) was added Et₃N (46.7 mg, 64.3 μL, 30 Eq, 461 μmol). The reaction mixture was stirred at room temperature for 60 hours and was purified by flash column chromatography over silicagel (0→40% MeOH in DCM) to afford compound 62 (11.4 mg, 14 μmol, 93%, 96% purity). LCMS (ESI−) calculated for C₃₈H₄₆ClFN₇O₇⁻ (M−H+HCOOH)⁻ 766.3. found 766.7.

To a solution of the compound 62 (11.4 mg, 96% Wt, 3.0 Eq, 14.3 μmol) in dry DMF (200 μL) was added compound 6 (4.3 mg, 1.0 Eq, 4.8 μmol), followed by DIPEA (6.15 mg, 8.3 μL, 10 Eq, 47.6 μmol). The reaction mixture was stirred at room temperature for 20 hours and was subsequently purified by prep-HPLC; (5%→95%, MeCN/Water+10 mM NH₄HCO₃, runtime 12 minutes, Column Xbridge prep C18 5 μm OBD, 30×100 mm) to afford compound 63 (4.4 mg, 1.8 μmol, 39%, 90% purity). LCMS (ESI+) calculated for C₈₆H₁₂₂Cl₂F₂N₁₈O₂₃S₂²⁺ (M/2+H)⁺ 1033.9. found 1034.4.

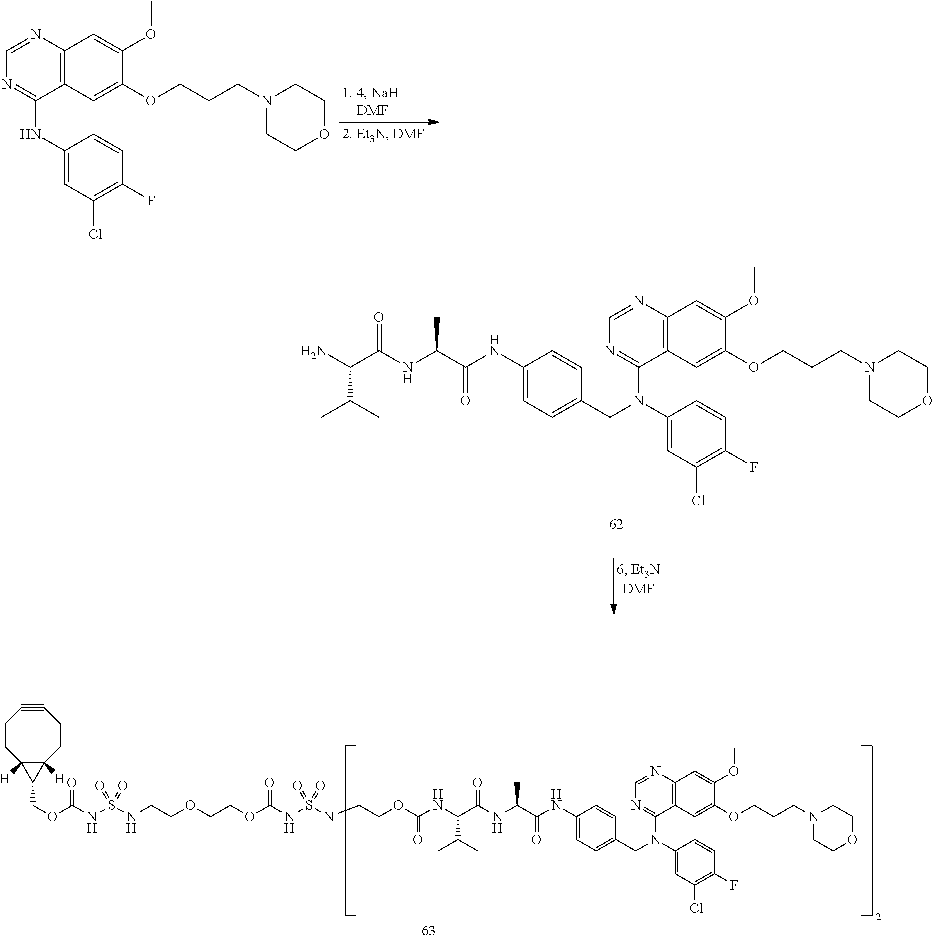

### Example 32. Preparation of Compound 67

To a solution of Paclitaxel (50.0 mg, 1.0 Eq, 58.6 μmol) in dry DMF (586 μL) was added bis(4-nitrophenyl) carbonate (35.6 mg, 2.0 Eq, 117 μmol), followed by triethylamine (17.8 mg, 24.5 μL, 3.0 Eq, 176 μmol). The reaction mixture was stirred at 0° C. for 3 hours and was subsequently purified by flash column chromatography over silicagel (0-100% EtOAc/Heptane) to afford compound 64 (32.9 mg, 30 μmol, 50%, 90% purity) as a white powder. LCMS (ESI+) calculated for C₅₄H₅₅N₂O₁₈⁺ (M+H)⁺ 1019.3. found 1019.7.

To a solution of compound 4 (750 mg, 1.0 Eq, 1.10 mmol) in dry THF (5.51 mL) was added tert-butyl (S)-methyl(pyrrolidin-2-ylmethyl)carbamate (394 mg, 1.7 Eq, 1.84 mmol), followed by DIPEA (427 mg, 576 μL, 3.0 Eq, 3.31 mmol). The reaction mixture was stirred at room temperature for 3 hours and was subsequently purified by flash column chromatography over silicagel (0→10% MeOH/DCM) to give the Boc-protected intermediate (861 mg, 1.14 mmol, 88%, 85% purity) as a pale-yellow solid. LCMS (ESI+) calculated for C₄₂H₅₇N₆O₈⁺ (M+NH₄)⁺ 773.4. found 773.7.

To a solution of the Boc-protected intermediate (833 mg, 85% Wt, 1.0 Eq, 937 μmol) in dry DCM (2.8 mL) at −15° C. was added TFA (2.14 g, 1.43 mL, 20 Eq, 18.7 mmol). The reaction mixture was stirred at −15° C. for 4 hours followed by precipitation with Et₂O. The solid was collected by filtration, washed with more Et₂O and concentrated in vacuo to afford compound 65 (737 mg, 947 μmol, 101%, 99% purity) as a brown solid. LCMS (ESI+) calculated for C₃₇H₄₆N₅O₆⁺ (M+H)⁺ 656.3. found 656.6.

To a solution of compound 65 (20.2 mg, 99% Wt, 1.0 Eq, 19.9 μmol) in dry DMF (100 μL) and dry DCM (200 μL) was added compound 64 (29.3 mg, 90% Wt, 1.3 Eq, 25.9 μmol), followed by DIPEA (7.7 mg, 10.4 μL, 3.0 Eq, 59.7 μmol). The reaction mixture was stirred at room temperature for 1 hour and was purified by flash column chromatography over silicagel (0→5% MeOH/DCM) to give the Fmoc-protected intermediate (26.5 mg, 16.0 μmol, 78%, 90% purity) as a pale-yellow solid. LCMS (ESI+) calculated for C₈₅H₉₅N₆O₂₁⁺ (M+H)⁺ 1535.7. found 1536.4.

To a solution of the Fmoc-protected intermediate (26.5 mg, 90% Wt, 1.0 Eq, 15.5 μmol) in dry DMF (200 μL) was added piperidine (4.0 mg, 4.6 μL, 3.0 Eq, 46.6 μmol). The reaction mixture was stirred at room temperature for 1 hour and was subsequently purified by flash column chromatography over silicagel (0→20% MeOH/DCM) to give compound 66 (18.9 mg, 14.4 μmol, 93%, 100% purity) as a white solid. LCMS (ESI+) calculated for C₇₀H₈₅N₆O₁₉⁺ (M+H)⁺ 1313.6. found 1314.1.

To a solution of compound 66 (5.0 mg, 2.5 Eq, 3.8 μmol) in dry DMF (90 μL) was added compound 6 (1.4 mg, 15 μL, 1.0 Eq, 1.5 μmol), followed by Et₃N (1.7 mg, 2.4 μL, 11.0 Eq, 17 μmol). The reaction mixture was stirred at room temperature for 30 hours and was then purified by prep-HPLC; (5%→95%, MeCN/Water+1% AcOH, runtime 12 minutes, Column Xbridge prep C18 5 μm OBD, 30×100 mm) to give compound 67 (2.6 mg, 0.8 μmol, 53%, 100% purity). LCMS (ESI−) calculated for C₁₆₂H₁₉₆N₁₆O₅₁S₂²⁻ (M/2−H)⁻ 1623.1. found 1623.5.

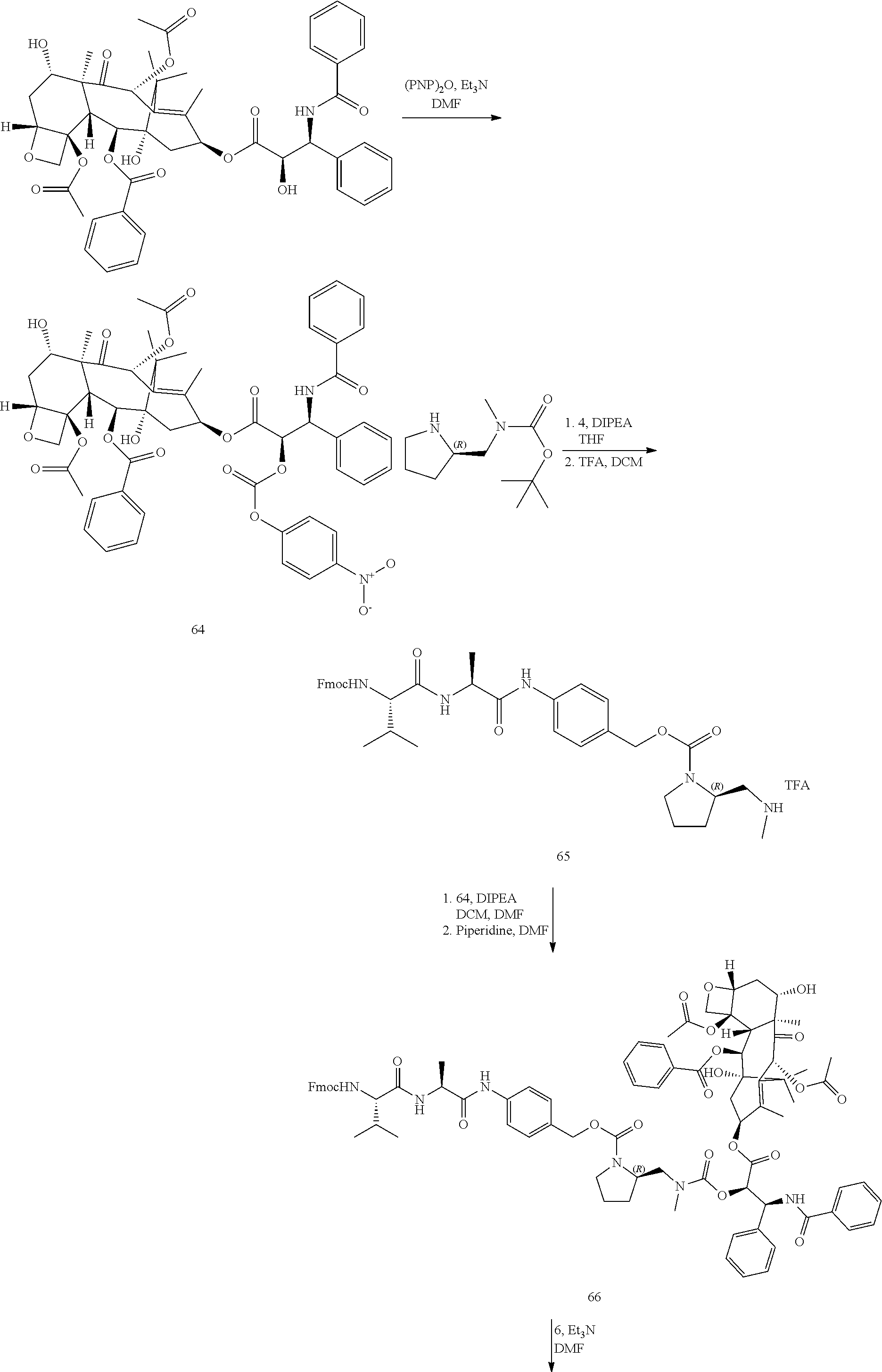

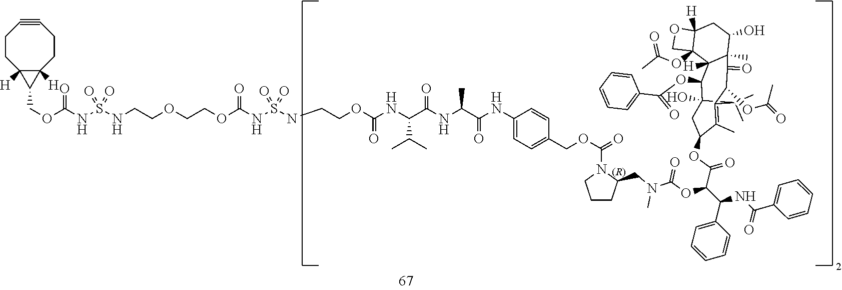

### Example 33. Preparation of Compound 70

A solution of Nemorubicin (150.0 mg, 1 Eq, 233.0 μmol) in DMF (1.2 mL) and DCM (2.0 mL) was cooled to 0° C., followed by the addition of bis(4-nitrophenyl)carbonate (286.6 mg, 4.04 Eq, 942.1 μmol) and triethylamine (94.33 mg, 130 μL, 4 Eq, 932.2 μmol). After 4.5 hours of stirring at 0° C., the reaction mixture was purified by flash column chromatography over silicagel (0%→7% MeOH in DCM) to afford compound 68 (170.1 mg, 0.16 mmol, 68%, 75% Purity) as a red oil. LCMS (ESI+) calculated for C₃₉H₄₁N₂O₁₇⁺ (M+H)⁺ 809.2. found 809.5.

To a solution of compound 68 (67.9 mg, 75% Wt, 1 Eq, 63.0 μmol) in anhydrous DMF (200 μL) and anhydrous DCM (500 μL) was added compound 65 (63.3 mg, 76% Wt, 0.992 Eq, 62.5 μmol) and DIPEA (48.8 mg, 65.8 μL, 6 Eq, 378 μmol). After stirring for reaction 17 hours at room temperature, piperidine (53.6 mg, 62.2 μL, 10 Eq, 630 μmol) was added. The reaction mixture was stirred for 1 hour at room temperature and subsequently purified by flash column chromatography over silicagel (0%→30% MeOH in DCM) to afford compound 69 (48.2 mg, 43.7 μmol, 69.4%) as a red oil. LCMS (ESI+) calculated for C₅₅H₇₁N₆O₁₈⁺ (M+H)⁺ 1103.5. found 1103.9.

To a solution of compound 69 (6.9 mg, 3 Eq, 6.3 μmol) in anhydrous DMF (150 μL) was added a solution of compound 6 in DMF (1.9 mg, 21 μL, 100 mmolar, 1.0 Eq, 2.1 μmol), followed by the addition of triethylamine (1.1 mg, 1.5 μL, 5 Eq, 10 μmol). After stirring for 22 hours at room temperature, N,N-dimethyl-1,2-ethanediamine (0.08 mg, 0.1 μL, 0.4 Eq, 0.9 μmol) was added to quench unreacted and mono-reacted compound 6. The reaction mixture was stirred for 15 minutes and subsequently purified by flash column chromatography over silicagel (0%→8% MeOH in DCM) to afford compound 70 (3.4 mg, 1.2 μmol, 58%) as a red oil. LCMS (ESI+) calculated for C₁₃₂H₁₇₂N₁₆O₄₉S₂²⁺ (M/2+H)⁺ 1415.0. found 1415.4.

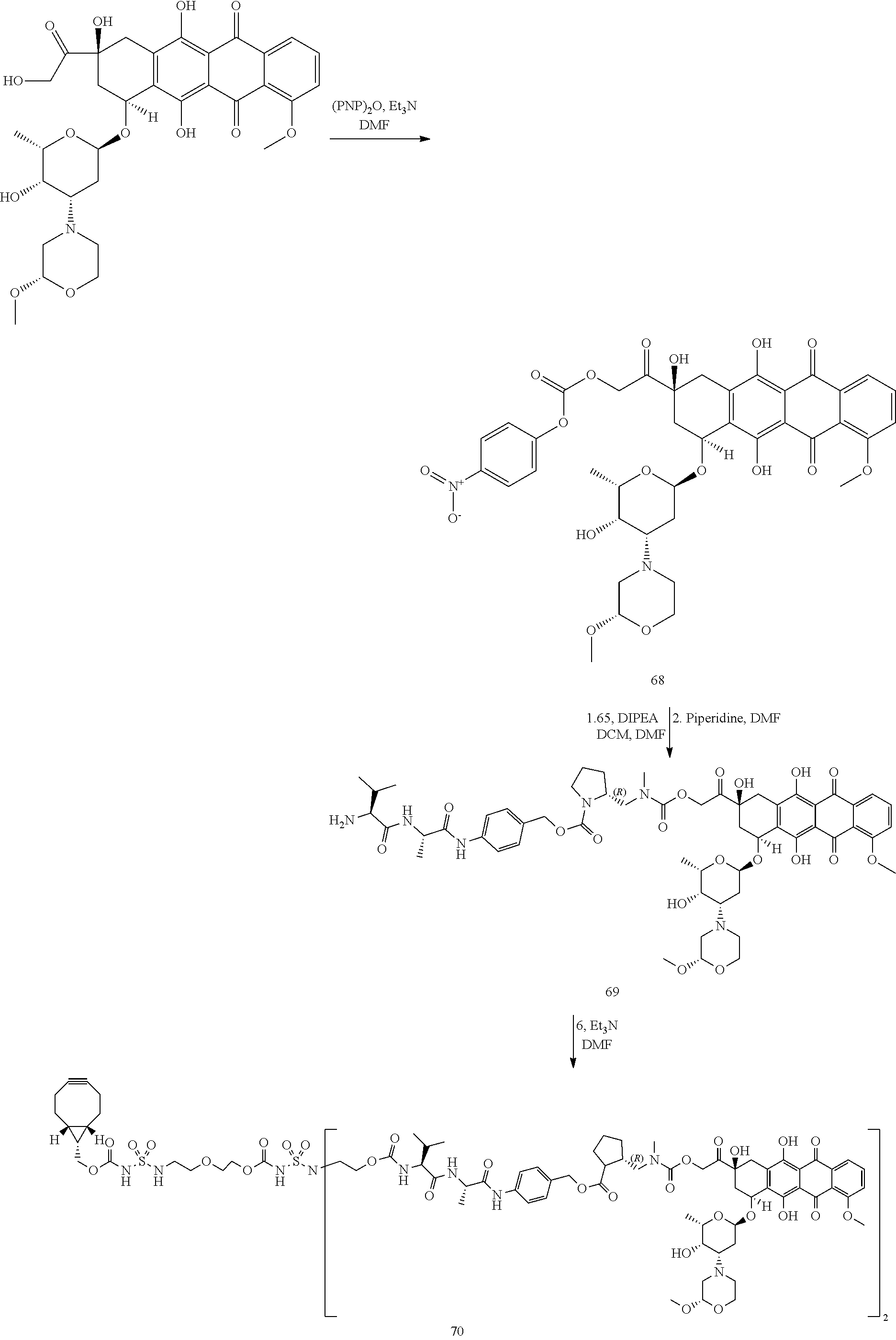

### Example 34. Preparation of Compound 72

The synthesis of compound 71 is described in PCT/EP2021/075401 (example 7), incorporated herein. Compound 58 (18.2 mg, 1 Eq, 27.8 μmol) was dissolved in DMF (150 ul) and to this was added compound 71 (20 mg, 91.5% Wt, 1.3 Eq, 35 μmol) (dissolved in 50 μl dry DMF) followed by triethylamine (7.0 mg, 9.6 μL, 2.5 Eq, 69 μmol). The reaction mixture was left to stand 20 hours and was subsequently purified by prep-HPLC; (5%→95%, MeCN/Water+1% AcOH, runtime 12 minutes, Column Xbridge prep C18 5 μm OBD, 30×100 mm) to afford compound 72 (24.6 mg, 23.3 μmol, 83.7%, 98.5% Purity) as a brown solid. LCMS (ESI+) calculated for C₄₇H₆₅N₁₀O₁₆S⁺ (M+NH₄)⁺ 1057.4. found 1057.8.

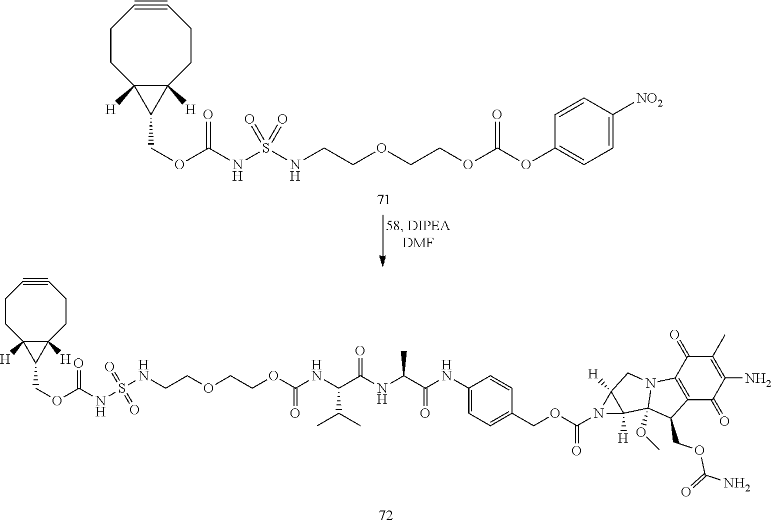 [SMILES: O=C(NS(=O)(=O)NCCOCCOC(=O)Oc1ccc([N+](=O)[O-])cc1)OC[C@@H]1[C@@H]2CCC#CCC[C@@H]21 ; CO[C@@]12[C@H](COC(N)=O)C3=C(C(=O)C(C)=C(N)C3=O)N1C[C@H]1[C@@H]2N1C(=O)OCc1ccc(NC(=O)[C@H](C)NC(=O)[C@@H](NC(=O)OCCOCCNS(=O)(=O)NC(=O)OC[C@@H]2[C@@H]3CCC#CCC[C@@H]32)C(C)C)cc1]

### Example 35. Preparation of Compound 75

To a solution of β-lapachone (100 mg, 1.0 Eq, 405 μmol) in THF (3000 μL) and water (3000 μL) was added Na₂S₂O₄ (356 mg, 4.3 Eq, 1.74 mmol), followed by TBAB (659 mg, 5.0 Eq, 2.02 mmol). The reaction mixture was heated up to 80° C. A solution of KOH (504 mg, 20 Eq, 8.09 mmol) in water (800 μL) was added, followed by a solution of Boc-para-aminobenzyl bromide (656 mg, 5.5 Eq, 2.23 mmol). After refluxing the reaction mixture for 150 minutes, ethyl acetate (10 mL) was added. The organic layer was washed with water (3×10 mL), dried with anhydrous magnesium sulfate, and concentrated in vacuo. The residue was then dissolved in heptane (5 mL) and purified by flash column chromatography over silicagel (0→20% EtOAc in Heptane) to afford the Boc-protected intermediate (123 mg, 203 μmol, 50%, 74% purity) as a pale-yellow oil. LCMS (ESI+) calculated for C₂₇H₃₂NO₅⁺ (M+H)⁺ 450.2. found 450.3.

To a solution of the Boc-protected intermediate (123 mg, 74% Wt, 1.0 Eq, 203 μmol) in dry DCM (2000 μL) cooled to 0° C. was added TFA (500 μL). The reaction mixture was stirred at 0° C. for 3 hours and was subsequently concentrated in vacuo to afford compound 73 (94 mg, 203 μmol, 100%, 100% purity). Yield was assumed quantitative. LCMS (ESI−) calculated for C₂₂H₂₄NO₃⁻ (M+H)⁻ 350.1. found 350.1.

To a solution of Fmoc-Val-Ala-OH (117 mg, 1.5 Eq, 284 μmol) in dry DMF (350 μL) and dry DCM (2400 μL) was added DIPEA (74 mg, 99 μL, 3.0 Eq, 568 μmol) and HATU (108 mg, 1.5 Eq, 284 μmol). After stirring reaction mixture for 30 minutes, a solution of compound 73 (88 mg, 1.0 Eq, 189 μmol) in dry DMF (750 μL) was added. The reaction mixture was stirred for 10 minutes and was subsequently purified by flash column chromatography over silicagel (0→3% MeOH in DCM) to afford compound 74 (193 mg, 0.2 mmol, 110%, 79% purity) as a pale-yellow oil. LCMS (ESI+) calculated for C₄₅H₄₈N₃O₇⁺ (M+H)⁺ 742.4. found 742.6.

To a solution of compound 74 (193 mg, 79% Wt, 1.0 Eq, 205 μmol) in dry DMF (2500 μL) was added piperidine (52 mg, 61 μL, 3.0 Eq, 616 μmol). The reaction mixture was stirred for 1 hour and was purified by flash column chromatography over silicagel (0→15% MeOH/DCM) to give the Fmoc-deprotected intermediate (64 mg, 98 μmol, 47%, 78% purity). LCMS (ESI−) calculated for C₃₀H₃₅N₃O₅⁻ (M−H)⁻ 518.3. found 518.3.

To a solution of the Fmoc-deprotected intermediate (31.2 mg, 78% Wt, 4.9 Eq, 48.6 μmol) in dry DMF (200 μL) was added compound 6 (8.9 mg, 1.0 Eq, 9.9 μmol), followed by Et₃N (15.0 mg, 20.6 μL, 15 Eq, 148 μmol). The reaction mixture was stirred at room temperature for 49 hours and subsequently was purified by prep-HPLC; (30%→95%, MeCN/Water+1% AcOH, runtime 12 minutes, Column Xbridge prep C18 5 μm OBD, 30×100 mm) to afford compound 75 (5.2 mg, 3.1 μmol, 32%, 100% purity). LCMS (ESI+) calculated for C₈₂H₁₀₅N₁₀O₂₃S₂⁺ (M+H)⁺ 1661.7. found 1662.1.

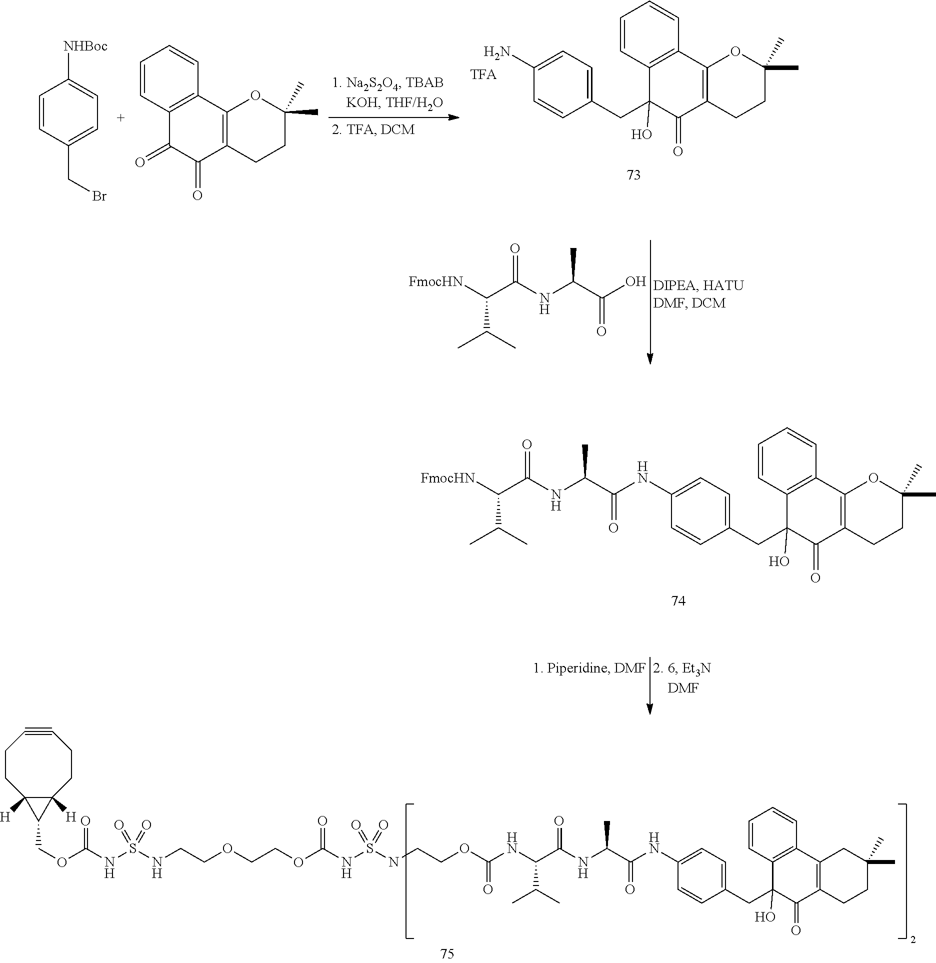

### Example 36. Preparation of Compound 77

A solution of Midostaurine (22.4 mg, 1 Eq, 39.3 μmol) in anhydrous THF (200 μL) was cooled to 0° C., followed by the addition of sodium hydride (3.3 mg, 60% Wt, 2.1 Eq, 83 μmol). After 20 minutes of stirring at 0° C., compound 4 (53.6 mg, 2.01 Eq, 78.7 μmol) was added. The reaction mixture was stirred for 10 minutes, and subsequently quenched by water (50 μL) and left stirring at 0° C. for 1 hour. Then piperidine (66.8 mg, 77.6 μL, 20 Eq, 785 μmol) was added to the reaction mixture. After 2.5 hours of stirring at 0° C., the reaction mixture was diluted with DCM (700 μL) and purified by flash column chromatography over silicagel (0%→35% MeOH in DCM). The residue was dissolved in DCM (500 μL) and was precipitated by the addition of cold diethyl ether (1.5 mL). The precipitate was washed with cold diethyl ether (3×1.5 mL) and concentrated in vacuo to afford compound 76 (23.2 mg, 25 μmol, 64%, 96% Purity) as a white solid. LCMS (ESI+) calculated for C₅₁H₅₂N₇O₈⁺ (M+H)⁺ 890.4. found 890.8.

To a solution of compound 76 (11.6 mg, 3 Eq, 13.0 μmol) in anhydrous DMF (250 μL) were added compound 6 (4.1 mg, 95% Wt, 1.0 Eq, 4.3 μmol) and triethylamine (2.20 mg, 3.03 μL, 5 Eq, 21.7 μmol). After stirring for 18 hours at room temperature, the reaction mixture was diluted with DCM (2 mL) and purified by flash column chromatography over silicagel (0%→10% MeOH in DCM) to afford compound 77 (6.4 mg, 2.3 μmol, 53%, 86% Purity). LCMS (ESI+) calculated for C₁₂₄H₁₃₇N₁₉O₂₉S₂⁺² ((M+NH₄+H)/2)⁺ 1210.5. found 1210.9.

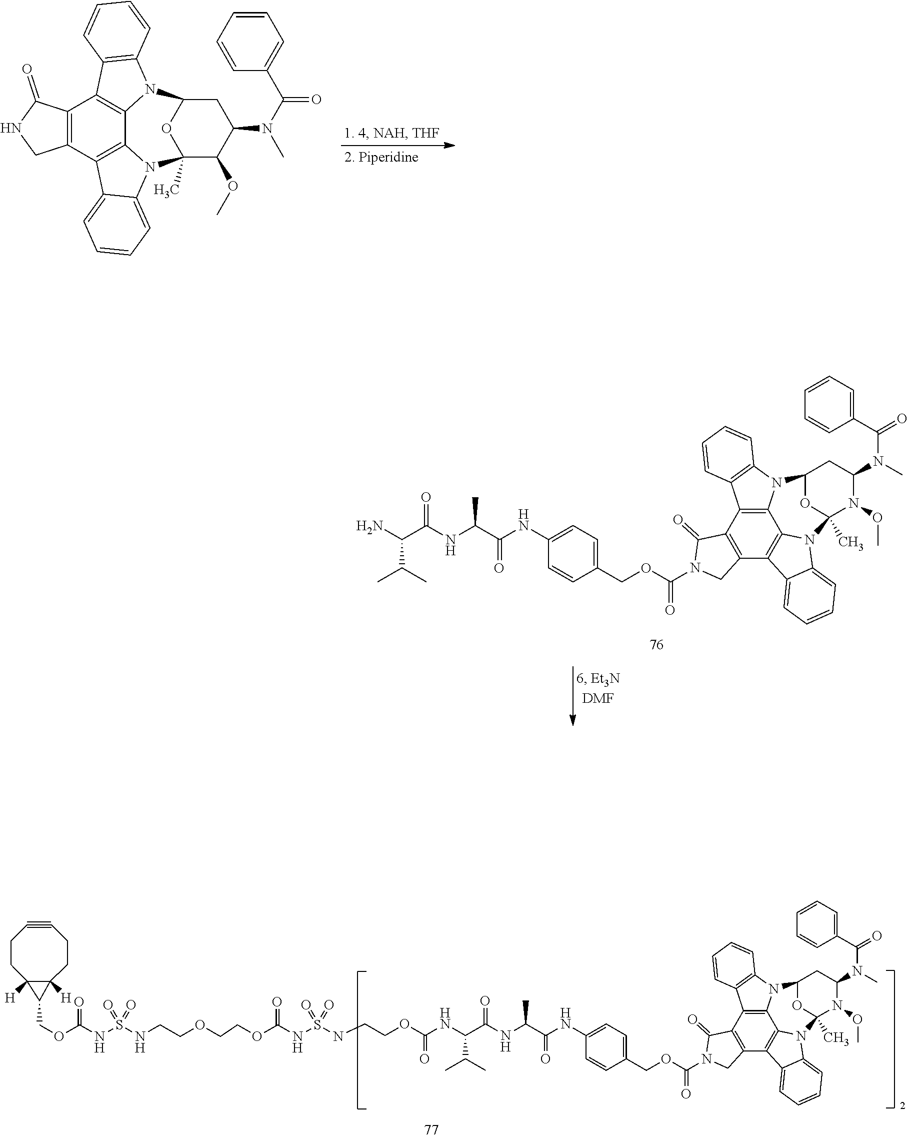

### Example 37. Preparation of Compound 79

Fmoc-Val-Ala-OH (40.0 mg, 1.5 Eq, 97.4 μmol) was dissolved in dry DMF (0.500 mL) and was added to a stock solution of Lenalidomide (16.8 mg, 325 μL, 200 mmolar, 1 Eq, 65.0 μmol) in DMF followed by DIPEA (33.6 mg, 45.3 μL, 4 Eq, 260 μmol) and HATU (49.4 mg, 2 Eq, 130 μmol). The reaction mixture was stirred for 20 hours and subsequently purified by flash column chromatography over silicagel (0→10% MeOH in DCM) to afford compound 78 (23 mg, 35 μmol, 54%) as a colorless oil. LCMS (ESI+) calculated for C₃₆H₃₈N₅O₇⁺ (M+H)⁺ 652.3. found 652.6.

To a solution of compound 78 (23 mg, 1 Eq, 35 μmol) in DCM (1.0 mL) was added piperidine (60 mg, 70 μL, 20 Eq, 0.71 mmol) and stirring was continued for 1 hours. The reaction mixture was purified by flash column chromatography over silicagel (0→30% MeOH in DCM) to afford Fmoc-deprotected intermediate (18 mg, 34 μmol, 95%, 80% Purity) as a white solid. LCMS (ESI−) calculated for C₂₁H₂₆N₅O₅⁻ (M−H)⁻ 428.2. found 428.3.

To a solution of Fmoc-deprotected intermediate (12.00 mg, 3 Eq, 27.94 μmol) in anhydrous DMF (279.4 μL) were added compound 6 (8.390 mg, 1 Eq, 9.314 μmol) and triethylamine (9.424 mg, 13.0 μL, 10 Eq, 93.14 μmol). After 16 hours of stirring at room temperature, Fmoc-deprotected intermediate (6.000 mg, 1.5 Eq, 13.97 μmol) and triethylamine (4.712 mg, 6.49 μL, 5 Eq, 46.57 μmol) were added. After 3 hours of stirring at room temperature, the reaction mixture was purified by prepHPLC; (5%→95%, MeCN/Water+10 mM NH₄HCO₃, runtime 12 minutes, Column Xbridge prep C18 5 μm OBD, 30×100 mm) to afford compound 79 (6 mg, 3 μmol, 30%, 80% Purity). LCMS (ESI+) calculated for C₆₄H₈₅N₁₄O₂₃S₂⁺ (M+H)⁺ 1481.5. found 1481.9.

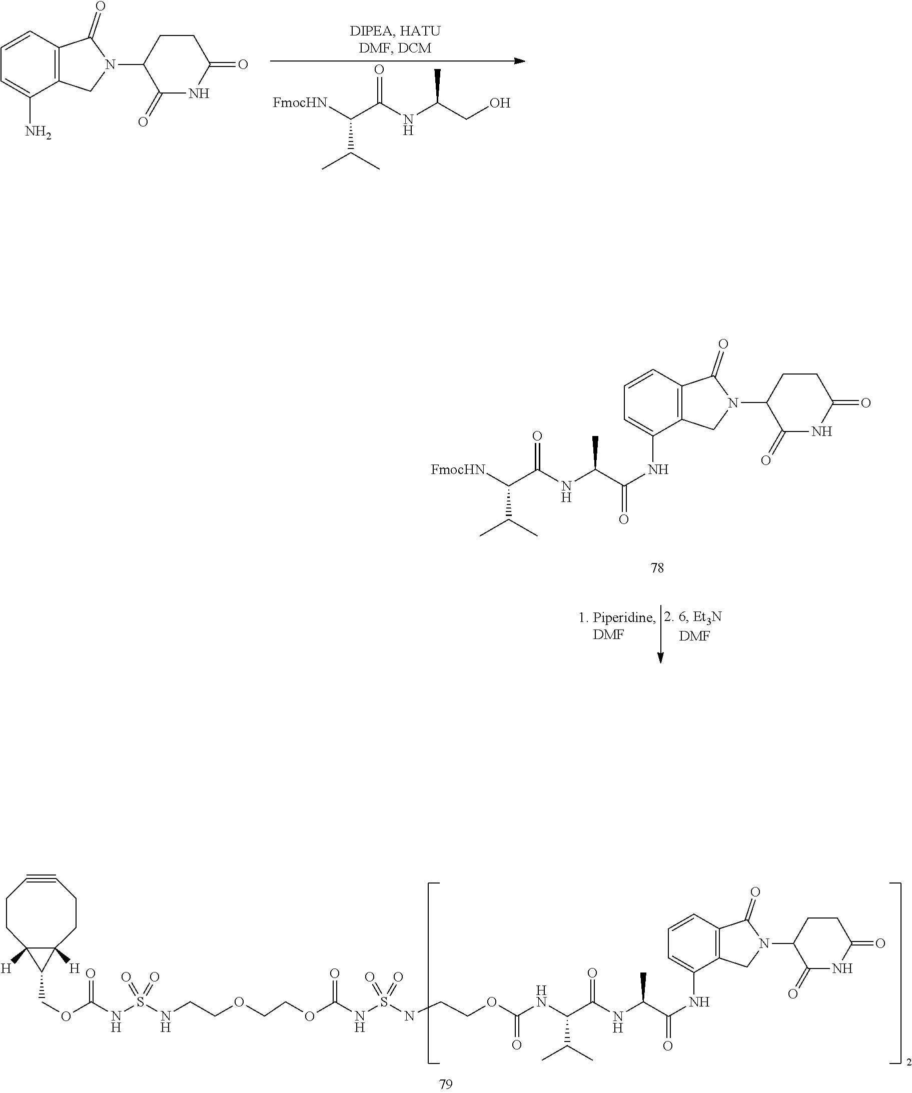

### Example 38. Preparation of Compound 82

A solution of Gemcitabine (50 mg, 2 Eq, 0.19 mmol) in DMF (0.95 mL) was cooled to −15° C. (ice salt bath). To this was added bis(4-nitrophenyl) carbonate (29 mg, 1 Eq, 95 μmol) and DIPEA (37 mg, 50 μL, 3 Eq, 0.28 mmol). After 3 hours of stirring at −15° C., compound 65 (88 mg, 1.2 Eq, 0.11 mmol) and DIPEA (37 mg, 50 μL, 3 Eq, 0.28 mmol) were added. The ice bath was removed and after stirring at room temperature for 15 minutes, the reaction mixture was purified by flash column chromatography over silicagel (0→20% MeOH in DCM) to afford compound 80 (57.4 mg, 59.0 μmol, 62%, 97% Purity) as a white solid. LCMS (ESI+) calculated for C₄₇H₅₅F₂N₈O₁₁⁺ (M+H)⁺ 945.4. found 945.8.

To a solution of compound 80 (57.4 mg, 1 Eq, 60.7 μmol) in anhydrous DMF (607 μL) was added piperidine (15.5 mg, 18.0 μL, 3 Eq, 182 μmol). After stirring for 1 hour at room temperature, the reaction mixture was purified by prepHPLC; (10%→60% MeCN/Water+10 mM NH₄HCO₃, runtime 23 min, column Xbridge prep C18 5 μm OBD, 30×100 mm) to afford compound 81 (13.2 mg, 18.3 μmol, 30%, 100% Purity) as a white solid. LCMS (ESI+) calculated for C₃₂H₄₅F₂N₈O₉⁺ (M+H)⁺ 723.3. found 723.6.

To a solution of compound 81 (8.00 mg, 3 Eq, 11.1 μmol) in anhydrous DMF (221 μL) was added compound 6 (3.32 mg, 1 Eq, 3.69 μmol) and triethylamine (3.73 mg, 5.14 μL, 10 Eq, 36.9 μmol). After stirring for 18 hours at room temperature, the reaction mixture was purified by prepHPLC; (20%→70% MeCN/Water+10 mM NH₄HCO₃, runtime 23 min, column Xbridge prep C18 5 μm OBD, 30×100 mm) to afford compound 82 (2.8 mg, 1.3 μmol, 35%, 95% Purity). LCMS (ESI+) calculated for C₈₆H₁₂₀F₄N₂₀O₃₁S₂⁺² (M/2+H)⁺ 1034.4. found 1034.7.

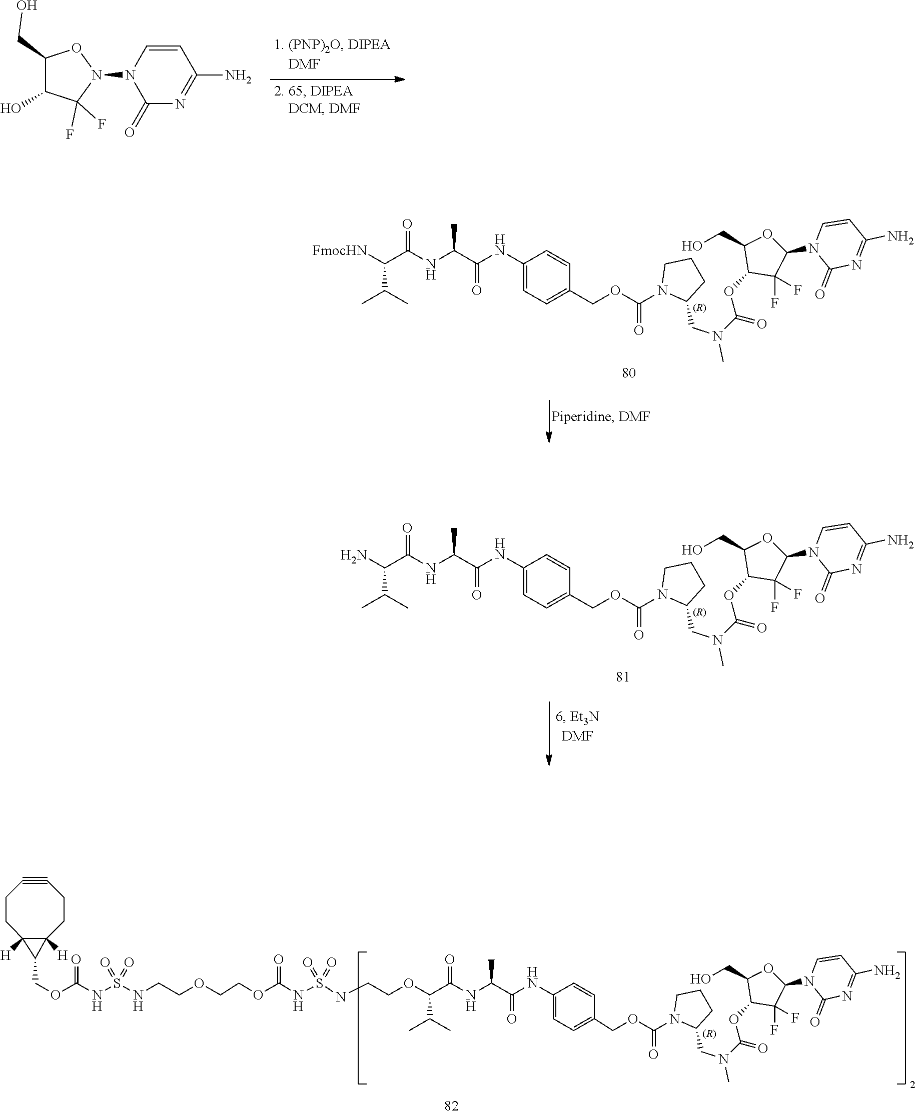

### Example 3. Preparation of Compound 86

To a solution of Gemcitabine (96 mg, 1 Eq, 0.36 mmol) in 1,4-dioxane (6.65 mL) and water (1.65 mL) was added sodium carbonate (0.15 g, 4 Eq, 1.5 mmol) and di-tert-butyl dicarbonate (88 mg, 89 μL, 1.1 Eq, 0.40 mmol). After stirring for 24 hours at room temperature for, additional di-tert-butyl dicarbonate (40 mg, 41 μL, 0.5 Eq, 0.18 mmol) was added. After stirring for 18 hours at room temperature, additional di-tert-butyl dicarbonate (40 mg, 41 μL, 0.5 Eq, 0.18 mmol) was added. After stirring for 6 hours at room temperature, water (20 mL) was added and the reaction mixture was extracted with EtOAc (2×25 mL). The combined organic layers were washed with brine, dried with anhydrous Na₂SO₄, filtered and concentrated in vacuo. The residue was purified by flash column chromatography over silicagel (20→100% Acetone in DCM) to afford the Boc-protected intermediate (83 mg, 0.23 mmol, 62%, 99% Purity) as a white solid. LCMS (ESI+) calculated for C₁₄H₂₀F₂N₃O₆⁺ (M+H)⁺ 364.1. found 364.4.

To a solution of Boc-protected intermediate (83 mg, 1 Eq, 0.23 mmol) in 1,4-dioxane (4.6 mL) was added di-tert-butyl dicarbonate (640 mg, 0.67 mL, 13 Eq, 2.9 mmol). After stirring for 24 hours at 37° C., the reaction mixture was allowed to reach room temperature and was stirred for an additional 72 hours. Water (20 mL) was added and the reaction mixture was extracted with EtOAc (2×25 mL). The combined organic layers were washed with brine, dried with anhydrous Na₂SO₄, filtered and concentrated in vacuo. The residue was purified by flash column chromatography over silicagel (0→20% Acetone in DCM) to afford compound 83 (93.3 mg, 201 μmol, 88%, 100% Purity) as a white solid. LCMS (ESI+) calculated for C₁₉H₂₈F₂N₃O₈⁺ (M+H)⁺ 464.2. found 464.5.

To a solution of compound 83 (21 mg, 1 Eq, 45 μmol) in MeCN (0.45 mL) was added triethylamine (14 mg, 19 μL, 3 Eq, 0.14 mmol) and bis(4-nitrophenyl) carbonate (28 mg, 2 Eq, 91 μmol). After stirring for 18 hours at room temperature, the reaction mixture was purified by flash column chromatography over silicagel (0→10% MeOH in DCM) to afford compound 84 (28 mg, 43 μmol, 95%, 97% Purity) as a yellow solid. LCMS (ESI+) calculated for C₂₆H₃₁F₂N₄O₁₂⁺ (M+H)⁺ 629.2. found 629.5.

To a solution of compound 84 (14 mg, 1.3 Eq, 22 μmol) in anhydrous DCM (0.13 mL) was added a solution of compound 65 (18 mg, 73% Wt, 1.0 Eq, 17 μmol) in anhydrous DMF (66 μL) and DIPEA (6.6 mg, 8.9 μL, 3.0 Eq, 51 μmol). After stirring for 1 hour at room temperature, the reaction mixture was purified by flash column chromatography over silicagel (0→20% MeOH in DCM) to afford Fmoc-protected intermediate (25 mg, 18 μmol, 99%, 81% Purity) as a white solid. LCMS (ESI+) calculated for C₅₇H₇₁F₂N₈O₁₅⁺ (M+H)⁺ 1145.5. found 1145.9.

To a solution of Fmoc-protected intermediate (21 mg, 1 Eq, 18 μmol) in anhydrous DMF (0.2 mL) was added piperidine (4.6 mg, 5.3 μL, 3 Eq, 54 μmol). After stirring for 4 hours at room temperature, the reaction mixture was purified by flash column chromatography over silicagel (0→20% MeOH in DCM) to afford compound 85 (9.6 mg, 10 μmol, 57%, 98% Purity). LCMS (ESI+) calculated for C₄₂H₆₁F₂N₈O₁₃⁺ (M+H)⁺ 923.4. found 923.8.

A solution of compound 85 (9.6 mg, 1 Eq, 10 μmol) in DCM (0.16 mL) was cooled to 0° C. and TFA (52 μL, 68 Eq, 0.68 mmol) was added. After stirring for 3 hours at 0° C., the reaction mixture was concentrated in vacuo to afford Boc-deprotected intermediate (12.9 mg, 7.9 μmol, 76%, 58% Purity) as an off-white solid. LCMS (ESI+) calculated for C₃₂H₄₅F₂N₈O₉⁺ (M+H)⁺ 723.3. found 723.6.

To a solution of Boc-deprotected intermediate (9.9 mg, 1 Eq, 10 μmol) in anhydrous DMF (0.21 mL) was added triethylamine (11 mg, 15 μL, 10 Eq, 0.10 mmol) and compound 6 (8.2 mg, 1.5 Eq, 16 μmol). After stirring for 18 hours at room temperature, triethylamine (11 mg, 15 μL, 10 Eq, 0.10 mmol) was added. After stirring at for 6 hours room temperature, HOBt (1.6 mg, 1 Eq, 10 μmol) was added. After stirring for 18 hours at room temperature, DMAP (1.3 mg, 1 Eq, 10 μmol) was added and the reaction mixture was heated to 40° C. After stirring for 6 hours at 40° C., the reaction mixture was purified by prep-HPLC; (5%→95%, MeCN/Water+1% AcOH, runtime 12 minutes, Column Xbridge prep C18 5 μm OBD, 30×100 mm) and second time by prepHPLC; (10%→60%, MeCN/Water+10 mM NH₄HCO₃, runtime 23 minutes, Column Xbridge prep C18 5 μm OBD, 30×100 mm) to afford compound 86 (1.2 mg, 1.1 μmol, 10%, 100% Purity). LCMS (ESI+) calculated for C₄₈H₆₇F₂N₁₀O₁₆S⁺ (M+H)⁺ 1109.4. found 1109.8.

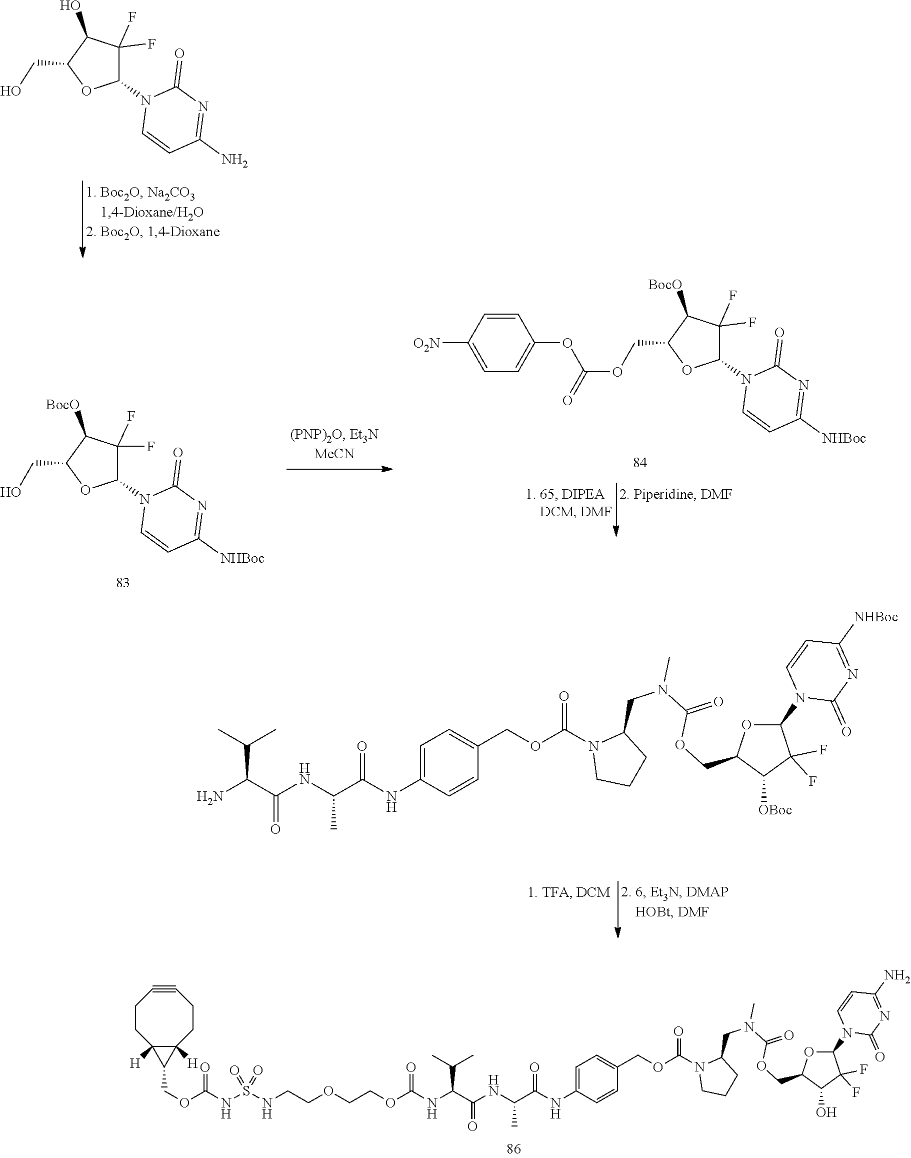

### Example 40. Preparation of Compound 87

To a solution of compound 56 (10.5 mg, 1.0 Eq, 6.8 μmol) in dry DMF (270 μL) was added a HCl salt of Daunorubicin (9.6 mg, 170 μL, 2.5 Eq, 17 μmol), followed by Et₃N (6.9 mg, 9.5 μL, 10 Eq, 68 μmol) and DMAP (0.8 mg, 1.0 Eq, 6.8 μmol). The reaction mixture was stirred at room temperature for 22 hours and was subsequently purified by prep-HPLC; (5%→95%, MeCN/Water+1% AcOH, runtime 12 minutes, Column Xbridge prep C18 5 μm OBD, 30×100 mm) to afford compound 87 (5.7 mg, 2.5 μmol, 36%, 100% purity). LCMS (ESI−) calculated for C₁₀₈H₁₂₈N₁₂O₄₁S₂²⁻ (M/2−H)⁻ 1156.4. found 1156.8.

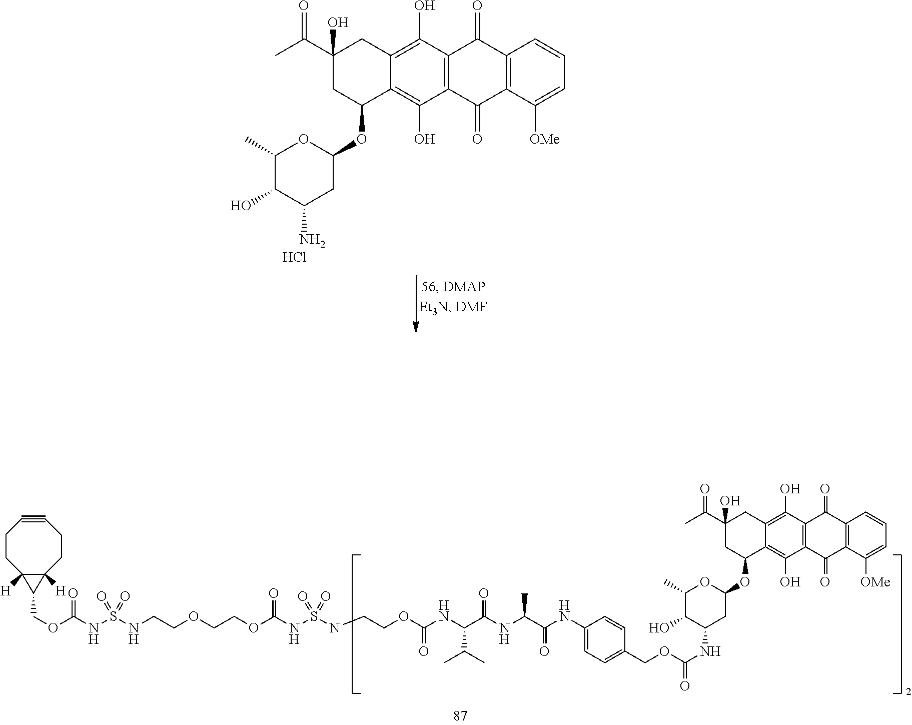

### Example 41. Preparation of Compound 88

To a solution of compound 71 (4.79 mg, 1.3 Eq, 8.34 μmol) in dry DMF (100 μL) was added compound 66 (8.43 mg, 1.0 Eq, 6.42 μmol) and triethylamine (1.95 mg, 2.68 μL, 3.0 Eq, 19.3 μmol). The reaction mixture was left to stand at room temperature for 8 hours and was subsequently diluted with DMF (500 μl) and purified by prep-HPLC (injection volume 650 μL DMF; 30%→95%, MeCN/Water+1% AcOH, runtime 23 minutes, Column Xbridge prep C18 5 μm OBD, 30×100 mm). The obtained crude product was re-dissolved in 400 μl DMF and purified again by prep-HPLC (injection volume 450 μL DMF; 30%→95%, MeCN/Water+1% AcOH, runtime 23 minutes, Column Xbridge prep C18 5 μm OBD, 30×100 mm) to afford compound 88 (3.5 mg, 2.0 μmol, 31%, 97.77% Purity). LCMS (ESI+) calculated for C₈₆H₁₀₇N₈O₂₆S⁺ (M+H)⁺ 1699.7. found 1700.2.

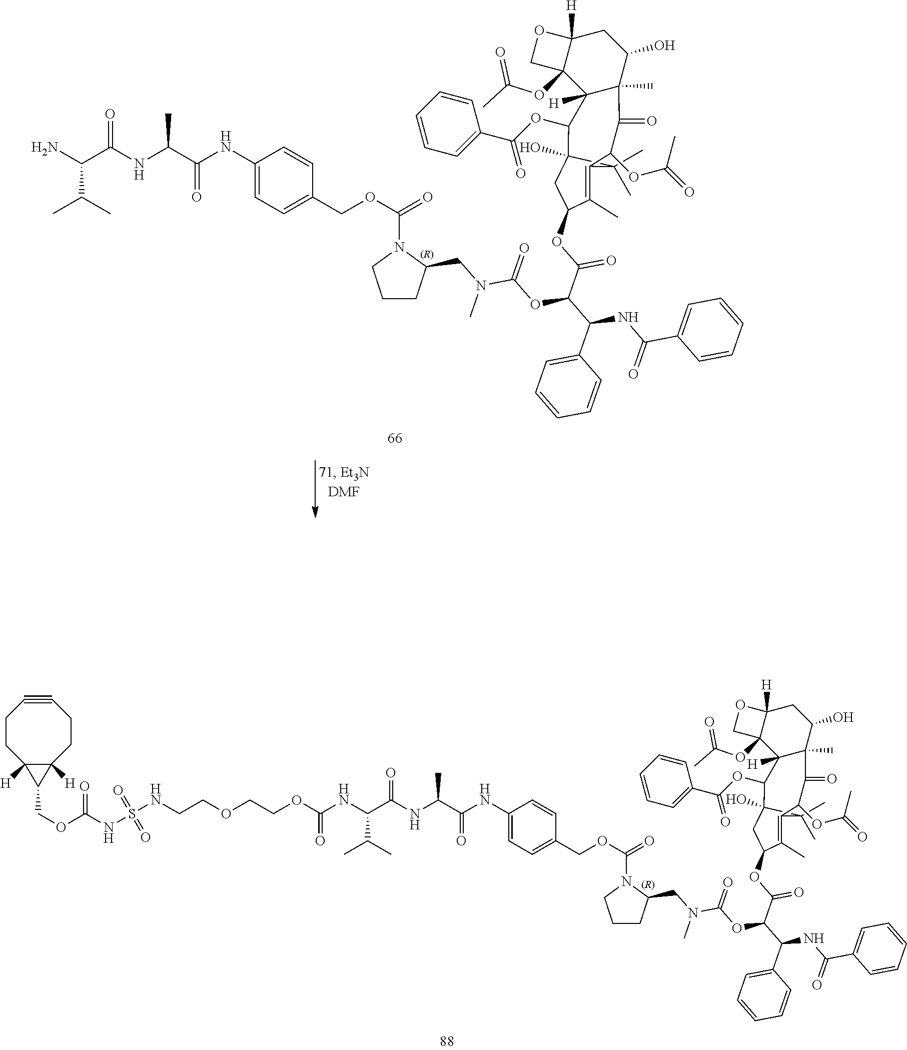

### Example 42. Preparation of Compound 91

To a solution of Fmoc-Val-Ala-PABC-OPNP 4 (35.0 mg, 1.0 Eq, 51.4 μmol) in dry THF (2.57 mL) was added 3-[(2R)-pyrrolidin-2-yl]propanoic acid hydrochloride (23.1 mg, 2.5 Eq, 129 μmol) and DIPEA (19.9 mg, 26.9 μL, 3.0 Eq, 154 μmol). The reaction mixture was stirred at room temperature under N₂ atmosphere. After 40 minutes, 300 μl dry DMF was added to aid in solubility. After 6.5 hours the reaction mixture was concentrated in vacuo and a clear pale-yellow oil was obtained. The crude was re-dissolved in DCM (3 ml) and the solution was purified by flash column chromatography over silicagel (0%-20% MeOH in DCM) to afford an unactivated acid intermediate (47.4 mg, 60.2 μmol, 117%, 86.93% Purity) as a pale-yellow solid. LCMS (ESI+) calculated for C₃₈H₄₅N₄O₈⁺ (M+H)⁺ 685.3. found 685.8.

To a solution of an unactivated acid intermediate (47.4 mg, 1.0 Eq. 60.2 μmol) in dry DCM (661 μL) was added 3-(((ethylimino)methylene)amino)-N,N-dimethylpropan-1-amine hydrochloride (15.0 mg, 1.3 Eq. 78.2 μmol) and 2,3,5,6-tetrafluorophenol (13.0 mg, 1.3 Eq. 78.2 μmol). 50 μl of dry DMF was added to aid in solubility. The reaction mixture was stirred at room temperature for 2 hours and concentrated in vacuo. The crude was re-dissolved in DCM (1 ml) and purified by flash column chromatography over silicagel (0%→30% Acetone in DCM) to afford compound 89 (60 mg, 31 μmol, 52%, 43.64% Purity). LCMS (ESI+) calculated for C₄₄H₄₈F₄N₅O₈⁺ (M+NH₄)⁺ 850.3. found 850.7.

To a solution of compound 89 (60.0 mg, 1.0 Eq, 31 μmol) in a mixture of dry DCM (187 μL) and dry DMF (100 μl) was added Paclitaxel (34.0 mg, 1.25 Eq, 39.0 μmol). After stirring the reaction mixture 50 minutes, DMAP (4.2 mg, 1.1 Eq, 35.0 μmol) was added. After 30 minutes, the reaction mixture was diluted with DCM (3 m) and purified by flash column chromatography over silicagel (0%→20% MeOH in DCM) to afford Fmoc-protected intermediate (49.3 mg, 26.5 μmol, 84%, 81.77% Purity). LCMS (ESI+) calculated for C₈₅H₉₄N₅O₂₁⁺ (M+H)⁺ 1520.6. found 1521.2.

To a solution of Fmoc-protected intermediate (49.3 mg, 1.0 Eq, 26.5 μmol) in DMF (200 μL) was added piperidine (6.77 mg, 7.86 μl, 3.0 Eq, 79.5 μmol). The reaction mixture was stirred at room temperature for 30 minutes and subsequently diluted with DCM (4 ml). The solution was then purified by flash column chromatography over silicagel (0%→15% MeOH in DCM) to afford compound 90 (15.7 mg, 11.9 μmol, 45%, 98.65% Purity). LCMS (ESI+) calculated for C₇₀H₈₄N₅O₁₉⁺ (M+H)⁺ 1298.6. found 1299.0.

To a solution of compound 90 (7.8 mg, 3.0 Eq, 5.93 μmol) in dry DMF (100 μL) was added compound 6 (1.78 mg, 1.0 Eq, 1.98 μmol) and triethylamine (1.0 mg, 1.38 μL, 5.0 Eq, 9.88 μmol). The reaction mixture was left to stand at room temperature for 7.5 hours after which it was kept at −20° C. for 17 hours. The reaction was warmed to room temperature for 5 more hours, before it was diluted with DMF (500 μl) and subsequently purified by prep-HPLC (injection volume 550 μL DMF; 30%→95%, MeCN/Water+1% AcOH, runtime 23 minutes, Column Xbridge prep C18 5 μm OBD, 30×100 mm) to afford compound 91 (3.3 mg, 1.0 μmol, 52%, 100% Purity). LCMS (ESI+) calculated for C₁₆₂H₁₉₈N₁₄O₅₁S₂⁺² (M/2+H)⁺ 1609.1. found 1609.6.

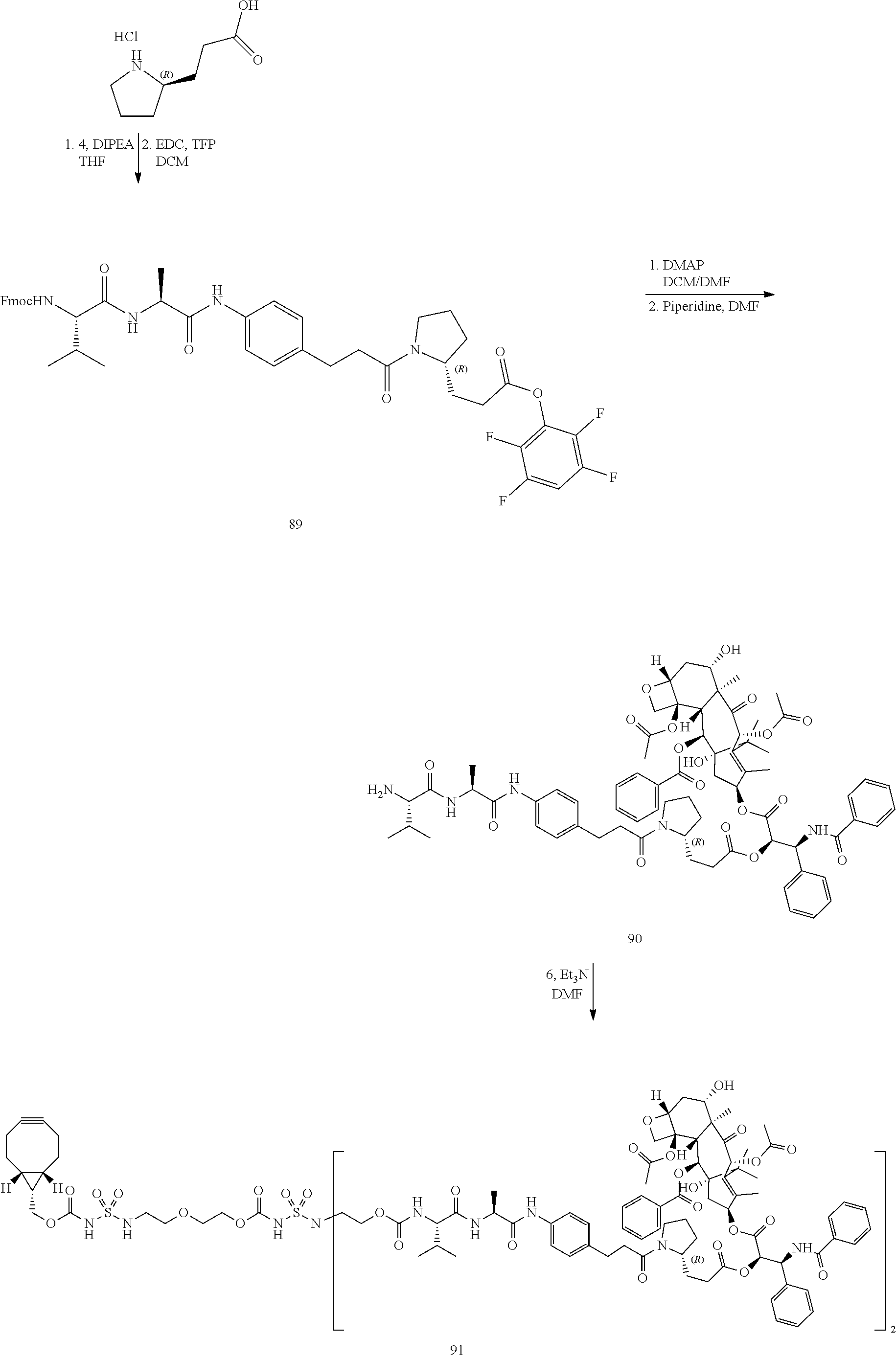

### Example 43. Preparation of Compound 92

To a solution of compound 71 (4.45 mg, 1.3 Eq, 7.75 μmol) in dry DMF (30 μL) was added compound 90 (7.85 mg, 1.0 Eq, 5.96 μmol) in dissolved dry DMF (70 μl) followed by triethylamine (1.81 mg, 2.49 μL, 3.0 Eq, 17.9 μmol). The reaction mixture was left to react at room temperature for 7 hours after which it was cooled to −20° C. for 17 hours. The reaction was then warmed to room temperature for 5 more hours, before it was diluted with DMF (400 μl) and subsequently purified by prep-HPLC (injection volume 550 μL DMF; 30%→95%, MeCN/Water+1% AcOH, runtime 23 minutes, Column bridge prep C18 5 μm OBD, 30×100 mm) to afford compound 92 (6.6 mg, 3.8 μmol, 64%, 97.87% Purity). LCMS (ESI+) calculated for C₈₆H₁₀₆N₇O₂₆S⁺ (M+H)⁺ 1684.7. found 1685.1.

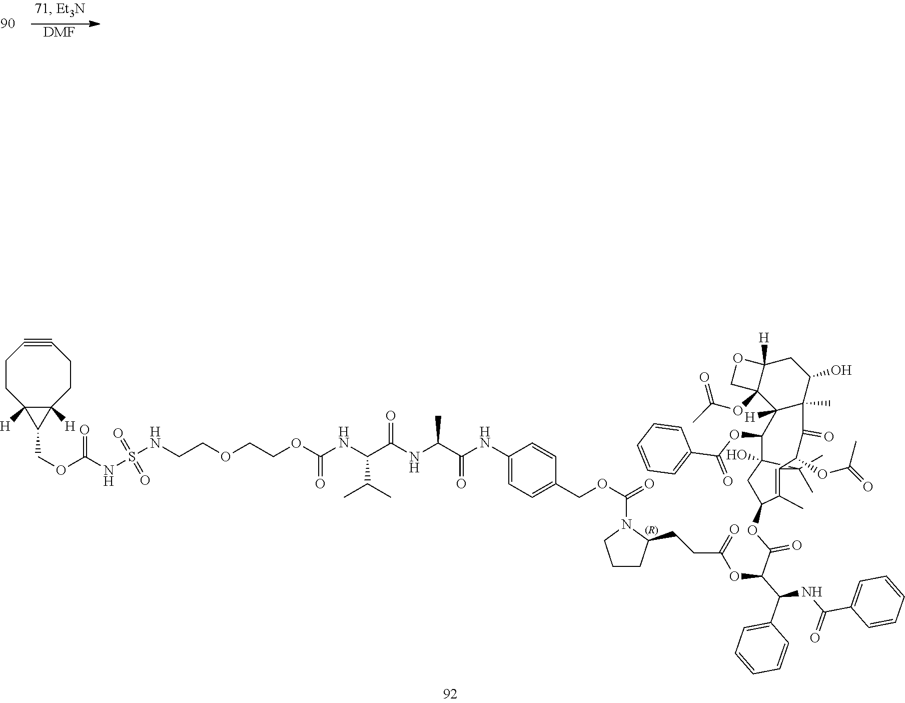

### Example 44. Preparation of Compound 96

To a solution of 2-(Fmoc-amino)ethanol (250 mg, 1.0 Eq, 882 μmol) in dry DCM (20 mL) cooled to −35° C. was added CSI (147 mg, 90 μL, 1.2 Eq, 1.1 mmol). After 20 minutes of stirring at −35° C., Et₃N (268 mg, 369 μL, 3.0 Eq, 2.7 mmol) was added, followed by 2-(2-aminoethoxy)ethan-1-ol (111 mg, 106 μL, 1.2 Eq, 1.1 mmol). The reaction mixture was allowed to warm up to room temperature and was stirred for 1 hour. PNP chloroformate (267 mg, 1.5 Eq, 1.3 mmol) was added, followed by additional Et₃N (268 mg, 369 μL, 3.0 Eq, 2.7 mmol). The reaction mixture was stirred at room temperature for 1 hour, concentrated in vacuo, dissolved in DCM (6 mL) and purified by flash column chromatography over silicagel (0%→3% MeOH in DCM) to afford compound 93 (387 mg, 560 μmol, 63%, 95% purity). LCMS (ESI+) calculated for C₂₉H₃₁N₄O₁₂S (M+H)⁺ 659.2. found 659.3.

To a solution of compound 93 (20.6 mg, 95% Wt, 1.5 Eq, 31.3 μmol) in dry DCM (400 μL) and dry DMF (200 μL) was added compound 94 (50.0 mg, 1.0 Eq, 20.8 μmol, commercially obtained from Levena Biopharma), followed by DIPEA (26.9 mg, 36.2 μL, 10 Eq, 208 μmol) and HOBt (3.2 mg, 1.0 Eq, 20.8 μmol). After 20 hours of stirring at room temperature, piperidine (17.7 mg, 20.5 μL, 10 Eq, 208 μmol) was added. The reaction mixture was stirred for 3 hours and purified by flash column chromatography over silicagel (0%→30% MeOH in DCM) to afford compound 95 (48.8 mg, 18.1 μmol, 87%, 100% purity) as a white solid. LCMS (ESI+) calculated for C₁₃₀H₂₁₂N2₄O₃₅S²⁺ (M/2+H)⁺ 1350.8 found 1351.2.

To a solution of compound 95 (12.2 mg, 1.0 Eq, 4.5 μmol) in dry DMF (100 μL) was added methylcyclopropene-PNP (1.3 mg, 1.2 Eq, 5.4 μmol), followed by DIPEA (2.9 mg, 3.9 μL, 5.0 Eq, 22.6 μmol). The reaction mixture was stirred at room temperature for 2 hours and was subsequently purified by prep-HPLC; (30%→95%, MeCN/Water+1% AcOH, runtime 12 minutes, Column Xbridge prep C18 5 μm OBD, 30×100 mm) to afford compound 96 (4.9 mg, 1.7 μmol, 39%, 100% purity). LCMS (ESI+) calculated for C₁₃₆H₂₁₈N₂₄O₃₇S²⁺ (M/2+H)⁺ 1406.8. found 1406.7.

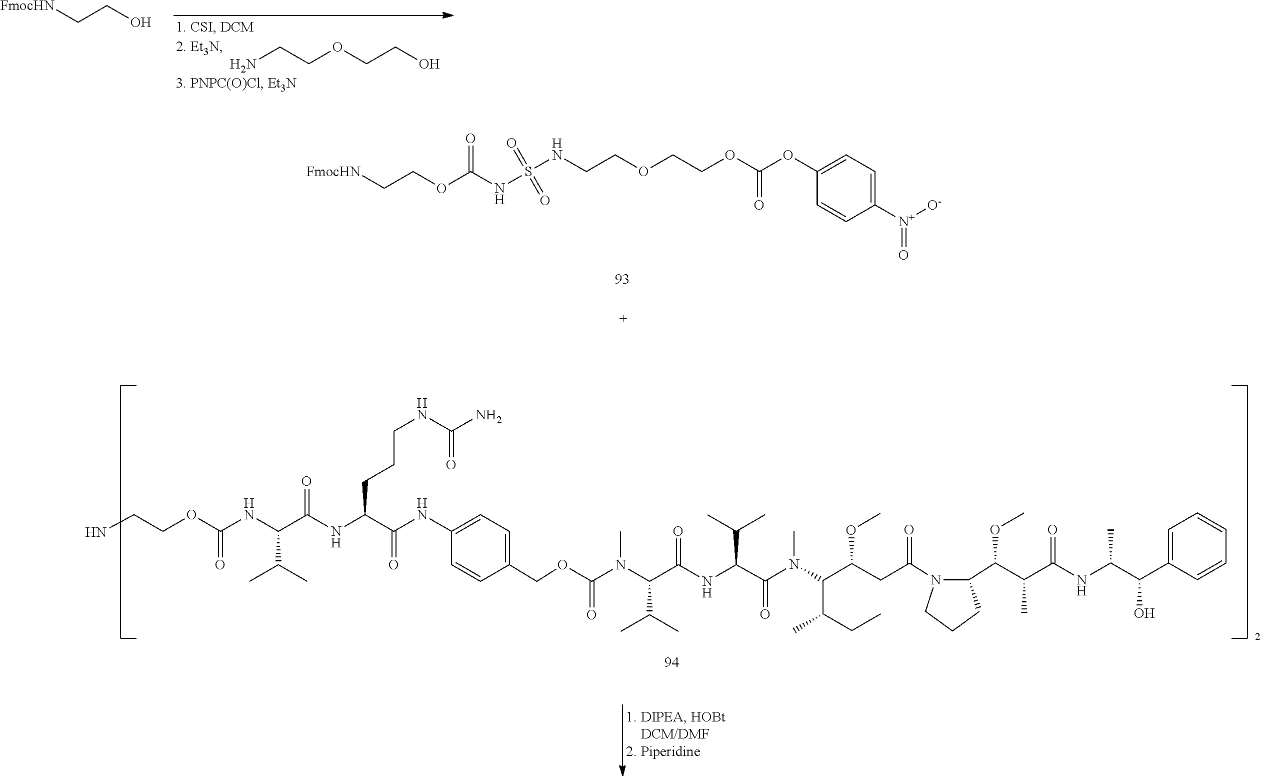

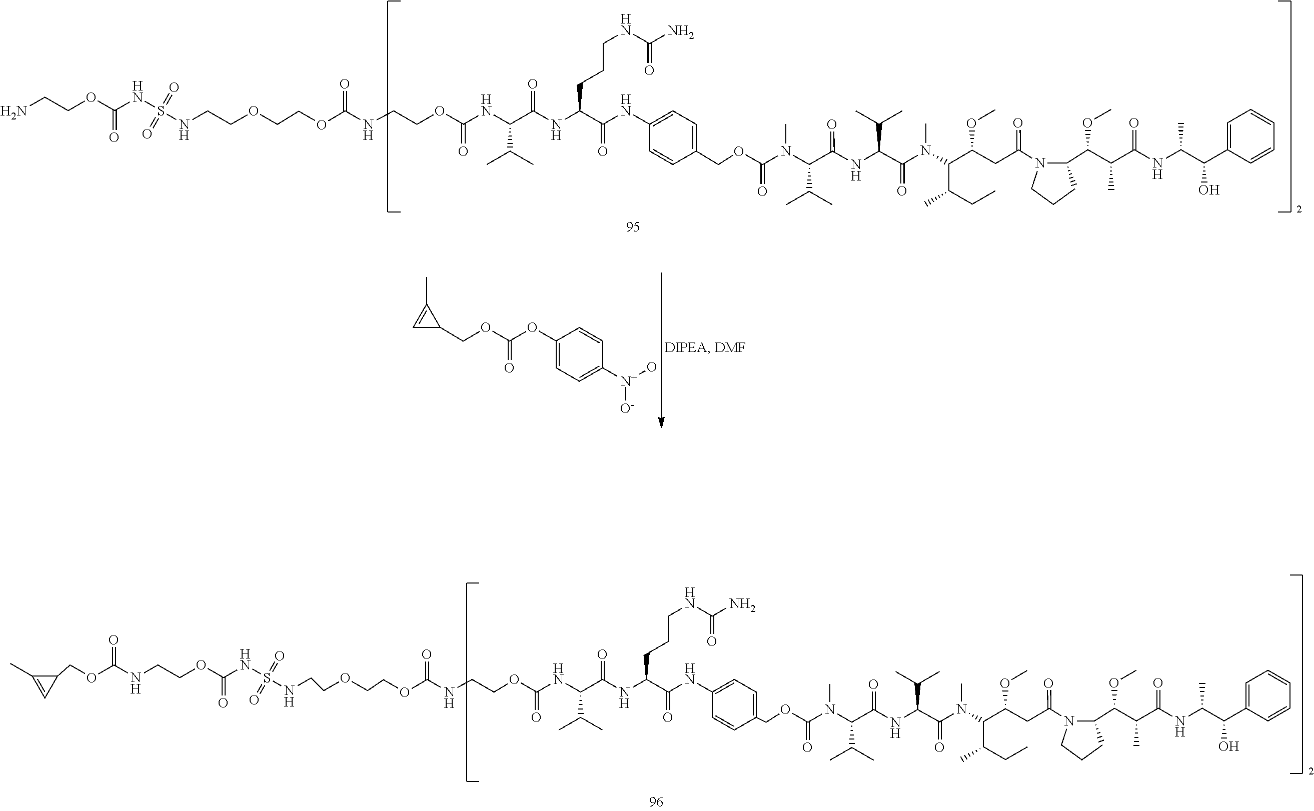

### Example 45. Preparation of Compound 97

To a solution of compound 95 (12.2 mg, 1.0 Eq, 4.5 μmol) in dry DMF (100 μL) was added TCO-NHS ester (1.5 mg, 1.2 Eq, 5.4 μmol), followed by DIPEA (2.9 mg, 3.9 μL, 5.0 Eq, 22.6 μmol). The reaction mixture was stirred at room temperature for 2 hours and was subsequently purified by prep-HPLC; (30%→95%, MeCN/Water+1% AcOH, runtime 12 minutes, Column Xbridge prep C18 5 μm OBD, 30×100 mm) to afford compound 97 (5.5 mg, 1.9 μmol, 43%, 100% purity). LCMS (ESI+) calculated for C₁₃₉H₂₂₄N₂₄O₃₇S²⁺ (M/2+H)⁺ 1427.8. found 1427.7.

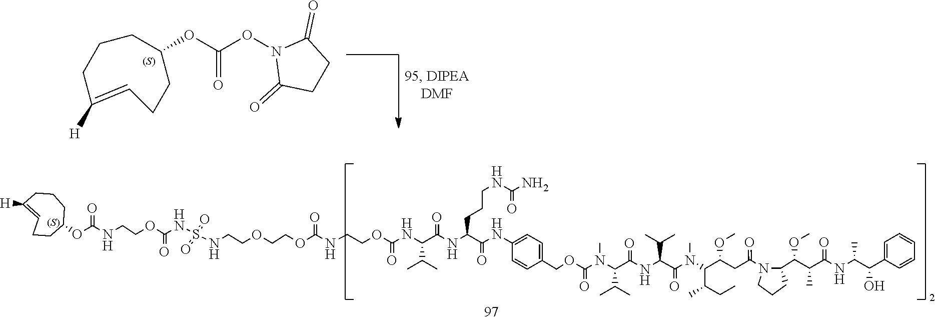

### Example 48. Preparation of Compound 96

To a solution of compound 76 (1.4 mg, 1.0 eq, 1.6 μmol) in anhydrous DMF (80 μL) was added compound 71 (0.83 mg, 1.0 eq, 22 μL, 71 mM, 1.6 μmol) and triethylamine (0.48 mg, 0.7 μL, 3.0 eq, 4.7 μmol). After standing for 46 hours at room temperature, the reaction mixture was diluted with DCM (500 μl) and purified by flash column chromatography over silicagel (0%→10% MeOH in DCM). The combined impure batches were re-purified by prep-HPLC (injection volume 110 μL DMF; 30%→95%, MeCN/Water+1% AcOH, runtime 12 minutes, Column Xbridge prep C18 5 μm OBD, 10×100 mm) to afford compound 98 (0.7 mg, 0.5 μmol, 30%). LCMS (ESI+) calculated for C₆₇H₇₇N₁₀O₁₅S⁺ (M+NH₄)⁺ 1293.5. found 1293.9.

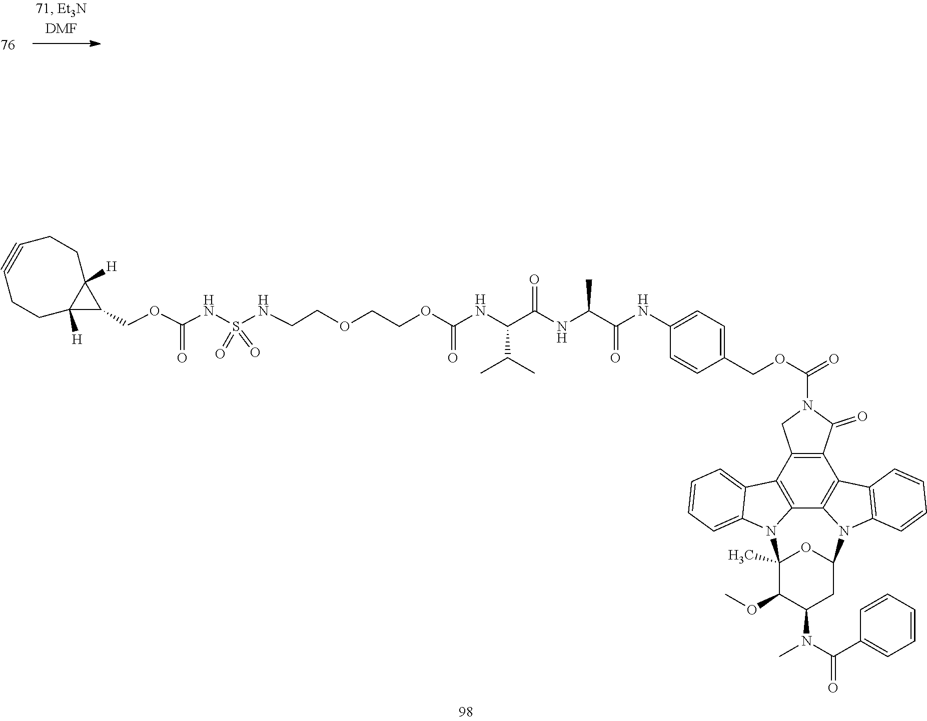 [SMILES: CO[C@@H]1[C@H](N(C)C(=O)c2ccccc2)C[C@H]2O[C@]1(C)n1c3ccccc3c3c4c(c5c6ccccc6n2c5c31)C(=O)N(C(=O)OCc1ccc(NC(=O)[C@H](C)NC(=O)[C@@H](NC(=O)OCCOCCNS(=O)(=O)NC(=O)OC[C@@H]2[C@@H]3CCC#CCC[C@@H]32)C(C)C)cc1)C4]

### Example 47. Preparation of Compound 100

To a solution of UCN-01 (10 mg, 1.0 eq, 21 μmol) in anhydrous DMF (60 μL) and DCM (200 μL) were added compound 4 (21 mg, 1.5 eq, 31 μmol), DIPEA (8.0 mg, 11 μL, 3.0 eq, 62 μmol) and HOBt (2.8 mg, 43 μL, 447 mM, 1.0 eq, 21 μmol). After 22 h at room temperature, the reaction mixture was purified by flash column chromatography over silicagel (0%→15% MeOH in DCM) to afford the Fmoc-protected intermediate as an off-white solid (16.0 mg, 15 μmol, 73%, 97% purity). LCMS (ESI+) calculated for C₅₉H₅₆N₇O₉⁺ (M-⁻OH)⁺ 1006.41. found 1006.75.

To a solution of Fmoc-protected intermediate (8.4 mg, 1.0 eq, 8.2 μmol) in DMF (200 μL) was added triethylamine (8.3 mg, 11 μL, 10 eq, 82 μmol). After 21 hours at room temperature, the reaction mixture was concentrated in vacuo to afford compound 99 in quantitative yield (6.6 mg, 8.2 μmol). LCMS (ESI+) calculated for C₄₄H₄₆N₇O₇⁺ (M-⁻OH)⁺ 784.4. found 784.6.

To a solution of compound 99 (6.6 mg, 1.0 eq, 8.2 μmol) in anhydrous DMF (200 μL) were added compound 71 (4.8 mg, 1.1 eq, 9.1 μmol) and triethylamine (2.5 mg, 3.4 μL, 3.0 eq, 25 μmol). After 23 hours at room temperature, the reaction mixture was diluted with DCM (1.0 ml) and purified by flash column chromatography over silicagel (0%→15% MeOH in DCM) to afford compound 100 (9.0 mg, 8.0 μmol, 90%). LCMS (ESI+) calculated for C₆₀H₆₈N₉O₁₄S⁺ (M−OH⁻)⁺ 1170.5. found 1170.8.

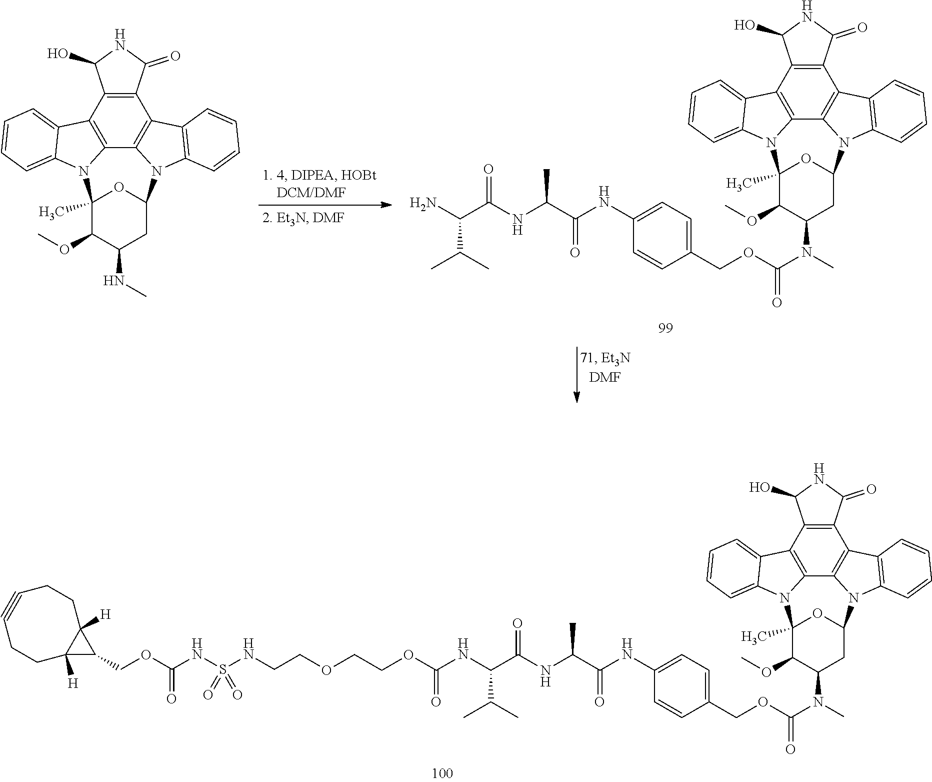

### Example 48. Preparation of Compound 102

To a solution of staurosporine (21.3 mg, 1.0 eq, 45.7 μmol) in anhydrous DMF (450 μL) was added Fmoc-Val-Ala-PABC-OPFP (36.4 mg, 1.1 eq, 50.2 μmol) and DIPEA (17.7 mg, 23.9 μL, 3.0 eq, 137 μmol). After 6.5 h at room temperature, the reaction mixture was diluted with DCM (1 mL) and purified by flash column chromatography over silicagel (0%→10% MeOH in DCM) to afford the Fmoc-protected intermediate (44.2 mg, 43.0 μmol, 93%). LCMS (ESI+) calculated for C₅₉H₅₈N₇O₉S⁺ (M+H)⁺ 1008.4. found 1008.7.

To a solution of the Fmoc-protected intermediate (44.2 mg, 1.0 eq, 42.5 μmol) in DMF (500 μL) was added triethylamine (43.0 mg, 59.3 μL, 10.0 eq, 425 μmol). After 22 hours at room temperature, the reaction mixture was concentrated in vacuo to afford compound 101 in quantitative yield. LCMS (ESI+) calculated for C₄₄H₄₈N₇O₇⁺ (M+H)⁺ 786.4. found 786.6.

To a solution of compound 101 (12.8 mg, 3.0 eq, 16.3 μmol) in anhydrous DMF (150 μL) were added compound 6 (5.0 mg, 1.0 eq, 5.0 μmol) and triethylamine (3.0 mg, 4 μL, 5.0 eq, 30 μmol). After 20.5 h at room temperature, the reaction mixture was diluted with DCM (1.0 ml) and purified by flash column chromatography over silicagel (0%→20% MeOH in DCM) to afford compound 102 (10.7 mg, 4.88 μmol, 90%). LCMS (ESI+) calculated for C₁₁₀H₁₂₆N₁₈O₂₇S₂²⁺ (M/2+H)⁺ 1098.4. found 1098.4.

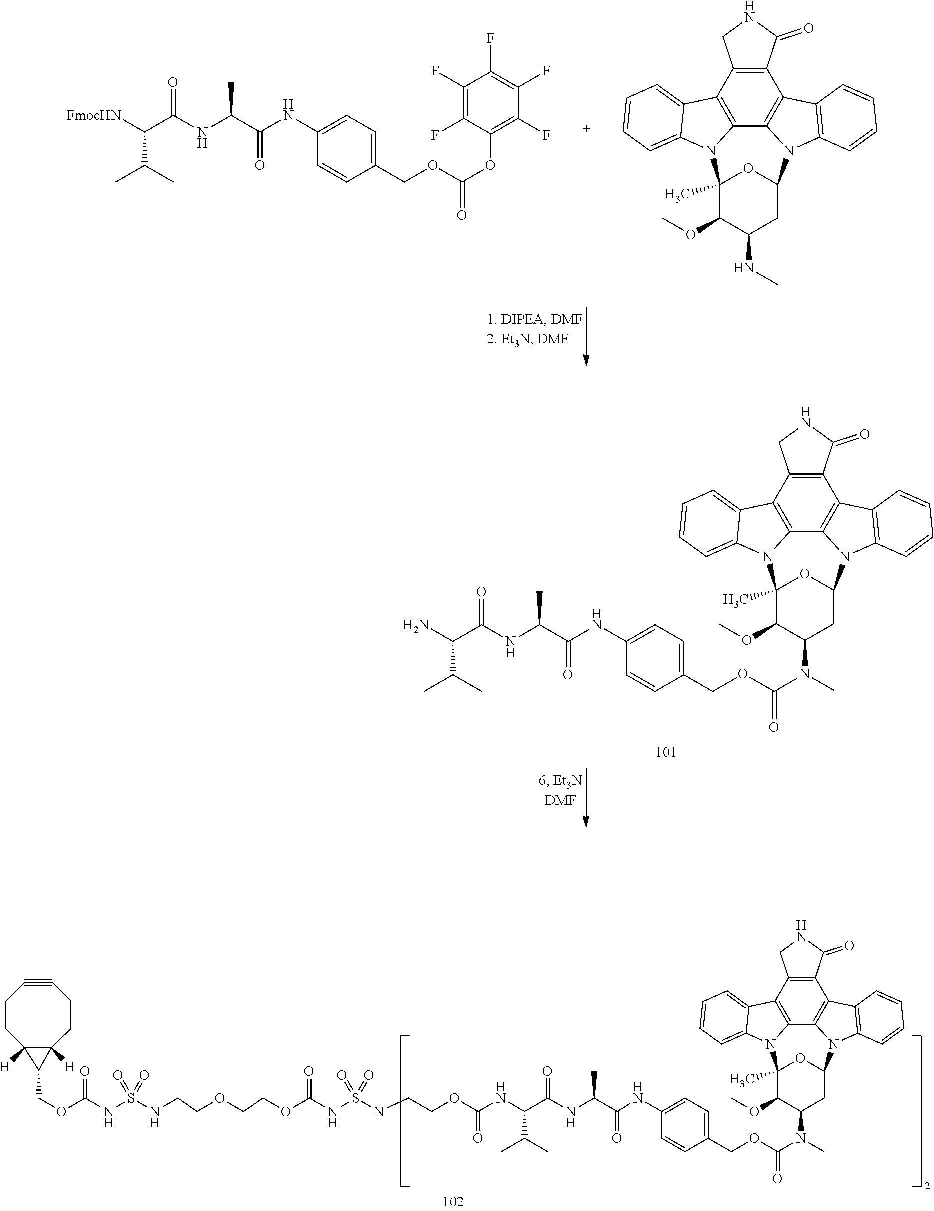

### Example 49. Preparation of Compound 106

To TMTHSI-OSu (200.0 mg, 1 Eq, 587.6 μmol) was added a solution of 2-aminoethan-1-ol (107.7 mg, 106.4 μL, 3 Eq, 1.763 mmol) in dry DCM (5 mL). The resulting suspension was stirred for 18 hours after which the reaction mixture was purified by flash column chromatography over silicagel (0%→20% Acetone in DCM) to afford compound 103 (175 mg, 0.46 mmol, 79%, 76% Purity) as an white solid. LCMS (ESI+) calculated for C₁₃H₂₃N₂O₃S⁺ (M+H)⁺ 287.1. found 286.9.

Compound 103 (100.0 mg, 80% Wt, 1 Eq, 279.3 μmol) was dissolved in dry DCM (8 mL) and cooled to −35° C. after which CSI (57.7 mg, 35.5 μL, 1.46 Eq, 407.7 μmol) was added. After stirring for 10 minutes, triethylamine (84.80 mg, 117 μL, 3 Eq, 838.0 μmol) was added followed by 2-(2-aminoethoxy)ethan-1-ol (44.05 mg, 42.04 μL, 1.5 Eq, 419.0 μmol). The icebath was removed and stirring was continued at room temperature for 3 hours. Subsequently, the reaction mixture was purified by flash column chromatography over silicagel (0%→10% MeOH in DCM) to afford compound 104 (61 mg, 0.12 mmol, 42%, 95% Purity) as a white solid. LCMS (ESI+) calculated for C₁₈H₃₃N₄O₈S₂⁺ (M+H)⁺ 497.2. found 497.5.

Compound 104 (60.0 mg, 1 Eq, 121 μmol) was dissolved in dry DCM (4.00 mL) and this was cooled to −40° C. Next CSI (17.1 mg, 10.5 μL, 1 Eq, 121 μmol) was added. After 5 minutes, triethylamine (61.1 mg, 84.2 μL, 5 Eq, 604 μmol) was added followed by a stock solution of diethanolamine (14.6 mg, 13.3 μL, 1.15 Eq, 139 μmol) in dry THF (130 μL). After 5 hours, additional triethylamine (36.7 mg, 50.5 μL, 3 Eq, 362 μmol) was added followed by PNP chloroformate (60.9 mg, 2.5 Eq, 302 μmol). The reaction mixture was stirred at room temperature for a total of 42 hours and purified by flash column chromatography over silicagel (0%→80% Acetone in DCM). The obtained, impure batch was re-purified by prep-HPLC (injection volume 750 μL DMF; 30%→95%, MeCN/Water+1% AcOH, runtime 12 minutes, Column Xbridge prep C18 5 μm OBD, 30×100 mm) to afford compound 105 (8.3 mg, 7.2 μmol, 6.0%, 90% Purity) as a white solid. LCMS (ESI+) calculated for C₃₇H₄₉N₈O₂₁S₃⁺ (M+H)⁺ 1037.2. found 1037.6.

To compound 105 (8.30 mg, 1 Eq, 8.00 μmol) was added a stock solution compound 5b (13.3 mg, 176 μL, 100 mmolar, 2.2 Eq, 17.6 μmol) in DMF followed by triethylamine (4.05 mg, 5.58 μL, 5 Eq, 40.0 μmol). The reaction mixture was left to stand for 18 hours and was subsequently purified by prep-HPLC (injection volume 550 μL DMF; 30%→95%, MeCN/Water+1% AcOH, runtime 12 minutes, Column Xbridge prep C18 5 μm OBD, 30×100 mm) to afford compound 106 (6.0 mg, 2.1 μmol, 26%, 80% Purity) as a white solid. LCMS (ESI+) calculated for C₁₀₅H₁₂₆F₂N₁₈O₃₁S₃⁺² (M/2+H)⁺ 1134.9. found 1135.2.

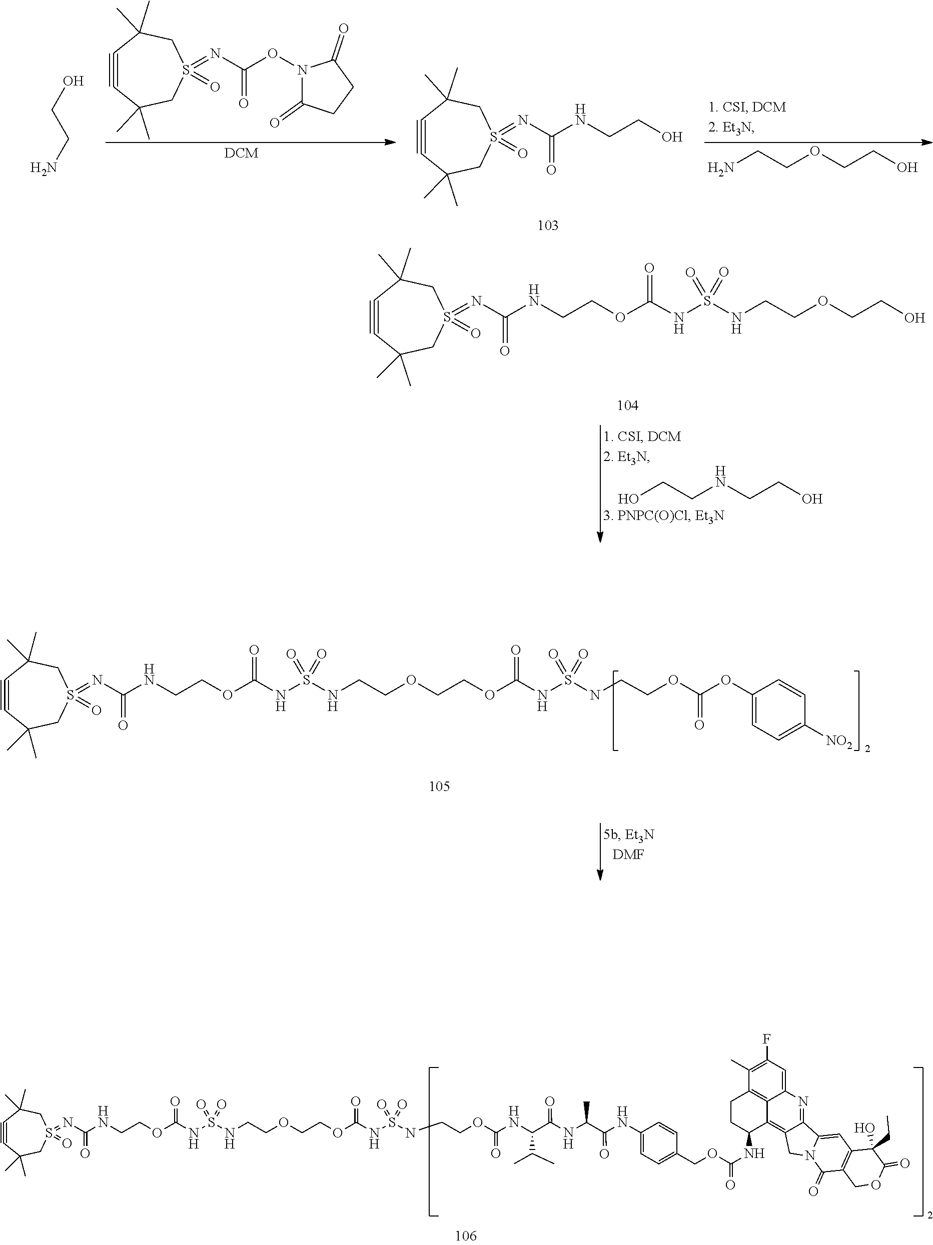

### Examples 50-71: Preparation of Click Probe-Functionalized Antibodies

### Example 50: Preparation of Trastuzumab-(6-N₃-GalNAc)₂

According to the general procedure for enzymatic remodeling, trastuzumab (obtained from the pharmacy) was converted to trastuzumab-(6-N₃-GalNAc)₂. Mass spectral analysis of a sample after IdeS treatment showed one major Fc/2 product (observed mass 24366 Da, approximately 85% of total Fc/2 and observed mass for minor-fucose product 24220 Da, approximately 10% of the total Fc/2), corresponding to the expected product.

### Example 51: Preparation of Trastuzumab-(Tetrazine)₄ [Trastuzumab-(3)₂]

To a solution of trastuzumab-(6-N₃-GalNAc)₂ (1.2 mL, 25 mg, 20.61 mg/mL in TBS pH 7.5) was added 167 μL bistetrazine compound 3 (10 mM stock solution in DMF) in propylene glycol (1.08 mL, 50% of final volume). The reaction was incubated overnight at rt. To remove the excess of bistetrazine compound 3, a HiTrap 26-10 desalting column (Cytiva) was used. The column was rinsed with 0.2M NaOH and equilibrated with TBS pH 7.5 before the injection and elution of trastuzumab-(tetrazine)₄. Subsequently the product was concentrated to 27.9 mg/mL using a Vivaspin Turbo 4 10 kDa MWCO ultrafiltration unit (Sartorius). Mass spectral analysis of the sample after IdeS treatment showed one major Fc/2 product (calculated mass 25445, observed mass 25446 Da, approximately 95% of total Fc/2), corresponding to the pink coloured trastuzumab-(3)₂.

### Example 52: Preparation of Palivizumab-(Tetrazine)₄ [Palivizumab-(3)₂]

To a solution of palivizumab-(6-N₃-GalNAc)₂ (204 μL, 5 mg, 24.51 mg/mL in TBS pH 7.5) was added 13.3 μL bistetrazine compound 3 (10 mM stock solution in DMF) in propylene glycol (136.7 μL, 30% of final volume). The reaction was incubated in the dark overnight at rt. To remove the excess of bistetrazine compound 3, a 5 mL HiTrap desalting column (Cytiva) was used. The column was rinsed with 0.2M NaOH and equilibrated with TBS pH 7.5 before the injection and elution of palivizumab-(tetrazine 3)₂. Subsequently the product was concentrated using a Vivaspin Turbo 4 10 kDa MWCO ultrafiltration unit (Sartorius). Mass spectral analysis of the sample after IdeS treatment showed one major Fc/2 product (calculated mass 25442 Da, observed mass 25445 Da, approximately 95% of total Fc/2), corresponding to the pink coloured palivizumab-(3)₂.

### Example 53: Preparation of B12-(Tetrazine)₄[B12-(3)₂]

To a solution of B12-(6-N₃-GalNAc)₂ (204 μL, 5 mg, 24.51 mg/mL in TBS pH 7.5) was added 13.3 μL bistetrazine compound 3 (10 mM stock solution in DMF) in propylene glycol (136.7 μL, 30% of final volume). The reaction was incubated in the dark overnight at rt. To remove the excess of bistetrazine compound 3, a 5 mL HiTrap desalting column (Cytiva) was used. The column was rinsed with 0.2M NaOH and equilibrated with TBS pH 7.5 before the injection and elution of B12-(tetrazine 3)₂. Subsequently the product was concentrated using a Vivaspin Turbo 4 10 kDa MWCO ultrafiltration unit (Sartorius). Mass spectral analysis of the sample after IdeS treatment showed one major Fc/2 product (calculated mass 25413 Da, observed mass 25412 Da, approximately 85% of total Fc/2, other 15% corresponded to the m/z+Lys, calculated mass 25541 Da, observed mass 25540 Da), corresponding to the pink coloured B12-(3)₂.

### Example 54: Preparation of sacituzumab-(tetrazine)₄[sacituzumab-(3)₂]

To a solution of sacituzumab-(6-N₃-GalNAc)₂ (250 μL, 4.9 mg, 19.64 mg/mL in TBS pH 7.5) was added 13.1 μL bistetrazine compound 3 (10 mM stock solution in DMF) in propylene glycol (134 μL, 30% of final volume). The reaction was incubated in the dark overnight at rt. To remove the excess of bistetrazine compound 3, a 5 mL HiTrap desalting column (Cytiva) was used. The column was rinsed with 0.2M NaOH and equilibrated with TBS pH 7.5 before the injection and elution of sacituzumab-(tetrazine 3)₂. Subsequently the product was concentrated using a Vivaspin Turbo 4 10 kDa MWCO ultrafiltration unit (Sartorius). Mass spectral analysis of the sample after IdeS treatment showed one major Fc/2 product (calculated mass 25444 Da, observed mass 25444 Da, approximately 95% of total Fc/2), corresponding to the pink coloured sacituzumab-(3)₂.

### Example 55: Preparation of Rituximab-(Tetrazine)₄[Rituximab-(3)₂]

To a solution of rituximab-(6-N₃-GalNAc)₂ (168 μL, 3.6 mg, 21.39 mg/mL in TBS pH 7.5) was added 9.6 μL bistetrazine compound 3 (10 mM stock solution in DMF) in propylene glycol (98 μL, 30% of final volume). The reaction was incubated in the dark overnight at rt. To remove the excess of bistetrazine compound 3, a 5 mL HiTrap desalting column (Cytiva) was used. The column was rinsed with 0.2M NaOH and equilibrated with TBS pH 7.5 before the injection and elution of rituximab-(tetrazine 3)₂. Subsequently the product was concentrated using a Vivaspin Turbo 4 10 kDa MWCO ultrafiltration unit (Sartorius). Mass spectral analysis of the sample after IdeS treatment showed one major Fc/2 product (calculated mass 25411 Da, observed mass 25411 Da, approximately 95% of total Fc/2), corresponding to the pink coloured rituximab-(3)₂.

### Example 56: Preparation of Trastuzumab-(Tetrazine)₄ [Trastuzumab-(23)₂]

To a solution of trastuzumab-(6-N₃-GalNAc)₂ (565 μL, 13 mg, 23.02 mg/mL in TBS pH 7.5) was added 35 μL bistetrazine compound 23 (10 mM stock solution in DMF) in propylene glycol (355 μL, 30% of final volume). The reaction was incubated in the dark overnight at rt. To remove the excess of bistetrazine compound 23, a HiTrap 26-10 desalting column (Cytiva) was used. The column was rinsed with 0.2M NaOH and equilibrated with TBS pH 7.5 before the injection and elution of trastuzumab-(tetrazine 23)₂. Subsequently the product was concentrated to 26.18 mg/mL using a Vivaspin Turbo 4 10 kDa MWCO ultrafiltration unit (Sartorius). Mass spectral analysis of the sample after IdeS treatment showed one major Fc/2 product (calculated mass 25465 Da, observed mass 25462 Da, approximately 90% of total Fc/2), corresponding to the pink coloured trastuzumab-(23)₂.

### Example 57: Preparation of Trastuzumab-(Tetrazine)₄ [Trastuzumab-(25)₂]

To a solution of trastuzumab-(6-N₃-GalNAc)₂ (652 μL, 15 mg, 23.02 mg/mL in TBS pH 7.5) was added 40 μL bistetrazine compound 25 (10 mM stock solution in DMF) in propylene glycol (410 μL, 30% of final volume). The reaction was incubated in the dark overnight at rt. To remove the excess of bistetrazine compound 25, a HiTrap 26-10 desalting column (Cytiva) was used. The column was rinsed with 0.2M NaOH and equilibrated with TBS pH 7.5 before the injection and elution of trastuzumab-(tetrazine 25)₂. Subsequently the product was concentrated to 26.41 mg/mL using a Vivaspin Turbo 4 10 kDa MWCO ultrafiltration unit (Sartorius). Mass spectral analysis of the sample after IdeS treatment showed one major Fc/2 product (calculated mass 25506 Da, observed mass 25502 Da, approximately 95% of total Fc/2), corresponding to the pink coloured trastuzumab-(25)₂.

### Example 58: Preparation of Trastuzumab-(Tetrazine)₄ [Trastuzumab-(26)₂]

To a solution of trastuzumab-(6-N₃-GalNAc)₂ (172 μL, 4 mg, 23.23 mg/mL in TBS pH 7.5) was added 16 μL bistetrazine compound 26 (10 mM stock solution in DMF) in propylene glycol (144 μL, 40% of final volume). The reaction was incubated overnight in the dark at rt. To remove the excess of bistetrazine compound 26, a HiTrap desalting column (Cytiva) was used. The column was rinsed with 0.2M NaOH and equilibrated with TBS pH 7.5 before the injection and elution of trastuzumab-(tetrazine 26)₂. Subsequently the product was concentrated to 22.7 mg/mL using a Vivaspin Turbo 4 10 kDa MWCO ultrafiltration unit (Sartorius). Mass spectral analysis of the sample after IdeS treatment showed one major Fc/2 product (calculated mass 25679 Da, observed mass 25681 Da, approximately 90% of total Fc/2), corresponding to the pink coloured trastuzumab-(26)₂.

### Example 59: Preparation of Trastuzumab-(Tetrazine)₄ [Trastuzumab-(27)₂]

To a solution of trastuzumab-(6-N₃-GalNAc)₂ (172 μL, 4 mg, 23.23 mg/mL in TBS pH 7.5) was added 16 μL bistetrazine compound 27 (10 mM stock solution in DMF) in propylene glycol (144 μL, 40% of final volume). The reaction was incubated overnight in the dark at rt. To remove the excess of bistetrazine compound 27 a HiTrap desalting column (Cytiva) was used. The column was rinsed with 0.2M NaOH and equilibrated with TBS pH 7.5 before the injection and elution of trastuzumab-(tetrazine 27)₂. Subsequently the product was concentrated to 16.16 mg/mL using a Vivaspin Turbo 4 10 kDa MWCO ultrafiltration unit (Sartorius). Mass spectral analysis of the sample after IdeS treatment showed one major Fc/2 product (calculated mass 25354 Da, observed mass 25357 Da, approximately 95% of total Fc/2), corresponding to the pink coloured trastuzumab-(27)₂.

### Example 60: Preparation of Trastuzumab-(Tetrazine)₄ [Trastuzumab-(29)₂]

To a solution of trastuzumab-(6-N₃-GalNAc)₂ (652 μL, 15 mg, 23.02 mg/mL in TBS pH 7.5) was added 40 μL bistetrazine compound 29 (10 mM stock solution in DMF) in propylene glycol (410 μL, 30% of final volume). The reaction was incubated in the dark overnight at rt. To remove the excess of bistetrazine compound 29, a HiTrap 26-10 desalting column (Cytiva) was used. The column was rinsed with 0.2M NaOH and equilibrated with TBS pH 7.5 before the injection and elution of trastuzumab-(tetrazine 29)₂. Subsequently the product was concentrated to 21.32 mg/mL using a Vivaspin Turbo 4 10 kDa MWCO ultrafiltration unit (Sartorius). Mass spectral analysis of the sample after IdeS treatment showed one major Fc/2 product (calculated mass 25368 Da, observed mass 25369 Da, approximately 95% of total Fc/2), corresponding to the pink coloured trastuzumab-(29)₂.

### Example 61: Preparation of Trastuzumab-(Tetrazine)₄ [Trastuzumab-(31)₂]

To a solution of trastuzumab-(6-N₃-GalNAc)₂ (172 μL, 4 mg, 23.23 mg/mL in TBS pH 7.5) was added 11 μL bistetrazine compound 31 (10 mM stock solution in DMF) in propylene glycol (149 μL, 40% of final volume). The reaction was incubated in the dark at rt for 4 h. To remove the excess of bistetrazine compound 31, a HiTrap desalting column (Cytiva) was used. The column was rinsed with 0.2M NaOH and equilibrated with TBS pH 7.5 before the injection and elution of trastuzumab-(tetrazine 31)₂. Subsequently the product was concentrated to 17.07 mg/mL using a Vivaspin Turbo 4 10 kDa MWCO ultrafiltration unit (Sartorius). Mass spectral analysis of the sample after IdeS treatment showed one major Fc/2 product (calculated mass 25540 Da, observed mass 25539 Da, approximately 90% of total Fc/2), corresponding to the pink coloured trastuzumab-(31)₂.

### Example 62: Preparation of Trastuzumab-(Tetrazine)₄ [Trastuzumab-(33)₂]

To a solution of trastuzumab-(6-N₃-GalNAc)₂ (172 μL, 4 mg, 23.23 mg/mL in TBS pH 7.5) was added 11 μL bistetrazine compound 33 (10 mM stock solution in DMF) in propylene glycol (149 μL, 40% of final volume). The reaction was incubated in the dark at rt for 4 h. To remove the excess of bistetrazine compound 33, a HiTrap desalting column (Cytiva) was used. The column was rinsed with 0.2M NaOH and equilibrated with TBS pH 7.5 before the injection and elution of trastuzumab-(tetrazine 33)₂. Subsequently the product was concentrated to 16.81 mg/mL using a Vivaspin Turbo 4 10 kDa MWCO ultrafiltration unit (Sartorius). Mass spectral analysis of the sample after IdeS treatment showed one major Fc/2 product (calculated mass 25584 Da, observed mass 25583 Da, approximately 90% of total Fc/2), corresponding to the pink coloured trastuzumab-(33)₂.

### Example 63: Preparation of Trastuzumab-(Tetrazine)₄ [Trastuzumab-(35)₂]

To a solution of trastuzumab-(6-N₃-GalNAc)₂ (652 μL, 15 mg, 23.02 mg/mL in TBS pH 7.5) was added 50 μL bistetrazine compound 35 (10 mM stock solution in DMF) in propylene glycol (475 μL, 35% of final volume). The reaction was incubated in the dark overnight at rt. To remove the excess of bistetrazine compound 35, a HiTrap 26-10 desalting column (Cytiva) was used. The column was rinsed with 0.2M NaOH and equilibrated with TBS pH 7.5 before the injection and elution of trastuzumab-(tetrazine 35)₂. Subsequently the product was concentrated to 25.14 mg/mL using a Vivaspin Turbo 4 10 kDa MWCO ultrafiltration unit (Sartorius). Mass spectral analysis of the sample after IdeS treatment showed one major Fc/2 product (calculated mass 25330 Da, observed mass 25326 Da, approximately 95% of total Fc/2), corresponding to the pink coloured trastuzumab-(35)₂.

### Example 64: Preparation of Trastuzumab-(Serine)₄ [Trastuzumab-(37)₂]

To a solution of trastuzumab-(6-N₃-GalNAc)₂ (337 μL, 8 mg, 23.72 mg/mL in TBS pH 7.5) was added 43 μL bis-serine compound 37 (10 mM stock solution in MQ). The reaction was incubated in the dark overnight at rt. To remove the excess of bis-serine compound 37, a 5 mL HiTrap desalting column (Cytiva) was used. The column was rinsed with 0.2M NaOH and equilibrated with PBS pH 7.5 before the injection and elution of trastuzumab-(serine 37)₂. Subsequently the product was concentrated using a Vivaspin Turbo 4 10 kDa MWCO ultrafiltration unit (Sartorius). Mass spectral analysis of the sample after IdeS treatment showed one major Fc/2 product (calculated mass 25248 Da, observed mass 25247 Da, approximately 95% of total Fc/2), corresponding to the pink coloured trastuzumab-(37)₂.

### Example 65: Oxidation and Nitrone Incorporation of Trastuzumab-(37)₂

Oxidation of the serine containing trastuzumab-(37)₂ (274 μL, 2.5 mg, 9.1 mg/mL in PBS) was performed with NaIO₄ (3 eq. 5 μL of 10 mM stock). The reaction was incubated at room temperature for 5 minutes before MS was measured. Mass spectral analysis of the sample after IdeS treatment showed one major Fc/2 product (double hydrate calculated mass 25220 Da, observed mass 25220 Da, approximately 90% of total Fc/2) and the reaction was subsequently quenched with 6.6 eq. p-methoxybenzenethiol (14.52 uL, 10 mM in DMF).

2-oxoacetic acid hydrate (1.24 g, 1 Eq, 13.5 mmol) and hydroxylamine hydrochloride (936 mg, 1 Eq, 13.5 mmol) were stirred in water (60 mL) at room temperature. 1M NaOH was added to raise the pH to 5 before addition of sodium cyanoborohydride (2.12 g, 2.5 Eq, 33.7 mmol). The reaction was left to stir for 3h. The mixture was concentrated in vacuo and the resulting crude was purified by crystallization from a minimum of water to afford N-hydroxylglycine HO2 (754 mg, 8.28 mmol, 62%) as a white solid. 1H NMR (400 MHz, D2O) δ 3.77 (s, 1H). 13C NMR (101 MHz, DMSO) δ 172.40, 55.10.

To the trastuzumab with oxidized serine linker was added 160 eq. hydroxylamine HO02 (53 μL of 100 mM stock in PBS and 160 eq. p-anisidine (53 μL of 100 mM stock in PBS) to introduce two nitrones. The reaction was incubated overnight and purified using a HiTrap desalting column (Cytiva). The column was rinsed with 0.2M NaOH and equilibrated with TBS pH 7.5 before the injection and elution of trastuzumab-(nitrone)₄ [trastuzumab-(37′)₂]. Subsequently the product was concentrated to 20.30 mg/mL using a Vivaspin Turbo 4 10 kDa MWCO ultrafiltration unit (Sartorius). Mass spectral analysis of the sample after IdeS treatment showed two Fc/2 products (bis nitrone calculated mass 25330 Da, observed mass 25328 Da, approximately 35% of total Fc/2, mono nitrone calculated mass 25288 Da, observed mass 25286 Da, approximately 50% of total Fc/2), corresponding to trastuzumab-(37′)₂.

### Example 66: Preparation of Trastuzumab-(Tetrazine)₄ [Trastuzumab-(41)₂]

To a solution of trastuzumab-(6-N₃-GalNAc)₂ (172 μL, 4 mg, 23.23 mg/mL in TBS pH 7.5) was added 16 μL bistetrazine compound 41 (10 mM stock solution in DMF) in propylene glycol (144 μL, 40% of final volume). The reaction was incubated overnight in the dark at rt. To remove the excess of bistetrazine compound 41, a HiTrap desalting column (Cytiva) was used. The column was rinsed with 0.2M NaOH and equilibrated with TBS pH 7.5 before the injection and elution of trastuzumab-(tetrazine 41)₂. Subsequently the product was concentrated to 14.43 mg/mL using a Vivaspin Turbo 4 10 kDa MWCO ultrafiltration unit (Sartorius). Mass spectral analysis of the sample after IdeS treatment showed one major Fc/2 product (calculated mass 26140 Da, observed mass 26139 Da, approximately 60% of total Fc/2), corresponding to the pink coloured trastuzumab-(41)₂.

### Example 67: Preparation of Trastuzumab-(Tetrazine)₆ [Trastuzumab-(45)₂]

To a solution of trastuzumab-(6-N₃-GalNAc)₂ (843 μL, 20 mg, 23.72 mg/mL in TBS pH 7.5) was added 107 μL tristetrazine compound 45 (10 mM stock solution in DMF) in propylene glycol (493 μL, 30% of final volume). The reaction was incubated in the dark overnight at rt. To remove the excess of tristetrazine compound 45, a HiTrap 26-10 desalting column (Cytiva) was used. The column was rinsed with 0.2M NaOH and equilibrated with TBS pH 7.5 before the injection and elution of trastuzumab-(45)₂. Subsequently the product was concentrated using a Vivaspin Turbo 4 10 kDa MWCO ultrafiltration unit (Sartorius). Mass spectral analysis of the sample after IdeS treatment showed one major Fc/2 product (calculated mass 25698 Da, observed mass 25698 Da, approximately 95% of total Fc/2), corresponding to the pink coloured trastuzumab-(45)₂.

### Example 68: Preparation of Trastuzumab-(Tetrazine)₄ [Trastuzumab-(49)₂]

To a solution of trastuzumab-(6-N₃-GalNAc)₂ (211 μL, 5 mg, 23.72 mg/mL in TBS pH 7.5) was added 40 μL bistetrazine compound 49 (5 mM stock solution in MQ). The reaction was incubated in the dark overnight at rt. To remove the excess of bistetrazine compound 49, a 5 mL HiTrap desalting column (Cytiva) was used. The column was rinsed with 0.2M NaOH and equilibrated with TBS pH 7.5 before the injection and elution of trastuzumab-(tetrazine 49)₂. Subsequently the product was concentrated using a Vivaspin Turbo 4 10 kDa MWCO ultrafiltration unit (Sartorius). Mass spectral analysis of the sample after IdeS treatment showed one major Fc/2 product (calculated mass 25637 Da, observed mass 25637 Da, approximately 95% of total Fc/2), corresponding to the pink coloured trastuzumab-(49)₂.

### Example 69: Preparation of Trastuzumab-(Tetrazine)₄ [Trastuzumab-(51)₂]

To a solution of trastuzumab-(6-N₃-GalNAc)₂ (211 μL, 5 mg, 23.72 mg/mL in TBS pH 7.5) was added 20 μL bistetrazine compound 51 (10 mM stock solution in DMF) in propylene glycol (130 μL, 30% of final volume) and 50 μL sodium deoxycholate (110 mM). The reaction was incubated in the dark at rt overnight. To remove the excess of bistetrazine compound 51, a HiTrap desalting column (Cytiva) was used. The column was rinsed with 0.2M NaOH and equilibrated with TBS pH 7.5 before the injection and elution of trastuzumab-(tetrazine 51)₂. Subsequently the product was concentrated to 22.65 mg/mL using a Vivaspin Turbo 4 10 kDa MWCO ultrafiltration unit (Sartorius). Mass spectral analysis of the sample after IdeS treatment showed one major Fc/2 product (calculated mass 26341 Da, observed mass 26340 Da, approximately 95% of total Fc/2), corresponding to the pink coloured trastuzumab-(51)₂.

### Example 70: Preparation of Trastuzumab-(Tetrazine)₆ [Trastuzumab-(54)₂]

To a solution of trastuzumab-(6-N₃-GalNAc)₂ (632 μL, 15 mg, 23.72 mg/mL in TBS pH 7.5) was added 400 μL tristetrazine compound 54 (2.5 mM stock solution in DMF) in propylene glycol (50 μL). The reaction was incubated in the dark overnight at rt. To remove the excess of tristetrazine compound 54, a 5 mL HiTrap desalting column (Cytiva) was used. The column was rinsed with 0.2M NaOH and equilibrated with TBS pH 7.5 before the injection and elution of trastuzumab-(tetrazine 54)₂. Subsequently the product was concentrated using a Vivaspin Turbo 4 10 kDa MWCO ultrafiltration unit (Sartorius). Mass spectral analysis of the sample after IdeS treatment showed one major Fc/2 product (calculated mass 26123 Da, observed mass 26122 Da, approximately 95% of total Fc/2), corresponding to the pink coloured trastuzumab-(54)₂.

### Example 71: Preparation of Trastuzumab-(Tetrazine)₆ [Trastuzumab-(55)₂]

To a solution of trastuzumab-(6-N₃-GalNAc)₂ (169 μL, 4 mg, 23.72 mg/mL in TBS pH 7.5) was added 64 μL tristetrazine compound 55 (10 mM stock solution in DMF) in propylene glycol (56 μL, 30% of final volume) and 40 μL sodium deoxycholate (110 mM). The reaction was incubated in the dark at rt overnight. To remove the excess of tristetrazine compound 55, a HiTrap desalting column (Cytiva) was used. The column was rinsed with 0.2M NaOH and equilibrated with TBS pH 7.5 before the injection and elution of trastuzumab-(tetrazine 55)₂. Subsequently the product was concentrated to 25.29 mg/mL using a Vivaspin Turbo 4 10 kDa MWCO ultrafiltration unit (Sartorius). Mass spectral analysis of the sample after IdeS treatment showed one major Fc/2 product (calculated mass 27177 Da, observed mass 27176 Da, approximately 95% of total Fc/2), corresponding to the pink coloured trastuzumab-(55)₂.

### Examples 72-125: Conjugation of Linker-Payloads to (Modified) Monoclonal Antibodies

### Example 72: Conjugation of Trastuzumab(6-N₃-GalNAc)₂ with BCN—HS-PEG₂-HS—(Va-PABC-Ex)₂ 7 to Obtain DAR4 Conjugate Trastuzumab-(7)₂

To a solution of trastuzumab(6-N₃-GalNAc)₂ (2846 μL, 60.0 mg, 21.08 mg/ml in TBS pH 7.5) was added TBS (754 μL), sodium deoxycholate (600 μL, 110 mM) and BCN—HS-PEG₂-HS-(va-PABC-Ex)₂ 7 (160 μL, 10 mM solution in DMF) in propylene glycol (1640 μL). The reaction was incubated overnight at rt. Next the conjugate was purified on a HiLoad 16/600 Superdex200 PG column (Cytiva) on an AKTA Pure (Cytiva). To remove the excess of free payload, 13 mg of active charcoal (Carbon RHC, Filtrox AG) was added and rotated for overnight. The charcoal was removed by centrifugation and subsequently filtered over a PES syringe filter (pore 0.20 μm, Corning). Subsequently the solution was dialysed three times to 20 mM histidine, 6% sucrose buffer pH 6.0, and concentrated using a Vivaspin Turbo 4 10 kDa MWCO ultrafiltration unit (Sartorius). 0.04% Tween-20 was added before filter sterilization. Mass spectral analysis of the sample after IdeS treatment showed one major Fc/2 product (observed mass 26493 Da, approximately 90% of total Fc/2 and observed mass for minor-fucose product 26347 Da, approximately 10% of the total Fc/2), corresponding to the conjugate DAR4 trastuzumab-(7)₂. RP-UPLC analysis of the sample under reducing conditions showed an average DAR of 3.77.

### Example 73: Conjugation of Trastuzumab(Tetrazine)₄ [Trastuzumab-(3)₂] with BCN—HS-PEG₂-HS—(Va-PABC-Ex)₂ 7 to Obtain DAR8 Conjugate Trastuzumab-(3)₂-(7)₄

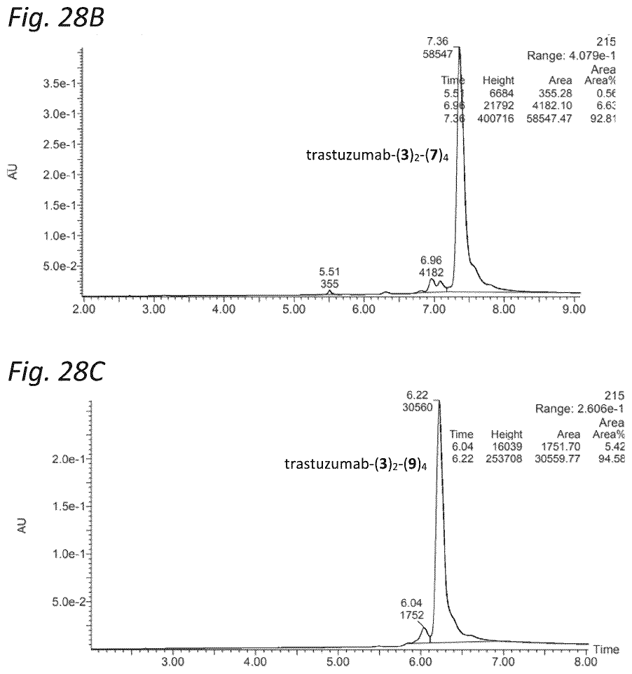

To a solution of trastuzumab-(tetrazine)₄ (269 μL, 7.5 mg, 27.88 mg/ml in TBS pH 7.5) was added sodium deoxycholate (75 μL, 110 mM) and BCN—HS-PEG₂-HS-(va-PABC-Ex)₂ 7 (30 μL, 10 mM solution in DMF) and propylene glycol (270 μL). The reaction was incubated at room temperature for five minutes, an immediate colour change from pink to yellow/colourless was observed. The precipitate of the reaction was removed by centrifugation, the pellet was rinsed with TBS (200 μL), centrifuged and combined. The mixture was diluted in 1 mL 2 M (NH₄)₂SO₄ in 50 mM sodium phosphate buffer pH 7.0 and subsequently filtered over a PES syringe filter (pore 0.20 μm, Corning). The product was then purified over a HIC, 4.6 mL Hi Screen butyl HP, column using an Akta Purifier 10. A gradient from 1 M (NH₄)₂SO₄ in 50 mM sodium phosphate buffer pH 7.0 to 10% MeCN in 50 mM phosphate pH 7.0 was used with a flow rate of 0.7 mL/min. First, the gradient was increased from 0-75% B in 70 minutes and subsequently from 75-100% B in another 60 minutes. The product was collected at 85% B. Subsequently the solution was buffer exchanged using a HiTrap 26-10 desalting column (Cytiva), rinsed with 0.2M NaOH and equilibrated with PBS. Lastly the product was concentrated to 3.28 mg/mL using a Vivaspin Turbo 4 10 kDa MWCO ultrafiltration unit (Sartorius). Mass spectral analysis of the sample after IdeS treatment showed one major Fc/2 product (calculated mass 29654, observed mass 29655 Da, approximately 95% of total Fc/2), corresponding to the DAR8 conjugate trastuzumab-(3)₂-(7)₄. RP-UPLC analysis of the sample showed an average DAR of 7.43, see FIG. 28B.

### Example 74: Conjugation of Trastuzumab(6-N₃-GalNAc)₂ with BCN—HS-PEG₂-(Eva-PABC-Ex)₂ 9 to Obtain DAR4 Conjugate Trastuzumab-(9)₂

To a solution of trastuzumab(6-N₃-GalNAc)₂ (2372 μL, 50.0 mg, 21.08 mg/ml in TBS pH 7.5) was added TBS (628 μL), sodium deoxycholate (500 μL, 110 mM) and BCN—HS-PEG₂-(eva-PABC-Ex)₂ 9 (133 μL, 10 mM solution in DMF) in propylene glycol (1367 μL). The reaction was incubated overnight at rt. Next the conjugate was purified on a HiLoad 16/600 Superdex200 PG column (Cytiva) on an AKTA Pure (Cytiva). To remove the excess of free payload, 11 mg of active charcoal (Carbon RHC, Filtrox AG) was added and rotated for overnight. The charcoal was removed by centrifugation and subsequently filtered over a PES syringe filter (pore 0.20 μm, Corning). Subsequently the solution was dialysed three times to 20 mM histidine, 6% sucrose buffer pH 6.0, and concentrated using a Vivaspin Turbo 4 10 kDa MWCO ultrafiltration unit (Sartorius). 0.04% Tween-20 was added before filter sterilization. Mass spectral analysis of the sample after IdeS treatment showed one major Fc/2 product (observed mass 26672 Da, approximately 90% of total Fc/2), corresponding to the DAR4 conjugate trastuzumab-(9)₂. RP-UPLC analysis of the sample under reducing conditions showed an average DAR of 3.94.

### Example 75: Conjugation of Trastuzumab(Tetrazine)₄ [Trastuzumab-(3)₂] with BCN—HS-PEG₂-(Eva-PABC-Ex)₂ 9 to Obtain DAR8 Conjugate Trastuzumab-(3)₂-(9)₄

To a solution of trastuzumab-(tetrazine)₄ (269 μL, 7.5 mg, 27.88 mg/ml in TBS pH 7.5) was added sodium deoxycholate (75 μL, 110 mM) and BCN—HS-PEG₂-(eva-PABC-Ex)₂ 9 (15 μL, 20 mM solution in DMF) and propylene glycol (285 μL). The reaction was incubated at room temperature for five minutes, an immediate colour change from pink to yellow/colourless was observed. The precipitate of the reaction was removed by centrifugation, the pellet was rinsed with TBS (200 μL), centrifuged and combined. The mixture was diluted in 1 mL 2 M (NH₄)₂SO₄ in 50 mM sodium phosphate buffer pH 7.0 and subsequently filtered over a PES syringe filter (pore 0.20 μm, Corning). The product was then purified over a HIC, 4.6 mL Hi Screen butyl HP, column using an Akta Purifier 10. A gradient from 1 M (NH₄)₂SO₄ in 50 mM sodium phosphate buffer pH 7.0 to 10% MeCN in 50 mM phosphate pH 7.0 was used with a flow rate of 0.7 mL/min. First the gradient was increased from 0-75% B in 70 minutes. The product was collected at 65% B. Subsequently the solution was buffer exchanged using a HiTrap 26-10 desalting column (Cytiva), rinsed with 0.2M NaOH and equilibrated with PBS. Lastly the product was concentrated to 4.77 mg/mL using a Vivaspin Turbo 4 10 kDa MWCO ultrafiltration unit (Sartorius). Mass spectral analysis of the sample after IdeS treatment showed one major Fc/2 product (calculated mass 30012, observed mass 30014 Da, approximately 95% of total Fc/2), corresponding to the DAR8 conjugate trastuzumab-(3)₂-(9)₄. RP-UPLC analysis of the sample showed an average DAR of 7.57, see FIG. 28C.

### Example 76: Conjugation of Trastuzumab(6-N₃-GalNAc)₂ with BCN—HS-PEG₂-HS—(Va-PABC-Mitomycin)₂ 59 to Obtain DAR4 Conjugate Trastuzumab-(59)₂

To a solution of trastuzumab(6-N₃-GalNAc)₂ (211 μL, 5.0 mg, 23.72 mg/ml in TBS pH 7.5) was added sodium deoxycholate (50 μL, 110 mM) and BCN—HS-PEG₂-HS-(va-PABC-mitomycin)₂ 59 (10 μL, 10 mM solution in DMF) and propylene glycol (140 μL). The reaction was incubated overnight at rt. Next the conjugate was purified on a Superdex200 10/300 increase column (Cytiva) on an AKTA Pure (Cytiva). Mass spectral analysis of the sample after IdeS treatment showed one major Fc/2 product (calcd mass 26295 Da, observed mass 26294 Da, approximately 95% of total Fc/2), corresponding to the conjugate trastuzumab-(59)₂. RP-UPLC analysis of the sample under reducing conditions showed an average DAR of 3.90.

### Example 77: Conjugation of Trastuzumab(6-N₃-GalNAc)₂ with BCN—HS-PEG₂-Va-PABC-Mitomycin 72 to Obtain DAR2 Conjugate Trastuzumab-(72)₂

To a solution of trastuzumab(6-N₃-GalNAc)₂ (211 μL, 5.0 mg, 23.72 mg/ml in TBS pH 7.5) was added sodium deoxycholate (50 μL, 110 mM) and BCN—HS-PEG₂-va-PABC-mitomycin 72 (10 μL, 10 mM solution in DMF) and propylene glycol (140 μL). The reaction was incubated overnight at rt. Next the conjugate was purified on a Superdex200 10/300 increase column (Cytiva) on an AKTA Pure (Cytiva). Mass spectral analysis of the sample after IdeS treatment showed one major Fc/2 product (calcd mass 25409 Da, observed mass 25405 Da, approximately 95% of total Fc/2), corresponding to the conjugate trastuzumab-(72)₂. RP-UPLC analysis of the sample under reducing conditions showed an average DAR of 1.95.

### Example 78: Conjugation of Trastuzumab(6-N₃-GalNAc)₂ with TMTH-SI—C(O)—NH(CH₂)₂—HS-PEG₂-HS—(Va-PABC-Ex)₂ 106 to Obtain DAR4 Conjugate Trastuzumab-(106)₂

To a solution of trastuzumab(6-N₃-GalNAc)₂ (434 μL, 10 mg, 23.02 mg/ml in TBS pH 7.5) was added TMTH-SI—C(O)—NH(CH₂)₂—HS-PEG₂-HS-(va-PABC-Ex)₂ 106 (20 μL, 10 mM solution in DMF) and propylene glycol (280 μL). The reaction was incubated overnight at rt. Next the conjugate was purified on a Superdex200 10/300 increase column (Cytiva) on an AKTA Pure (Cytiva). To remove the excess of free payload, 2 mg of active charcoal (Carbon RHC, Filtrox AG) was added and rotated for 4 h. The charcoal was removed by centrifugation and subsequently filtered over a PES syringe filter (pore 0.20 μm, Corning). Mass spectral analysis of the sample after IdeS treatment showed one major Fc/2 product (calcd mass 26633 Da, observed mass 26633 Da, approximately 95% of total Fc/2), corresponding to the conjugate trastuzumab-(106)₂. RP-UPLC analysis of the sample under reducing conditions showed an average DAR of 3.65.

### Example 79: Conjugation of Trastuzumab(Tetrazine)₄ [Trastuzumab-(3)₂] with BCN—HS-PEG₂-HS-Branched-(Va-PABC-O-Paclitaxel)2 57 to Obtain DAR8 Conjugate Trastuzumab-(3)₂-(57)₄

To a solution of trastuzumab-(3)₂ (63 μL, 2 mg, 31.7 mg/ml in TBS pH 7.5) was added with BCN—HS-PEG₂-HS-branched-(va-PABC-O-Paclitaxel)2 57 (13 μL, 10 mM solution in DMF) and propylene glycol (87 μL at a final concentration of 50%). The reaction was incubated at room temperature for 1.5 hours, a colour change from pink to yellow/colourless was observed. Next the conjugate was purified on a Superdex200 10/300 increase column (Cytiva) on an AKTA Pure (Cytiva). Intact mass spectral analysis of the sample product (calcd mass 160266 Da, observed mass 160263 Da), corresponding to the DAR8 conjugate trastuzumab-(3)₂-(57)₄. RP-UPLC analysis of the sample showed an average DAR of 7.51.

### Example 80: Conjugation of Trastuzumab(Tetrazine)₄ [Trastuzumab-(3)₂] with BCN—HS-PEG₂-HS-Branched-(Va-PABC-Mitomycin)₂ 59 to Obtain DAR8 Conjugate Trastuzumab-(3)₂-(59)₄

To a solution of trastuzumab-(3)₂ (1 mL, 20 mg, 20 mg/ml in TBS pH 7.5) was added sodium deoxycholate (200 μL, 110 mM) and BCN—HS-PEG₂-HS-branched-(va-PABC-Mitomycin)₂ 59 (80 μL, 10 mM solution in DMF) and propylene glycol (520 μL at a final concentration of 30%). The reaction was incubated at room temperature for 1 hour, a colour change from pink to yellow/colourless was observed. Next the conjugate was purified on a Superdex200 16/600 column (Cytiva) on an AKTA Pure (Cytiva). Mass spectral analysis of the sample after IdeS treatment showed one major Fc/2 product (calcd mass 29246 Da, observed mass 29246 Da, approximately 95% of total Fc/2), corresponding to the DAR8 conjugate trastuzumab-(3)₂-(59)₄. RP-UPLC analysis of the sample showed an average DAR of 7.67.

### Example 81: Conjugation of Trastuzumab(Tetrazine)₄ [Trastuzumab-(3)₂] with BCN—HS-PEG₂-HS-Branched-(Va-PABC-Gemcitabine)₂ 61 to Obtain DAR8 Conjugate Trastuzumab-(3)₂-(61)₄

To a solution of trastuzumab-(3)₂ (158 μL, 5 mg, 31.7 mg/ml in TBS pH 7.5) was added sodium cholate (50 μL, 110 mM) and BCN—HS-PEG₂-HS-branched-(va-PABC-Gemcitabine)₂ 61 (27 μL, 10 mM solution in DMF) and propylene glycol (123 μL at a final concentration of 30%). The reaction was incubated at room temperature for 1 hour, a colour change from pink to yellow/colourless was observed. Next the conjugate was purified on a Superdex200 10/300 increase column (Cytiva) on an AKTA Pure (Cytiva). Mass spectral analysis of the sample after IdeS treatment showed one major Fc/2 product (calcd mass 28962 Da, observed mass 28960 Da, approximately 95% of total Fc/2), corresponding to the DAR8 conjugate trastuzumab-(3)₂-(61)₄. RP-UPLC analysis of the sample showed an average DAR of 7.73.

### Example 82: Conjugation of Trastuzumab(Tetrazine)₄ [Trastuzumab-(3)₂] with BCN—HS-PEG₂-HS-Branched-(Va-PABC-Gefitinib)₂ 63 to Obtain DAR8 Conjugate Trastuzumab-(3)₂-(63)₄

To a solution of trastuzumab-(3)₂ (900 μL, 18 mg, 20 mg/ml in TBS pH 7.5) was added sodium deoxycholate (180 μL, 110 mM) and BCN—HS-PEG₂-HS-branched-(va-PABC-Gefitinib)₂ 63 (96 μL, 10 mM solution in DMF) and propylene glycol (444 μL at a final concentration of 30%). The reaction was incubated at room temperature for 1 hour, a colour change from pink to yellow/colourless was observed. Next the conjugate was purified on a Superdex200 16/600 column (Cytiva) on an AKTA Pure (Cytiva). Mass spectral analysis of the sample after DTT reduction showed one major HC product (calcd mass 54882 Da, observed mass 54883 Da, approximately 90% of total HC), corresponding to the DAR8 conjugate trastuzumab-(3)₂-(63)₄. RP-UPLC analysis of the sample showed an average DAR of 7.60.

### Example 83: Conjugation of Trastuzumab(Tetrazine)₄ [Trastuzumab-(3)₂] with BCN—HS-PEG₂-HS-Branched-(Va-PABC-Gennari-Paclitaxel)₂ 67 to Obtain DAR8 Conjugate Trastuzumab-(3)₂-(67)₄

To a solution of trastuzumab-(3)₂ (26 μL, 0.5 mg, 19.03 mg/ml in TBS pH 7.5) was added sodium cholate (5 μL, 110 mM) and BCN—HS-PEG₂-HS-branched-(va-PABC-Gennari-Paclitaxel)₂ 67 (2.7 μL, 10 mM solution in DMF) and propylene glycol (12.3 μL at a final concentration of 30%). The reaction was incubated at room temperature for 10 minutes, a colour change from pink to yellow/colourless was observed. Mass spectral analysis of the sample after IdeS treatment showed one major Fc/2 product (calcd mass 31886 Da, observed mass 31886 Da, approximately 95% of total Fc/2), corresponding to the DAR8 conjugate trastuzumab-(3)₂-(67)₄.

### Example 84: Conjugation of Trastuzumab(Tetrazine)₄ [Trastuzumab-(3)₂] with BCN—HS-PEG₂-HS-Branched-(Va-PABC-Gennari-Nemorubicin)₂ 70 to Obtain DAR8 Conjugate Trastuzumab-(3)₂-(70)₄

To a solution of trastuzumab-(3)₂ (712 μL, 17.3 mg, 24.23 mg/ml in TBS pH 7.5) was added sodium cholate (150 μL, 110 mM) and BCN—HS-PEG₂-HS-branched-(va-PABC-Gennari-Nemorubicin)₂ 70 (92 μL, 10 mM solution in DMF) and DMF (23 μL at a final concentration of 10%). The reaction was incubated at room temperature for 30 minutes, a colour change from pink to yellow/colourless was observed. Next the conjugate was purified on a Superdex200 16/600 column (Cytiva) on an AKTA Pure (Cytiva). Mass spectral analysis of the sample after IdeS treatment showed one major Fc/2 product (calcd mass 31046 Da, observed mass 31045 Da, approximately 90% of total Fc/2), corresponding to the DAR 8 conjugate trastuzumab-(3)₂-(70)₄. RP-UPLC analysis of the sample showed an average DAR of 7.32.

### Example 85: Conjugation of Trastuzumab(Tetrazine)₄ [Trastuzumab-(3)₂] with BCN—HS-PEG₂-HS-Branched-(Va-PABC-Beta-Lapa-Ketol)₂ 75 to Obtain DAR8 Conjugate Trastuzumab-(3)₂-(75)₄

To a solution of trastuzumab-(3)₂ (825 μL, 20 mg, 24.23 mg/ml in TBS pH 7.5) was added sodium cholate (133 μL, 110 mM) and BCN—HS-PEG₂-HS-branched-(va-PABC-beta-lapa-ketol)₂ 75 (80 μL, 10 mM solution in DMF) and DMF (53 μL at a final concentration of 10%). The reaction was incubated at room temperature for 30 minutes, a colour change from pink to yellow/colourless was observed. Next the conjugate was purified on a Superdex200 16/600 column (Cytiva) on an AKTA Pure (Cytiva). Mass spectral analysis of the sample after IdeS treatment showed one major Fc/2 product (calcd mass 28712 Da, observed mass 28711 Da, approximately 95% of total Fc/2), corresponding to the DAR8 conjugate trastuzumab-(3)₂-(75)₄. RP-UPLC analysis of the sample showed an average DAR of 7.69.

### Example 86: Conjugation of Trastuzumab(Tetrazine)₄ [Trastuzumab-(3)₂] with BCN—HS-PEG₂-HS-Branched-(Va-PABC-Gennari-Midostaurin)₂ 77 to Obtain DAR8 Conjugate Trastuzumab-(3)₂-(77)₄

To a solution of trastuzumab-(3)₂ (946 μL, 18 mg, 19.03 mg/ml in TBS pH 7.5) was added sodium deoxycholate (180 μL, 110 mM) and BCN—HS-PEG₂-HS-branched-(va-PABC-Gennari-Midostaurin)₂ 77 (96 μL, 10 mM solution in DMF) and propylene glycol (444 μL at a final concentration of 30%). The reaction was incubated at room temperature for 30 minutes, a colour change from pink to yellow/colourless was observed. Next the conjugate was purified on a Superdex200 16/600 column (Cytiva) on an AKTA Pure (Cytiva). Mass spectral analysis of the sample after IdeS treatment showed one major Fc/2 product (calcd mass 30194 Da, observed mass 30192 Da, approximately 95% of total Fc/2), corresponding to the DAR8 conjugate trastuzumab-(3)₂-(77)₄. RP-UPLC analysis of the sample showed an average DAR of 6.46.

### Example 87: Conjugation of Trastuzumab(Tetrazine)₄ [Trastuzumab-(3)₂] with BCN—HS-PEG₂-HS-Branched-(Val-Ala-Lenalidomide)₂ 79 to Obtain DAR8 Conjugate Trastuzumab-(3)₂-(79)₄

To a solution of trastuzumab-(3)₂ (751 μL, 18 mg, 24.23 mg/ml in TBS pH 7.5) was added sodium deoxycholate (121 μL, 110 mM) and BCN—HS-PEG₂-HS-branched-(va-Lenalidomide)₂ 79 (73 μL, 10 mM solution in DMF) and DMF (49 μL at a final concentration of 10%). The reaction was incubated at room temperature for 30 minutes, a colour change from pink to yellow/colourless was observed. Next the conjugate was purified on a Superdex200 16/600 column (Cytiva) on an AKTA Pure (Cytiva). Mass spectral analysis of the sample after IdeS treatment showed one major Fc/2 product (calcd mass 28351 Da, observed mass 28350 Da, approximately 95% of total Fc/2), corresponding to the DAR8 conjugate trastuzumab-(3)₂-(79)₄. RP-UPLC analysis of the sample showed an average DAR of 7.26.

### Example 88: Conjugation of Trastuzumab(Tetrazine)₄ [Trastuzumab-(3)₂] with BCN—HS-PEG₂-HS-Branched-(Va-PABC-Gennari-Gemcitabine)₂ 82 to Obtain DAR8 Conjugate Trastuzumab-(3)₂-(82)₄

To a solution of trastuzumab-(3)₂ (263 μL, 5 mg, 19.03 mg/ml in TBS pH 7.5) was added sodium deoxycholate (50 μL, 110 mM) and BCN—HS-PEG₂-HS-branched-(va-PABC-Gennari-Gemcitabine)₂ 82 (20 μL, 10 mM solution in DMF) and propylene glycol (130 μL at a final concentration of 30%). The reaction was incubated at room temperature for 1 hour, a colour change from pink to yellow/colourless was observed. Next the conjugate was purified on a Superdex200 10/300 increase column (Cytiva) on an AKTA Pure (Cytiva). Mass spectral analysis of the sample after IdeS treatment showed one major Fc/2 product (calcd mass 29521 Da, observed mass 29523 Da, approximately 95% of total Fc/2), corresponding to the DAR8 conjugate trastuzumab-(3)₂-(82)₄. RP-UPLC analysis of the sample showed an average DAR of 7.60.

### Example 89: Conjugation of Trastuzumab(Tetrazine)₄ [Trastuzumab-(3)₂] with BCN—HS-PEG₂-HS-Branched-(Va-PABC-O-Daunorubicin)₂ 87 to Obtain DAR8 Conjugate Trastuzumab-(3)₂-(87)₄

To a solution of trastuzumab-(3)₂ (900 μL, 18 mg, 20 mg/ml in TBS pH 7.5) was added sodium deoxycholate (180 μL, 110 mM) and BCN—HS-PEG₂-HS-branched-(va-PABC-O-Daunorubicin)₂ 87 (72 μL, 10 mM solution in DMF) and propylene glycol (468 μL at a final concentration of 30%). The reaction was incubated at room temperature for 30 minutes, a colour change from pink to yellow/colourless was observed. Next the conjugate was purified on a Superdex200 16/600 column (Cytiva) on an AKTA Pure (Cytiva). Mass spectral analysis of the sample after IdeS treatment showed one major Fc/2 product (calcd mass 30021 Da, observed mass 30020 Da, approximately 95% of total Fc/2), corresponding to the DAR8 conjugate trastuzumab-(3)₂-(87)₄. RP-UPLC analysis of the sample showed an average DAR of 7.51.

### Example 90: Conjugation of Trastuzumab(Tetrazine)₄ [Trastuzumab-(3)₂] with BCN—HS-PEG₂-HS—(Va-PABC-Pyrrolidine-CH₂CH₂—COO-Paclitaxel)₂ 91 to Obtain DAR8 Conjugate Trastuzumab-(3)₂-(91)₄

To a solution of trastuzumab-(3)₂ (26 μL, 0.5 mg, 19.03 mg/ml in TBS pH 7.5) was added sodium cholate (5 μL, 110 mM) and BCN—HS-PEG₂-HS-(va-PABC-pyrrolidine-CH₂CH₂—COO-Paclitaxel)₂ 91 (2.7 μL, 10 mM solution in DMF) and propylene glycol (12.3 μL at a final concentration of 30%). The reaction was incubated at room temperature for 10 minutes, a colour change from pink to yellow/colourless was observed. Mass spectral analysis of the sample after IdeS treatment showed one major Fc/2 product (calcd mass 31827 Da, observed mass 31826 Da, approximately 95% of total Fc/2), corresponding to the DAR8 conjugate trastuzumab-(3)₂-(91)₄.

### Example 91: Conjugation of Trastuzumab(Tetrazine)₄ [Trastuzumab-(3)₂] with Cyclopropene-HS-PEG₂-(ValCit-PABC-MMAE)₂ 96 to Obtain DAR8 Conjugate Trastuzumab-(3)₂-(96)₄

To a solution of trastuzumab-(3)₂ (26 μL, 0.5 mg, 19.03 mg/ml in TBS pH 7.5) was added sodium cholate (5 μL, 110 mM) and cyclopropene-HS-PEG₂-(ValCit-PABC-MMAE)₂ 96 (2.7 μL, 10 mM solution in DMF) and propylene glycol (12.3 μL at a final concentration of 30%). The reaction was incubated at room temperature for 10 minutes, a colour change from pink to yellow/colourless was observed. Mass spectral analysis of the sample after IdeS treatment showed one major Fc/2 product (calcd mass 31011 Da, observed mass 31010 Da, approximately 95% of total Fc/2), corresponding to the DAR8 conjugate trastuzumab-(3)₂-(96)₄.

### Example 92: Conjugation of Trastuzumab(Tetrazine)₄ [Trastuzumab-(3)₂] with TCO-HS-PEG₂-(ValCit-PABC-MMAE)₂ 97 to Obtain DAR8 Conjugate Trastuzumab-(3)₂(97)₄

To a solution of trastuzumab-(3)₂ (26 μL, 0.5 mg, 19.03 mg/ml in TBS pH 7.5) was added sodium cholate (5 μL, 110 mM) and TCO-HS-PEG₂-(ValCit-PABC-MMAE)₂ 97 (2.7 μL, 10 mM solution in DMF) and propylene glycol (12.3 μL at a final concentration of 30%). The reaction was incubated at room temperature for 10 minutes, a colour change from pink to yellow/colourless was observed. Mass spectral analysis of the sample after IdeS treatment showed one major Fc/2 product (calcd mass 31095 Da, observed mass 31094 Da, approximately 95% of total Fc/2), corresponding to the DAR8 conjugate trastuzumab-(3)₂-(97)₄.

### Example 93: Conjugation of Trastuzumab(Tetrazine)₄ [Trastuzumab-(3)₂] with BCN—HS-PEG₂-HS-Branched-(Va-PABC-Staurosporine)₂ 102 to Obtain DAR8 Conjugate Trastuzumab-(3)₂-(102)₄

To a solution of trastuzumab-(3)₂ (816 μL, 23.5 mg, 28.76 mg/ml in TBS pH 7.5) was added sodium deoxycholate (234 μL, 110 mM) and BCN—HS-PEG₂-HS-branched-(va-PABC-staurosporine)₂ 102 (125 μL, 10 mM solution in DMF) and propylene glycol (579 μL at a final concentration of 30%). The reaction was incubated at room temperature for 30 minutes, a colour change from pink to yellow/colourless was observed. Next the conjugate was purified on a Superdex200 16/600 column (Cytiva) on an AKTA Pure (Cytiva). Mass spectral analysis of the sample after IdeS treatment showed one major Fc/2 product (calcd mass 29777 Da, observed mass 29775 Da, approximately 95% of total Fc/2), corresponding to the DAR8 conjugate trastuzumab-(3)₂-(102)₄. RP-UPLC analysis of the sample showed an average DAR of 7.63.

### Example 94: Conjugation of Trastuzumab(Tetrazine)₄ [Trastuzumab-(3)₂] with BCN—HS—(Vc-PABC-MMAE)₂ 200 to Obtain DAR8 Conjugate Trastuzumab-(3)₂-(200)₄

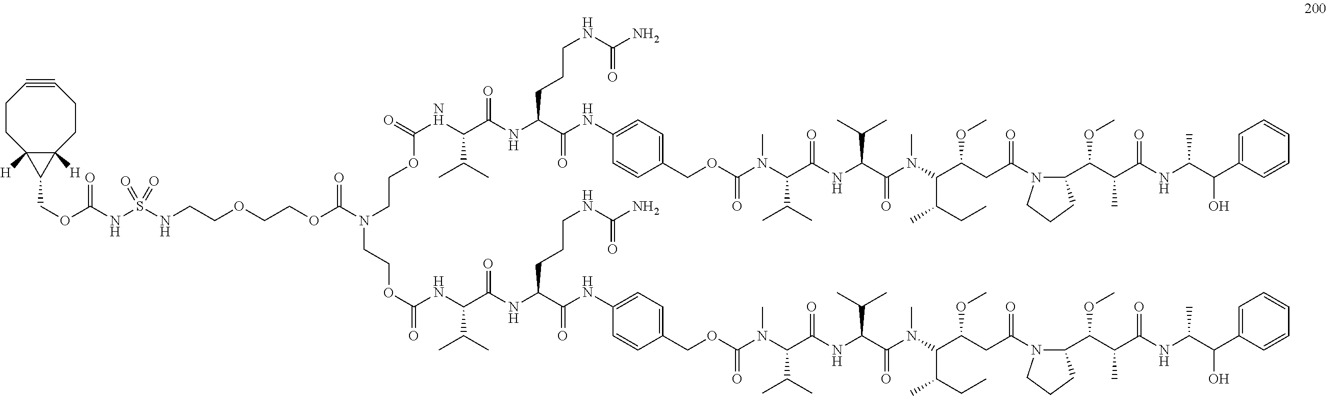

The synthesis of BCN—HS-(vc-PABC-MMAE)₂ (200) is described in WO 2017/137458 (example 25).

To a solution of trastuzumab-(3)₂ (16 μL, 0.5 mg, 31.7 mg/ml in TBS pH 7.5) was added sodium deoxycholate (5 μL, 110 mM) and BCN—HS-(vc-PABC-MMAE)₂ 200 (2.7 μL, 10 mM solution in DMF) and propylene glycol (12.3 μL at a final concentration of 30%). The reaction was incubated at room temperature for 10 minutes, a colour change from pink to yellow/colourless was observed. Mass spectral analysis of the sample after IdeS treatment showed one major Fc/2 product (calcd mass 30968 Da, observed mass 30969 Da, approximately 95% of total Fc/2), corresponding to the DAR8 conjugate trastuzumab-(3)₂-(200)₄.

### Example 95: Conjugation of Trastuzumab(Tetrazine)₄ [Trastuzumab-(3)₂] with BCN—HS-PEG₂-(Vc-PABC-MMAF)₂ 201 to Obtain DAR8 Conjugate Trastuzumab-(3)₂-(201)₄

The synthesis of BCN—HS-PEG₂-(vc-PABC-MMAF)₂ (201) is described in WO 2017/137457 (example 55).

To a solution of trastuzumab-(3)₂ (16 μL, 0.5 mg, 31.7 mg/ml in TBS pH 7.5) was added sodium deoxycholate (5 μL, 110 mM) and BCN—HS-PEG₂-(vc-PABC-MMAF)₂ 201 (2.7 μL, 10 mM solution in DMF) and propylene glycol (12.3 μL at a final concentration of 30%). The reaction was incubated at room temperature for 10 minutes, a colour change from pink to yellow/colourless was observed. Mass spectral analysis of the sample after IdeS treatment showed one major Fc/2 product (calcd mass 31025 Da, observed mass 31025 Da, approximately 95% of total Fc/2), corresponding to the DAR8 conjugate trastuzumab-(3)₂-(201)₄.

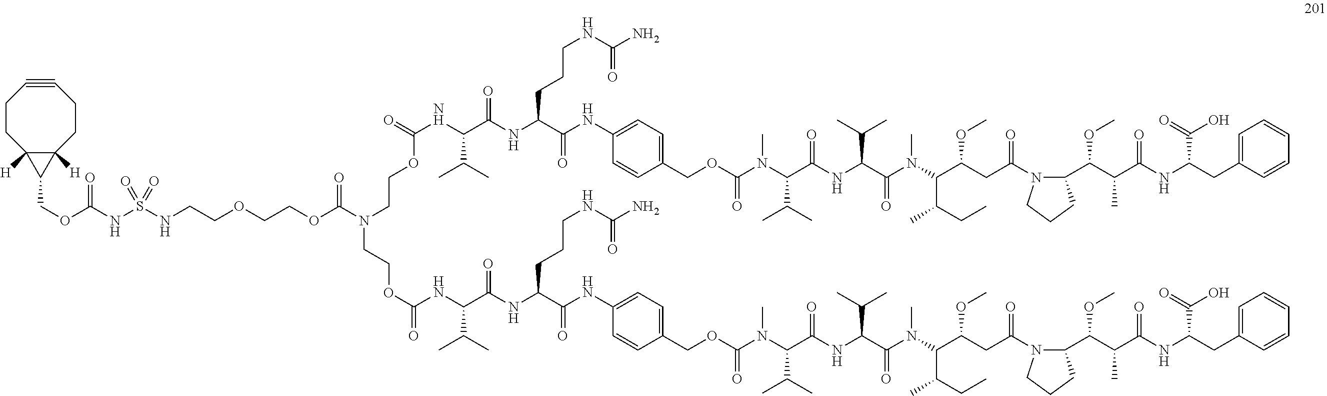

### Example 96: Conjugation of Trastuzumab(Tetrazine)₄ [Trastuzumab-(3)₂] with BCN—HS-PEG₂-HS-b-(Vc-PABC-Ahx-May)₂ 202 to Obtain DAR8 Conjugate Trastuzumab-(3)₂-(202)₄

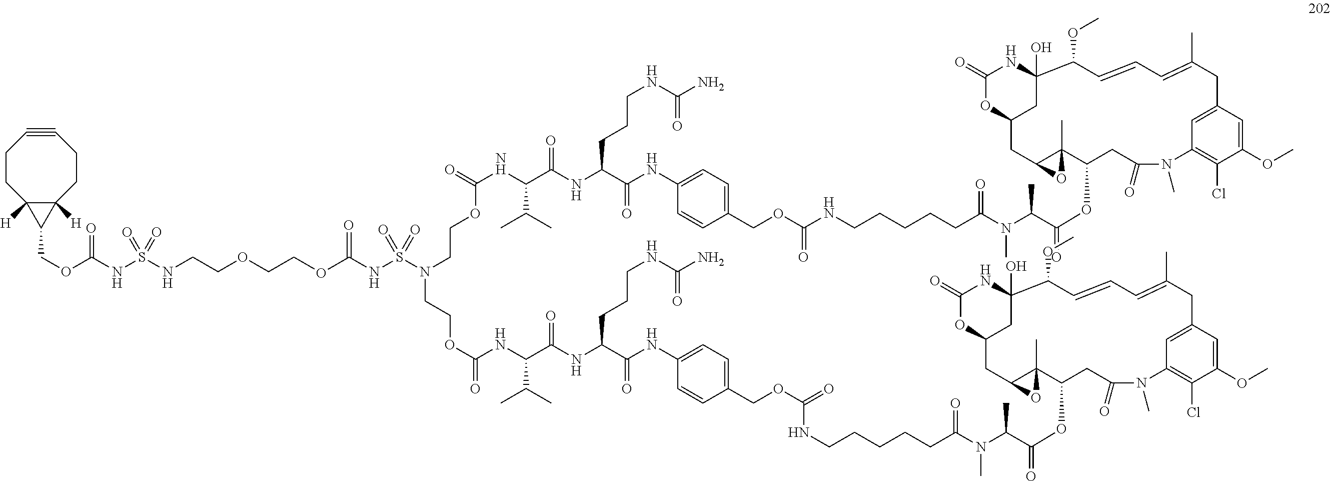 [SMILES: COc1cc2cc(c1Cl)N(C)C(=O)C[C@H](OC(=O)[C@H](C)N(C)C(=O)CCCCCNC(=O)OCc1ccc(NC(=O)[C@H](CCCNC(N)=O)NC(=O)[C@@H](NC(=O)OCCN(CCOC(=O)N[C@H](C(=O)N[C@@H](CCCNC(N)=O)C(=O)Nc3ccc(COC(=O)NCCCCCC(=O)N(C)[C@@H](C)C(=O)O[C@H]4CC(=O)N(C)c5cc(cc(OC)c5Cl)C/C(C)=C/C=C/[C@@H](OC)[C@@]5(O)C[C@@H](C[C@@H]6O[C@@]46C)OC(=O)N5)cc3)C(C)C)S(=O)(=O)NC(=O)OCCOCCNS(=O)(=O)NC(=O)OC[C@@H]3[C@@H]4CCC#CCC[C@@H]43)C(C)C)cc1)[C@]1(C)O[C@H]1C[C@@H]1C[C@@](O)(NC(=O)O1)[C@H](OC)/C=C/C=C(\C)C2]

The synthesis of BCN—HS-PEG₂-HS-(vc-PABC-Ahx-May)₂ (202) is described in WO 2016/053107 (example 55).

To a solution of trastuzumab-(3)₂ (16 μL, 0.5 mg, 31.7 mg/m in TBS pH 7.5) was added sodium deoxycholate (5 μL, 110 mM) and BCN—HS-PEG₂-HS-b-(vc-PABC-Ahx-May)₂ 202 (2.7 μL, 10 mM solution in DMF) and propylene glycol (12.3 μL at a final concentration of 30%). The reaction was incubated at room temperature for 10 minutes, a colour change from pink to yellow/colourless was observed. Mass spectral analysis of the sample after IdeS treatment showed one major Fc/2 product (calcd mass 31308 Da, observed mass 31308 Da, approximately 95% of total Fc/2), corresponding to the DAR8 conjugate trastuzumab-(3)₂-(202)₄.

### Example 97: Conjugation of Palivizumab(Tetrazine)₄ [Palivizumab-(3)₂] with BCN—HS-PEG₂-HS—(Va-PABC-Ex)₂ 7 to Obtain DAR8 Conjugate Palivizumab-(3)₂-(7)₄

To a solution of palivizumab-(3)₂ (17 μL, 0.5 mg, 29.8 mg/ml in TBS pH 7.5) was added sodium deoxycholate (5 μL, 110 mM) and BCN—HS-PEG₂-HS-(va-PABC-Ex)₂ 7 (2.7 μL, 10 mM solution in DMF) and propylene glycol (12.3 μL at a final concentration of 30%). The reaction was incubated at room temperature for 5 minutes, a colour change from pink to yellow/colourless was observed. Mass spectral analysis of the sample after IdeS treatment showed one major Fc/2 product (calcd mass 29653 Da, observed mass 29654 Da, approximately 95% of total Fc/2), corresponding to the DAR8 conjugate palivizumab-(3)₂-(7)₄. RP-UPLC analysis of the sample showed an average DAR of 7.56.

### Example 98: Conjugation of B12(Tetrazine)₄ [B12-(3)₂] with BCN—HS-PEG₂-HS—(Va-PABC-Ex)₂ 7 to Obtain DAR8 Conjugate B12-(3)₂-(7)₄

To a solution of B12-(3)₂ (25 μL, 0.5 mg, 20.2 mg/ml in TBS pH 7.5) was added BCN—HS-PEG₂-HS-(va-PABC-Ex)₂ 7 (3.3 μL, 10 mM solution in DMF) and propylene glycol (21.7 μL at a final concentration of 50%). The reaction was incubated at room temperature for 5 minutes, a colour change from pink to yellow/colourless was observed. Mass spectral analysis of the sample after IdeS treatment showed one major Fc/2 product (calcd mass 29620 Da, observed mass 29622 Da, approximately 85% of total Fc/2, other 15% corresponded to the m/z+Lys, calcd mass 29750 Da, observed mass 29748 Da), corresponding to the DAR8 conjugate B12-(3)₂-(7)₄. RP-UPLC analysis of the sample showed an average DAR of 7.61.

### Example 99: Conjugation of Sacituzumab(Tetrazine)₄ [Sacituzumab-(3)₂] with BCN—HS-PEG₂-HS—(Va-PABC-Ex)₂ 7 to Obtain DAR8 Conjugate Sacituzumab-(3)₂-(7)₄

To a solution of sacituzumab-(3)₂ (21 μL, 0.5 mg, 23.76 mg/ml in TBS pH 7.5) was added sodium deoxycholate (5 μL, 110 mM) and BCN—HS-PEG₂-HS-(va-PABC-Ex)₂ 7 (2.7 μL, 10 mM solution in DMF) and propylene glycol (12.3 μL at a final concentration of 30%). The reaction was incubated at room temperature for 15 minutes, a colour change from pink to yellow/colourless was observed. Mass spectral analysis of the sample after IdeS treatment showed one major Fc/2 product (calcd mass 29652 Da, observed mass 29651 Da, approximately 95% of total Fc/2), corresponding to the DAR 8 conjugate sacituzumab-(3)₂-(7)₄.

### Example 100: Conjugation of Rituximab(Tetrazine)₄ [Rituximab-(3)₂] with BCN—HS-PEG₂-HS—(Va-PABC-Ex)₂ 7 to Obtain DAR8 Conjugate Rituximab-(3)₂-(7)₄

To a solution of rituximab-(3)₂ (30 μL, 0.5 mg, 16.79 mg/ml in TBS pH 7.5) was added sodium deoxycholate (5 μL, 110 mM) and BCN—HS-PEG₂-HS-(va-PABC-Ex)₂ 7 (2.7 μL, 10 mM solution in DMF) and propylene glycol (12.3 μL at a final concentration of 30%). The reaction was incubated at room temperature for 15 minutes, a colour change from pink to yellow/colourless was observed. Mass spectral analysis of the sample after IdeS treatment showed one major Fc/2 product (calcd mass 29619 Da, observed mass 29619 Da, approximately 95% of total Fc/2), corresponding to the DAR 8 conjugate rituximab-(3)₂-(7)₄.

### Example 101: Conjugation of Trastuzumab(Tetrazine)₄ [Trastuzumab-(23)₂] with BCN—HS-PEG₂-HS—(Va-PABC-Ex)₂ 7 to Obtain DAR8 Conjugate Trastuzumab-(23)₂-(7)₄

To a solution of trastuzumab-(23)₂ (283 μL, 7.4 mg, 26.18 mg/ml in TBS pH 7.5) was added sodium cholate (74 μL, 110 mM) and BCN—HS-PEG₂-HS-(va-PABC-Ex)₂ 7 (40 μL, 10 mM solution in DMF) and propylene glycol (183 μL). The reaction was incubated at room temperature for 1 hour, a colour change from pink to yellow/colourless was observed. The precipitate of the reaction was removed by centrifugation, the pellet was rinsed with TBS (100 μL), centrifuged and combined. Next the conjugate was purified on a Superdex200 10/300 increase column (Cytiva) on an AKTA Pure (Cytiva). To remove the excess of free payload, 5 mg of active charcoal (Carbon RHC, Filtrox AG) was added and rotated for 4.5 h. The charcoal was removed by centrifugation and subsequently filtered over a PES syringe filter (pore 0.20 μm, Corning). Subsequently a HiTrap 26-10 desalting column (Cytiva) was used to final formulate the ADC. The column was rinsed with 0.2M NaOH and equilibrated with 20 mM histidine, 6% sucrose buffer pH 6.0. Lastly 0.04% Tween-20 (w/v) was added. Mass spectral analysis of the sample after IdeS treatment showed one major Fc/2 product (calcd mass 29673 Da, observed mass 29670 Da, approximately 95% of total Fc/2), corresponding to the DAR8 conjugate trastuzumab-(23)₂-(7)₄. RP-UPLC analysis of the sample showed an average DAR of 7.32.

### Example 102: Conjugation of Trastuzumab(Tetrazine)₄ [Trastuzumab-(25)₂] with BCN—HS-PEG₂-HS—(Va-PABC-Ex)₂ 7 to Obtain DAR8 Conjugate Trastuzumab-(25)₂-(7)₄

To a solution of trastuzumab-(25)₂ (292 μL, 7.7 mg, 26.41 mg/ml in TBS pH 7.5) was added sodium cholate (77 μL, 110 mM) and BCN—HS-PEG₂-HS-(va-PABC-Ex)₂ 7 (41 μL, 10 mM solution in DMF) and propylene glycol (190 μL). The reaction was incubated at room temperature for 40 minutes, a colour change from pink to yellow/colourless was observed. The precipitate of the reaction was removed by centrifugation, the pellet was rinsed with TBS (100 μL), centrifuged and combined. Next the conjugate was purified on a Superdex200 10/300 increase column (Cytiva) on an AKTA Pure (Cytiva). Subsequently the solution was dialysed to 20 mM histidine, 6% sucrose buffer pH 6.0 three times for one hour at rt. Lastly the product was concentrated to 3.11 mg/mL using a Vivaspin Turbo 4 10 kDa MWCO ultrafiltration unit (Sartorius) and 0.04% Tween-20 (w/v) was added. Mass spectral analysis of the sample after IdeS treatment showed one major Fc/2 product (calcd mass 29714 Da, observed mass 29712 Da, approximately 95% of total Fc/2), corresponding to the DAR8 conjugate trastuzumab-(25)₂-(7)₄. RP-UPLC analysis of the sample showed an average DAR of 7.26.

### Example 103: Conjugation of Trastuzumab(Tetrazine)₄ [Trastuzumab-(26)₂] with BCN—HS-PEG₂-HS—(Va-PABC-Ex)₂ 7 to Obtain DAR8 Conjugate Trastuzumab-(26)₂-(7)₄

To a solution of trastuzumab-(26)₂ (22 μL, 0.5 mg, 22.7 mg/ml in TBS pH 7.5) was added sodium deoxycholate (10 μL, 110 mM) and BCN—HS-PEG₂-HS-(va-PABC-Ex)₂ 7(2 μL, 10 mM solution in DMF) and propylene glycol (28 μL at a final concentration of 30%). The reaction was incubated at room temperature for 2 hours, a colour change from pink to yellow/colourless was observed. Mass spectral analysis of the sample after IdeS treatment showed one major Fc/2 product (calcd mass 29889 Da, observed mass 29887 Da, approximately 95% of total Fc/2), corresponding to the DAR8 conjugate trastuzumab-(26)₂-(7)₄ with a DAR of 7.73.

### Example 104: Conjugation of Trastuzumab(Tetrazine)₄ [Trastuzumab-(27)₂] with BCN—HS-PEG₂-HS—(Va-PABC-Ex)₂ 7 to Obtain DAR8 Conjugate Trastuzumab-(27)₂-(7)₄

To a solution of trastuzumab-(27)₂ (31 μL, 0.5 mg, 16.16 mg/ml in TBS pH 7.5) was added sodium deoxycholate (10 μL, 110 mM) and BCN—HS-PEG₂-HS-(va-PABC-Ex)₂ 7 (2 μL, 10 mM solution in DMF) and propylene glycol (28 μL at a final concentration of 30%). The reaction was incubated at room temperature for 2 hours, a colour change from pink to yellow/colourless was observed. Mass spectral analysis of the sample after IdeS treatment showed one major Fc/2 product (calcd mass 29565 Da, observed mass 29563 Da, approximately 95% of total Fc/2), corresponding to the DAR8 conjugate trastuzumab-(27)₂-(7)₄ with a DAR of 7.36.

### Example 105: Conjugation of Trastuzumab(Tetrazine)₄ [Trastuzumab-(29)₂] with BCN—HS-PEG₂-HS—(Va-PABC-Ex)₂ 7 to Obtain DAR8 Conjugate Trastuzumab-(29)₂-(7)₄

To a solution of trastuzumab-(29)₂ (359 μL, 7.7 mg, 21.32 mg/ml in TBS pH 7.5) was added sodium cholate (85 μL, 110 mM) and BCN—HS-PEG₂-HS-(va-PABC-Ex)₂ 7 (34 μL, 10 mM solution in DMF) and propylene glycol (221 μL). The reaction was incubated at room temperature for 30 minutes, a colour change from pink to yellow/colourless was observed. The precipitate of the reaction was removed by centrifugation, the pellet was rinsed with TBS (100 μL), centrifuged and combined. Next the conjugate was purified on a Superdex200 10/300 increase column (Cytiva) on an AKTA Pure (Cytiva). Subsequently the solution was dialysed to 20 mM histidine, 6% sucrose buffer pH 6.0 two times for one hour at rt and once overnight at 4° C. Lastly the product was concentrated using a Vivaspin Turbo 4 10 kDa MWCO ultrafiltration unit (Sartorius) and 0.04% Tween-20 (w/v) was added. Mass spectral analysis of the sample after IdeS treatment showed one major Fc/2 product (calcd mass 29581 Da, observed mass 29578 Da, approximately 95% of total Fc/2), corresponding to the DAR8 conjugate trastuzumab-(29)₂-(7)₄. RP-UPLC analysis of the sample showed an average DAR of 7.45.

### Example 106: Conjugation of Trastuzumab(Tetrazine)₄ [Trastuzumab-(31)₂] with BCN—HS-PEG₂-HS—(Va-PABC-Ex)₂ 7 to Obtain DAR8 Conjugate Trastuzumab-(31)₂-(7)₄

To a solution of trastuzumab-(31)₂ (59 μL, 1.0 mg, 17.07 mg/ml in TBS pH 7.5) was added sodium deoxycholate (20 μL, 110 mM) and BCN—HS-PEG₂-HS-(va-PABC-Ex)₂ 7 (4 μL, 10 mM solution in DMF) and propylene glycol (56 μL at a final concentration of 30%). The reaction was incubated at room temperature for 2 hours, a colour change from pink to yellow/colourless was observed. Mass spectral analysis of the sample after IdeS treatment showed one major Fc/2 product (calcd mass 29748 Da, observed mass 29747 Da, approximately 95% of total Fc/2), corresponding to the DAR8 conjugate trastuzumab-(31)₂-(7)₄ with a DAR of 5.57.

### Example 107: Conjugation of Trastuzumab(Tetrazine)₄ [Trastuzumab-(33)₂] with BCN—HS-PEG₂-HS—(Va-PABC-Ex)₂ 7 to Obtain DAR8 Conjugate Trastuzumab-(33)₂-(7)₄

To a solution of trastuzumab-(33)₂ (59 μL, 1.0 mg, 16.81 mg/ml in TBS pH 7.5) was added sodium deoxycholate (20 μL, 110 mM) and BCN—HS-PEG₂-HS-(va-PABC-Ex)₂ 7 (4 μL, 10 mM solution in DMF) and propylene glycol (56 μL at a final concentration of 30%). The reaction was incubated at room temperature for 2 hours, a colour change from pink to yellow/colourless was observed. Mass spectral analysis of the sample after IdeS treatment showed one major Fc/2 product (calcd mass 29792 Da, observed mass 29793 Da, approximately 95% of total Fc/2), corresponding to the DAR8 conjugate trastuzumab-(33)₂-(7)₄ with a DAR of 6.11.

### Example 108: Conjugation of Trastuzumab(Tetrazine)₄ [Trastuzumab-(35)₂] with BCN—HS-PEG₂-HS—(Va-PABC-Ex)₂ 7 to Obtain DAR8 Conjugate Trastuzumab-(35)₂-(7)₄

To a solution of trastuzumab-(35)₂ (310 μL, 7.8 mg, 25.14 mg/ml in TBS pH 7.5) was added sodium cholate (78 μL, 110 mM) and BCN—HS-PEG₂-HS-(va-PABC-Ex)₂ 7 (42 μL, 10 mM solution in DMF) and propylene glycol (232 μL). The reaction was incubated at room temperature for 45 minutes, a colour change from pink to yellow/colourless was observed. The precipitate of the reaction was removed by centrifugation, the pellet was rinsed with TBS (100 μL), centrifuged and combined. Next the conjugate was purified on a Superdex200 10/300 increase column (Cytiva) on an AKTA Pure (Cytiva). Subsequently the solution was dialysed to 20 mM histidine, 6% sucrose buffer pH 6.0 three times for one hour at rt. Lastly the product was concentrated using a Vivaspin Turbo 4 10 kDa MWCO ultrafiltration unit (Sartorius) and 0.04% Tween-20 (w/v) was added. Mass spectral analysis of the sample after IdeS treatment showed one major Fc/2 product (calcd mass 29538 Da, observed mass 29536 Da, approximately 95% of total Fc/2), corresponding to the DAR8 conjugate trastuzumab-(35)₂-(7)₄. RP-UPLC analysis of the sample showed an average DAR of 7.30.

### Example 109: Conjugation of Trastuzumab(Nitrone)₄ [Trastuzumab-(37′)₂] with BCN—HS-PEG₂-HS—(Va-PABC-Ex)₂ 7 to Obtain DAR8 Conjugate Trastuzumab-(37′)₂-(7)₄

To a solution of trastuzumab-(37′)₂ (25 μL, 0.5 mg, 20.3 mg/ml in TBS pH 7.5) was added sodium deoxycholate (5 μL, 110 mM) and BCN—HS-PEG₂-HS-(va-PABC-Ex)₂ 7 (3.3 μL, 10 mM solution in DMF) and propylene glycol (11.7 μL at a final concentration of 30%). The reaction was incubated overnight at room temperature. Mass spectral analysis of the sample after IdeS treatment showed one major Fc/2 product (calcd mass 29591 Da, observed mass 29600 Da, approximately 75% of total Fc/2), corresponding to the DAR8 conjugate trastuzumab-(37)₂-(7)₄.

### Example 110: Conjugation of Trastuzumab(Tetrazine)₄ [Trastuzumab-(41)₂] BCN—HS-PEG₂-HS—(Va-PABC-Ex)₂ 7 to Obtain DAR8 Conjugate Trastuzumab-(41)₂-(7)₄

To a solution of trastuzumab-(41)₂ (30 μL, 0.5 mg, 16.41 mg/ml in TBS pH 7.5) was added sodium deoxycholate (5 μL, 110 mM) and BCN—HS-PEG₂-HS-(va-PABC-Ex)₂ 7 (2.7 μL, 10 mM solution in DMF) and propylene glycol (12.3 μL at a final concentration of 30%). The reaction was incubated at room temperature for 2 hours, a colour change from pink to yellow/colourless was observed. Mass spectral analysis of the sample after IdeS treatment showed one major Fc/2 product (calcd mass 30347 Da, observed mass 30346 Da, approximately 95% of total Fc/2), corresponding to the DAR8 trastuzumab-(41)₂-(7)₄ with a DAR of 4.80.

### Example 111: Conjugation of Trastuzumab(Tetrazine)₆ [Trastuzumab-(45)₂] with BCN—HS-PEG₂-HS—(Va-PABC-Ex)₂ 7 to Obtain DAR12 Conjugate Trastuzumab-(45)₂-(7)₆

To a solution of trastuzumab-(45)₂ (50 μL, 0.8 mg, 16 mg/ml in TBS pH 7.5) was added sodium deoxycholate (8 μL, 110 mM) and BCN—HS-PEG₂-HS-(va-PABC-Ex)₂ 7 (5.3 μL, 10 mM solution in DMF) and propylene glycol (18.7 μL at a final concentration of 30%). The reaction was incubated at room temperature for 5 minutes, a colour change from pink to yellow/colourless was observed. Mass spectral analysis of the sample after IdeS treatment showed one major Fc/2 product (calcd mass 32010 Da, observed mass 32007 Da, approximately 95% of total Fc/2), corresponding to the DAR12 conjugate trastuzumab-(45)₂-(7)₆.

### Example 112: Conjugation of Trastuzumab(Tetrazine)₆ [Trastuzumab-(45)₂] with BCN—HS-PEG₂-HS—(Va-PABC-Mitomycin)₂ 59 to Obtain DAR12 Conjugate Trastuzumab-(45)₂-(59)₆

To a solution of trastuzumab-(45)₂ (263 μL, 5 mg, 19.03 mg/ml in TBS pH 7.5) was added sodium deoxycholate (50 μL, 110 mM) and BCN—HS-PEG₂-HS-(va-PABC-Mitomycin)₂ 59 (27 μL, 10 mM solution in DMF) and propylene glycol (123 μL). The reaction was incubated at room temperature for 45 minutes, a colour change from pink to yellow/colourless was observed. Next the conjugate was purified on a Superdex200 10/300 increase column (Cytiva) on an AKTA Pure (Cytiva). Mass spectral analysis of the sample after IdeS treatment showed one major Fc/2 product (calcd mass 31403 Da, observed mass 31404 Da, approximately 95% of total Fc/2), corresponding to the DAR12 conjugate trastuzumab-(45)₂-(59)₆.

### Example 113: Conjugation of Trastuzumab(Tetrazine)₆ [Trastuzumab-(45)₂] with BCN—HS-PEG₂-Va-PABC-Mitomycin 72 to Obtain DAR6 Conjugate Trastuzumab-(45)₂-(72)₆

To a solution of trastuzumab-(45)₂ (263 μL, 5 mg, 19.03 mg/ml in TBS pH 7.5) was added sodium deoxycholate (50 μL, 110 mM) and BCN—HS-PEG₂-va-PABC-Mitomycin 72 (27 μL, 10 mM solution in DMF) and propylene glycol (123 μL). The reaction was incubated at room temperature for 45 minutes, a colour change from pink to yellow/colourless was observed. Next the conjugate was purified on a Superdex200 10/300 increase column (Cytiva) on an AKTA Pure (Cytiva). Mass spectral analysis of the sample after IdeS treatment showed one major Fc/2 product (calcd mass 28733 Da, observed mass 28734 Da, approximately 95% of total Fc/2), corresponding to the DAR6 conjugate trastuzumab-(45)₂-(72)₆.

### Example 114: Conjugation of Trastuzumab(Tetrazine)₆ [Trastuzumab-(45)₂] with BCN—HS-PEG₂-Va-PABC-Gennari-Gemcitabine 86 to Obtain DAR6 Conjugate Trastuzumab-(45)₂-(86)₆

To a solution of trastuzumab-(tetrazine 45)₂ (26 μL, 0.5 mg, 19.04 mg/ml in TBS pH 7.5) was added sodium deoxycholate (5 μL, 110 mM) and BCN—HS-PEG₂-va-PABC-Gennari-Gemcitabine 86 (2.7 μL, 10 mM solution in DMF) and propylene glycol (12.3 μL at a final concentration of 30%). The reaction was incubated at room temperature for 5 minutes, a colour change from pink to yellow/colourless was observed. Mass spectral analysis of the sample after IdeS treatment showed one major Fc/2 product (calcd mass 28939 Da, observed mass 28941 Da, approximately 95% of total Fc/2), corresponding to the DAR6 conjugate trastuzumab-(45)₂-(86)₆.

### Example 115: Conjugation of Trastuzumab(Tetrazine)₆ [Trastuzumab-(45)₂] with BCN—HS-PEG₂-Va-PABC-Gennari-Paclitaxel 88 to Obtain DAR6 Conjugate Trastuzumab-(45)₂-(88)₆

To a solution of trastuzumab-(45)₂ (26 μL, 0.5 mg, 19.04 mg/ml in TBS pH 7.5) was added sodium deoxycholate (5 μL, 110 mM) and BCN—HS-PEG₂-va-PABC-Gennari-Paclitaxel 88 (2.7 μL, 10 mM solution in DMF) and propylene glycol (12.3 μL at a final concentration of 30%). The reaction was incubated at room temperature for 5 minutes, a colour change from pink to yellow/colourless was observed. Mass spectral analysis of the sample after IdeS treatment showed one major Fc/2 product (calcd mass 30714 Da, observed mass 30713 Da, approximately 95% of total Fc/2), corresponding to the DAR6 conjugate trastuzumab-(45)₂-(88)₆.

### Example 116: Conjugation of Trastuzumab(Tetrazine)₆ [Trastuzumab-(45)₂] with BCN—HS-PEG₂-Va-PABC-Pyrrolidine-CH₂CH₂—COO-Paclitaxel 92 to Obtain DAR6 Conjugate Trastuzumab-(45)₂-(92)₆

To a solution of trastuzumab-(45)₂ (26 μL, 0.5 mg, 19.04 mg/ml in TBS pH 7.5) was added sodium deoxycholate (5 μL, 110 mM) and BCN—HS-PEG₂-va-PABC-pyrrolidine-CH₂CH₂—COO-Paclitaxel 92 (2.7 μL, 10 mM solution in DMF) and propylene glycol (12.3 μL at a final concentration of 30%). The reaction was incubated at room temperature for 5 minutes, a colour change from pink to yellow/colourless was observed. Mass spectral analysis of the sample after IdeS treatment showed one major Fc/2 product (calcd mass 30669 Da, observed mass 30668 Da, approximately 95% of total Fc/2), corresponding to the DAR6 conjugate trastuzumab-(45)₂-(92)₆.

### Example 117: Conjugation of Trastuzumab(Tetrazine)₆ [Trastuzumab-(45)₂] with BCN—HS-PEG₂-Va-PABC-UCN-01 10 to Obtain DAR6 Conjugate Trastuzumab-(45)₂-(100)₆

To a solution of trastuzumab-(45)₂ (414 μL, 8.5 mg, 20.53 mg/ml in TBS pH 7.5) was added sodium cholate (85 μL, 110 mM) and BCN—HS-PEG₂-va-PABC-UCN-01 100 (45 μL, 10 mM solution in DMF) and propylene glycol (210 μL at a final concentration of 30%). The reaction was incubated at room temperature for 5 minutes, a colour change from pink to yellow/colourless was observed. Mass spectral analysis of the sample after IdeS treatment showed one major Fc/2 product (calcd mass 29179 Da, observed mass 29178 Da, approximately 95% of total Fc/2), corresponding to the DAR6 conjugate trastuzumab-(45)₂-(100)₆. RP-UPLC analysis of the sample showed an average DAR of 5.41.

### Example 118: Conjugation of Trastuzumab(Tetrazine)₆ [Trastuzumab-(45)₂] with BCN—HS-PEG₂-Va-PABC-Ex 203 to Obtain DAR6 Conjugate Trastuzumab-(45)₂-(203)₆

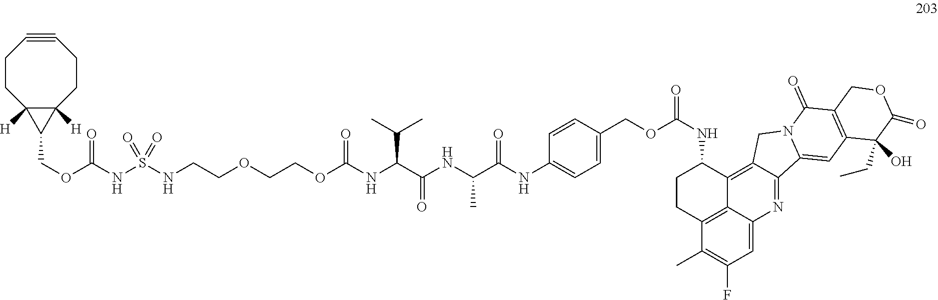 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)OCCOCCNS(=O)(=O)NC(=O)OC[C@@H]2[C@@H]4CCC#CCC[C@@H]42)C(C)C)cc1)CC3]

The synthesis of BCN—HS-PEG₂-va-PABC-Ex (203) is described in PCT/EP2021/075401 (example 11).

To a solution of trastuzumab-(45)₂ (15 μL, 0.5 mg, 32.6 mg/ml in TBS pH 7.5) was added sodium deoxycholate (5 μL, 110 mM) and BCN—HS-PEG₂-va-PABC-Ex 203 (2.7 μL, 10 mM solution in DMF) and propylene glycol (12.3 μL at a final concentration of 30%). The reaction was incubated at room temperature for 5 minutes, a colour change from pink to yellow/colourless was observed. Mass spectral analysis of the sample after IdeS treatment showed one major Fc/2 product (calcd mass 29039 Da, observed mass 29039 Da, approximately 95% of total Fc/2), corresponding to the DAR6 conjugate trastuzumab-(45)₂-(203)₆.

### Example 119: Conjugation of Trastuzumab(Tetrazine)₄ [Trastuzumab-(49)₂] with BCN—HS-PEG₂-HS—(Va-PABC-Ex)₂ 7 to Obtain DAR8 Conjugate Trastuzumab-(49)₂-(7)₄

To a solution of trastuzumab-(49)₂ (25 μL, 0.5 mg, 19.61 mg/ml in TBS pH 7.5) was added sodium deoxycholate (5 μL, 110 mM) and BCN—HS-PEG₂-HS-(va-PABC-Ex)₂ 7 (2.7 μL, 10 mM solution in DMF) and propylene glycol (12.3 μL at a final concentration of 30%). The reaction was incubated at room temperature for 5 minutes, a colour change from pink to yellow/colourless was observed. Mass spectral analysis of the sample after IdeS treatment showed one major Fc/2 product (calcd mass 29847 Da, observed mass 29845 Da, approximately 95% of total Fc/2), corresponding to the DAR8 conjugate trastuzumab-(49)₂-(7)₄.

### Example 120: Conjugation of Trastuzumab(Tetrazine)₄ [Trastuzumab-(51)₂] with BCN—HS-PEG₂-HS—(Va-PABC-Ex)₂ 7 to Obtain DAR8 Conjugate Trastuzumab-(51)₂-(7)₄

To a solution of trastuzumab-(51)₂ (22 μL, 0.5 mg, 22.65 mg/ml in TBS pH 7.5) was added sodium deoxycholate (5 μL, 110 mM) and BCN—HS-PEG₂-HS-(va-PABC-Ex)₂ 7 (2.7 μL, 10 mM solution in DMF) and propylene glycol (12.3 μL at a final concentration of 30%). The reaction was incubated at room temperature for 5 minutes, a colour change from pink to yellow/colourless was observed. Mass spectral analysis of the sample after IdeS treatment showed one major Fc/2 product (calcd mass 30548 Da, observed mass 30549 Da, approximately 95% of total Fc/2), corresponding to the DAR8 conjugate trastuzumab-(51)₂-(7)₄.

### Example 121: Conjugation of Trastuzumab(Tetrazine)₆ [Trastuzumab-(54)₂] with BCN—HS-PEG₂-Va-PABC-Mitomycin 72 to Obtain DAR6 Conjugate Trastuzumab-(54)₂-(72)₆

To a solution of trastuzumab-(54)₂ (8 μL, 0.1 mg, 12 mg/ml in TBS pH 7.5) was added sodium deoxycholate (1.3 μL, 110 mM) and BCN—HS-PEG₂-va-PABC-Mitomycin 72 (0.5 μL, 10 mM solution in DMF) and propylene glycol (3.5 μL at a final concentration of 30%). The reaction was incubated at room temperature for 30 minutes, a colour change from pink to yellow/colourless was observed. Mass spectral analysis of the sample after DTT reduction showed one major HC product (calcd mass 54517 Da, observed mass 54520 Da, approximately 95% of total HC), corresponding to the DAR6 conjugate trastuzumab-(54)₂-(72)₆ with a DAR of 4.30.

### Example 122: Conjugation of Trastuzumab(Tetrazine)₆ [Trastuzumab-(54)₂] with BCN—HS-PEG₂-Va-PABC-Midostaurin 98 to Obtain DAR6 Conjugate Trastuzumab-(54)₂-(98)₆

To a solution of trastuzumab-(54)₂ (8 μL, 0.1 mg, 12 mg/ml in TBS pH 7.5) was added sodium deoxycholate (1.3 μL, 110 mM) and BCN—HS-PEG₂-va-PABC-Midostaurin 98 (0.5 μL, 10 mM solution in DMF) and propylene glycol (3.5 μL at a final concentration of 30%). The reaction was incubated at room temperature for 30 minutes, a colour change from pink to yellow/colourless was observed. Intact RP-UPLC measurement corresponded to the DAR6 conjugate trastuzumab-(54)₂-(98)₆, with an average DAR of 4.44, see FIG. 18.

### Example 123: Conjugation of Trastuzumab(Tetrazine)₆ [Trastuzumab-(54)₂] with BCN—HS-PEG₂-Va-PABC-UCN-01 10 to Obtain DAR6 Conjugate Trastuzumab-(54)₂-(100)₆

To a solution of trastuzumab-(54)₂ (414 μL, 8.5 mg, 20.53 mg/ml in TBS pH 7.5) was added sodium cholate (85 μL, 110 mM) and BCN—HS-PEG₂-va-PABC-UCN-01 100 (45 μL, 10 mM solution in DMF) and propylene glycol (210 μL at a final concentration of 30%). The reaction was incubated at room temperature for 5 minutes, a colour change from pink to yellow/colourless was observed. Mass spectral analysis of the sample after DTT reduction showed one major HC product (calcd mass 54961 Da, observed mass 54963 Da, approximately 95% of total HC), corresponding to the DAR6 conjugate trastuzumab-(54)₂-(100)₆. RP-UPLC analysis of the sample showed an average DAR of 4.80.

### Example 124: Conjugation of Trastuzumab(Tetrazine)₆ [Trastuzumab-(54)₂] with BCN—HS-PEG₂-Va-PABC-Ex 203 to Obtain DAR6 Conjugate Trastuzumab-(54)₂-(203)₆

To a solution of trastuzumab-(54)₂ (23 μL, 0.5 mg, 21.53 mg/ml in TBS pH 7.5) was added sodium deoxycholate (5 μL, 110 mM) and BCN—HS-PEG₂-va-PABC-Ex 203 (2.7 μL, 10 mM solution in DMF) and propylene glycol (12.3 μL at a final concentration of 30%). The reaction was incubated at room temperature for 30 minutes, a colour change from pink to yellow/colourless was observed. Mass spectral analysis of the sample after IdeS treatment showed one major Fc/2 product (calcd mass 29462 Da, observed mass 29461 Da, approximately 95% of total HC), corresponding to the DAR 6 conjugate trastuzumab-(54)₂-(203)₆. RP-UPLC analysis of the sample showed an average DAR of 4.46.

### Example 125: Conjugation of Trastuzumab(Tetrazine)₆ [Trastuzumab-(55)₂] with BCN—HS-PEG₂-Va-PABC-Ex 203 to Obtain DAR6 Conjugate Trastuzumab-(55)₂-(203)₆

To a solution of trastuzumab-(55)₂ (20 μL, 0.5 mg, 25.29 mg/ml in TBS pH 7.5) was added sodium deoxycholate (5 μL, 110 mM) and BCN—HS-PEG₂-va-PABC-Ex 203 (2.7 μL, 10 mM solution in DMF) and propylene glycol (12.3 μL at a final concentration of 30%). The reaction was incubated at room temperature for 2 hours, a colour change from pink to yellow/colourless was observed. Mass spectral analysis of the sample after IdeS treatment showed one major Fc/2 product (calcd mass 30517 Da, observed mass 30516 Da, approximately 95% of total Fc/2), corresponding to the DAR 6 conjugate trastuzumab-(55)₂-(203)₆. RP-UPLC analysis of the sample showed an average DAR of 4.91.

### Examples 126-133: In Vitro Assays Determining Stability and Potency

### Example 126: Aggregation Study of DAR8 and DAR4 Constructs at Physiological Conditions and Comparison to Enhertu®

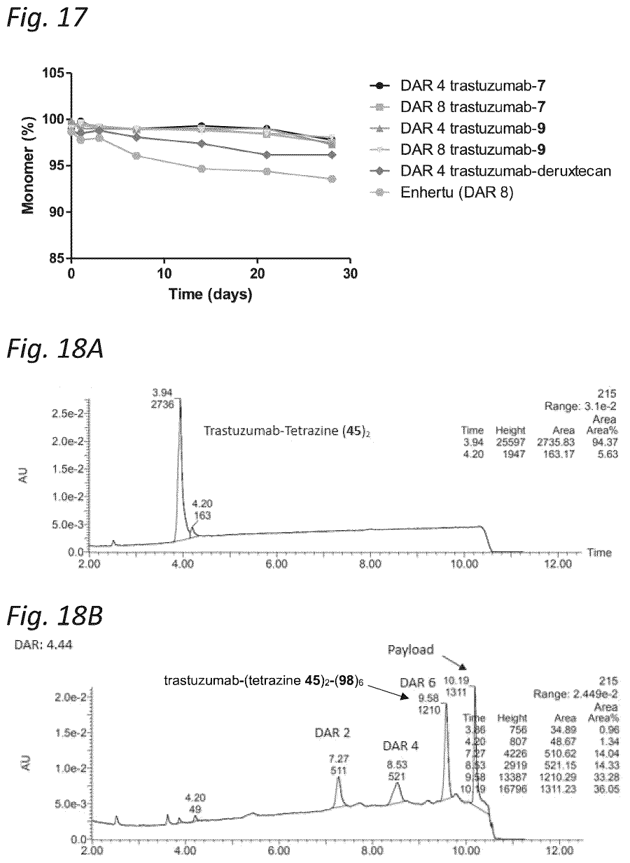

All samples were buffer exchanged using a HiTrap 26-10 desalting column (Cytiva), rinsed with 0.2M NaOH and equilibrated with PBS. Samples were diluted to 1 mg/mL and stored at 37° C. for 28 days. At designated time points (t=0, 1, 3, 7, 14, 21 and 28 days) a sample was taken and analyzed using SE-HPLC and RP-UPLC, as illustrated in FIG. 17.

### Example 127: Serum Stability Studies in Human and Mouse Serum

Stability of ADCs in mice and human serum was tested. Prior to the assay, the serum (obtained from Sigma-Aldrich) was depleted from all IgG using CaptivA® Protein A agarose (1 mL agarose/mL serum). ADCs were added to the depleted human/mouse serum to a final concentration of 0.1 mg/mL followed by incubation at 37° C. At each time point, 0.5 mL was snap frozen and stored at −80° C. until further analysis. To isolate the ADCs after incubation, 20 μl CaptivA® Protein A agarose resin was added to the samples and incubated for 1 hour at room temperature. The resin was washed three times with PBS (1 mL) and subsequently 0.1 M Glycine-HCl pH 2.7 (0.4 mL) was added to elute the ADCs. After elution, the samples were immediately neutralized with 1.0 M Tris pH 8.0 (0.1 mL). The samples were spin filtrated against PBS for three times using Amicon Ultra spin-filter 0.5 mL MWCO 10 kDa (Merck Millipore) and the volume was reduced to 40 μL, yielding a final ADC concentration of approximately 1 mg/mL. Samples were analyzed on RP-HPLC (DTT reduced) to determine the DAR, tables below.

TABLE 1
Human serum DAR (% from DAR at t = 0):

| | DAR t = 0 | DAR t = 3 | DAR t = 7 |
|---|---|---|---|
| trastuzumab-(3)₂-(7)₄ | 8.0 | 7.8 (98%) | 7.6 (95%) |
| trastuzumab-(3)₂-(9)₄ | 8.0 | 7.9 (98%) | 7.8 (97%) |
| Enhertu ® | 7.9 | 6.3 (80%) | 5.1 (65%) |

TABLE 2
Mouse serum DAR (% from DAR at t = 0):

| | DAR t = 0 | DAR t = 3 | DAR t = 7 |
|---|---|---|---|
| trastuzumab-(3)₂-(7)₄ | 8.0 | 7.9 (99%) | 7.8 (98%) |
| trastuzumab-(3)₂-(9)₄ | 8.0 | 7.2 (90%) | 7.4 (92%) |
| Enhertu ® | 7.7 | 6.3 (82%) | 4.2 (54%) |

### Example 128: HIC-HPLC Measurements for Relative Retention Time Determination

Samples were diluted to 1 mg/mL in PBS. HIC analysis is performed on an Agilent 1200 series using a TSKgel® Butyl-NPR HPLC Column (3.5 cm×4.6 mm, 2.5 μm). 10 μL sample is injected at a flow rate of 0.5 mL/min using a gradient starting from 100% buffer A (2 M ammonium sulfate in 50 mM potassium phosphate pH 6.0) to 1000/buffer B (50 mM potassium phosphate pH 6.0+20% isopropanol) in 20 minutes. The native mAb (trastuzumab) is measured and its retention time is set as 1. The other measured samples are relative retention times compared to the native mAb, as summarized in Table 3 and illustrated in FIG. 16.

TABLE 3
relative retention times to trastuzumab measured with HIC-HPLC:

| construct | relative retention time |
|---|---|
| trastuzumab | 1.00 |
| DAR8 trastuzumab-(3)₂-(7)₄ | 1.20 |
| DAR8 trastuzumab-(3)₂-(9)₄ | 1.14 |
| Enhertu ® | 1.25 |

### Example 129: Aggregation Study of DAR8 Constructs at Physiological Conditions

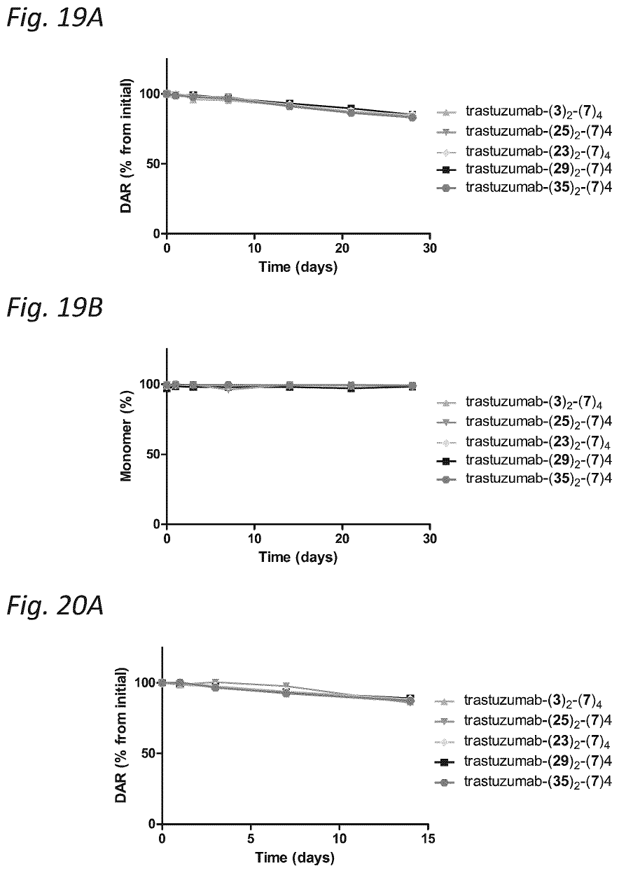

All samples not formulated in PBS were buffer exchanged using Amicon Ultra spin-filter 0.5 mL MWCO 10 kDa (Merck Millipore). Samples were diluted to 1 mg/mL and stored at 37° C. for 28 days. At designated time points (t=0, 1, 3, 7, 14, 21 and 28 days) a sample was taken and analyzed using SE-HPLC and RP-UPLC. Relative DAR compared to t=0 is measured by RP-UPLC (DTT reduced) and listed in table 4 and FIG. 19A. Monomer levels determined by SE-UPLC measurement are shown in table 5 and FIG. 19B.

### Example 130: Serum Stability Studies in Human and Mouse Serum

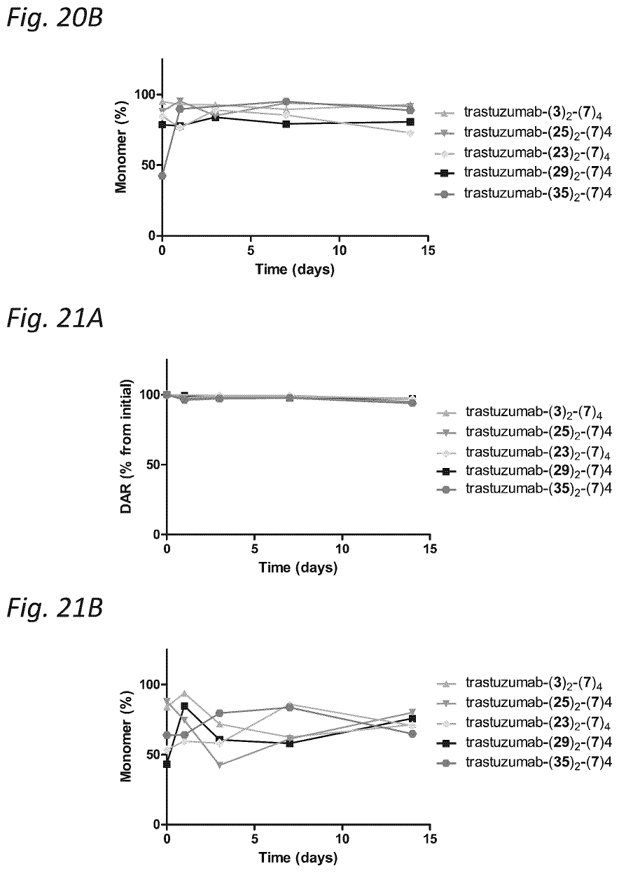

Stability of ADCs in mice and human serum was tested. Prior to the assay, the serum (obtained from Sigma-Aldrich) was depleted from all IgG using CaptivA® Protein A agarose (1 mL agarose/mL serum). ADCs were added to the depleted human/mouse serum to a final concentration of 0.1 mg/mL followed by incubation at 37° C. At each time point, 0.5 mL was snap frozen and stored at −80° C. until further analysis. To isolate the ADCs after incubation, 20 μl CaptivA® Protein A agarose resin was added to the samples and incubated for 1 hour at room temperature. The resin was washed three times with PBS (1 mL) and subsequently 0.1 M Glycine-HCl pH 2.7 (0.4 mL) was added to elute the ADCs. After elution, the samples were immediately neutralized with 1.0 M Tris pH 8.0 (0.1 mL). The samples were spin filtrated against PBS for three times using Amicon Ultra spin-filter 0.5 mL MWCO 10 kDa (Merck Millipore) and the volume was reduced to 40 μL, yielding a final ADC concentration of approximately 1 mg/mL. Samples were analyzed on RP-UPLC (DTT reduced) to determine the DAR, table 6 lists the data retrieved from human serum and 7 from mice serum. FIGS. 20A and 20B are graphs corresponding to the data retrieved from human samples and FIGS. 21A and 21B are graphs corresponding to the data retrieved from human samples.

TABLE 4
DAR value over time in PBS (% from DAR at t = 0):

| | DAR t = 0 | DAR t = 1 | DAR t = 3 | | DAR t = 7 | DAR t = 14 | DAR t = 21 | DAR t = 28 |
|---|---|---|---|---|---|---|---|---|
| trastuzumab-(3)₂-(7)₄ | 7.18 | 7.25 (100%) | 6.89 | (96%) | 6.85 (95%) | 6.57 (92%) | 6.27 (87%) | 6.06 (84%) |
| trastuzumab-(25)₂-(7)₄ | 7.25 | 7.2 (99%) | 7.11 | (98%) | 7.1 (98%) | 6.63 (91%) | 6.33 (87%) | 6.12 (84%) |
| trastuzumab-(23)₂-(7)₄ | 7.25 | 7.26 (100%) | 7.14 | (98%) | 7.03 (97%) | 6.69 (92%) | 6.34 (87%) | 6.15 (85%) |
| trastuzumab-(29)₂-(7)₄ | 7.59 | 7.55 (99%) | 7.52 | (99%) | 7.36 (97%) | 7.08 (93%) | 6.81 (90%) | 6.46 (85%) |
| trastuzumab-(35)₂-(7)₄ | 7.44 | 7.36 (99%) | 7.27 | (98%) | 7.17 (96%) | 6.77 (91%) | 6.43 (86%) | 6.18 (83%) |
| trastuzumab-(3)₂-(57)₄ | 7.23 | 6.90 (95%) | 7.30 | (100%)* | N.D. | N.D. | N.D. | N.D. |
| trastuzumab-(3)₂-(59)₄ | 7.71 | 7.57 (98%) | 7.34 | (95%)* | N.D. | N.D. | N.D. | N.D. |
| trastuzumab-(3)₂-(61)₄ | 7.70 | 7.63 (99%) | 7.49 | (97%)* | N.D. | N.D. | N.D. | N.D. |
| trastuzumab-(3)₂-(63)₄ | 7.63 | 7.59 (99%) | 7.42 | (97%)* | N.D. | N.D. | N.D. | N.D. |

*measured at t = 5 days

TABLE 5
Monomer level (%) over time in PBS

| | T = 0 | T = 1 | T = 3 | T = 7 | T = 14 | T = 21 | T = 28 |
|---|---|---|---|---|---|---|---|
| trastuzumab-(3)₂-(7)₄ | 99.0% | 99.7% | 99.7% | 99.4% | 99.5% | 99.0% | 99.4% |
| trastuzumab-(25)₂-(7)₄ | 99.6% | 99.8% | 99.8% | N.D. | 99.6% | 99.7% | 99.5% |
| trastuzumab-(23)₂-(7)₄ | 99.1% | 99.8% | 99.7% | 99.0% | 99.5% | 99.3% | 99.0% |
| trastuzumab-(29)₂-(7)₄ | 98.5% | 98.5% | 98.1% | 98.1% | 98.0% | 97.2% | 98.3% |
| trastuzumab-(35)₂-(7)₄ | 99.5% | 99.8% | 99.8% | 99.6% | 99.5% | 99.3% | 99.0% |
| trastuzumab-(3)₂-(57)₄ | 60.5% | N.D. | N.D. | N.D. | N.D. | N.D. | N.D. |
| trastuzumab-(3)₂-(59)₄ | >99% | >99% | >99%* | N.D. | N.D. | N.D. | N.D. |
| trastuzumab-(3)₂-(61)₄ | >99% | >99% | >99%* | N.D. | N.D. | N.D. | N.D. |
| trastuzumab-(3)₂-(63)₄ | >99% | >99% | >99%* | N.D. | N.D. | N.D. | N.D. |

TABLE 6
Human serum DAR (% from DAR at t = 0)

| | t = 0 | t = 1 | t = 3 | t = 7 | t = 14 |
|---|---|---|---|---|---|
| trastuzumab-(3)₂-(7)₄ | 7.15 | 7.06 (99%) | 6.95 (97%) | 6.60 (92%) | 6.21 (87%) |
| trastuzumab-(25)₂-(7)₄ | 7.17 | 7.09 (99%) | 7.20 (100%) | 7.02 (98%) | 6.16 (86%) |
| trastuzumab-(23)₂-(7)₄ | 7.14 | 7.16 (100%) | 6.97 (98%) | 6.71 (94%) | 6.31 (88%) |

TABLE 7
Mice serum DAR (% from DAR at t = 0)

| | t = 0 | t = 1 | t = 3 | t = 7 | t = 14 |
|---|---|---|---|---|---|
| trastuzumab-(3)₂-(7)₄ | 7.25 | 7.10 (98%) | 7.17 (99%) | 7.15 (99%) | 7.09 (98%) |
| trastuzumab-(25)₂-(7)₄ | 7.21 | 7.05 (98%) | 7.03 (98%) | 7.05 (98%) | 6.85 (95%) |
| trastuzumab-(23)₂-(7)₄ | 7.21 | N.D. | 7.18 (100%) | 7.18 (100%) | 6.97 (97%) |
| trastuzumab-(29)₂-(7)₄ | 7.62 | 7.58 (99%) | 7.47 (98%) | 7.45 (98%) | 7.39 (97%) |
| trastuzumab-(35)₂-(7)₄ | 7.34 | 7.07 (96%) | 7.13 (97%) | 7.17 (98%) | 6.89 (94%) |

### Example 131: In Vitro Cytotoxicity Compared to Free Payload

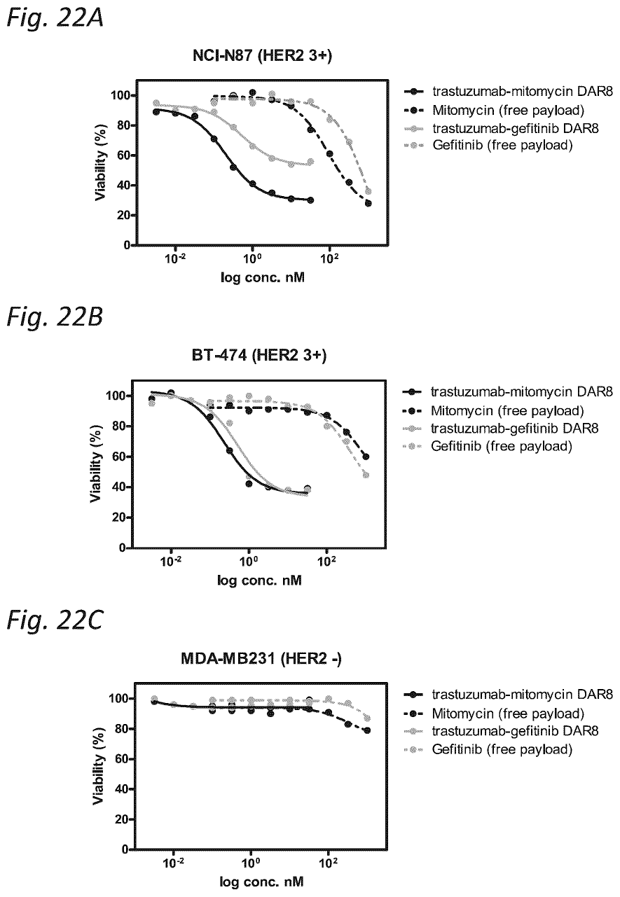

NCI-N87 (HER2 3+, LGC standards CRL 5822) or MDA-MB-231 (HER2−, LGC standards HTB-26) cells were plated in 96-well plates (5000 cells/well) in RPMI 1640 GlutaMAX (Invitrogen) supplemented with 10% fetal bovine serum (FBS) (Invitrogen, 150 μL/well) and incubated overnight in a humidified atmosphere at 37° C. and 5% CO₂. ADCs were added in triplo in a square root of 10 dilution series to obtain a final concentration ranging from 31 nM-0.03 nM. The cells were incubated for 5 days in a humidified atmosphere at 37° C. and 5% CO₂. The culture medium was replaced by 0.01 mg/mL resazurin (Sigma Aldrich) in RPMI 1640 GlutaMAX supplemented with 10% FBS (200 μL/well). After approximately 4 hours in a humidified atmosphere at 37° C. and 5% CO₂ the fluorescence was detected with a fluorescence plate reader (Infinite® M1000 Tecan) at 560 nm excitation and 590 nm emission. The relative fluorescent units (RFU) were normalized to cell viability percentage by setting wells without cells at 0% viability and wells with untreated cells at 100% viability (see FIGS. 22A-22C). ICs values for ADCs on the cells were calculated by non-linear regression using Graphpad prism software and are shown in the table 8 below.

TABLE 8
IC₅₀ values for two DAR8 ADCs and corresponding free payloads.

| | IC₅₀ | IC₅₀ | IC₅₀ |
|---|---|---|---|
| | (NCI-N87 cells) | (BT-474 cells) | (MDA-MB231 cells) |
| trastuzumab-(3)₂-(59)₄ | 0.2 nM | 0.2 nM | >100 nM |
| Mitomycin (free payload) | 94 nM | 905 nM | >100 nM |
| trastuzumab-(3)₂-(63)₄ | 0.5 nM | 0.5 nM | >100 nM |
| Gefitinib (free payload) | 997 nM | 410 nM | >100 nM |

### Example 132: In Vitro Cytotoxicity of Several Low Potency Linker Payloads

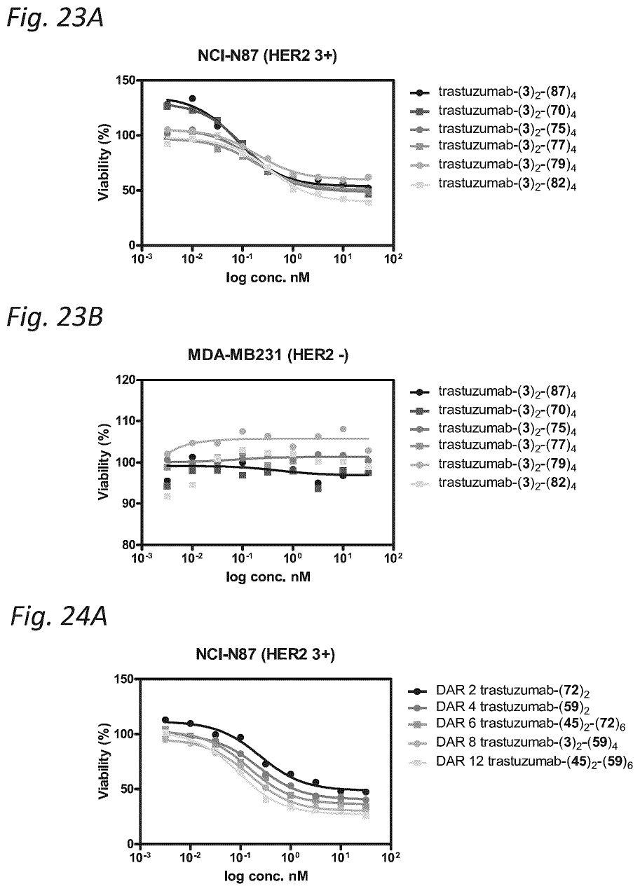

NCI-N87 (HER2 3+, LGC standards CRL 5822) or MDA-MB-231 (HER2−, LGC standards HTB-26 cells were plated in 96-well plates (5000 cells/well) in RPMI 1640 GlutaMAX (Invitrogen) supplemented with 10% fetal bovine serum (FBS) (Invitrogen, 150 μL/well) and incubated overnight in a humidified atmosphere at 37° C. and 5% CO₂. ADCs were added in triplo in a square root of 10 dilution series to obtain a final concentration ranging from 31 nM-0.03 nM. The cells were incubated for 5 days in a humidified atmosphere at 37° C. and 5% CO₂. The culture medium was replaced by 0.01 mg/mL resazurin (Sigma Aldrich) in RPMI 1640 GlutaMAX supplemented with 10% FBS (200 μL/well). After approximately 4 hours in a humidified atmosphere at 37° C. and 5% CO₂ the fluorescence was detected with a fluorescence plate reader (Infinite® M1000 Tecan) at 560 nm excitation and 590 nm emission. The relative fluorescent units (RFU) were normalized to cell viability percentage by setting wells without cells at 0% viability and wells with untreated cells at 100% viability (see FIGS. 23A-23B). IC₅₀ values for ADCs on the cells were calculated by non-linear regression using Graphpad prism software and are shown in the table 9 below.

TABLE 9
IC50 values for several DAR8 ADCs.

| | IC₅₀ | IC₅₀ |
|---|---|---|
| | (NCI-N87 cells) | (MDA-MB231 cells) |
| trastuzumab-(3)₂-(87)₄ | 0.08 nM | >100 nM |
| trastuzumab-(3)₂-(70)₄ | 0.11 nM | >100 nM |
| trastuzumab-(3)₂-(75)₄ | 0.18 nM | >100 nM |
| trastuzumab-(3)₂-(77)₄ | 0.20 nM | >100 nM |
| trastuzumab-(3)₂-(79)₄ | 0.19 nM | >100 nM |
| trastuzumab-(3)₂-(82)₄ | 0.34 nM | >100 nM |

### Example 133: In Vitro Cytotoxicity of Several DAR Formats

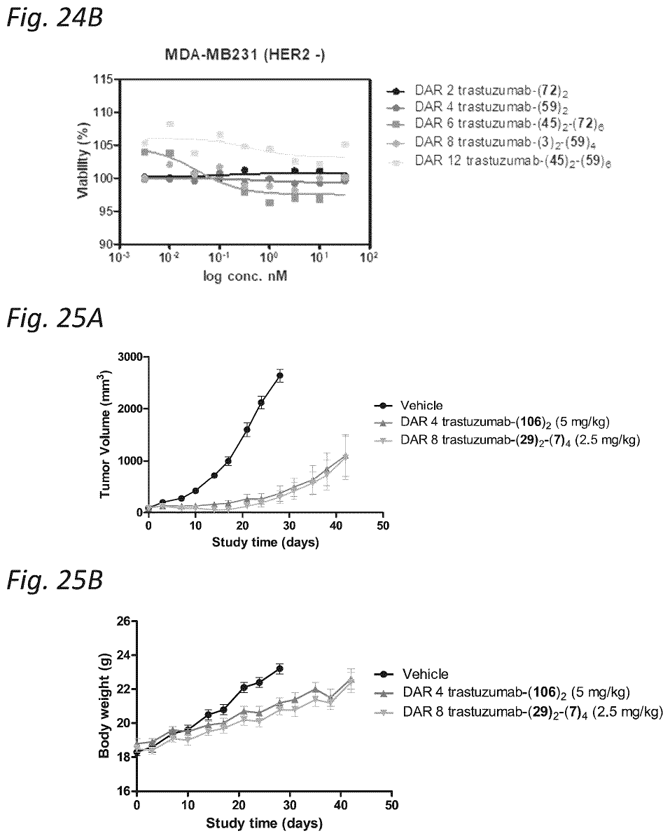

NCI-N87 (HER2 3+, LGC standards CRL 5822) or MDA-MB-231 (HER2−, LGC standards HTB-26) cells were plated in 96-well plates (5000 cells/well) in RPMI 1640 GlutaMAX (Invitrogen) supplemented with 10% fetal bovine serum (FBS) (Invitrogen, 150 μL/well) and incubated overnight in a humidified atmosphere at 37° C. and 5% CO₂. ADCs were added in triplo in a square root of 10 dilution series to obtain a final concentration ranging from 31 nM-0.03 nM. The cells were incubated for 5 days in a humidified atmosphere at 37° C. and 5% CO₂. The culture medium was replaced by 0.01 mg/mL resazurin (Sigma Aldrich) in RPMI 1640 GlutaMAX supplemented with 10% FBS (200 μL/well). After approximately 4 hours in a humidified atmosphere at 37° C. and 5% CO₂ the fluorescence was detected with a fluorescence plate reader (Infinite® M1000 Tecan) at 560 nm excitation and 590 nm emission. The relative fluorescent units (RFU) were normalized to cell viability percentage by setting wells without cells at 0% viability and wells with untreated cells at 100% viability (see FIGS. 24A-24B). ICs values for ADCs on the cells were calculated by non-linear regression using Graphpad prism software and are shown in table 10 below.

TABLE 10
IC₅₀ values for ADCs with varying DAR.

| | IC₅₀ | IC₅₀ |
|---|---|---|
| | (NCI-N87 | (MDA-MB231 |
| | cells) | cells) |
| DAR2 trastuzumab-(72)₂ | 0.25 nM | >100 nM |
| DAR4 trastuzumab-(59)₂ | 0.22 nM | >100 nM |
| DAR6 trastuzumab-(45)₂-(72)₆ | 0.14 nM | >100 nM |
| DAR8 trastuzumab-(3)₂-(59)₄ | 0.15 nM | >100 nM |
| DAR12 trastuzumab-(45)₂-(59)₆ | 0.09 nM | >100 nM |

### Examples 134-136: In Vivo Efficacy and Tolerability Studies

### Example 134: In Vivo Efficacy Study (Performed at Crown Bioscience, Zhongshan, China)

EMT6-hHER2, murine breast cancer cell line was maintained in vitro using DMEM medium supplemented with 10% FBS and 1 μg/mL puromycin in humidified cell culture incubator at 37° C. with standard 5% CO₂ specs. The cells in an exponential growth phase were harvested and counted for tumor inoculation.

BALB/c mice (female) of 6-8 weeks old received a subcutaneous injection in the right front flank region with 5×10⁵ tumor cells in 0.1 ml of PBS for tumor development. When tumors reached an average size of 80-120 mm³, randomization was performed into 3 groups of 10 mice and treatment began. Randomization will be performed based on “Matched distribution” method (Study Director™ software, version 3.1.399.19). The date of randomization will be denoted as day 0.

Electronic caliper measurement were performed 2 times a week. Daily observations for clinical signs, food and water consumption, behavioral changes, animals were weighed 2 times per week. The endpoint of the experiment is a tumor volume of 3,000 mm³, body weight loss over 20% or 41 days, whichever comes first.

Group assignment. Before grouping and treatment, all animals are weighed and the tumor volumes confirmed (80-120 mm³) using electronic caliper. Since the tumor volume can affect the effectiveness of any given treatment, mice assigned into groups using randomized block design as following: First, the experimental animals are divided into homogeneous blocks based on their tumor volume. Secondly, within each block, randomization of experimental animals to different groups conducted. By using randomized block design to assign experimental animals, we ensure that each animal has the same probability of being assigned to any given treatment groups and therefore systematic error is minimized.

Test article administration. Administration of compounds performed using Genie Touch Syringe Pump (Kent Scientific). Metered syringe pump programmed to deliver the injection. Terumo Surshield safety winged infusion sets (S25BLS, 25G×3/4) are used for the administration. Twenty percent (20%) of extra volume prepared and loaded into syringe (Kent Scientific) to ensure sufficient injection volume once the tubing is filled with the compound. All work performed in a Biological Safety Cabinet to prepare the samples and load sample into syringe. New syringe used for each individual test article and control group samples.

Observation and data collection. After tumor cells inoculation, the animals are checked daily for morbidity and mortality. At the time of routine monitoring, the animals are checked for any adverse effects of tumor growth and treatments on normal behavior such as mobility, visual estimation of food and water consumption, body weight gain/loss, eye/hair matting and any other abnormal effects. Tumor volumes are measured every 3-4 days in two dimensions using an caliper, and the volume data are expressed in mm3 using the formula: V=(L×W×W)/2, where V is tumor volume, L is tumor length (the longest tumor dimension) and W is tumor width (the longest tumor dimension perpendicular to L). Dosing as well as tumor and body weight measurements will be conducted in a Laminar Flow Cabinet.

TABLE 11
Overview of test items and dose levels included in in vivo study

| | Dose | Group | Administra- | |
|---|---|---|---|---|
| Test item | (mg/kg) | (N = 10) | tion | Schedule |
| vehicle | — | 1 | IV | Single dose |
| trastuzumab-(29)₂-(7)₄ | 2.5 | 2 | IV | Single dose |
| trastuzumab-(106)₂ | 5 | 3 | IV | Single dose |

Data for efficacy study with the test items above is depicted in FIG. 25A-25B.

### Example 135: In Vivo Efficacy Study (Performed at Crown Bioscience, Zhongshan, China)

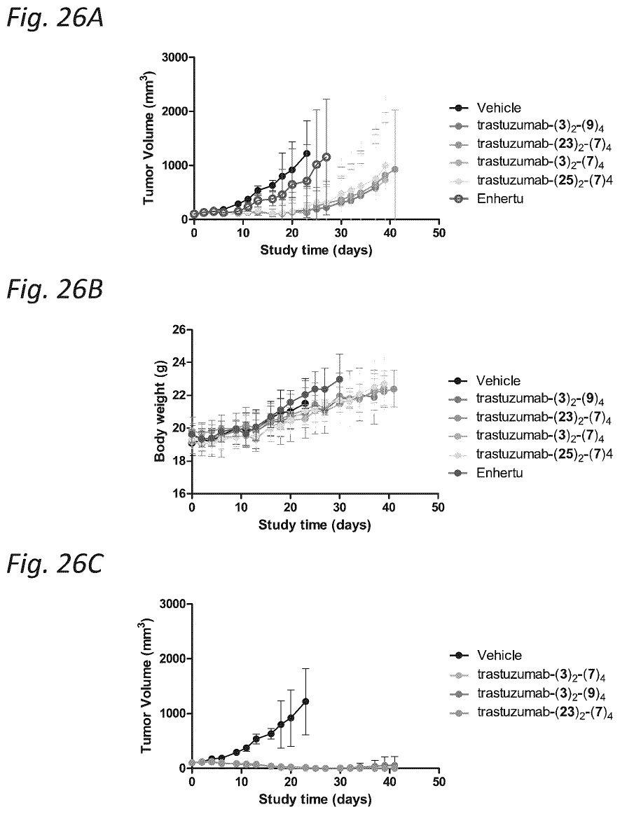

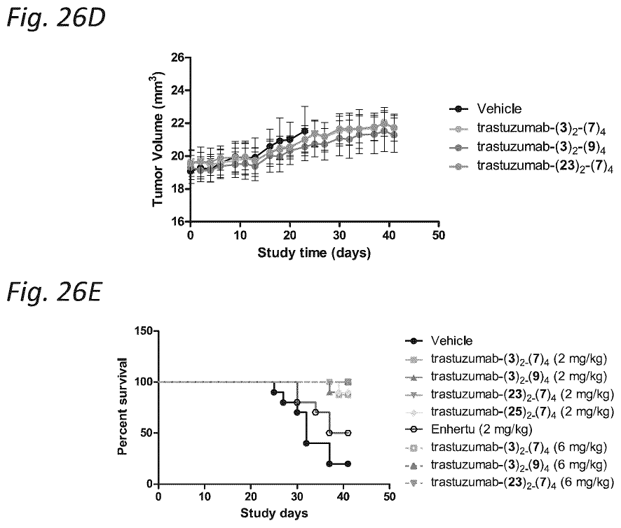

In this experiment, the protocol as described above for Example 134 is used, for 9 groups of 10 mice. Test items are depicted in table 12, and the data for efficacy study with the test items is depicted in FIG. 26A-26E.

TABLE 12
Overview of test items and dose levels included in in vivo study

| | Dose | Group | Administra- | |
|---|---|---|---|---|
| Test item | (mg/kg) | (N = 10) | tion | Schedule |
| vehicle | — | 1 | IV | Double dose |
| trastuzumab-(3)₂-(7)₄ | 2 | 2 | IV | Double dose |
| | 6 | 3 | IV | Double dose |
| trastuzumab-(3)₂-(9)₄ | 2 | 4 | IV | Double dose |
| | 6 | 5 | IV | Double dose |
| trastuzumab-(23)₂-(7)₄ | 2 | 6 | IV | Double dose |
| | 6 | 7 | IV | Double dose |
| trastuzumab-(25)₂-(7)₄ | 2 | 8 | IV | Double dose |
| Enhertu | 2 | 9 | IV | Double dose |

* Double dose was performed on day t = 0 and day t = 7

### Example 136: In Vivo Tolerability Study (Performed at Crown Bioscience, Zhongshan, China)

CD-1 mice (female) of 5-8 weeks old acclimatized for a week before being enrolled in the study.

Randomization: In the tolerability study totally 20 mice were enrolled and randomly allocated to 4 study groups with 5 mice per group. Randomization was performed based on “Matched distribution” method (StudyDirector™ software, version 3.1.399.19). The date of randomization was denoted as day 0.

Test Article Administration: The treatment was initiated on the same day of randomization (day 0) per study design. Chosen dose levels were 60, 80 and 100 mg/kg, single dose. All animals were dosed via slow intravenous injection (10 mL/kg).

Observation and Data Collection After randomization: The animals were checked daily for morbidity and mortality. During routine monitoring, the animals were checked for any effects of treatments on behavior such as mobility, food and water consumption, body weight gain/loss, eye/hair matting and any other abnormalities. Mortality and observed clinical signs were recorded for individual animals in detail. Dosing as well as body weight measurements were conducted in a Laminar Flow Cabinet. The body weights were measured daily by using StudyDirector™ software (version 3.1.399.19).

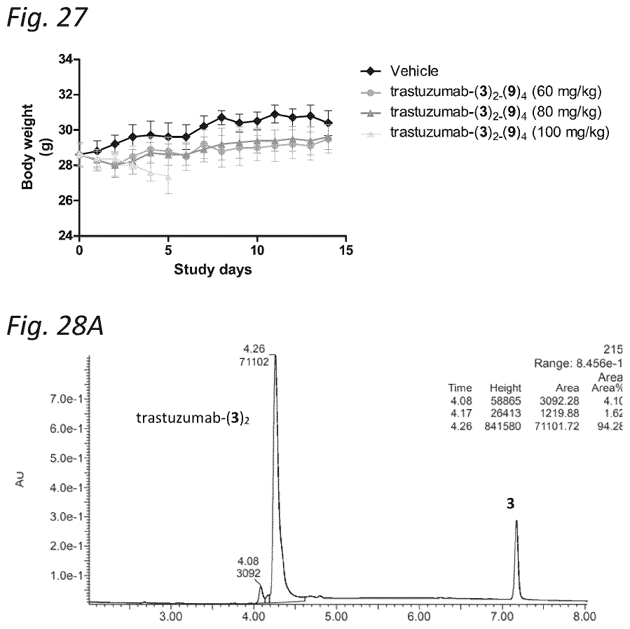

Body weight of the mice over time with trastuzumab-(3)₂-(9)₄ as test item at several dose levels are depicted in FIG. 27.

What is claimed is:

1. A process for preparing an antibody-payload-conjugate having a DAR of 6 or higher, the process comprising:
(a) providing a modified antibody having the structure Ab(F¹)_{z}, wherein Ab is an antibody, z is 2 or 4, and F¹ is a click probe;
(b) reacting the modified antibody with z equivalents of Q¹L^{A}(F²)_{y}, wherein Q¹ is a click probe that is reactive towards F¹, L^{A} is a heterobifunctional (y+1)-valent linker, y is 2, 3, or 4, and F² is a click probe that is not reactive towards Q¹, to obtain an antibody-linker construct of structure Ab(Z¹L^{A}(F²)_{y})_{z}, wherein Z¹ is a connecting group obtained by reaction of F¹ and Q¹;
(c) reacting the antibody-linker construct with z×y equivalents of Q²(L^{B})Dₓ, wherein Q² is a click probe that is reactive towards F², L^{B} is a (x+1)-valent linker, x is 1, 2, 3, or 4, provided that x+y is at least 4, and D is a payload molecule, to obtain the conjugate of structure Ab(Z¹L^{A}(Z²(L^{B})Dₓ)_{y})_{z}, wherein Z² is a connecting group obtained by reaction of F² and Q².

2. The process according to claim 1, wherein y is 2 and L^{A} is a heterobifunctional trivalent linker of structure 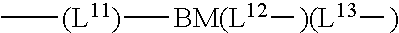
wherein:
L¹¹ is connected to Q¹ or Z¹, and L¹² and L¹³ are connected to F² or Z², wherein L¹¹, L¹², and L¹³ each individually consist of one or more building blocks selected from CH₂, CH═CH, C≡C, C(O), NR¹³, O, S, S(O), S(O)₂, PR¹³, and P(O)R¹³, preferably the building blocks are selected from CH₂, CH═CH, C(O), NR³, O, S, S(O), and S(O)₂; and
BM is a branching moiety, preferably selected from a carbon atom, a nitrogen atom, a phosphorus atom, a (hetero)aromatic ring, a (hetero)cycle, or a polycyclic moiety, more preferably BM is a nitrogen atom.

3. The process according to claim 1, wherein x is 2 and L^{B} is a heterobifunctional trivalent linker of structure 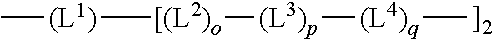
wherein:
L¹ is connected to Q² or Z², and both occurrences of L⁴ are connected to D;
L¹, L², L³, and L are each individually linkers that together link Q² or Z² to D;
o, p, and q are each individually 0 or 1, preferably wherein o=p=1;
and preferably wherein:
(a) linker L¹ is represented by: 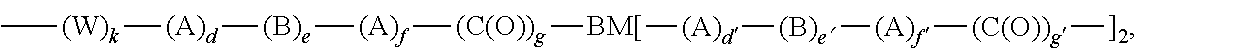
wherein:
d and d′ are individually 0 or 1;
e and e′ are individually an integer in the range 1-10;
f and f′ are individually 0 or 1;
g and g′ are individually an integer in the range 0-10;
k=0 or 1 with the proviso that if k=1, then d=0;
A is a carbamoyl or acyl sulfamide group according to structure (23) 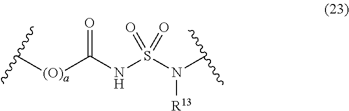
wherein a=0 or 1, and R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups, and C₃-C₂₄ (hetero)arylalkyl groups optionally substituted and optionally interrupted by one or more heteroatoms selected from O, S, and NR¹⁴, wherein R⁴ is independently selected from the group consisting of hydrogen and C₁-C₄ alkyl groups, or R¹³ is D connected to N via a spacer moiety, preferably wherein the spacer moiety is —(B)_{g}—(C(O))_{g}-(L²)ₒ-(L³)ₚ-(L⁴)_{q}-;
W is —OC(O)—, —C(O)O—, —C(O)NH—, —NHC(O)—, —OC(O)NH—, —NHC(O)O—, —C(O)(CH₂)ₘC(O)—, —C(O)(CH₂)ₘC(O)NH—, or -(4-Ph)CH₂NHC(O)(CH₂)ₘC(O)NH—, wherein m is an integer in the range 0-10;
B is a —CH₂—CH₂—O— or a —O—CH₂—CH₂— moiety, or (B)ₑ is a —(CH₂—CH₂—O)ₑ₁—CH₂—CH₂— moiety, wherein e1 is an integer in the range 1-10;
BM is a branching moiety, to which two instances of -(A)_{d}-(B)ₑ-(A)_{f}-(C(O))_{g}— are connected and both (C(O))_{g} moieties are connected to -(L²)ₒ-(L³)ₚ-(L⁴)_{q}-D, wherein L², L³, L⁴, o, p, q, and D are each selected individually;
and/or
(b) linker L² is a peptide spacer, preferably comprising 1-5 amino acids, more preferably a dipeptide, tripeptide or tetrapeptide spacer;
and/or
(c) linker L³ is a self-immolative spacer, preferably:
when o=1, L³ is a para-aminobenzyloxycarbonyl (PABC) derivative according to structure (26): 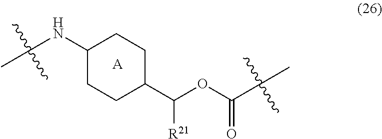
when o=1, L³ is a glucuronide derivative according to structure (27): 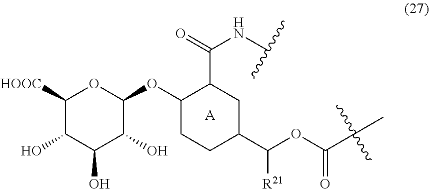
wherein R²¹ is H, R²⁶ or C(O) R²⁶, wherein R²⁶ is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups, and C₃-C₁₀ (hetero)arylalkyl groups, which are optionally substituted and optionally interrupted by one or more heteroatoms selected from O, S and NR²⁸ wherein R²⁸ is independently selected from the group consisting of hydrogen and C₁-C₄ alkyl groups, preferably wherein R²¹ is H or C(O)R²⁶, wherein R²⁶=4—N-methyl-piperazine or morpholine, most preferably wherein R²¹ is H;
and/or
(d) linker L⁴ is a spacer selected from:
an aminoalkanoic acid spacer according to the structure —NR²²—(Cₓ-alkylene)-C(O)—, wherein x is an integer in the range 1-20 and R²² is H or C₁-C₄ alkyl; or
an ethyleneglycol spacer according to the structure —NR²²—(CH₂—CH₂—O)ₑ₆—(CH₂)ₑ₇—C(O)—, wherein e6 is an integer in the range 1-10, e7 is an integer in the range 1-3, and R²² is H or C₁-C₄ alkyl; or
a diamine spacer according to the structure —NR²²—(Cₓ-alkylene)-NR²²—(C(O))ₕ—, wherein h is 0 or 1, x is an integer in the range 1-10 and R²² is H or C₁-C₄ alkyl.

4. The process according to claim 1, wherein the reaction of step (b) and the reaction of step (c) are both cycloadditions, preferably wherein the reaction of step (b) is a 1,3-dipolar cycloaddition and the reaction of step (c) is an inverse electron-demand Diels-Alder cycloaddition.

5. The process according to claim 1, wherein:
Q¹ and Q² are both individually click probes comprising a (hetero)cycloalkyne moiety or a (hetero)cyclo-E-alkene moiety, preferably wherein the click probes are selected from the group consisting of (Q2)-(Q20) according to the following structures: 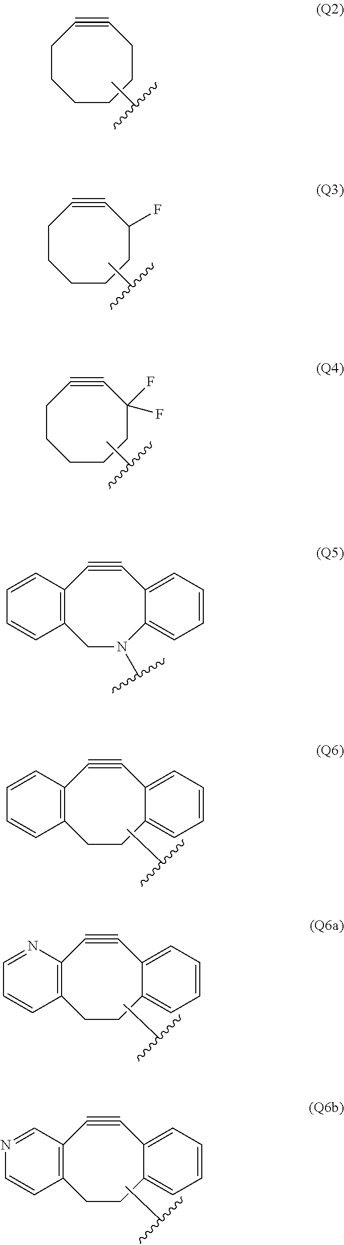 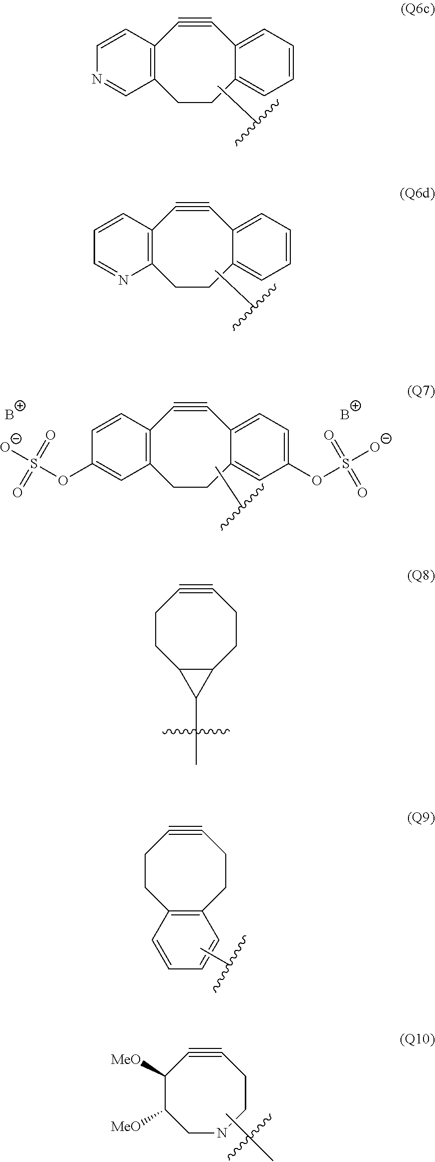 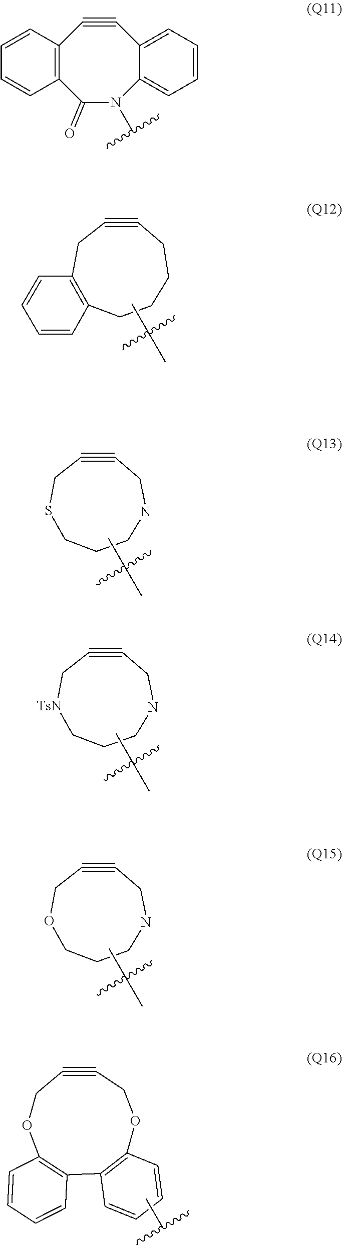 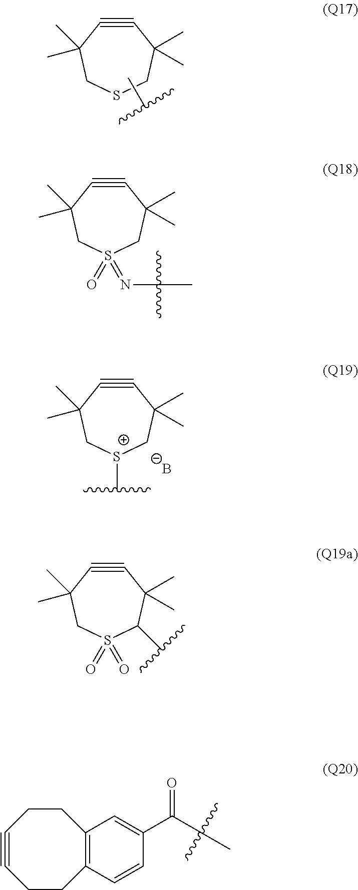
wherein:
the wavy bond represents the connection to L^{A} or L^{B}, and is connected to any available carbon or nitrogen atom;
the nitrogen atom of (Q10), (Q13), (Q14), and (Q15) may bear the connection to L^{A} or L^{B}, or contains a hydrogen atom or is substituted;
B⁽⁻⁾ is an anion;
B⁽⁺⁾ is a cation;
and/or
F¹ and F² are both individually click probes selected from the group consisting of azide, tetrazine, triazine, nitrone, nitrile oxide, nitrile imine, diazo compound, dioxothiophene, sydnone, iminosydnone and catechol, preferably wherein the click probes are selected from the group consisting of (F1)-(F10) according to the following structures: 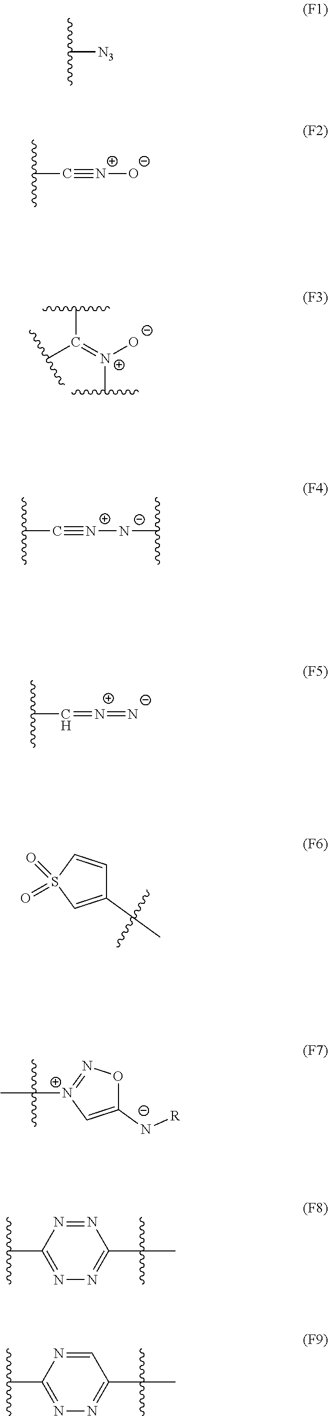 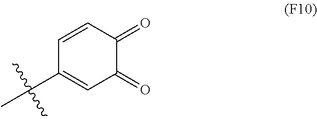 [SMILES: *C1=CC(=O)C(=O)C=C1]
wherein:
the wavy bond represents the connection to Ab or L^{A}, where for (F3), (F4), (F8), and (F9), the connection can be via any one of the wavy bonds, and the other wavy bond may then be connected to a group selected from hydrogen, C₁-C₂₄ alkyl groups, C₂-C₂₄ acyl groups, C₃-C₂₄ cycloalkyl groups, C₂-C₂₄ (hetero)aryl groups, C₃-C₂₄ alkyl(hetero)aryl groups, C₃-C₂₄ (hetero)arylalkyl groups, and C₁-C₂₄ sulfonyl groups, which may optionally be substituted and optionally be interrupted by one or more heteroatoms selected from O, S, and NR³², wherein R³² is independently selected from the group consisting of hydrogen and C₁-C₄ alkyl groups.

6. The process according to claim 1, wherein:
F¹ is an azide or a nitrone and Q¹ is a benzoannulated or tetramethylated (hetero)cycloalkyne, preferably selected from (Q5), (Q6), (Q6a), (Q6b), (Q6c), (Q6d), (Q7), (Q17), (Q18), (Q19), and (Q19a) as defined in claim 5; and
F² is a tetrazine and Q² is a bicyclononyne or cycloalkene, preferably wherein Q² is according to (Q8), (Q44), (Q47), (Q48), (Q49), (Q54), (Q55) or (Q56) as defined in claim 5, and F² is according to (F8a): 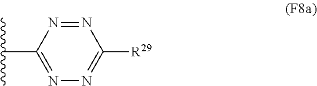
wherein:
R² is selected from hydrogen, C₁₋₆ alkyl, aryl, C(O)—C₁₋₆ alkyl, C(O)-aryl, C(O)—O—C₁₋₄ alkyl, C(O)—O-aryl, C(O)—NR³³—C¹⁻⁶ alkyl, and C(O)—NR³³-aryl, wherein R³³ is H or C₁₋₄ alkyl, preferably wherein R²⁹ is selected from hydrogen, methyl, phenyl, pyridyl, pyridinyl, and pyrimidinyl.

7. The process according to claim 1, wherein the payload D is a pharmaceutically active compound, preferably a cytotoxin.

8. The process according to claim 1, wherein the antibody provided in step (a) is according to structure: 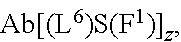
wherein:
z is 2 or 4;
L⁶ is -GlcNAc(Fuc)_{w}-(G)ⱼ-S-(L⁷)_{w′}-, wherein G is a monosaccharide, j is an integer in the range of 0-10, S is a sugar or a sugar derivative, GlcNAc is N-acetylglucosamine, and Fuc is fucose, w is 0 or 1, w′ is 0 or 1, and L⁷ is —N(H)C(O)CH₂—, —N(H)C(O)CF₂—, or —CH₂—.

9. The process according to claim 6, wherein F¹ is an azide or a nitrone, and Q¹ is a benzoannulated or tetramethylated (hetero)cycloalkyne, and F² is a tetrazine, and Q² is a bicyclononyne or cycloalkene.

10. The process according to claim 8, wherein F¹ is an azide or a nitrone, and Q¹ is a benzoannulated or tetramethylated (hetero)cycloalkyne, and F² is a tetrazine, and Q² is a bicyclononyne or cycloalkene.

11. A compound selected from:
a conjugate obtained by the process according to claim 1;
a conjugate having structure Ab(Z¹L^{A}(Z²(L^{B})Dₓ)_{y})_{z}, wherein:
Ab is an antibody;
Z¹ and Z² are connecting groups obtainable by reaction between two click probes;
L^{A} is a heterobifunctional (y+1)-valent linker;
L^{B} is a (x+1)-valent linker;
x is 1, 2, 3, or 4;
y is 2, 3, or 4, provided that x+y is at least 4;
z is 2 or 4;
D is a payload; and
a linker-construct having structure Q¹L^{A}(F²)_{y}, wherein:
Q¹ is a click probe;
L^{A} is a heterobifunctional (y+1)-valent linker;
F² is a click probe that is not reactive towards Q¹; and
y is 2, 3, or 4.

12. The compound according to claim 11, which is a conjugate having structure Ab(Z¹L^{A}(Z²(L^{B})Dₓ)_{y})_{z}, wherein Z¹ and Z² are individually selected from the structures (Z2)-(Z38a): 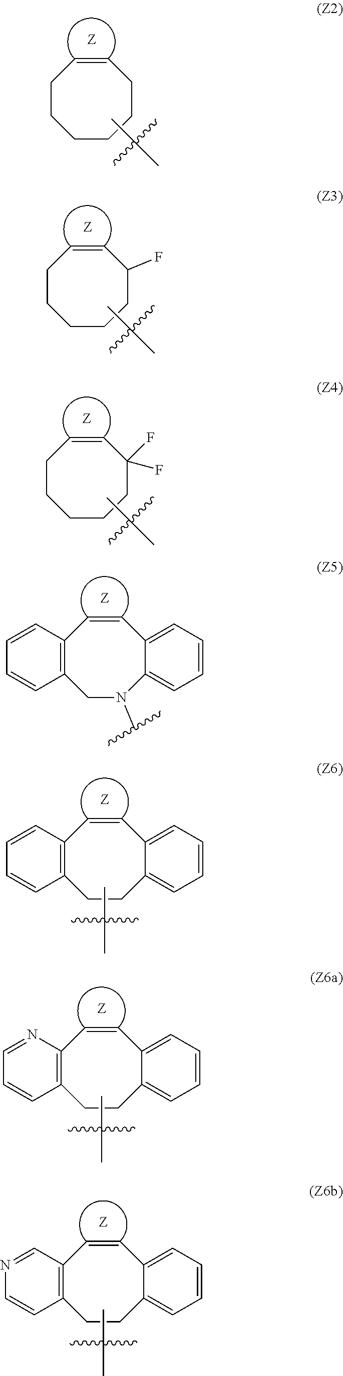 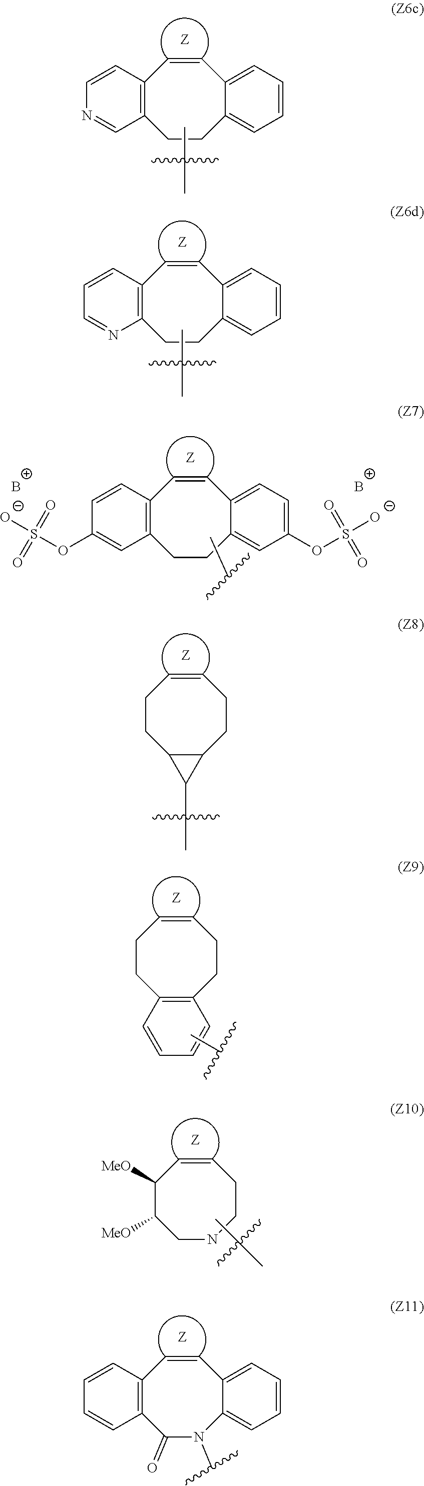 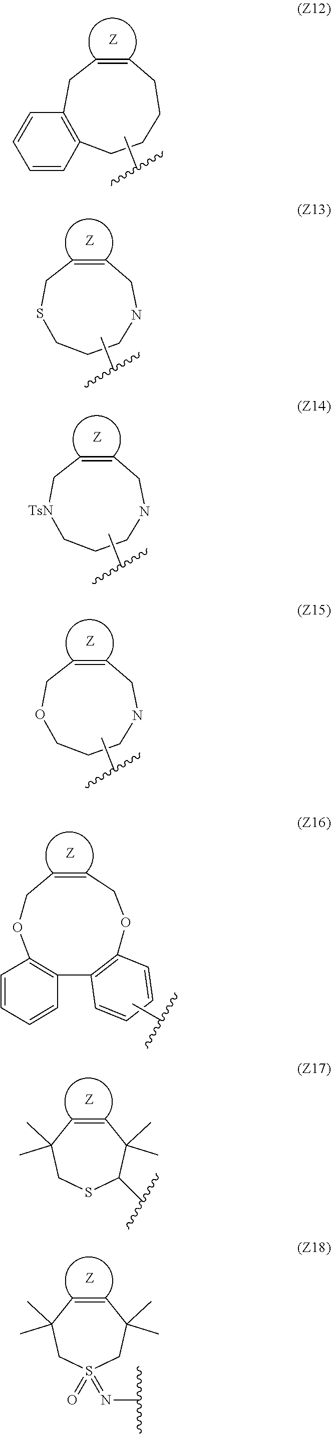 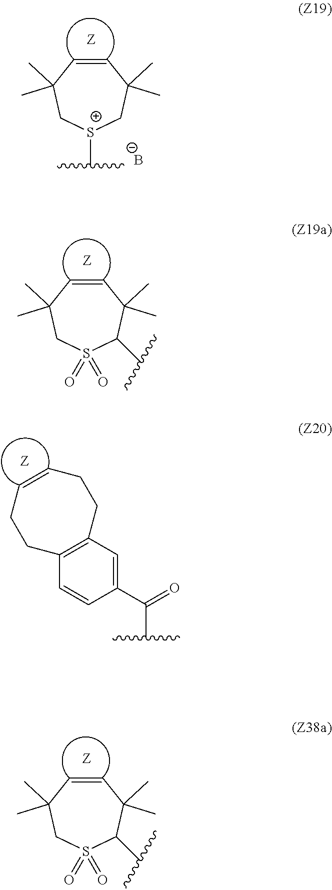
wherein:
the wavy bond represents the connection to L^{A} or L^{B}, and is connected to any available carbon or nitrogen atom;
ring Z is connected to Ab or L^{A} and is preferably selected from a triazole, a cyclohexene, a cyclohexadiene, a [2.2.2]-bicyclooctadiene, a [2.2.2]-bicyclooctene, an oxazoline, an isoxazolidine, a pyrazoline, a piperazine, or a pyridazine;
the nitrogen atom of (Z10), (Z13), (Z14), and (Z15) may bear the connection to L^{A} or L^{B}, or contains a hydrogen atom or is substituted;
B⁽⁻⁾ is an anion; and
B⁽⁺⁾ is a cation.

13. The compound according to claim 11, which is a conjugate having structure Ab(Z¹L^{A}(Z²(L^{B})Dₓ)_{y})_{z}, wherein:
Z¹ is according to a structure selected from (Z37+Za), (Z38+Za), (Z38a+Za) and (Z43): 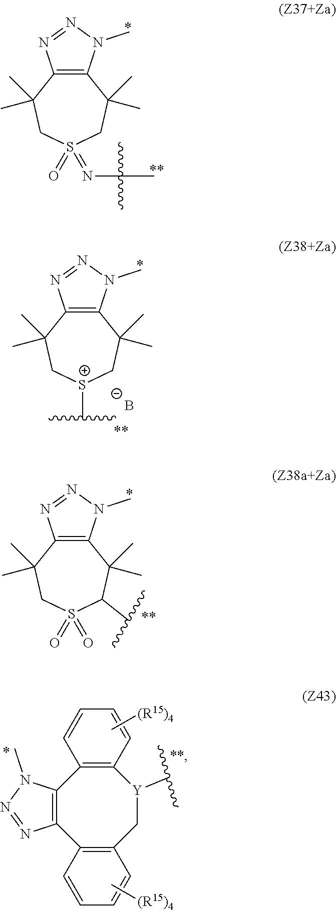
wherein:
the bond labelled with * is connected to Ab and the wavy bond labelled with ** is connected to L^{A};
B⁽⁻⁾ is an anion;
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups, and C₇-C₂₄ (hetero)arylalkyl groups, and wherein the alkyl groups, (hetero)aryl groups, alkyl(hetero)aryl groups, and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups, and C₇-C₂₄ (hetero)arylalkyl groups;
Y is N or CR¹⁵;
a carbon atom in the fused aromatic rings of (Z43) may be replaced by a nitrogen atom;
and
Z² is according to a structure selected from (Z39) and (Z44)-(Z56): 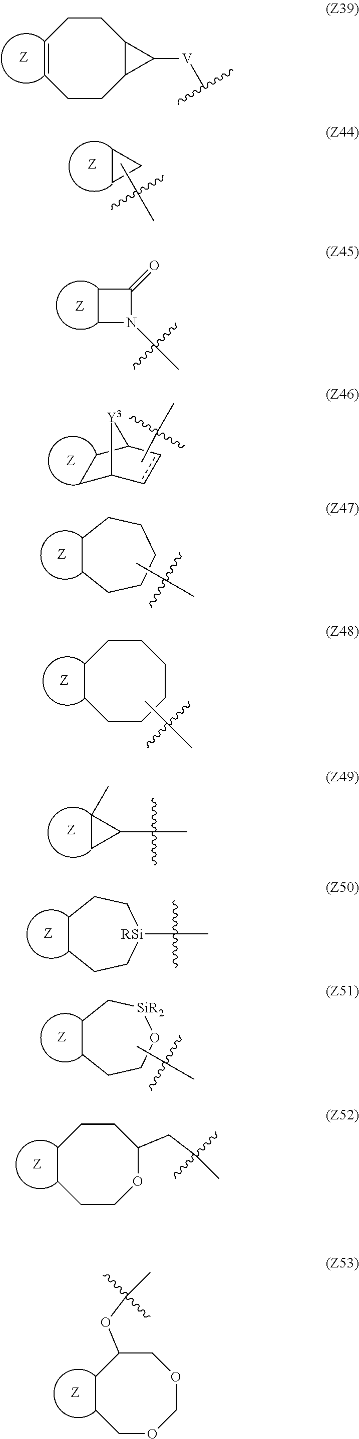 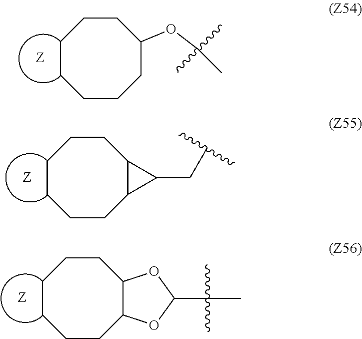
wherein:
the wavy bond is connected to L^{B};
ring Z has structure (Zu): 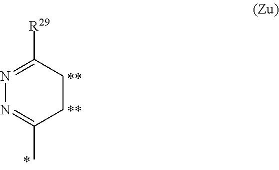
wherein the carbon atoms labelled with ** correspond to the two carbon atoms of (Z39) and (Z44)-(Z56) to which ring Z is fused, and the carbon a carbon labelled with * is connected to L^{A};
R group(s) on Si in (Z50) and (Z51) are alkyl or aryl; and
R²⁹ is selected from hydrogen, C₁₋₆ alkyl, aryl, C(O)—C₁₋₆ alkyl, C(O)-aryl, C(O)—O—C₁₋₆ alkyl, C(O)—O-aryl, C(O)—NR³³—C₁₋₆ alkyl, and C(O)—NR³³-aryl, wherein R³³ is H or C₁₋₄ alkyl.

14. The compound according to claim 11, which is a linker-construct having structure Q¹L^{A}(F²)_{y}, wherein:
Q¹ is a benzoannulated or tetramethylated (hetero)cycloalkyne, preferably selected from (Q5), (Q6), (Q6a), (Q6b), (Q6c), (Q6d), (Q7), (Q17), (Q18), (Q19), and (Q19a) as defined in claim 5;
F² is a tetrazine, preferably according to (F8a): 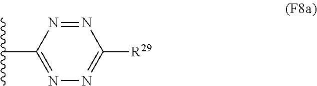
wherein:
R²⁹ is selected from hydrogen, C₁₋₆ alkyl, aryl, C(O)—C₁₋₆ alkyl, C(O)-aryl, C(O)—O—C₁₋₆ alkyl, C(O)—O-aryl, C(O)—NR³³—C¹⁻⁶ alkyl, and C(O)—NR³³-aryl, wherein R³³ is H or C₁₋₄ alkyl, preferably wherein R²⁹ is selected from hydrogen, methyl, phenyl, pyridyl, pyridinyl and pyrimidinyl; and
y is 2, 3, or 4, preferably y is 2 or 3, most preferably y is 2.

15. The compound according to claim 13, wherein Q¹ is a benzoannulated or tetramethylated (hetero)cycloalkyne, and F² is a tetrazine.

16. A method for:
targeting a tumour cell expressing a specific extracellular receptor, comprising contacting the conjugate as defined in claim 11 with cells that may possibly express the extracellular receptor, wherein the antibody specifically targets the extracellular receptor; and/or
the treatment of cancer, comprising administering to a subject in need thereof the conjugate as defined in claim 11, wherein the cancer cells specifically express an extracellular receptor,
preferably wherein the extracellular receptor is selected from the group consisting of 5T4, ADAM-9, AMHRII, ASCT2, ASLG659, ASPHD1, av-integrin, Axl, B7-H3, B7-H4, BAFF-R, BCMA, BMPR1B, Brevican, c-KIT, c-Met, C4.4a, CA-IX, cadherin-6, CanAg, CD123, CD13, CD133, CD138/syndecan-1, CD166, CD19, CD20, CD203c, CD205, CD21, CD22, CD228, CD25, CD30, CD324, CD33, CD37, CD38, CD45, CD46, CD48a, CD56, CD70, CD71, CD72, CD74, CD79a, CD79b, CEACAM5, claudin-18.2, claudin-6, CLEC12A, CLL-1, Cripto, CRIPTO, CS1, CXCR5, DLK-1, DLL3, DPEP3, E16, EGFR, ENPP3, EpCAM, EphA2, EphB2R, ETBR, FAP, FcRH1, FcRH2, FcRH5, FGFR2, fibronectin, FLT3, folate receptor alpha, Gal-3BP, GD3, GDNF-Ral, GEDA, GFRA1, Globo H, gpNMB, GPR172A, GPR19, GPR54, guanyl cyclase C, HER2, HER3, HLA-DOB, IGF-1R, IL13R, IL20Rα, Lewis Y, LGR5, LIV-1, LRRC15, LY64, Ly6E, Ly6G6D, LY6K, MDP, MFI2, MICA/B, MOSPD2, MPF, MSG783, MUC1, MUC16, NaPi2b, NCA, nectin-4, Notch3, P-cadherin, P2X5, PD-L1, PMEL17, PRLR, PSCA, PSCA hlg, PSMA, PTK7, RET, RNF43, RON, ROR1, ROR2, Sema 5b, SLITRK6, SSTR2, STEAP1, STEAP2, TAG72, TENB2, TF, TIM-1, TM4SF, TMEFF, TMEM 18, TMEM46, transferrin, TROP-2, TrpM4, TWEAKR, receptor tyrosine kinases (RTK), tenascin, or antibody is specific to an extracellular protein resulting from a viral infection and/or for a tumour-associated carbohydrate antigen (TACA).


SEQUENCE LISTING (8 sequences)

SEQ ID NO: 1 - protein, 4 aa, synthetic construct
  source 1..4: note = peptide spacer 1
GGGG

SEQ ID NO: 2 - protein, 4 aa, synthetic construct
  source 1..4: note = peptide spacer 2
GGFG

SEQ ID NO: 3 - protein, 4 aa, synthetic construct
  source 1..4: note = peptide spacer 3
GFGG

SEQ ID NO: 4 - protein, 4 aa, synthetic construct
  source 1..4: note = peptide spacer 4
FGGG

SEQ ID NO: 5 - protein, 4 aa, synthetic construct
  source 1..4: note = peptide spacer 5
GGGF

SEQ ID NO: 6 - protein, 4 aa, synthetic construct
  source 1..4: note = peptide spacer 6
FFGG

SEQ ID NO: 7 - protein, 4 aa, synthetic construct
  source 1..4: note = peptide spacer 7
GGFF

SEQ ID NO: 8 - protein, 5 aa, synthetic construct
  source 1..5: note = peptide spacer 8
GGGFG